(19) United States
(12) Patent Application Publication
(10) Pub. No.: US 2025/0230478 A1
(43) Pub. Date: Jul. 17, 2025

(54) COMBINATION OF NUCLEIC ACID SEQUENCES ENCODING PROTEINS DERIVED FROM HELICHRYSUM UMBRACULIGERUM, AND ANY TRANSGENIC CELL, TISSUE, AND ORGANISM COMPRISING SAME

(71) Applicant: YEDA RESEARCH AND DEVELOPMENT CO. LTD., Rehovot (IL)

(72) Inventors: Asaph AHARONI, Tel Aviv (IL); Prashant SONAWANE, Rehovot (IL); Adam JOZWIAK, Rehovot (IL); Paula BERMAN, Petach Tikva (IL); Luis DE-HARO, Rehovot (IL)

(21) Appl. No.: 19/072,119

(22) Filed: Mar. 6, 2025

Related U.S. Application Data
(63) Continuation of application No. PCT/IL2023/050968, filed on Sep. 7, 2023.
(60) Provisional application No. 63/404,645, filed on Sep. 8, 2022.
(60) Provisional application No. 63/453,112, filed on Mar. 19, 2023.

(51) Int. Cl. C12P 17/06; C12N 5/04; C12N 9/00; C12N 9/02; C12N 9/10; C12N 9/88; C12N 9/90; C12P 7/40

(52) U.S. Cl. CPC C12P 17/06; C12N 5/04; C12N 9/0004; C12N 9/1029; C12N 9/1051; C12N 9/88; C12N 9/90; C12N 9/93; C12P 7/40; C12Y 121/03008; C12Y 203/01084; C12Y 204/01017; C12Y 602/01003

(57) ABSTRACT

The present invention provides an isolated DNA molecule including at least a first nucleic acid sequence encoding a first protein and at least a second nucleic acid sequence encoding a second protein, wherein the first protein and the second protein are derived from Helichrysum umbraculigerum and belonging to an enzyme family selected from: acyl activating enzyme (AAE), polyketide synthase (PKS), polyketide cyclase (PKC), prenyltransferase (PT), or cannabichromenic acid synthase (CBCAS), and wherein the first protein and the second protein belong to different enzyme families. Further provided are an artificial nucleic acid molecule including the isolated DNA molecule, a transgenic cell, a tissue, or a plant including same. Further provided is a method for synthesizing a cannabinoid, a precursor thereof, or any combination thereof.

### CROSS-REFERENCE TO RELATED APPLICATIONS

This application is a ByPass Continuation of PCT Patent Application No. PCT/IL2023/050968 having international filing date Sep. 7, 2023, which claims the benefit of priority of U.S. Provisional Patent Application No. 63/404,645, titled “COMBINATION OF NUCLEIC ACID SEQUENCES ENCODING PROTEINS DERIVED FROM Helichrysum umbraculigerum, AND ANY TRANSGENIC CELL, TISSUE, AND ORGANISM COMPRISING SAME”, filed 8 Sep. 2022, and of U.S. Provisional Patent Application No. 63/453,112, titled “COMBINATION OF NUCLEIC ACID SEQUENCES ENCODING PROTEINS DERIVED FROM Helichrysum umbraculigerum, AND ANY TRANSGENIC CELL, TISSUE, AND ORGANISM COMPRISING SAME”, filed 19 Mar. 2023. The contents of the above applications are all incorporated herein by reference in their entirety.

### REFERENCE TO AN ELECTRONIC SEQUENCE LISTING

The contents of the electronic sequence listing (YEDA-P-010-PCT.xml; size: 251,312 bytes; and date of creation: Aug. 20, 2023) is herein incorporated by reference in its entirety.

### FIELD OF INVENTION

The present invention relates to combinations of enzymes derived from Helichrysum umbraculigerum including polynucleotides encoding same, and methods of using same, such as for producing cannabinoids.

### BACKGROUND

Cannabinoids are terpenophenolic compounds found in Cannabis sativa, an annual plant belonging to the Cannabaceae family. The plant contains more than 400 chemicals and approximately 70 cannabinoids. The latter accumulate mainly in the glandular trichomes. Of the naturally occurring cannabinoids, tetrahydrocannabinol (THC), for example, is used for treating a wide range of medical conditions, including glaucoma, AIDS wasting, neuropathic pain, treatment of spasticity associated with multiple sclerosis, fibromyalgia, and chemotherapy-induced nausea. THC is also effective in the treatment of allergies, inflammation, infection, epilepsy, depression, migraine, bipolar disorders, anxiety disorder, drug dependency and drug withdrawal syndromes.

Additional active cannabinoids include cannabidiol (CBD), an isomer of THC, which is a potent antioxidant and anti-inflammatory compound known to provide protection against acute and chronic neuro-degeneration; cannabigerol (CBG), found in high concentrations in hemp, which acts as a high affinity α2-adrenergic receptor agonist, moderate affinity 5-HT1A receptor antagonist and low affinity CB1 receptor antagonist, and possibly has anti-depressant activity; and cannabichromene (CBC), which possesses anti-inflammatory, anti-fungal and anti-viral properties. Many phytocannabinoids have therapeutic potential in a variety of diseases and may play a relevant role in plant defense as well as in pharmacology. Accordingly, biotechnological production of cannabinoids and cannabinoid-like compounds with therapeutic properties is of uttermost importance. Thus, cannabinoids are considered to be promising agents for their beneficial effects in the treatment of various diseases.

Despite their known beneficial effects, therapeutic use of cannabinoids is hampered by the high costs associated with the growing and maintenance of the plants in large scale and the difficulty in obtaining high yields of cannabinoids. Extraction, isolation and purification of cannabinoids from plant tissue is particularly challenging as cannabinoids oxidize easily and are sensitive to light and heat.

Therefore, there is a need for developing methodologies that allow large-scale production of cannabinoids for therapeutic use.

### SUMMARY

According to a first aspect, there is provided an isolated DNA molecule comprising at least a first nucleic acid sequence encoding a first protein and at least a second nucleic acid sequence encoding a second protein, wherein the first protein and the second protein are derived from Helichrysum umbraculigerum and belonging to an enzyme family selected from the group consisting of: acyl activating enzyme (AAE), polyketide synthase (PKS), polyketide cyclase (PKC), prenyltransferase (PT), and cannabichromenic acid synthase (CBCAS), and wherein the first protein and the second protein belong to different enzyme families.

According to another aspect, there is provided an artificial nucleic acid molecule comprising the isolated DNA molecule disclosed herein.

According to another aspect, there is provided a plasmid or an agrobacterium comprising the artificial nucleic acid molecule disclosed herein.

According to another aspect, there is provided a transgenic cell comprising: (a) the isolated DNA molecule of the invention; (b) the artificial nucleic acid molecule disclosed herein; (c) the plasmid or agrobacterium disclosed herein; or (d) any combination of (a) to (c).

According to another aspect, there is provided an extract derived from the transgenic cell of disclosed herein, or any fraction thereof.

According to another aspect, there is provided transgenic plant, a transgenic plant tissue or a plant part, comprising: (a) the isolated DNA molecule of the invention; (b) the artificial nucleic acid molecule disclosed herein; (c) the plasmid or agrobacterium disclosed herein; (d) the transgenic cell disclosed herein; or (e) any combination of (a) to (d).

According to another aspect, there is provided a composition comprising: (a) the isolated DNA molecule of the invention; (b) the artificial nucleic acid disclosed herein; (c) the plasmid or agrobacterium disclosed herein; (d) the transgenic cell disclosed herein; (e) the extract disclosed herein; (f) the transgenic plant tissue or plant part disclosed herein; or (g) any combination of (a) to (f), and an acceptable carrier.

According to another aspect, there is provided a method for synthesizing a cannabinoid, a precursor thereof, or any combination thereof, comprising the steps: (a) providing a transgenic cell or a cell transfected with the isolated DNA molecule of the invention or the artificial nucleic acid molecule disclosed herein; and (b) culturing the transgenic cell or the transfected cell from step (a) such that at least the first protein and the second protein encoded by the artificial nucleic acid molecule are expressed, thereby synthesizing the cannabinoid, a precursor thereof, or any combination thereof.

According to another aspect, there is provided an extract of a transgenic cell or a transfected cell obtained according to the herein disclosed method.

According to another aspect, there is provided a composition comprising the extract disclosed herein, and an acceptable carrier.

In some embodiments, the isolated DNA molecule further comprises at least a third nucleic acid sequence encoding a third protein derived from H. umbraculigerum and belonging to an enzyme family selected from the group consisting of: AAE, PKS, PKC, PT, and CBCAS, and wherein the first protein, the second protein, and the third protein, belong to different enzyme families.

In some embodiments, the isolated DNA molecule further comprises at least a fourth nucleic acid sequence encoding a fourth protein derived from H. umbraculigerum and belonging to an enzyme family selected from the group consisting of: AAE, PKS, PKC, PT, and CBCAS, and wherein the first protein, the second protein, the third protein, and the fourth protein, belong to different enzyme families.

In some embodiments, the isolated DNA molecule further comprises at least a fifth nucleic acid sequence encoding a fifth protein derived from H. umbraculigerum and belonging to an enzyme family selected from the group consisting of: AAE, PKS, PKC, PT, and CBCAS, and wherein the first protein, the second protein, the third protein, the fourth protein, and the fifth protein, belong to different enzyme families.

In some embodiments, the isolated DNA further comprises a nucleic acid sequence encoding a protein derived from H. umbraculigerum and belonging to an enzyme family selected from the group consisting of: uridine diphosphate (UDP)-glycosyltransferase (UGT), alcohol acyltransferase (AAT), and both.

In some embodiments: (a) the AAE is encoded by a nucleic acid sequence having at least 89% homology to any one of SEQ ID Nos.: 1-11, and any combination thereof; (b) PKS is encoded by a nucleic acid sequence having at least 83% homology to any one of: SEQ ID Nos.: 23-26, and any combination thereof; (c) PKC is encoded by a nucleic acid sequence having at least 88% homology to any one of: SEQ ID Nos.: 31-38, and any combination thereof; (d) PT is encoded by a nucleic acid sequence having at least 91% homology to any one of: SEQ ID Nos.: 47-58, and any combination thereof; (e) CBCAS is encoded by a nucleic acid sequence having at least 82% homology to any one of: SEQ ID Nos.: 71-79, and any combination thereof; or (f) any combination of (a) to (e).

In some embodiments: (a) the UGT is encoded by a nucleic acid sequence having at least 87% homology to any one of: SEQ ID Nos.: 89-101, and any combination thereof; (b) the AAT is encoded by a nucleic acid sequence having at least 87% homology to any one of: SEQ ID Nos.: 115-129, and any combination thereof; or (c) both (a) and (b).

In some embodiments: (a) AAE comprises an amino acid sequence with at least 93% homology to any one of SEQ ID Nos.: 12-22; (b) PKS comprises an amino acid sequence with at least 93% homology to any one of: SEQ ID Nos.: 27-30; (c) PKC comprises an amino acid sequence with at least 87% homology to any SEQ ID Nos.: 39-46; (d) PT comprises an amino acid sequence with at least 92% homology to any one of: SEQ ID Nos.: 59-70; (e) CBCAS comprises an amino acid sequence with at least 86% homology to any one of: SEQ ID Nos.: 80-88; (f) or any combination of (a) to (e).

In some embodiments: (a) the UGT comprises an amino acid sequence with at least 90% homology to any one of: SEQ ID Nos.: 102-114; (b) the AAT comprises an amino acid sequence with at least 91% homology to any one of: SEQ ID Nos.: 130-144; or (c) both (a) and (b).

In some embodiments: (a) the AAE consists of an amino acid sequence of any one of SEQ ID Nos.: 12-22; (b) the PKS consists of an amino acid sequence of any one of SEQ ID Nos.: 27-30; (c) the PKC consists of an amino acid sequence of any one of SEQ ID Nos.: 39-46; (d) the PT consists of an amino acid sequence of any one of SEQ ID Nos.: 59-70; (e) the CBCAS consists of an amino acid sequence of any one of SEQ ID Nos.: 80-88; (f) or any combination of (a) to (e).

In some embodiments: (a) the UGT consists of an amino acid sequence of any one of: SEQ ID Nos.: 102-114; (b) the AAT consists of an amino acid sequence of any one of: SEQ ID Nos.: 130-144; or (c) both (a) and (b).

In some embodiments, the isolated DNA molecule comprises a plurality of isolated DNA molecule types.

In some embodiments, each type of the plurality of isolated DNA molecule types encodes a protein or a plurality of proteins belonging to a different enzyme family.

In some embodiments, the transgenic cell is any one of: a unicellular organism, a cell of a multicellular organism, and a cell in a culture.

In some embodiments, the unicellular organism comprises a fungus or a bacterium. In some embodiments, the fungus is a yeast cell.

In some embodiments, the transgenic cell is a transgenic Cannabis sativa cell.

In some embodiments, the extract comprises a cannabinoid, a precursor thereof, or a combination thereof.

In some embodiments, the precursor is selected from the group consisting of: acyl coenzyme A (CoA), a polyketide, a resorcinoid precursor, and any combination thereof.

In some embodiments, the acyl is C1-C8 alkyl.

In some embodiments, the acyl CoA is hexanoyl CoA.

In some embodiments, the polyketide is a tetraketide.

In some embodiments, the tetraketide is a linear tetraketide.

In some embodiments, the resorcinoid precursor is olivetolic acid.

In some embodiments, the cannabinoid is cannabigerolic acid (CBGA), CBCA, or both.

In some embodiments, the artificial nucleic acid molecule is an expression vector.

In some embodiments, the transgenic cell or the transfected cell is a prokaryote cell or a eukaryote cell.

In some embodiments, the transgenic cell or the transfected cell is a C. sativa cell.

In some embodiments, the method further comprises a step preceding step (a), comprising introducing or transfecting a cell with the artificial nucleic acid molecule, thereby obtaining the transgenic cell or the transfected cell.

In some embodiments, the method further comprises a step of extracting the transgenic cell or the transfected cell, thereby obtaining an extract from the transgenic cell or the transfected cell.

In some embodiments, the extract comprises a cannabinoid, a precursor thereof, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1I include structures of chemical compounds, images, a chromatogram, a table, and micrographs showing that H. umbraculigerum biosynthesizes CBGA 1 and other terpenophenols in all aerial plant parts. (1A) Proposed biosynthetic pathways of CBGA 1 and heliCBGA 2. (1B) Photographs of the H. umbraculigerum plant inflorescence (up) and shoot (down). (1C) Total ion chromatogram of an ethanolic extract of H. umbraculigerum fresh leaves. The most abundant peaks of identified metabolites are marked on the Figure and color-coded according to the class of terpenophenol. CBGA 1 and heliCBGA 2 are highlighted in red and blue, respectively. (1D) Absolute quantification of CBGA 1 in different plant tissues [% w/w per fresh weight, n=3; for lyophilized leaves % w/w per dry weight (DW), n=5]. Reported Cannabis values were added for comparison. (1E) Chemical structures and names of selected terpenophenols with similar chemical formulas as 1-3. Representative (1F) cryo-SEM and (1G) confocal micrographs of the adaxial top view domain of leaves showing stalked glandular trichomes (marked by arrows). (1H) TEM micrograph showing the multicellular structure of the different cell types in a stalked glandular trichome at secretory stage. BC, basal cell; SC, stalk cell; NC, neck cell; DC, disk cell; SCv, secretory cavity. The dashed line marks the surface of the SCv. (1I). High magnification image shows the ultrastructure of DCs. CW, cell wall; M, mitochondria; N, nucleus; P, plastid; PSP, periplasmic space; V, vacuole; Vs, vesicle. Arrows mark active secretions from vesicles to the periplasmic space by exocytosis.

FIGS. 2A-2E include fluorescent micrographs, graphs, and a scheme showing that cannabinoid-associated gene expression is correlated with cannabinoid metabolites accumulation in H. umraculigerum glandular trichomes. (2A) Optical image and (2B) MALDI-MSI of m/z 361.23±0.01 Da of a cross-sectioned leaf showing that CBGA 1 accumulates in stalked glandular trichomes of leaves. Glandular trichomes in (2A) are marked to improve interpretation. The signals in (2B) correspond with the protonated m/z of CBGA 1 and geranylphlorocaprophenone 4. (2C) Normalized Enrichment Score (NES) of each co-expressed module in each tissue. Module M4 is highlighted as it is highly expressed in trichomes and leaves. (2D) Spaghetti chart showing the expression profile of module M4. The expression levels of individual genes are shown in gray lines. Colored lines highlight the expression of candidate genes from the pathway. (2E) Genomic landscape of the eight longest scaffolds of H. umbraculigerum assembly. Track i represents the gene density; ii represents repeat element density; iii represents 3′ Tran-Seq coverage; iv represents TrueSeq coverage. These metrics are calculated in 0.1 Mb non-overlapping windows. Magnification of the marked area in scaffold 1 reveals a tandem gene cluster containing seven PKSs. The enzymes HuPKS1-3 and HuTKS4 were cloned and functionally characterized in this study.

FIGS. 3A-3F include a heatmap, graphs, and a table showing the discovery of the core cannabinoid biosynthetic pathway enzymes. (3A) Gene expression in young leaves, roots and trichomes of the putative enzymes characterized in this study [log (cpm+1), n=3]. The most active enzymes in this study were highlighted in pink. AAE, acyl activating enzyme; PKS, type III polyketide synthase; PKC, polyketide cyclase; PT, prenyl-transferase. (3B) Products of recombinant enzyme assays of purified HuAAE proteins using various alkyl (short- and medium-chain FAs) and aromatic (cinnamic and coumaric acids) substrates. Peak areas were used for the comparisons (mean±s.d.; n=3). CoAT, acyl-CoA-transferase; EV, empty. (3C) Products of coupled recombinant enzyme assays of HuPKSs with either an EV or Cannabis olivetolic acid cyclase (CsOAC), in the presence of hexanoyl-CoA and malonyl-CoA. PDAL, pentyl diacetic acid lactone; HTAL, hexanoyl triacetic acid lactone; OA 92, olivetolic acid; PCP 95, phlorocaprophenone. Peak areas were used for the comparisons (mean±s.d.; n=3). OA 92 and PCP 95 were identified using analytical standards ([M−H]⁻=223.097 Da). (3D) Activity assay of microsomal fractions expressing prenyltransferases (PTs) using an array of aromatic substrates and either geranyl pyrophosphate (GPP) or isopentenyl pyrophosphate (IPP) as the isoprenoid donors. Circles represent observed mono- or iso-prenylated products in H. umbraculigerum or in vitro assays. VA, divarinolic acid; DHSA 93, dihyrostilbenic acid; ND, not detected; CBGAS, cannabigerolic acid synthase. (3E) Steady state kinetic analysis of HuPT1, HuPT3 and HuCBGAS4 with OA 92 and GPP. The Michaelis-Menten Km values were calculated using varying (0.5 μM-3 mM) and constant (1 mM) concentrations of each substrate (n=3). The literature Km value of Cannabis CsGOT4 was added for comparison. (3F) Phylogenetic analysis of PT proteins from H. umbraculigerum and other plants. The selection of the proteins was based on functionally characterized enzymes as described by de Bruijn et al. (2020). The clades according to the different substrates are marked in colored circles. HuPT proteins are highlighted in red, while Cannabis and Rhododendron dauricum PTs which prenylate cannabinoids are highlighted in blue. A H. umbraculigerum flower and a Cannabis leaf highlight the active HuCBGA4 and CsGOT4, respectively. A full list of protein IDs is available in Berman et al., “Parallel evolution of cannabinoid biosynthesis”; Nature Plants 9 817-831 (2023).

FIGS. 4A-4F include a phylogenetic tree, a heatmap, a table, chromatograms, and structure of chemical compounds showing the functional characterization of cannabinoid tailoring enzymes. (4A) Phylogenetic analysis of selected uridine diphosphate-glycosyltransferase (UGT) proteins from H. umbraculigerum, Arabidopsis thaliana, Oryza sativa and Stevia rebaudiana. The clades were annotated according to Arabidopsis thaliana UGT family classification (numbers in colored circles). HuUGT proteins are highlighted in red, while other proteins from plant species not producing cannabinoids that were shown previously to be able to glycosylate cannabinoids are highlighted in blue. A full list of protein IDs is available in Berman et al., “Parallel evolution of cannabinoid biosynthesis”; Nature Plants 9 817-831 (2023). H. umbraculigerum flowers mark the active HuCBGT1, HuCBGT6 and HuOAGT11. 4-Hydroxybenzoic acid (4-HBA) and 2,4-dihydroxybenzoic acid (2,4-DHBA) which are structurally similar to OA 92 and CBGA 1 are located next to the UGT enzymes that glycosylate them. Glycosylated hydroxyls are highlighted. (4B) Gene expression in young leaves, roots and trichomes of the putative UGT and alcohol acyl transferase (AAT) enzymes characterized in this study [log (cpm+1), n=3]. The enzymes found most active in this study were highlighted in pink. (4C) Comparison of steady state kinetic analysis of HuOAGT11 and HuUGT13 versus OsUGT and SrUGT, with OA 92 and uridine diphosphate glucose (UDP-Glc). Assays were performed using varying (0.5 μM-3 mM) and constant (1 mM) concentrations of each substrate (n=3). (4D) Extracted ion chromatograms of monoglucosides according to the theoretical m/z values, following enzymatic assays with the purified enzymes in the presence of UDP-Glc and an array of aromatic substrates (additional assays appear in FIG. 12B). One to three glucosylated compounds were observed for each substrate. The peaks were putatively assigned by MS/MS fragmentation patterns (Berman et al., “Parallel evolution of cannabinoid biosynthesis”; Nature Plants 9 817-831 (2023)). Compounds naturally observed in H. umbraculigerum were marked with a green asterisk. Chromatograms were normalized to the highest value. (4E) Extracted ion chromatograms of the O-acylated cannabinoids following enzymatic assays with purified HuCoAT5 in the presence of different acyl donors and aromatic substrates as acceptors. Major ion products were selected in each LC-MS/MS chromatogram. A single peak was observed for each pair of substrates. The detected analog peaks shifted in retention time depending on their change in hydrophobicity relative to the acyl group. Identification was performed according to MS/MS fragmentation (FIG. 13, and Berman et al., “Parallel evolution of cannabinoid biosynthesis”; Nature Plants 9 817-831 (2023)) and retention time. Compounds naturally observed in H. umbraculigerum were marked with a purple asterisk. Chromatograms were normalized to the highest value. (4F) Potential glucosylation and observed O-acylation sites were highlighted in blue and/or purple on each chemical structure, respectively.

FIGS. 5A-5D include combination diagrams and graphs showing in vivo reconstruction of the core cannabinoid pathway in heterologous systems. Co-expression of different combinations of HuCoAT6, HuTKS4, and HuCBGAS4, along with CsOAC and CsOLS from Cannabis in (5A-5B) N. benthamiana leaves and (5C-5D) S. cerevisiae yeasts. Grey, yellow, and green boxes to the left of the graphs indicate biosynthetic genes that are included in a co-expression experiment; blue boxes mark supplementation of geranyl pyrophosphate (GPP) and either (5A and 5C) sodium hexanoate (HexNa) or (5B and 5D) OA 92. Peak areas were used for the comparisons (mean±s.d.; n=3-6). N. benthamiana produced mainly glycosylated products identified according to the previously conducted in vitro UGT enzyme assays (FIGS. 4D and 12B). All the metabolites were identified by exact mass, retention time and MS/MS spectra (Berman et al., “Parallel evolution of cannabinoid biosynthesis”; Nature Plants 9 817-831 (2023)). EV, empty vector.

FIG. 6 includes a scheme showing parallel and divergent evolution of the cannabinoid biosynthetic pathway. The scheme provides a side-by-side comparison of the cannabinoid biosynthetic routes in H. umbraculigerum and Cannabis. On the top part, the phylogenetic relationship between Arabidopsis thaliana, Solanum lycopersicum, Helianthus annuus, Letuca sativa, Cannabis sativa and Helicrysum umbraculigerum illustrates the evolutionary distances between Cannabis and Helicrysum. The tree was constructed based on the whole proteomes of each species using the word-based software Prot-SpaM. Hybrid, yet unreported metabolites were produced in this study by reacting cannabinoids naturally biosynthesized in Cannabis (marked in green) with uridine diphosphate glucose (UDP-Gle) or acyl-CoAs in the presence of HuCoAT5, HuCBGT1 or HuCBGT6 enzymes from H. umbraculigerum (represented by blue). AAE, acyl activating enzyme; OLS, olivetol synthase; OAC, olivetolic acid cyclase; GOT, geranylpyrophosphate: olivetolate geranyltransferase; CBDAS, cannabidiolic acid synthase; CBCAS, cannabichromenic acid synthase; THCAS, (−)-Δ⁹-trans-tetrahydrocannabinolic acid synthase; AAE, acyl activating enzyme; PT, prenyl-transferase; UGT, uridine diphosphate-glycosyltransferase; AAT, alcohol acyl-transferase. The active enzymes identified in this study are marked by their names. CoAT, acyl-CoA-transferase; TKS, tetraketide synthase; PKC, polyketide cyclase; CBGAS, cannabigerolic acid synthase; OAGT, olivetolic acid UGT; CBGT, cannabinoid UGT; CBAT, cannabinoid acyl-transferase; BBE-like, berberine bridge enzyme-like; Cyc, cyclase; CYP, cytochrome P450.

FIGS. 7A-7B include chromatograms and structures of chemical compounds showing LC-MS/MS fingerprinting of CBGA 1, heliCBGA 2 and APHA 3 in H. umbraculigerum. (7A) Extracted ion chromatograms and MS/MS spectral matching of cannabigerolic acid (CBGA 1 [M−H]⁻=359.222 Da), heli-cannabigerolic acid (heliCBGA 2 [M−H]⁻=393.206 Da), and pre-amorphastilbol (APHA 3 [M−H]⁻=391.191 Da) standards or authentic metabolites versus a H. umbraculigerum leaf extract. To confirm the assignment, CBGA 1 and heliCBGA 2 were purified and analyzed by NMR. (7B) Stable isotope labeling of CBGA 1, heliCBGA 2 and APHA 3 via feeding of H. umbraculigerum leaves with hexanoic-D₁₁ acid, phenylalanine-D₅ or phenylalanine-¹³C₉. The MS/MS spectra of the non-labeled versus the labeled forms show similar fragmentation patterns with mass shifts corresponding with the labeled parts of the molecule.

FIGS. 8A-8J include micrographs and images showing stalked glandular trichomes in leaves and flowers of H. umbraculigerum. (8A-8B) Representative cryo-SEM micrographs of the lateral view of flower samples showing stalked glandular trichomes (marked by arrows). (8C) Light micrograph showing the biseriate structure of stalked glandular trichomes of H. umbraculigerum leaves. (8D-8F) Selected TEM micrographs of trichomes of H. umbraculigerum leaves at different stages of secretion. High magnification images show the ultrastructure of disk cells (DCs). CW, cell wall; M, mitochondria; N, nucleus; P, plastid; PSP, periplasmic space; SCv, secretory cavity; V, vacuole; Vs, vesicle. Arrows mark active secretions from the vesicles to the PSP by exocytosis. (8D) In the presecretory stage, DCs contained a very dense cytoplasm covered by ER and multiple ribosomes. There was no SCv or PSP and plastids were large and resembled pro-plastids. (8E) In the secretory stage, delamination of the apical DC wall led to the formation of the SCv. Electron transparent secretions were exuded out of plastids in vesicles delimited by an electron-dense layer. The vesicles released their contents to the PSP by exocytosis where the secretory product accumulated prior to secretion into the SCv. (8F) DCs of mature trichomes at the post-secretion stage were largely vacuolated with a cytoplasm restricted to the small remaining area. Plastids at this stage had degenerated and no vesicles were observed. The cell wall had a largely cutinized layer with a large SCv. MALDI-MSI of m/z 361.23±0.01 Da signals of the (8G) abaxial and (8H) adaxial leaf domains, following partial removal of trichomes by duct tape (the peeled area is outlined by green line). The areas with partially/fully removed trichomes show less or no signals compared to the untouched parts. (8I) Optical image and (8J) MALDI-MSI of m/z 361.23±0.01 Da of a cross-sectioned flower receptacle. Glandular trichomes in i are marked to improve interpretation. The signals in 8G-8H, and 8J. correspond with the protonated m/z of CBGA 1 and geranylphlorocaprophenone 4. The white broken lines in 8G-8J. mark the regions analyzed.

FIG. 9 include a scheme showing the predicted parallel metabolic pathways for the biosynthesis of cannabinoids and other terpenophenols present in H. umbraculigerum. The predicted types of enzymes catalyzing each reaction are marked by 1-8. Additional functional groups and rearrangements include hydroxylation, double bond isomerization or reduction, cyclization, and others. Alkyl chains can be linear/branched with one to seven carbons length; AAE, acyl activating enzyme; PKS, type III polyketide synthase; PKC, polyketide cyclase; PT, prenyl-transferase; UGT, uridine diphosphate-glycosyltransferase; AAT, alcohol acyl transferase; DBR, double bond reductase; CHI, chalcone isomerase. The active enzymes identified in this study are marked by their names. CoAT, acyl-CoA-transferase; TKS, tetraketide synthase; CBGAS, cannabigerolic acid synthase; OAGT, olivetolic acid UGT; CBGT, cannabinoid UGT; CBAT, cannabinoid acyl-transferase.

FIGS. 10A-10E include chromatograms, a scheme, structures of chemical compounds, and curves showing functional characterization of HuAAE, HuPKS and HuPTs. (10A) Ion abundances from triple-Quad analyses of acyl-CoAs produced in vitro by the HuAAEs versus analytical standard (Std). (10B) A scheme showing the steps and types of products and by-products synthesized in vitro by the recombinant HuPKSs with or without the Cannabis olivetolic acid cyclase (CsOAC). (10C) Ion abundances from triple-Quad analyses of OA 92 and olivetol products from coupled recombinant enzyme assays of HuPKSs with either an empty vector (EV) or Cannabis olivetolic acid cyclase (CsOAC), in the presence of hexanoyl-CoA and malonyl-CoA. (10D) MS/MS spectra of prenylated OA 92 products with cannabigerolic acid synthase (HuCBGAS4) and either isopentenyl pyrophosphate (IPP), geranyl pyrophosphate (GPP) or farnesyl pyrophosphate (FPP) as the prenyl donors. CBPA 19, cannabiprenylic acid; CBGA 1, cannabigerolic acid; SesquiCBGA, sesqui cannabigerolic acid (MS/MS spectrum corresponds to published data from Cannabis¹⁵). (10E) Steady state kinetic analysis of H. umbraculigerum prenyl-transferases HuPT1, HuPT3 and HuCBGAS4 with OA 92 and GPP. The Michaelis-Menten Km value of each enzyme was calculated using varying (0.5 μM-3 mM) and constant (1 mM) concentrations of each substrate (n=3 technically independent samples; measurements were plotted individually).

FIGS. 11A-11D include phylogenetic trees showing phylogenetic analyses of enzymes and whole proteome from H. umbraculigerum and different plant species. Phylogenetic analysis of (11A) AAE, (11B) PKS and (11C) PT proteins from H. umbraculigerum and other plants. H. umbraculigerum and Cannabis proteins are highlighted in red and blue, respectively, and the active enzymes were marked by a flower and a leaf, respectively. A full list of protein IDs is available in Berman et al., “Parallel evolution of cannabinoid biosynthesis”; Nature Plants 9 817-831 (2023). Bootstrap values are indicated at the nodes of each branch. (11A) The selection of the proteins was based on (11A) Arabidopsis thaliana enzymes or (11B-11C) functionally tested enzymes. Clades according to substrates or functionalities are marked by different colors. None of the active H. umbraculigerum enzymes clustered with any of the known Cannabis proteins. (11D) phylogenetic relationship between Arabidopsis thaliana, Solanum lycopersicum, Helianthus annuus, Letuca sativa, Cannabis sativa and Helicrysum umbraculigerum illustrate the evolutionary distance between the last two species (marked by a flower and a leaf, respectively). The tree was constructed based on the whole proteomes of each species using the word-based software Prot-SpaM.

FIGS. 12A-12C include graphs, chromatograms, structures of chemical compounds, and curves showing functional characterization of HuUGTs. (12A) Activities of lysates containing HuUGTs with olivetolic acid (OA 92), cannabigerolic acid (CBGA 1) and helicannabigerolic acid (heliCBGA 2) as substrates and uridine diphosphate glucose (UDP-Glc) as the sugar donor (n=1). Reactions show differing substrate specificities and type of products. Representative peaks correspond to chromatograms obtained for HuCBUGT1. The most abundant products in each assay are marked with asterisks. EV, empty vector. (12B) In vitro production of monoglucosides with the purified UGTs and additional substrates. Extracted ion chromatograms of the observed monoglucosides using UDP-Glc and either DHSA 93, olivetol, CBG, CBD, A9-THC, PCP 95, naringenin chalcone 97 or pinocembrin chalcone 100. The substrates naringenin chalcone 97 and pinocembrin chalcone 100 contained mixtures of the chalcones and respective flavanones. All LC-MS chromatograms were selected for the theoretical m/z values of the respective metabolites of interest. (12C) Comparison of steady state kinetics of UGTs with OA 92 and UDP-Glc. HuOAUGT11 and HuUGT13 were compared with UGTs from rice (OsUGT) and stevia (SrUGT). Kinetic values were calculated using varying (0.5 Mm-3 mM) and constant (1 mM) concentrations of each substrate (n=3 technically independent samples; measurements were plotted individually). V₀ and Vmax were calculated using the calibration curve of OA 92 since there was no analytical standard available for Glc-OA 102.

FIGS. 13A-13C include structures of chemical compounds, chromatograms, and a phylogenetic tree showing functional characterization of HuAATs. (13A) Stable dual isotope labeling of O-MeButCBGA 120 via feeding of H. umbraculigerum leaves with either 2-methyl butyric-D₉ acid or hexanoic-D₁₁ acid. The MS/MS spectra of the non-labeled versus the two-labeled forms show fragmentation patterns with mass shifts corresponding with the labeled parts of the molecule. Fragments colored in red, or purple correspond to the m/z of the specific fragment with labeled alkyl chain or acyl group, respectively. (13B) Activities of lysates containing HuAATs with different acyl donors and cannabinoid acceptors. Extracted ion chromatograms were selected for the theoretical m/z values of the respective metabolites. Only HuCBAT5 and HuAAT14 (red and blue, respectively) acylated CBGA 1 and heliCBGA 2 with both acyl-CoAs. EV, empty vector; Std, standard; ButCoA, butyryl-CoA; HexCoA, hexanoyl-CoA. (13C) Phylogenetic analysis of HuAAT proteins and identified BAHD AATs from other plants. The Maximum Likelihood tree was constructed with 100 bootstrap tests based on a MUSCLE multiple alignment using the MEGA11 software. The evolutionary distances were computed using the JTTmatrix-based method. Bootstrap values are indicated at the nodes of each branch. The clades of the different AAT types are marked in circles based on Tuominen et al. (2011). The active HuCBAT5 and HuAAT14 were clustered in clade IIIa which represents BAHDs of diverse catalytic functions. A full list of protein IDs is available in Berman et al., “Parallel evolution of cannabinoid biosynthesis”; Nature Plants 9 817-831 (2023).

FIG. 14 includes chromatograms and structure of chemical compounds showing MS/MS spectra of observed acylated cannabinoids following enzymatic assays with the purified HuCBAT5. OA 92, olivetolic acid; CBGA 1, cannabigerolic acid; HeliCBGA 2, helicannabigerolic acid; CBDA, cannabidiolic acid. Full data of MS/MS products appears in Berman et al., “Parallel evolution of cannabinoid biosynthesis”; Nature Plants 9 817-831 (2023). MS/MS fragmentation and retention times correspond to the O-acylated cannabinoids found in the plant.

FIGS. 15A-15F include schemes, chromatograms, and a table showing the reconstruction of the core cannabinoid pathway in heterologous systems. Schematic representation of products observed in (15A) N. benthamiana leaves and (15D) S. cerevisiae yeasts following co-expression of different combinations of HuCoAT6, HuTKS4, and HuCBGAS4, along with CsOAC from Cannabis. NbUGT, N. benthamiana uridine diphosphate-glycosyltransferase; HexNa, sodium hexanoate; GPP, geranyl pyrophosphate; OA 92, olivetolic acid. Extracted ion chromatograms and MS/MS spectra showing (15B) glycosylated OA (Glc-OA 102), glycosylated polycaprophenone (Glc-PCP1/2) and glycosylated naringenin chalcone (Glc-Naringenin chalcone 1/2) following feeding with HexNa and GPP (I); and (15C) glycosylated cannabigerolic acid (Glc-CBGA 109) following feeding with OA 92 and GPP (II). Glycosylated metabolites synthesized by the recombinant stevia (SrUGT) or rice (OsUGT) enzymes were used as reference for identification of N. benthamiana products according to exact mass, retention time and MS/MS spectra. EV, empty vector; UDP-Glc, uridine diphosphate glucose. (15E) Extracted ion chromatograms of OA 92, PCP 95 and CBGA 1 products observed in yeasts without any feeding. Identification was according to analytical standards. (15F) Summary of the observed products in each assay. PDAL, pentyl acyl diacetic acid lactone; HTAL, hexanoyl acyl triacetic acid lactone.

### DETAILED DESCRIPTION

The present invention, in some embodiments, is directed to a DNA molecule comprising at least a first nucleic acid sequence encoding a first protein and at least a second nucleic acid sequence encoding a second protein, wherein the first protein and the second protein are derived from Helichrysum umbraculigerum, including methods of using same.

In some embodiments, any one of the first protein and the second protein belongs to an enzyme family selected from: acyl activating enzyme (AAE), polyketide synthase (PKS), polyketide cyclase (PKC), prenyltransferase (PT), cannabichromenic acid synthase (CBCAS), uridine diphosphate (UDP)-glycosyltransferase (UGT), alcohol acyltransferase (AAT).

In some embodiments, the DNA molecule further comprises at least a third nucleic acid sequence encoding a third protein derived from H. umbraculigerum and belonging to an enzyme family selected from: AAE, PKS, PKC, PT, CBCAS, UGT, and AAT.

In some embodiments, the DNA molecule further comprises at least a fourth nucleic acid sequence encoding a third protein derived from H. umbraculigerum and belonging to an enzyme family selected from: AAE, PKS, PKC, PT, CBCAS, UGT, and AAT.

In some embodiments, the DNA molecule further comprises at least a fifth nucleic acid sequence encoding a third protein derived from H. umbraculigerum and belonging to an enzyme family selected from: AAE, PKS, PKC, PT, CBCAS, UGT, and AAT.

In some embodiments, the DNA molecule further comprises at least a sixth nucleic acid sequence encoding a third protein derived from H. umbraculigerum and belonging to an enzyme family selected from: AAE, PKS, PKC, PT, CBCAS, UGT, and AAT.

In some embodiments, the DNA molecule further comprises at least a seventh nucleic acid sequence encoding a third protein derived from H. umbraculigerum and belonging to an enzyme family selected from: AAE, PKS, PKC, PT, CBCAS, UGT, and AAT.

In some embodiments, the first protein and the second protein belong to different enzyme families.

In some embodiments, the first protein, the second protein, and the third protein belong to different enzyme families.

In some embodiments, the first protein, the second protein, the third protein, and the fourth protein belong to different enzyme families.

In some embodiments, the first protein, the second protein, the third protein, the fourth protein, and the fifth protein belong to different enzyme families.

In some embodiments, the first protein, the second protein, the third protein, the fourth protein, the fifth protein, and the sixth protein belong to different enzyme families.

In some embodiments, the first protein, the second protein, the third protein, the fourth protein, the fifth protein, the sixth protein, and the seventh protein belong to different enzyme families.

According to some embodiments: (a) an AAE protein is encoded by a nucleic acid sequence having at least 89% homology or identity to any one of SEQ ID Nos.: 1-11; (b) PKS is encoded by a nucleic acid sequence having at least 83% homology or identity to SEQ ID Nos.: 23-26; (c) PKC is encoded by a nucleic acid sequence having at least 88% homology or identity to SEQ ID Nos.: 31-38; (d) PT is encoded by a nucleic acid sequence having at least 91% homology or identity to SEQ ID Nos.: 47-58; (e) CBCAS is encoded by a nucleic acid sequence having at least 82% homology or identity to SEQ ID Nos.: 71-79; or (f) any combination of (a) to (e).

In some embodiments, the DNA molecule further comprises a nucleic acid sequence being derived from Helichrysum umbraculigerum and encoding one or more protein(s) or enzyme(s) belonging to the uridine diphosphate (UDP)-glycosyltransferase (UGT) family; the alcohol acyltransferase (AAT) family, or both.

In some embodiments: (a) UGT is encoded by a nucleic acid sequence having at least 87% homology to any one of: SEQ ID Nos.: 89-101, and any combination thereof; (b) AAT is encoded by a nucleic acid sequence having at least 87% homology to any one of: SEQ ID Nos.: 115-129, and any combination thereof; or (c) both (a) and (b).

In some embodiments, the DNA molecule comprises at least two nucleic acid sequence encoding at least two enzyme, wherein each enzyme belongs to a different family, wherein the at least two families are selected from: AAE, PKS, PKC, PT, CBCAS, UGT, and AAT.

In some embodiments, the DNA molecule is an isolated DNA molecule. In some embodiments, the DNA molecule is a complementary DNA (cDNA) molecule.

As used herein, the term “DNA molecule” refers to a polynucleotide comprising or consisting of deoxyribonucleotides.

As used herein, the terms “isolated polynucleotide” and “isolated DNA molecule” refer to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art of molecular biology. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the DNA molecule comprises the nucleic acid sequence:

(SEQ ID NO: 1)
ATGACGTCGTCAAAGAAGTTTACAGTTGAAGTTGAACCGGCGATTCCGGC
CAAGGATGGAAAACCGTCGGCTGGACCGGTTTACCGTAGTATCTTTGCTA
AAGACGGTTTTCCAGCTCATATTGACGGTTTAGATTCATGTTGGGATATT
TTCCGCCTATCTGTGGAGAAATACCCCAATAATCGAATGCTTGGCACCCG
TGAATTTGTGAATGGAAAGCATGGACCATATGTATGGTCGACTTACAAAC
AAGTATACGACAAGGTGATAAAGGTTGGAAATGCTATCCGTGCGTGTGGT
GTCGAGCCAGGTGGTCGGTGTGGGATCTATGGTGCCAATTGTGCAGAATG
GATTATGAGCATGGAGGCATGTAATGCTCATGGGCTTTACTGTGTACCTT
TATACGATACCTTAGGTGCTGGTGCAATTGAATTCATTCTTTGCCATGCC
GAGGTTACAATTGCTTTTGTAGAAGAGAAAAAGATCCCTGAGTTGTTGAA
AACATTTCCGAAAGCTGGAGAATTTCTGAAAACAATTGTGAGCTTTGGAA
AAGTTACTCCTGAACAAAGAGAACAAGCTGAAAACTTTGGTTTAAAAATA
CATTCATGGGATGAATTCTTGACATTGGGTGATGATAAAAACTTTGACCT
GCCACTGAAGGAAAAAACTGATATCTGTACAATAATGTACACTAGTGGAA
CAACTGGTGATCCTAAGGGTGTTCTGATTTCAAATAACAGCATGGCAACA
CTTATAGCTGGCGTCAATCGTCTACTAGATAGTGCAAAAGAATCTTTGAA
TCAACATGATGTCTATCTCTCGTTTTTACCTCTGGCACATATATTTGACC
GTGTGATTGAAGAATGTTTTATCAATCATGGAGCATCTATAGGATTCTGG
CGTGGGGATGTTAAATTGCTGATTGAAGACATAGGGGAGCTGAAACCTAC
TATTTTCTGCGCTGTTCCTCGAGTGTTGGATAGGATTTATTCAGGTTTGC
AACAGAAAATTTCTGCGGGGGGTTTTATCAAACGTAACTTATTTAATCTA
GCCTATTCATACAAATTACGTAATATGAAGGGAGGGAAAACACATTCAGA
GGCATCTCCATTGAGTGACAAAATCGTCTTCAGTAAGGTTAAGCAGGGCC
TAGGAGGAAATGTACGAATTATTCTATCTGGAGCTGCTCCACTAGCTCCA
CATGTAGAAGCTTACCTGAAAGTAGTGGCATGTAGTCACGTCCTGCAAGG
ATATGGCCTGACAGAAACTTGTGCTGGATCATTTGTCTCACTGCCAAACG
AAATGGAGATGCTGGGTACAGTGGGCCCACCTGTACCAGTTTTGGATGCC
CGACTGGAGTCTGTTCCGGAGATGAACTATGATGCTTGTTCAAGCAAACC
ACAAGGAGAAATATGTATTAGAGGGGATGTTCTGTTTTCAGGATACTACA
AGCGTGAGGACCTTACAAAAGAAGTCTTTGTTGATGGGTGGTTCCATACA
GGTGATATCGGTGAGTGGCAACCAGATGGAAGCATGAAAATTATTGACCG
AAAGAAAAACATTTTTAAGCTCTCACAAGGAGAGTACGTCGCAGTTGAAA
ATCTGGAGAATGTTTATGGAAATGTTTCTGACATTGACACGATATGGATA
TATGGGAACAGCTTCGAGTTTTGTCTTGTTGCTGTGGTCAACCCAAATGA
GCCAGCAATCAAACGTTATGCTGAAGCAAATAATATTTCTGGGGATTTTG
ATTCATTATGTGAAAATCCCAAAATTAAAGAATACATACTCGGAGAGCTC
GCTAGAATTGGAAAAGAGAAAAAGTTAAAAGGTTTTGAATTCGTCAAAGC
TGTTCACCTTGACCCTGTCCCTTTCGACATGGAACGTGACCTTCTGACCC
CAACATTCAAGAAGAAAAGGCCCCAGATGCTTAAGTACTACCAGGATGTA
ATTGATAACATGTACAAGACTATTAACAAGAAGTGA.

In some embodiments, the DNA molecule comprises a nucleic acid sequence with at least 89%, at least 92%, at least 95%, or at least 97% homology or identity to SEQ ID NO: 1, or any value and range therebetween. Each possibility represents a separate embodiment of the invention. In some embodiments, the DNA molecule comprises a nucleic acid sequence with 89% to 95%, 90% to 97%, 95% to 99%, or 90% to 100% homology or identity to SEQ ID NO: 1. Each possibility represents a separate embodiment of the invention.

In some embodiments, the DNA molecule comprises the nucleic acid sequence:

(SEQ ID NO: 2)
ATGGATGCATTGAGGAAGCCTAATTCTGCGAATTCAAGCCCTTTAACTCC
TATCGGATTCCTTGAAAGGGCAGCCGTCGTATTTGCCAACTCTCCTTCGA
TCGTATACAACAATCTCATCTACACTTGGAGCGATACTTTTCATCGTTGT
CTACGATTAGCTTCATCCATCTCTCGTCTCGCTATACGAAAAGGCGACGT
TGTTTCAGTACTCGCACCAAACATCCCTGCCATTTATGAGCTTCATTTTG
GCATCACTATGACTGGGGCCATAATCAACACCATCAATACCCGTTTGGAT
GCGCGTACTATCTCAATACTCCTTTGTCACAGTGAATCCAAGCTCGTCTT
TGTTGATTACCAGTTGACTCGTCTTATACGAGAAGCGGTTTCTTTGATGC
CAGATGCTTGTGTTCCCCCACAACTCGTCCTCATCGTAGATGACGGACAT
AATCTATCTTTACTTTCTGATCAATTTATCAATACTTATGAAGCTATGGT
TGAAACAGGGGATCCTGGGTTCAATTGGGTTCGTCCAGATAGCGATTGGG
ACCCTCTAACGTTGAATTACACTTCTGGGACGACTTCTTCCCCCAAAGGT
GTTGTTAACAGCCACCGTGGATCGTTCATAGTAGCGTTTGATTCTTTACT
GGAGTGGCACGTACCGAAACAGCCGATCATGCTGTGGACTCTACCAATGT
TCCACGCAAATGGGTGGAGCTTCGTTTGGGGTATGGCAGCTGTTGGTGGC
ACCAATGTTTGCCTTCGTAAATTCGATGCTACTATTATTTATGACACCAT
TCGTAACCACCATGTGACGCACATGTGTGGCGCCCCTGTTGTACTCAACA
TGTTATCAGAAGGTAAGCCACTTGAACACACGGTTCACATAATGACAGCA
GGAGCACCACCTCCAGCGGCCGTTTTGTTGCGAACCGAGTCGCTAGGGTT
TGAGGTGACTCATGGGTTCGGGATGACAGAAACAGGCGGGTTAGTTGTGT
CATGCTCATGGAAGAAAGAATGGAATCGTCTGCCCGTGACTGAGAAAGCG
AGATTGAAAGCGAGACAAGGAGTTAGAACACTTGGGATGACGGAAGTGGA
TATTGTGGATCCCGAGTCAGGAGTAAGTGTGACTCGAGACGGGTTAACTC
AGGGGGAATTAGTGTTGCGAGGTGGGTCTATTATGTTGGGTTACTTAAAA
GATCCGGAAACAACAAATAAATCCGTTAAAAACGGGTGGTTTTATACCGG
CGACGTGGCGGTGATGCATCCAGATGGATATCTGGAAATAAAAGATAGAT
CAAAAGATGTAATAATAAGTGGTGGTGAGAATATAAGTAGTGTGGAGGTT
GAGTCAATCTTGTATCAGCATCCTGCGATTAACGAGGCCGCGGTGGTGGG
ACGGCCTGATGAGTTTTGGGGCGAGTCGCCGTGTGCTTTCGTGAGTTTGA
AAGATGATAACGGGAAGGTGGCTGTGCCAACAGCGGATGAGATAATGAAG
TTTTGTAAAGGAAAGTTGCCGGGTTACATGGTACCCAAATCGGTTGTGTT
TAAGAAGGATCTTCCGAAGACATCTACCGGTAAGATTCAGAAATATGTGC
TTAGAAAACTTGCTAAAGATTTGGGTTTTGCTGTAAAAAGTCGAATTTA
G.

In some embodiments, the DNA molecule comprises a nucleic acid sequence with at least 79%, at least 83%, at least 87%, at least 89%, at least 92%, at least 95%, at least 97%, or at least 99% homology or identity to SEQ ID NO: 2, or any value and range therebetween. Each possibility represents a separate embodiment of the invention. In some embodiments, the DNA molecule comprises a nucleic acid sequence with 79% to 85%, 80% to 92%, 82% to 99%, or 80% to 100% homology or identity to SEQ ID NO: 2. Each possibility represents a separate embodiment of the invention.

In some embodiments, the DNA molecule comprises the nucleic acid sequence:

(SEQ ID NO: 3)
ATGACCGAAGAGGAAAAAAATAAAGCAGAGTCCATGGGGATAAAAACGTA
TGCATGGAGCGACTTCCTTCATCTGGGGAGTAAAAATCCTTCAGAACTGC
AAACGCCTAAAGCAACTGATATATGTACAATCATGTACACTAGTGGCACT
AGTGGAGACCCAAAAGGTGTTATATTGACACATGAAAATGCTACAACAAA
CATACGAGGGGTTGATCTTTTCATGGAACAATTCGAGGACAAGATGACCG
TGGATGACGTTTATATATCTTTCTTGCCTCTTGCTCACATTCTTGATCGT
ATGATTGAAGAATACTTTTTCCGTAGTGGTGCCTCTGTCGGCTTCTATCA
TGGGGATATCAATGCGTTGAAGGAGGATTTGGCAGAGCTAAAGCCTACTT
TTTTGGCTGGAGTACCTCGAGTTTTGGAAAAGATTCACGAAGGTGTGCTT
AAAGGACTAGAAGAAGTTAATCCAAGGAGAAGGAAAATATTTAGCATTTT
ATACAATCACAAACTAAAATACATGAAAGCAGGTTACAAGCATAAATATG
CATCACCACTTGCAGATCTGCTTGCTTTTAGAAAGGTTAAGAACAGGCTT
GGTGGGCGAATTCGTCTTATGGTATCTGGAGGAGCTCCGTTAAGCACTGA
GATTGAAGAGTTCATGAGGGTTACTTCATGTGCTTTTGTGGCGCAAGGAT
ATGGTTTGACGGAAACATGTGGTTTGGCTACTTTAGGATTTCCAGATGAG
ATGTGCATGATTGGAACAGTTGGTTCGCCCTTCGTGTATACAGAATTACG
CCTCGAAGAAGTTTCAGATATGGGCTATGACCCGTTGGCCAATCCACCAC
GTGGTGAAATATGTGTTAAGGGAAAAACGCCTTTCGCAGGTTACTACAAG
AATCCAGAACTCACTAATGAGGTCATGAAAGATGGGTGGTTTCATACAGG
TGACATAGGAGAGATGCAACCAAACGGGGTATTGAAAATCATCGACAGAA
AGAAACATCTGATAAAACTATCTCAAGGGGAGTATATCGCGCTTGAATAT
CTAGAGAAAGTTTACTGCATCACTCCCATTCTTGAAGACATCTGGGTATA
TGGGGATAGCTTCAAGTCATCATTGGTCGCGGTAGCTGTACCAAACAAAG
AAAACGCAGAAAAGTGGGCCGATCAAAAGGGCCTTAAAGTTTCTTACTCT
GAGCTCTGCACACTAACACAGTTCAGAGATTATATCCAATCTGAACTGAA
ATCTACCGCGGAGAGAAACAAGCTAAGAGGTTTTGAGCATATAAAGGCTA
TAATTGTGGAGCCACGGACGTTTGAAGGAGACCAGGAATTGTTGACTGCA
ACAATGAAGAAACGTAGAAATAAACTGCTTAACCGTTACAAGGAGGGGAT
CGACAACCTTTACAAGAACTTGGCTGCAAACAAACGCTGA.

In some embodiments, the DNA molecule comprises a nucleic acid sequence with at least 86%, at least 88%, at least 90%, at least 95%, at least 97%, or at least 99% homology or identity to SEQ ID NO: 3, or any value and range therebetween. Each possibility represents a separate embodiment of the invention. In some embodiments, the DNA molecule comprises a nucleic acid sequence with 86% to 94%, 88% to 97%, 86% to 100%, or 92% to 99% homology or identity to SEQ ID NO: 3. Each possibility represents a separate embodiment of the invention.

In some embodiments, the DNA molecule comprises the nucleic acid sequence:

(SEQ ID NO: 4)
ATGGTGTACAAGTCTTTGAATTCAATATCCATATCAGATATAGTAAATCT
TGGTATATCACCTGAAACTGCAACTCAACTTCATCAGAAACTAACTGAAA
TCATTCAGATTTATGGTTTTGATGCTCCTCAAACATGGACCCAGATATCC
ACCCGGATTCTTCATCCGGACCTTCCCTTTTGTTTTCATCAGATGATGTA
TTATGGATGCTATGTTGATTTTGGACCGGATCCTCCTGCTTGGTCACCCG
ACCCGAAGGATGCAAAGTTAACAAACATAGGTAGTTTATTAGAGAGACGC
GGAAAGGAGTTCTTGGGGCCTAGTTATAAAGATCCCATTTCAAGCTACTC
TGCTCTTCAGGAATTTTCAGCCTTAAATCTAGAGGTGTTTTGGAAAACAA
TATTGGATGAAATGAATATAACATTTTCTGTGCCTCCAAAACGCATATTA
GTTGATGACCTGTCTAAAGAAAGCCAGTTATTGCATCCAGGTGGTCGATG
GCTTCCCGGAGCTTATGTAAATCCAGCTAGAAATTGTTTGAGTTTAAGTA
GCAAGAGAAGGTTAAGTGATATAGCAGTTATATGGCGTGATGAAGGAAAT
GATGATATGCCGGTCAACAAAATGACGTTTCAGCAGTTGCGCTCAGAGGT
TTGGTTAGTTGCATATGCACTTGATACATTGGGAGTGGAAAAAGGATCTG
CAATTGCAATCGATATGCCTATGGATGTCAAATCTGTGGTGATTTATCTA
GCCATTGTTTTAGCAGGCTATGTGGTTGTATCTATTGCAGATAGTTTTGC
TGCTGGTGAAATTTCGACCAGACTTGTATTATCAAAAGCAAAAGCAATTT
TTACTCAGGATTTGATCATTCGTGGTGACAGAAGCCATCCCTTGTACAGC
CGAGTTGTTGATGCTCAATCACCTCTAGCAATTGTCATTCCTACGAGAGG
CTCAAGTTTTAGTATAAAATTACGTGACGGTGATATTTCTTGGCATGATT
TTCTGGAACGAGCTAACACTTACAGGAATGTTGAGTTTGTTGCTGTTGAA
CGACCCGTTGAAGCTTTCTCAAATATCCTTTTCTCATCAGGAACTACAGG
GGAACCGAAGGCAATTCCATGGACCCTTGCAACACCTTTCAAGGCTGGTG
CAGACGCTTGGTGCCACATGGATGTCCACAAAGGTGATGTTGTTGCATGG
CCTACTAATCTTGGATGGATGATGGGTCCTTGGCTAATATATGCTTCATT
GTTAAATGGGGGCTCACTTGCATTATACAACGGATCTCCCCTGACTTCTG
GATTTGCCAAGTTTGTTCAGGATGCAAAAGTAACATTGTTGGGAGTGATA
CCAAGTATTGTGAGGGCATGGAGAACAAACAATAGTACAGCCGGCTTTGA
CTGGTCAACCATCCGGTGCTTTGGATCGACCGGTGAGGCCTCTAATACTG
ATGAATGTCTTTGGCTGATGGGAAGAGCTCATTACAAACCGGTCATCGAG
TATTGCGGTGGCACAGAGATTGGTGGTGGTTTTATTACAGGATCTTTACT
GCAGCCTCAGTGTTTGTCTGCTTTCAGCACACCAAGTTTGGGTTGTAAAC
TGTTAATTCTTGGCGAAGATGGAATCCCTATACCACAAAACGCTCCTGGA
ATTGGTGAATTGGCTCTGAATCCCCTCATGTTTGGGGCATCGAGCACACT
ACTAAATGCAAACCACTATGATGTCTACTTTAAAGGCATGCCCTCTTGGA
ATGGTAAGGTTCTAAGAAGGCATGGAGATGTATTTGAGCGCACGTCTAAA
GGATACTATCGTGCCCATGGTCGTGCAGATGATACTATGAATCTTGGGGG
TATTAAGGTAAGTTCGGTTGAGATTGAACGTGTATGCAACTCGATTGATG
ACAGAATTCTCGAGACAGCGGCTATAGGGGTTACACCTTCTGGTGGCGGG
CCAGAGAGGTTGGTAATTGTTGTTGCTTTTAAAGATGGCAGTGGTTCGAA
ACCCGACTTAATCAAGTTGAAGGTCACACTGAATTCAGCTTTACAAAAGA
ATCTGAACCCTTTGTTTAAGGTTTCTGATGTGGTGCCCTTTCCATCACTT
CCTAGGACAGCAACAAACAAGGTAATGAGAAGGGTTTTGCGACAGCAGTT
GACTCAAATTGGTCAAAATAGCAAGCTATAA.

In some embodiments, the DNA molecule comprises a nucleic acid sequence with at least 88%, at least 90%, at least 92%, at least 95%, at least 97%, or at least 99% homology or identity to SEQ ID NO: 4, or any value and range therebetween. Each possibility represents a separate embodiment of the invention. In some embodiments, the DNA molecule comprises a nucleic acid sequence with 88% to 95%, 89% to 99%, 91 to 98%, or 88% to 100% homology or identity to SEQ ID NO: 4. Each possibility represents a separate embodiment of the invention.

In some embodiments, the DNA molecule comprises the nucleic acid sequence:

(SEQ ID NO: 5)
ATGGGTGATTCAGAGGGAAGCAGCATTAGTACTCCTACAACTGAACAAGT
TGGTTTCTTGTCAAATATCATGGAAGACAAATCTTATAGTGCTGCAGTTG
CAATTATGGTTGCCATTGCTGTACCGTTGGTTCTTTCTTCAGTGTTTGCA
GCGAAGAAGAAAGTGAAACAACGAGGCGTTCCCGTTCAAGTTGGTGGTGA
GCCAGGTTTTGCCATGCGTAACTCTAGATCAAACAAATTAGTTGATGTCC
CATGGGAAGGAGCTAGAACAATGGCTGCTCTTTTTGAGCAGTCTTGTAAG
AAGCATTCACAGCTTCGGTTTCTTGGTACAAGGAAGTTGATTGAAAGAAG
CTTTGTGAGTGGTAGTGATGGGAGAAAATTCGAGAAGTTACATCTTGGGG
AGTATCAGTGGGAGACATATGGGCAGATATTTGAACGTGTTTGCAACTTT
GCATCTGGACTTATTCAGCTTGGTCATGACCCTGATACTCGTATTGCCAT
CTTTTCTGACACACGAGCTGAATGGTTAATTGCATTTGAGGGATGCTTCA
GGCAGAACATCACTGTGGTTACCATATATGCATCATTAGGTGATGATGCC
CTCATTCACTCTCTTAACGAGACTAAAGTATCGACCTTGATTTGTGATTC
CAAACTATTGAAAAAAGTGGCTGCAGTTAGTTCAAGCCTGAAAACTGTAG
AAAACTTCATCTACTTTGAAAGTGACAACACTGAAGCTTTAAATGAAATC
GGTGATTGGAAAATATCTTCTTTTTCTGAAGTCGAGAGCTTGGGACAGAA
GAGTCCAGTAAGTGCTAGACTGCCTATCAAGAAAGACGTTGCAGTGATCA
TGTATACAAGTGGCAGCACAGGTTTACCAAAGGGGGTGATGATGACTCAT
GGGAATGTAGTAGCAACTGCAGCTGCGGTTATGACTGTAATCCCAAATAT
TGGGACCAATGATGTTTATCTGGCATACTTACCATTGGCTCATATTTTCG
AGTTGGCTGCTGAGACTGTGATGGTAACTGCAGGTATTCCAATTGGTTAT
GGTTCAGCACTCACTTTAACAGACACATCAAATAAAATCAAGAAAGGAAC
CTTGGGAGATGCATCCATCTTGAAGCCAACGTTAATGGCAGCTGTTCCAG
CTATTTTAGATCGTGTCCGAGATGGAGTATTAAAGAAGGTTGAGGAAAAG
GGAGGTTTGACAACAAAAATATTCAATATAGCCTACAAAAGGCGTTTGCT
AGCAGTAGATGGAAGTTGGCTGGGTGCATGGGGGTTAGAGAAGCTATTGT
GGGATGCCATTGTTTTTAAGAAGATTCGTTCTGTACTTGGAGGAGATATC
CGTTTCATGCTCTGTGGTGGTGCTCCTTTAGCTGCAGATACTCAGCGATT
TATAAATGTCTGCGTTGGGGCTCCAATTGGACAAGGATATGGGCTGACCG
AAACATGCGCTGGAGCTGCTTTCTCTGAGGCAGATGATAATTCTGTTGGG
CGTGTTGGTCCACCACTTCCTTGTGTCTATATTAAACTTGTTTCATGGGA
TGAAGGTGGGTATTTAACATCAGACAAACCAATGCCGCGAGGCGAAGTTG
TAGTTGGTGGGTACAGTGTAACCGCTGGTTACTTTAATAATGAGGAAAAG
ACCAATGAGGTTTACAAGGTTGATGAAAGTGGGATGCGTTGGTTCTACAC
TGGGGACATTGGAAGGTTTCATCCTGATGGATGCCTTGAAATCATTGACA
GGAAGAAGGATATTGTAAAACTTCAACATGGAGAGTACATCTCCTTGGGG
AAGGTTGAGGCAGCACTTGCGTCAAGCAAGTATGTAGAGAATGTAATGTT
ACATGCCGACCCCTTCCACACTTATTGTGTCGCCTTAGTTGTCCCTGCGC
GTCAGGTTATAGAACAGTGGGCTCAAGATGCGGGTATTAGTTACCAAGAT
TTTGCTGAGTTGTGTGATAAAAAGGAAACTGTCTCTGAGGTTCAGCAATC
CCTTACCAAGGTAGCAAAAGATGCAAAACTAGACAAGTTTGAAACGCCTG
CAAAGATAAAGCTGATGCCAGATCCATGGACTCCTGAATCTGGATTAGTA
ACAGCGGCTCTTAAGTTAAAAAGGGAACAACTGAAGTCCAAATTTAAGGA
TGATCTGGATAAGCTATATGGGTGA.

In some embodiments, the DNA molecule comprises a nucleic acid sequence with at least 88%, at least 90%, at least 92%, at least 95%, at least 97%, or at least 99% homology or identity to SEQ ID NO: 5, or any value and range therebetween. Each possibility represents a separate embodiment of the invention. In some embodiments, the DNA molecule comprises a nucleic acid sequence with 88% to 95%, 89% to 99%, 91 to 98%, or 88% to 100% homology or identity to SEQ ID NO: 5. Each possibility represents a separate embodiment of the invention.

In some embodiments, the DNA molecule comprises the nucleic acid sequence:

(SEQ ID NO: 6)
ATGTCGGTTTACACCGTTAAAGTCGAGGATTCACGGGCAGCTTCCGGAGA
AACCCCGTCAGCAGGGCCGGTTTACAGGTGCATTTATGCCAAGGATGCTC
TCATGGAACTGCCCCCCGGTTATGAATCTCCCTGGGACTTCTTTAGTGAG
TCTGTTAAAAGAAACCCAAAGAACCCAGCACTAGGTCGTCGTCAAGTCAT
CGATGGAAAGGCTGGTGGTTATTCATGGCTTTCATATCAAGAAGCCTACA
ATTCTGCTCTACGCATTGCTTCTGCCATCAGAAGCCGATCTGTTAATCCT
GGGGATCGGTGTGGTATATATGGACCTAACTGTCCTGAATGGATAATCTC
AATGGAGGCTTGTAACAGCAATGGCATAACCTATGTTCCCCTATATGATA
CACTTGGTGCTAATGCGGTTGAATACATCATCAACCATGCAGAAATTTCT
TTAGTTTTTGTTCAAGAGAACAAGTTGTCTGCTATTTTATCATGTCTTCC
AAATTGCTCATCAAATCTTAAAACAATCGTCAGCTTTGGGAAGTTCTCTG
AATCACAAAAGAACGAAGCCATGGAACATGGCGTCGATTGCTTCTCTTGG
GAAGAGTTTTCTTCGATGGGGAATTTGGAAGATGAACTTCCTGCAAAAAA
TAAGACTGACATTTGCACCATAATGTATACAAGTGGAACAACGGGAGAGC
CTAAGGGTGTCGTACTAAGTAACAGAGCTTTCATGTCCGAAGTCTTGTCT
ATGCATGAACTACTCATAGAAACAGACAAACCGGGCACAGAAGAAGATAC
CTACTTCTCTTTTCTTCCTTTGGCACATATATTTGATCAAATAATGGAGA
CGTATTTCATCTACAGTGGTGCTTCGATAGGGTTTTGGCAAGGAGATATC
AGATACTTGATTGAAGACCTTCTTGTGTTGCAGCCAACCATATTTTGTGG
TGTTCCAAGAGTTTATGACCGCATTTATACGGGCATAATGGCTAAGATTT
CAACTGGAGGTGCTATTCGGAAGGCATTATTTGATTTTGCATACAACTAT
AAATTAAGGAACCTTGAAAAGGGAATACAACAAGACAAATCAGCTCCTCT
TTTGGACAAGCTGGTCTTCGATAAGATTAAACAAGGGTTTGGAGGAAGGG
TTCGTCTTATGTTATCTGGAGCCGCACCTTTGCCAAAACACGTGGAGGAA
TTTTTAAGAGTGACGTGCTGTACCGTTCTCTCACAAGGATACGGACTTAC
TGAAAGTTGTGGTGGATGCTTTACATCCATTGCGAATGTGTACTCTATGA
TCGGGACTGTTGGTGTACCCATGACAACTATTGAAGCAAGACTTGAGTCA
GTGCCAGAGATGGGATATGATGCACTCAGTAGTGTGCCATGTGGCGAAAT
TTGCCTCAGGGGAAACACACTATTTTCTGGGTACCACAAACGAGACGATC
TAACTGATGCTGTCCTTGTAGATGGCTGGTTCCATACAGGTGACATTGGG
GAATGGCAGGCAGATGGAGCAATGAAAATCATTGACAGGAAAAAGAATAT
ATTCAAATTGTCTCAAGGAGAATATGTTGCAGTTGAAAGTATTGAAAGCA
CCTATTCACGGTGTCCTTTGGTTACCTCGATTTGGGTGTACGGCAATAGT
TTTGAATCTTTTCTAGTTGCGGTTGTGGTTCCCGATAGAGTAGCAGTTGA
AGAGTTTGCTGCAAAGAACAATGAATCAGGAGATTATGCATCGTTGTGCA
AGAACCCAAATGTCAGGAAATATGTTCTTGAAGAGCTGAATGCTGAAGCT
CAATGCAATAAACTTCGCGGGTTTGAGATGCTAAAAGCAGTTCATTTGGA
TCCAGTCCCATTTGACTTCGAGAGGGATTTAATAACACCAACCTTTAAAC
TAAAAAGACAGCAGCTTCTAAAATACTATAAGGATTGCGTTGAACAACTA
TATGCTGAAGCAAAGACATCCAAGAAATGA.

In some embodiments, the DNA molecule comprises a nucleic acid sequence with at least 89%, at least 90%, at least 92%, at least 95%, at least 97%, or at least 99% homology or identity to SEQ ID NO: 6, or any value and range therebetween. Each possibility represents a separate embodiment of the invention. In some embodiments, the DNA molecule comprises a nucleic acid sequence with 89% to 95%, 90% to 99%, 91 to 98%, or 89% to 100% homology or identity to SEQ ID NO: 6. Each possibility represents a separate embodiment of the invention.

In some embodiments, the DNA molecule comprises the nucleic acid sequence:

(SEQ ID NO: 7)
ATGGAAACTCATGGACCAAGGCTTCTAGGTGCAGCTTACAAAGATCCTAT
CACGAGTTATAAACAGTTCCAAAAGTTCTCTGTTCAACATCTAGAGGTGT
ATTGGTCTCTTGTGTTAGAAAAGCTTTCAATCCAATTTCAGGAACGTCCA
AAATGTATAGTAGATACTTCTGACAAATCAAAACACGGGGGCACATGGCT
TCCCGGTTCAGTTTTGAACATTGCGGAGTGTTGTATATTGTCAACTACTG
AAACAGATGAAAAGGTTGCGATTGTGTGGCGGGATGAAAGATGTGATAAT
CTGGATGTAAACAAGATGACATTCAAAGAATTGCGACAACAAGTAATGTT
GGTTGCAAATGCATTGAAGTTATTGTTTTCAAAAGGAGATCCTATTGCAA
TTGATATGCCAATGACAGTTACTGCAGTAATTCTATATTTGGCGATTGTA
TATTCTGGATTTGTGGTTGTATCTATAGCTGACAGTTTTGCAGCTAAAGA
GATTGCAACACGATTACGTGTATCTAATGCAAAGGCTATCTTTACTCAAG
ATTACATTGTTCGAGGTGGTCGAAGATTTCCTTTGTACAGTCGAGTTATT
GAAGCCACCCAATGTAGAGCCATCGTGGTTCCTGCGATAGGGGAAAACGT
AGAAGTTATTTTAAGAAAACAGGACATTTCATGGGGCGATTTTCTTTCTG
GTGCAAAACAGCTTCCTAGCCCGGATTATTGCTCTCCAGTCTATCAATCC
ATAGACACGTTGACAAACATACTCTTCTCTTCGGGAACAACAGGAGACCC
AAAAGCTATACCATGGACGCAAATATCTCCAATGAGATGTGCTGCTGACG
GATGGGCTCATATGGATATTCAGGCTGGAGATGTTTATTGTTGGCCCACA
AATCTGGGATGGGTCATGGGACCCATTGTACTTTACTCGAGTTTTCTTAC
CGGTGCAACATTGGCTCTTTATAATGGCTCCCCTCTTGGTCATGGTTTTG
GAAAATTTGTTCAGGATGCAGGAGTGACAATTTTGGGCACGGTTCCAAGC
ATAGTCAAGTCTTGGAAGAGTACAAGATGTATGGAAGGACTGGACTGGAC
AAAGATAAAGGCATTTGGGTCGACTGGTGAAGCTTCTAATGTCGACGATG
ACCTTTGGCTTTCCTCAAAGGCCTACTACAAACCTGTTCTTGAATGCTGT
GGAGGTACCGAGCTTGCATCTTCTTATGTTCAAGGGAATCTTCTACAGCC
ACAAGCCTTTGGAGCATTAAGCTCTGCTTCAATGGGAACCGGATTTGTCA
TATTTGACGATCATGGAGTTCCTTACCCGGACGATGAACCCTGTGTTGGT
GAAGTGGGTTTGTTTCCAGTATATATGGGAGCATCTGATAGACTACTGAA
TGCAGATCATGAAAAAATTTACTTCAAGGGAATGCCGAGTTACAAAGGAA
TGCAACTAAGGAGACATGGAGATATCATCAAGAGAACAATTGGAGGATAT
TTGGTTGTACAAGGCAGGGCTGATGATACCATGAACCTTGGTGGCATAAA
GACGAGCTCAATAGAAATTGAGCGTGTTTGTGAACAAGCTGATGGAAGCA
TCATGGAAACTGCTGCAGTCAGTGTTGCACCTGCAACCGGTGGTCCAGAA
CTATTAGCCATATTTGTGGTACTAAAGAACGGTTGCAACACTCAACCACA
GGACCTAAAGATGATATTTTCAAAGGCCATTCAAAAAAACCTCAACCCAT
TGTTCAAGGTGAGCTTTGTAAAGGTTGTTCCAGAGTTCCCTCGAACCGCT
TCTAACAAGTTATTGAGAAGAGTTTTAAGGAATCAAGTGAAGGAAGAGCT
TCAAACTCGAAGTAAAATATAA.

In some embodiments, the DNA molecule comprises a nucleic acid sequence with at least 85%, at least 87%, at least 90%, at least 95%, at least 97%, or at least 99% homology or identity to SEQ ID NO: 7, or any value and range therebetween. Each possibility represents a separate embodiment of the invention. In some embodiments, the DNA molecule comprises a nucleic acid sequence with 85% to 94%, 88% to 97%, 85% to 100%, or 92% to 99% homology or identity to SEQ ID NO: 7. Each possibility represents a separate embodiment of the invention.

In some embodiments, the DNA molecule comprises the nucleic acid sequence:

(SEQ ID NO: 8)
ATGGAGATCACTAAAAGCATCCAAGAATTAGGATTACAAGATCTACTAAA
CACTGGATTAACACCTAATGATGCAAAATCACTGCAAATCGAGATTAAAC
ACATCATTAATAGTCAAACTACTAATTCAAACCCAGTTGAGTTATGGCGT
CAAATCACTTCTGCAAAGCTGCTTAAACCCTCTTATCCTCATTCGTTGCA
CCAGCTCATCTACTACGCGGTGTACTGTAACTATGATGCATCCATCTATG
GTCCTCCCCTGTATTGGTTTCCATCTGAAATTGATTCTAAAAGGTCAAAC
TTGGGGAACATTATGGAAACTCATGGACCAAGGCTTCTAGGTGCAGCTTA
CAAAGATCCTATCACGAGTTATAAACAGTTCCAAAAGTTCTCTGTTCAAC
ATCTAGAGGTGTATTGGTCTCTTGTGTTAGAAAAGCTTTCAATCCAATTT
CAGGAACGTCCAAAATGTATAGTAGATACTTCTGACAAATCAAAACACGG
GGGCACATGGCTTCCCGGTTCAGTTTTGAACATTGCGGAGTGTTGTATAT
TGTCAACTAGTGAAACAGATGATAAGGTTGCGATTGTATGGCGGGATGAA
AGATGTGATAATCTGGATGTAAACAAGATGACATTCAAAGAATTGCGACA
ACAAGTAATGTTGGTTGCAAATGCATTGAAGTTATTGTTTTCAAAAGGAG
ATCCTATTGCAATTGATATGCCAATGACAGTTACTGCAGTAATTCTATAT
TTGGCGATTGTATATTCTGGATTTGTGGTTGTATCTATAGCTGACAGTTT
TGCAGCTAAAGAGATTGCAACACGATTACGTGTATCTAATGCAAAGGCTA
TCTTTACTCAAGATTACATTGTTCGAGGTGGTCGAAGATTTCCTTTGTAC
AGTCGAGTTATTGAAGCCACCCAATGTAGAGCCATCGTGGTTCCTGCGAT
AGGGGAAAACGTAGAAGTTATTTTAAGAAAACAGGACATTTCATGGGGCG
ATTTTCTTTCTGGTGCAAAACAGCTTCCTAGCCCGGATTATTGCTCTCCA
GTCTATCAATCCATAGACACGTTGACAAACATACTCTTCTCTTCGGGAAC
AACAGGAGACCCAAAAGCTATACCATGGACGCAAATATCTCCAATGAGAT
GTGCTGCTGACGGATGGGCTCATATGGATATTCAGGCTGGAGATGTTTAT
TGTTGGCCCACAAATCTGGGATGGGTCATGGGACCCATTGTACTTTACTC
GAGTTTTCTTACCGGTGCAACATTGGCTCTTTATAATGGCTCCCCTCTTG
GTCATGGTTTTGGAAAATTTGTTCAGGATGCAGGAGTGACAATTTTGGGC
ACGGTTCCAAGCATAGTCAAGTCTTGGAAGAGTACAAGATGTATGGAAGG
ACTGGACTGGACAAAGATAAAGGCATTTGGGTCGACTGGTGAAGCTTCTA
ATGTCGACGATGACCTTTGGCTTTCCTCAAAGGCCTACTACAAACCTGTT
CTTGAATGCTGTGGAGGTACCGAGCTTGCATCTTCTTATGTTCAAGGGAA
TCTTCTACAGCCACAAGCCTTTGGAGCATTAAGCTCTGCTTCAATGGGAA
CCGGATTTGTCATATTTGACGATCATGGAGTTCCTTACCCGGACGATGAA
CCCTGTGTTGGTGAAGTGGGTTTGTTTCCAGTATATATGGGAGCATCTGA
TAGACTACTGAATGCAGATCATGAAAAAATTTACTTCAAGGGAATGCCGA
GTTACAAAGGAATGCAACTAAGGAGACATGGAGATATCATCAAGAGAACA
ATTGGAGGATATTTGGTTGTACAAGGCAGGGCTGATGATACCATGAACCT
TGGTGGCATAAAGACGAGCTCAATAGAAATTGAGCGTGTTTGTGAACAAG
CTGATGGAAGCATCATGGAAACTGCTGCAGTCAGTGTTGCACCTGCAACC
GGTGGTCCAGAACTATTAGCCATATTTGTGGTACTAAAGAACGGTTGCAA
CACTCAACCACAGGACCTAAAGATGATATTTTCAAAGGCCATTCAAAAAA
ACCTCAACCCATTGTTCAAGGTTTTCTCCTAA.

In some embodiments, the DNA molecule comprises a nucleic acid sequence with at least 84%, at least 87%, at least 90%, at least 95%, at least 97%, or at least 99% homology or identity to SEQ ID NO: 8, or any value and range therebetween. Each possibility represents a separate embodiment of the invention. In some embodiments, the DNA molecule comprises a nucleic acid sequence with 84% to 94%, 88% to 97%, 84% to 100%, or 92% to 99% homology or identity to SEQ ID NO: 8. Each possibility represents a separate embodiment of the invention.

In some embodiments, the DNA molecule comprises the nucleic acid sequence:

(SEQ ID NO: 9)
ATGGTGTACAAGTCTTTGAATTCAATATCCATATCAGATATAGTAAATCT
TGGTATATCACCTGAAACTGCAACTCAACTTCATCAGAAACTAACTGAAA
TCATTCAGATTTATGGTTTTGATGCTCCTCAAACATGGACCCAGATATCC
ACCCGGATTCTTCATCCGGACCTTCCCTTTTGTTTTCATCAGATGATGTA
TTATGGATGCTATGTTGATTTTGGACCGGATCCTCCTGCTTGGTCACCCG
ACCCGAAGGATGCAAAGTTAACAAACATAGGTAGTTTATTAGAGAGACGC
GGAAAGGAGTTCTTGGGGCCTAGTTATAAAGATCCCATTTCAAGCTACTC
TGCTCTTCAGGAATTTTCAGCCTTAAATCTAGAGGTGTTTTGGAAAACAA
TATTGGATGAAATGAATATAACATTTTCTGTGCCTCCAAAACGCATATTA
GTTGATGACCTGTCTAAAGAAAGCCAGTTATTGCATCCAGGTGGTCGATG
GCTTCCCGGAGCTTATGTAAATCCAGCTAGAAATTGTTTGAGTTTAAGTA
GCAAGAGAAGGTTAAGTGATATAGCAGTTATATGGCGTGATGAAGGAAAT
GATGATATGCCGGTCAACAAAATGACGTTTCAGCAGTTGCGCTCAGAGGT
TTGGTTAGTTGCATATGCACTTGATACATTGGGAGTGGAAAAAGGATCTG
CAATTGCAATCGATATGCCTATGGATGTCAAATCTGTGGTGATTTATCTA
GCCATTGTTTTAGCAGGCTATGTGGTTGTATCTATTGCAGATAGTTTTGC
TGCTGGTGAAATTTCGACCAGACTTGTATTATCAAAAGCAAAAGCAATTT
TTACTCAGGATTTGATCATTCGTGGTGACAGAAGCCATCCCTTGTACAGC
CGAGTTGTTGATGCTCAATCACCTCTAGCAATTGTCATTCCTACGAGAGG
CTCAAGTTTTAGTATAAAATTACGTGACGGTGATATTTCTTGGCATGATT
TTCTGGAACGAGCTAACACTTACAGGAATGTTGAGTTTGTTGCTGTTGAA
CGACCCGTTGAAGCTTTCTCAAATATCCTTTTCTCATCAGGAACTACAGG
GGAACCGAAGGCAATTCCATGGACCCTTGCAACACCTTTCAAGGCTGGTG
CAGACGCTTGGTGCCACATGGATGTCCACAAAGGTGATGTTGTTGCATGG
CCTACTAATCTTGGATGGATGATGGGTCCTTGGCTAATATATGCTTCATT
GTTAAATGGGGGCTCACTTGCATTATACAACGGATCTCCCCTGACTTCTG
GATTTGCCAAGTTTGTTCAGGATGCAAAAGTAACATTGTTGGGAGTGATA
CCAAGTATTGTGAGGGCATGGAGAACAAACAATAGTACAGCCGGCTTTGA
CTGGTCAACCATCCGGTGCTTTGGATCGACCGGTGAGGCCTCTAATACTG
ATGAATGTCTTTGGCTGATGGGAAGAGCTCATTACAAACCGGTCATCGAG
TATTGCGGTGGCACAGAGATTGGTGGTGGTTTTATTACAGGATCTTTACT
GCAGCCTCAGTGTTTGTCTGCTTTCAGCACACCAAGTTTGGGTTGTAAAC
TGTTAATTCTTGGCGAAGATGGAATCCCTATACCACAAAACGCTCCTGGA
ATTGGTGAATTGGCTCTGAATCCCCTCATGTTTGGGGCATCGAGCACACT
ACTAAATGCAAACCACTATGATGTCTACTTTAAAGGCATGCCCTCTTGGA
ATGGTAAGGTTCTAAGAAGGCATGGAGATGTATTTGAGCGCACGTCTAAA
GGATACTATCGTGCCCATGGTCGTGCAGATGATACTATGAATCTTGGGGG
TATTAAGGTAAGTTCGGTTGAGATTGAACGTGTATGCAACTCGATTGATG
ACAGAATTCTCGAGACAGCGGCTATAGGGGTTACACCTTCTGGTGGCGGG
CCAGAGAGGTTGGTAATTGTTGTTGCTTTTAAAGATGGCAGTGGTTCGAA
ACCCGACTTAATCAAGTTGAAGGTCACACTGAATTCAGCTTTACAAAAGA
ATCTGAACCCTTTGTTTAAGGTTTCTGATGTGGTGCCCTTTCCATCACTT
CCTAGGACAGCAACAAACAAGGTAATGAGAAGGGTTTTGCGACAGCAGTT
GACTCAAATTGGTCAAAATAGCAAGCTATAA.

In some embodiments, the DNA molecule comprises a nucleic acid sequence with at least 88%, at least 90%, at least 92%, at least 95%, at least 97%, or at least 99% homology or identity to SEQ ID NO: 9, or any value and range therebetween. Each possibility represents a separate embodiment of the invention. In some embodiments, the DNA molecule comprises a nucleic acid sequence with 88% to 95%, 89% to 99%, 91 to 98%, or 88% to 100% homology or identity to SEQ ID NO: 9. Each possibility represents a separate embodiment of the invention.

In some embodiments, the DNA molecule comprises the nucleic acid sequence:

(SEQ ID NO: 10)
ATGACGTTTCAGCAGTTGCGCTCAGAGGTTTGGTTAGTTGCATAT
GCACTTGATACATTGGGAGTGGAAAAAGGATCTGCAATTGCAATC
GATATGCCTATGGATGTCAAATCTGTGGTGATTTATCTAGCCATT
GTTTTAGCAGGCTATGTGGTTGTATCTATTGCAGATAGTTTTGCT
GCTGGTGAAATTTCGACCAGACTTGTATTATCAAAAGCAAAAGCA
ATTTTTACTCAGGATTTGATCATTCGTGGTGACAGAAGCCATCCC
TTGTACAGCCGAGTTGTTGATGCTCAATCACCTCTAGCAATTGTC
ATTCCTACGAGAGGCTCAAGTTTTAGTATAAAATTACGTGACGGT
GATATTTCTTGGCATGATTTTCTGGAACGAGCTAACACTTACAGG
AATGTTGAGTTTGTTGCTGTTGAACGACCCGTTGAAGCTTTCTCA
AATATCCTTTTCTCATCAGGAACTACAGGGGAACCGAAGGCAATT
CCATGGACCCTTGCAACACCTTTCAAGGCTGGTGCAGACGCTTGG
TGCCACATGGATGTCCACAAAGGTGATGTTGTTGCATGGCCTACT
AATCTTGGATGGATGATGGGTCCTTGGCTAATATATGCTTCATTG
TTAAATGGGGGCTCACTTGCATTATACAACGGATCTCCCCTGACT
TCTGGATTTGCCAAGTTTGTTCAGGATGCAAAAGTAACATTGTTG
GGAGTGATACCAAGTATTGTGAGGGCATGGAGAACAAACAATAGT
ACAGCCGGCTTTGACTGGTCAACCATCCGGTGCTTTGGATCGACC
GGTGAGGCCTCTAATACTGATGAATGTCTTTGGCTGATGGGAAGA
GCTCATTACAAACCGGTCATCGAGTATTGCGGTGGCACAGAGATT
GGTGGTGGTTTTATTACAGGATCTTTACTGCAGCCTCAGTGTTTG
TCTGCTTTCAGCACACCAAGTTTGGGTTGTAAACTGTTAATTCTT
GGCGAAGATGGAATCCCTATACCACAAAACGCTCCTGGAATTGGT
GAATTGGCTCTGAATCCCCTCATGTTTGGGGCATCGAGCACACTA
CTAAATGCAAACCACTATGATGTCTACTTTAAAGGCATGCCCTCT
TGGAATGGTAAGGTTCTAAGAAGGCATGGAGATGTATTTGAGCGC
ACGTCTAAAGGATACTATCGTGCCCATGGTCGTGCAGATGATACT
ATGAATCTTGGGGGTATTAAGGTAAGTTCGGTTGAGATTGAACGT
GTATGCAACTCGATTGATGACAGAATTCTCGAGACAGCGGCTATA
GGGGTTACACCTTCTGGTGGCGGGCCAGAGAGGTTGGTAATTGTT
GTTGCTTTTAAAGATGGCAGTGGTTCGAAACCCGACTTAATCAAG
TTGAAGGTCACACTGAATTCAGCTTTACAAAAGAATCTGAACCCT
TTGTTTAAGGTTTCTGATGTGGTGCCCTTTCCATCACTTCCTAGG
ACAGCAACAAACAAGGTAATGAGAAGGGTTTTGCGACAGCAGTTG
ACTCAAATTGGTCAAAATAGCAAGCTATAA.

In some embodiments, the DNA molecule comprises a nucleic acid sequence with at least 89%, at least 92%, at least 95%, or at least 97% homology or identity to SEQ ID NO: 10, or any value and range therebetween. Each possibility represents a separate embodiment of the invention. In some embodiments, the DNA molecule comprises a nucleic acid sequence with 89% to 95%, 90% to 97%, 95% to 99%, or 90% to 100% homology or identity to SEQ ID NO: 10. Each possibility represents a separate embodiment of the invention.

In some embodiments, the DNA molecule comprises the nucleic acid sequence:

(SEQ ID NO: 11)
ATGAATATAACATTTTCTGTGCCTCCAAAACGCATATTAGTTGAT
GACCTGTCTAAAGAAAGCCAGTTATTGCATCCAGGTGGTCGATGG
CTTCCCGGAGCTTATGTAAATCCAGCTAGAAATTGTTTGAGTTTA
AGTAGCAAGAGAAGGTTAAGTGATATAGCAGTTATATGGCGTGAT
GAAGGAAATGATGATATGCCGGTCAACAAAATGACGTTTCAGCAG
TTGCGCTCAGAGGTTTGGTTAGTTGCATATGCACTTGATACATTG
GGAGTGGAAAAAGGATCTGCAATTGCAATCGATATGCCTATGGAT
GTCAAATCTGTGGTGATTTATCTAGCCATTGTTTTAGCAGGCTAT
GTGGTTGTATCTATTGCAGATAGTTTTGCTGCTGGTGAAATTTCG
ACCAGACTTGTATTATCAAAAGCAAAAGCAATTTTTACTCAGGAT
TTGATCATTCGTGGTGACAGAAGCCATCCCTTGTACAGCCGAGTT
GTTGATGCTCAATCACCTCTAGCAATTGTCATTCCTACGAGAGGC
TCAAGTTTTAGTATAAAATTACGTGACGGTGATATTTCTTGGCAT
GATTTTCTGGAACGAGCTAACACTTACAGGAATGTTGAGTTTGTT
GCTGTTGAACGACCCGTTGAAGCTTTCTCAAATATCCTTTTCTCA
TCAGGAACTACAGGGGAACCGAAGGCAATTCCATGGACCCTTGCA
ACACCTTTCAAGGCTGGTGCAGACGCTTGGTGCCACATGGATGTC
CACAAAGGTGATGTTGTTGCATGGCCTACTAATCTTGGATGGATG
ATGGGTCCTTGGCTAATATATGCTTCATTGTTAAATGGGGGCTCA
CTTGCATTATACAACGGATCTCCCCTGACTTCTGGATTTGCCAAG
TTTGTTCAGGATGCAAAAGTAACATTGTTGGGAGTGATACCAAGT
ATTGTGAGGGCATGGAGAACAAACAATAGTACAGCCGGCTTTGAC
TGGTCAACCATCCGGTGCTTTGGATCGACCGGTGAGGCCTCTAAT
ACTGATGAATGTCTTTGGCTGATGGGAAGAGCTCATTACAAACCG
GTCATCGAGTATTGCGGTGGCACAGAGATTGGTGGTGGTTTTATT
ACAGGATCTTTACTGCAGCCTCAGTGTTTGTCTGCTTTCAGCACA
CCAAGTTTGGGTTGTAAACTGTTAATTCTTGGCGAAGATGGAATC
CCTATACCACAAAACGCTCCTGGAATTGGTGAATTGGCTCTGAAT
CCCCTCATGTTTGGGGCATCGAGCACACTACTAAATGCAAACCAC
TATGATGTCTACTTTAAAGGCATGCCCTCTTGGAATGGTAAGGTT
CTAAGAAGGCATGGAGATGTATTTGAGCGCACGTCTAAAGGATAC
TATCGTGCCCATGGTCGTGCAGATGATACTATGAATCTTGGGGGT
ATTAAGGTAAGTTCGGTTGAGATTGAACGTGTATGCAACTCGATT
GATGACAGAATTCTCGAGACAGCGGCTATAGGGGTTACACCTTCT
GGTGGCGGGCCAGAGAGGTTGGTAATTGTTGTTGCTTTTAAAGAT
GGCAGTGGTTCGAAACCCGACTTAATCAAGTTGAAGGTCACACTG
AATTCAGCTTTACAAAAGAATCTGAACCCTTTGTTTAAGGTTTCT
GATGTGGTGCCCTTTCCATCACTTCCTAGGACAGCAACAAACAAG
GTAATGAGAAGGGTTTTGCGACAGCAGTTGACTCAAATTGGTCAA
AATAGCAAGCTATAA.

In some embodiments, the DNA molecule comprises a nucleic acid sequence with at least 89%, at least 92%, at least 95%, or at least 97% homology or identity to SEQ ID NO: 11, or any value and range therebetween. Each possibility represents a separate embodiment of the invention. In some embodiments, the DNA molecule comprises a nucleic acid sequence with 89% to 95%, 90% to 97%, 95% to 99%, or 90% to 100% homology or identity to SEQ ID NO: 11. Each possibility represents a separate embodiment of the invention.

In some embodiments, the DNA molecule comprises or consists of the nucleic acid

(SEQ ID NO: 23)
ATGGCATCCTCAATTAATATCTCCAAGATCAGAGAGGCTCAACGA
GCACAAGGTCCAGCCTCTATTCTTGCTGTCGGTACCGCGAATCCG
TCTAATTGCGTGTATCAAGCTGATTATCCTGATTACTACTTTCGA
ATCACTAAAAGTGAACACATGGTTGATCTCAAACGGAAATTCAAG
CGCATGTGTGACCAATCTATGATAAGAAAGCGGTACATGCAAATT
ACGGAGGAGTATCTGAAAGAAAACCCCAACATTTGTGAATACATG
GCTCCATCACTTGACGCCCGTCAAGACGTTGTAGTCGTCGAAGTC
CCAAAACTCGGTAAAGAAGCCGCAACAAAAGCCATCAAAGAATGG
GGCCAACCAAAATCCAAAATTACCCATCTCATCTTTTGTACCACG
TCCGGTGTCGACATGCCCGGAGCAGATTACCAGCTCACCAAACTC
CTCGGTCTTTGTCCTTCAGTCAAACGCTTTATGATGTACCAACAA
GGTTGTTTTGCTGGTGGCACGGTTCTTCGTCTAGCTAAGGACATC
GCTGAGAACAATAAAGGTGCTCGTGTACTTGTCGTTTGTTCCGAG
ATTACAGCTGTCATTTTTCGTGGACCCAACGACACTCACCTTGAT
TCACTTATCGGTCAAGCGTTATTTGGGGATGGGGCATCTTCGGTT
ATCGTGGGGTCTGACCCAGACTTGACAACCGAGCGGCCATTGTTT
GAAATCATATCGGCTGCACAAACGATTTTACCGGACTCTGAAGGT
GCGATAGATGGACACTTGAGGGAAGCTGGGTTAACTTTTCATCTA
CTTAAAGACGTACCGAGGTTGATTTCGAAGAATATAGAGAAAGCT
TTAACACAAGCATTTTCTCCCCTGGGAATTAGTGACTGGAACTCT
ATCTTTTGGGTCACGCACCCTGGTGGTCCAGCTATACTGGACCAA
GTGGAACTCAAACTTGGACTCAAAGAGGAGAAGATGAGAACCACT
AGACATGTTCTCAGTGAATATGGGAACATGTCTAGTGCATGTGTT
TTTTTTGTACTTGATGAAATGAGAAAGAGATCGGCTAAAGGCGGT
GCGAGGACCACCGGAGAAGGGTTAGATTGGGGTGTTCTGTTTGGG
TTTGGTCCGGGTTTAACGGTTGAGACTGTGGTCCTTCATAGTCTC
CCAACTACTATGTCGATTGCGACTTAA.

In some embodiments, the DNA molecule comprises a nucleic acid sequence with at least 83%, at least 85%, at least 87%, at least 95%, or at least 99% homology or identity to SEQ ID NO: 23, or any value and range therebetween. Each possibility represents a separate embodiment of the invention. In some embodiments, the DNA molecule comprises a nucleic acid sequence with 83% to 100%, 88% to 100%, 90% to 100%, or 95% to 100% homology or identity to SEQ ID NO: 23. Each possibility represents a separate embodiment of the invention.

In some embodiments, the DNA molecule comprises or consists of the nucleic acid

(SEQ ID NO: 24)
ATGGCATCCTCAATTAATATCTCCAAGATCAGAGAGGCTCAACGA
GCACAAGGTCCAGCCTCTATTCTTGCTGTCGGTACTGCGAATCCG
TCTAATTGTGTGTATCAAGCTGATTATCCTGATTACTACTTTCGA
ATCACTAAAAGTGAACACATGGTTGATTTGAAAGAGAAATTCCAG
CGCATGTGTGACAAATCTATGATAAGAAAGCGGCACATTCACATT
ACGGAGGAGTTTTTGAAAGAAAACCCAAACCTTTGTGAATACATG
GCTCCATCACTTGACACCCGTCAAGACGTTGTAGTCGTCGAAGTC
CCAAAACTCGGTAAAGAAGCCGCAACAAAAGCCATCAAAGAATGG
GGCCAACCAAAATCCAAAATTACCCATCTCATCTTTTGTACCACG
TCCGGTGTCGACATGCCCGGAGCAGATTACCAGCTCACCAAACTC
CTCGGTCTCCATCCTTCAGTCAAACGCTTTATGATGTACCAACAA
GGTTGTTTTGCTGGTGGCACGGTTCTTCGTCTAGCTAAGGACCTC
GCTGAGAACAATAAAGGTGCTCGTGTACTTGCCGTTTGTTCCGAG
ATTACAGCTGTCACGTTTCGTGGACCCAACGACACTCACATTGAT
TCACTTGTCGGTCAAGCATTATTTGGGGACGGGGCAGCTGCGGTT
ATCGTGGGGTCTGATCCTGACTTGACAACTGAGCGGCCGTTGTTT
GAAATCATATCGGCTGCACAAACGATTTTACCGAACTCTGAAGGT
GCGATAGATGGACATGTGAGGGAAGTTGGGGTAACTATTCATATA
CTTAAAGACGTCCCGGTGTTGATTTCGAAGAATATAGAGAAAGCT
TTAACACAAGCATTTTCTCCCTTAGGAATTAGTGACTGGAACTCG
ATCTTTTGGGTCGTACACCCTGGTGGTCCAGCTATACTGGACCAA
GTGGAACTCAAACTTGGACTCAAAGAGGAGAAAATGAGAACCACT
AGACATGTTCTCAGTGAATATGGGAACATGTCTAGTGCATGTGTT
TTTTTTGTACTTGATGAAATGAGAAAGAGATCGGCTAAAGGCGGT
GCGAGGACCACCGGAGAAGGGTTAGATTGGGGTGTTCTGTTTGGG
TTTGGTCCAGGTTTAACGGTTGAGACGGTGGTCCTTCATAGTCTC
CCAACTACTATGTCGATTGCAACTTAA.

In some embodiments, the DNA molecule comprises a nucleic acid sequence with at least 83%, at least 85%, at least 87%, at least 89%, at least 92%, at least 95%, at least 97%, or at least 99% homology or identity to SEQ ID NO: 24, or any value and range therebetween. Each possibility represents a separate embodiment of the invention. In some embodiments, the DNA molecule comprises a nucleic acid sequence with 83% to 100%, 87% to 100%, 90% to 100%, or 93% to 100% homology or identity to SEQ ID NO: 24. Each possibility represents a separate embodiment of the invention.

In some embodiments, the DNA molecule comprises or consists of the nucleic acid

(SEQ ID NO: 25)
ATGGCATCCTCAATTAATATCTCCAAGATCAGAGAGGCTCAACGA
GCACAAGGTCCAGCCTCTATTCTTGCTGTCGGTACCGCGAATCCG
TCTAATTGCGTGTATCAAGCTGATTATCCTAATTACTACTTTCGA
ATCACTAAAAGTGAACACATGGTTGATCTCAAACGGAAATTCAAG
CGCATGTGTGACCAATCTATGATAAGAAAGCGGTACATGCAAATT
ACGGAGGAGTATCTGAAAGAAAACCCCAACATTTGTGAATACATG
GCTCCATCACTTGACGCCCGTCAAGACGTTGTAGTCGTCGAAGTC
CCAAAACTCGGTAAAGAAGCCGCAACAAAAGCCATCAAAGAATGG
GGCCAACCAAAATCCAAAATTACCCATCTCATCTTTTGTACCACG
TCCGGTGTCGACATGCCCGGAGCAGATTACCAGCTCACCAAACTC
CTCGGTCTCTGTCCTTCAGTCAAACGCTTTATGATGTACCAACAA
GGTTGTTTTGCTGGTGGCACGGTTCTTCGTCTAGCTAAGGACATC
GCTGAGAACAATAAAGGTGCTCGTGTACTTGTCGTTTGTTCCGAG
ATTACAGCTGTCATTTTTCGTGGACCCAACGACACTCACCTTGAT
TCACTTATCGGTCAAGCGTTATTTGGGGATGGGGCATCTTCGGTT
ATCGTGGGGTCTGACCCAGACTTGACAACCGAGCGGCCATTGTTT
GAAATCATATCGGCTGCACAAACGATTTTACCGGACTCTGAAGGT
GCGATAGATGGACACTTGAGGGAAGCTGGGTTAACTTTTCATCTA
CTTAAAGACGTACCGGGGTTGATTTCGAAGAATATAGAGAAAGCT
TTAACACAAGCATTTTCTCCCTTGGGAATTAGTGACTGGAACTCT
ATCTTTTGGGTCACGCACCCTGGTGGTCCAGCTATACTGGACCAA
GTGGAACTCAAACTTGGACTCAAAGAGGAGAAGATGAGAGCCTCT
AGACATGTTCTCAGTGAATACGGGAACATGTCTAGTGCATGTGTT
TTTTTTATACTTGATGAAATGAGAAAGAAATCGGATGAAGATGGT
GCGCCGACCACTGGAGAAGGGTTAGATTGGGGTGTTCTGTTTGGG
TTTGGTCCGGGTTTAACGGTTGAGACGGTGGTCCTTCATAGTCTC
CCAACTACTATGTCGATTGCGACTTAA.

In some embodiments, the DNA molecule comprises a nucleic acid sequence with at least 83%, at least 87%, at least 89%, at least 90%, at least 92%, at least 95%, at least 97%, or at least 99% homology or identity to SEQ ID NO: 25, or any value and range therebetween. Each possibility represents a separate embodiment of the invention. In some embodiments, the DNA molecule comprises a nucleic acid sequence with 83% to 100%, 88% to 100%, 93% to 100%, or 95% to 100% homology or identity to SEQ ID NO: 25. Each possibility represents a separate embodiment of the invention.

In some embodiments, the DNA molecule comprises or consists of the nucleic acid sequence:

(SEQ ID NO: 26)
ATGGCATCCTCAATTAATATCTCTAAGATCAGAGAGGCTCAACGA
GCACAAGGTCCAGCCTCTATTCTTGCTGTCGGTACTGCGAATCCA
TCTAATTATGAGATTCAAGCTGATTTTCCTGATTACTACTTTCGA
GTCACTAAAAGTGAACACATGGCTGATATGAAAGGGACATTCCAG
CGCATGTGTGACAAATCTATGATAAGAAAGCGGCACATGCTCATT
ACGGAGGAGTTTTTGAAAGAAAACCCAAACCTTTGTGAATACATG
GCTCCATCACTTGACACCCGTCAAGACGTTGTAGTCGTCGAAGTC
CCAAAACTCGGTAAAGAAGCCGCAACAAAAGCCATCAAAGAATGG
GGCCAACCAAAATCCAAAATTACCCATCTCATCTTTTGTACTACA
ACTGGTGTCGACATGCCTGGAGCCGATTACCAGCTCACCAAGCTC
CTCGGCCTCGCTCCTTCAGTCAAACGCTTTATGATATACCAACAA
GGTTGTTTTGCTGGTGGCACGGTTCTTCGTCTTGCTAAAGACATA
GCTGAGAACAATAAAGGTGCTCGTGTACTTGCCGTATGTTCAGAG
ATTACAGCTATGTCGTTTCGTGGGCCCAATGACACTCACGTTGAT
TCACTTGTCGGTCAAGCATTATTTGGGGACGGGGCAGCTGCAGTT
ATCGTGGGGTCTGATCCTGACTTGACAACCGAGCGGCCGTTGTTT
GAAATCATATCGGCTGCACAAACGATTTTACCAAACTCTGAAGGT
GCGATAGATGGACATGTGAGGGAAGTTGGTTTAACTATTCATATA
CTTAAAGACGTCCCGGTGTTGATATCGAAGAATATAGAGAAAGCT
TTGACACAAGCATTTTCTCCCTTAGGAATTAGTGACTGGAACTCG
ATCTTTTGGATCGTACACCCTGGTGGTCCAGCTATACTGGACCAA
GTGGAACTCAAAGTTGGACTCAAAAAGGAGAAAATGGCAACCAGT
AGACATGTTCTAAGTGAATACGGGAACATGTCTAGTGCATGTGTT
TTTTTTATAATGGATGAAATGAGAAAGAGATCGGCTAAAGGCGGT
GCGAGGACCACCGGAGAAGGGTTAGATTGGGGTGTTTTGTTTGGG
TTTGGTCCAGGTTTAACGGTTGAGACGGTGGTCCTTCATAGTCTC
CCAACTACAATGTAG.

In some embodiments, the DNA molecule comprises a nucleic acid sequence with at least 82%, at least 85%, at least 89%, at least 92%, at least 95%, at least 97%, or at least 99% homology or identity to SEQ ID NO: 26, or any value and range therebetween. Each possibility represents a separate embodiment of the invention. In some embodiments, the DNA molecule comprises a nucleic acid sequence with 82% to 100%, 86% to 100%, 90% to 100%, or 95% to 100% homology or identity to SEQ ID NO: 26. Each possibility represents a separate embodiment of the invention.

In some embodiments, the DNA molecule comprises or consists of the nucleic acid sequence:

(SEQ ID NO: 31)
ATGGCGGAGTTCACACATTTAGTGGTGGTTAAGTTCAAAGAAGAG
GTGGTTGTGGAGGATATTATGAAAGGGTTGGAGAAACTTGTATCT
CAACTTGATAGTGTCAAGTCCTTTGTTTGGGGAAAGGATATTGAA
AGCATGGAGATGTTAAGGCAAGGATTCACCCATGCAATCATGATG
ACATTTGGTTCTAAAGAAGATTTTACTGCATTTCAATCCCACCCA
AACCATGTTGAATTCTCGGCTACGTTTTCAGCAGCAATCGAAAAG
ATCGTTCTTCTTGATTTCCCAGTTGTTGCTGTCAAGACTGCAACT
GCTTGA.

In some embodiments, the DNA molecule comprises a nucleic acid sequence with at least 72%, at least 75%, at least 85%, at least 95%, or at least 99% homology or identity to SEQ ID NO: 31, or any value and range therebetween. Each possibility represents a separate embodiment of the invention. In some embodiments, the DNA molecule comprises a nucleic acid sequence with 72% to 95%, 72% to 100%, 75% to 99%, or 80% to 100% homology or identity to SEQ ID NO: 31. Each possibility represents a separate embodiment of the invention.

In some embodiments, the DNA molecule comprises or consists of the nucleic acid

(SEQ ID NO: 32)
ATGTCGTCCTTACAAAACAAATTTATCGAACACATTGCTCTTATC
AAAATCAAACCCGGTGTTGAGTCTACCACCTTGATAGATAAACTC
AACGGCCTTTCTTCGATTGAGGTGTTACTGCACTTCAGCGCGGGT
GAACTCCTGGGATCATCCCACGGCTTCACTCACATCGTTCACTGC
CGTGTCAGATCAAAGGATGATCTCCAAATCTACCTTACACATCCT
ATCCACTTGCATCTGGCTGATGATACTTTACCCTTACTTGATGAC
GTCACCGTCGTTGACTGGTTTTCATCCAACTCTGATATTGTGGAT
CCTCCTAAACCAGGATCTGCAATGAGAGTTACGCTGCTGAAGTTG
AAACACGATTCGACTGAAAGTAATAAGTTAGTAGTGATTGAAGGA
ATTAAAAATCAGTTTAAAGGAATTGAAGACGTGATAGTTACAACT
ACTTTTGGTGAGAATTTGTTTCATGAAATGCATGAGAATTTCTCG
ATTGAAATTGACAAAGGATACTCGATTGGTTCGATTGCCTTTGTT
CCTGGATCTGCAGATTTCCAGGTTTTAAATTCAAAGGTAGATAAT
AATAAACTCAATGATTTAACAGAAAGTGAAGTGGTGGTTGATTAT
GTGTTTCCATCAGCCAATTAA.

In some embodiments, the DNA molecule comprises a nucleic acid sequence with at least 50%, at least 60%, at least 70%, at least 80%, at least 90%, at least 95%, at least 97%, or at least 99% homology or identity to SEQ ID NO: 32, or any value and range therebetween. Each possibility represents a separate embodiment of the invention. In some embodiments, the DNA molecule comprises a nucleic acid sequence with 50% to 95%, 55% to 98%, 60% to 99%, or 50% to 100% homology or identity to SEQ ID NO: 32. Each possibility represents a separate embodiment of the invention.

In some embodiments, the DNA molecule comprises or consists of the nucleic acid sequence:

(SEQ ID NO: 33)
ATGTCCTCTGAAGAGCAGATCGTGGAACACGTGGTCCTGTTCAAA
GTGAAACCTGATGCTGATCCTAGTAAAGTCGCGGCTTGGGTCAAT
GGGCTCAACGGTTTGACCTCACTCCAGCTCGCCCTCCACCTCTCC
GCTGGACAACTCATCCGGTGTCGGTCGTCGTCGCTCACCTTCACT
CACATGCTTCACAGTCGTTACAGATCAAAGGAGCATCTCCGGCAG
TACACCGTTCATCCCGAGCACGTGCGCGTGGTTACAGAGGGTAAA
TCCATCATTGATGACGTCATGGCCCTTGATTGGATGATATCTAAC
GGCGCTGCTAGTAGCGTCTGTCCTAAGCCTGGATCAGCGGTGAGA
GTTGGGTTTTATAAGTTAATGGAGAGTTTGGGGGAAATTGAGAAA
GCTAGGGTTTTGGAAGTGATGGGAGGGATTGAAGAGTTAAGTGTT
GGTGAGAGTTTTTGTGATGACAGGGCCAAGGGTTATACGATTGCT
TCAACCGCCGTGTTTCCCAATGGCAATCCTGCTGCTGATTTGGAT
TTATATCATTCCGGTGACCAGCTCCTGCTGAAAGAGGAAGTGATG
AAGGATTCTATACAAAGTGTGGTGGTTGTTGATTACGTAATTCCA
TCTCCCTGA.

In some embodiments, the DNA molecule comprises a nucleic acid sequence with at least 67%, at least 72%, at least 78%, at least 85%, at least 92%, at least 95%, at least 97%, or at least 99% homology or identity to SEQ ID NO: 33, or any value and range therebetween. Each possibility represents a separate embodiment of the invention. In some embodiments, the DNA molecule comprises a nucleic acid sequence with 67% to 95%, 70% to 98%, 75% to 99%, or 67% to 100% homology or identity to SEQ ID NO: 33. Each possibility represents a separate embodiment of the invention.

In some embodiments, the DNA molecule comprises or consists of the nucleic acid

(SEQ ID NO: 34)
ATGGGAGAAGTGAAGCACATACTTTTAGCGAAGTTTAAGGATGGA
ATCTCGGAACAACAGATCCAGCATCTCATCACAGGTTATGCTAAC
CTCGTCAATCTCGTTGAACCCATGAAGTCTTTTCGATGGGGAAAA
GATGTGAGCATTGAGAATCTGCACCAAGGCTTTACTCATGTGTTC
GAGTCAACCTTTGAAACCACTGAAGGCATTGCAACTTATATATCT
CATCCTGCTCATGTCGAGTTCGCCACTGGTTTCCTGGATCAACTG
GAAAAAGTCATAGTCATCGACTACAAACCTACATCAGTTGACCCG
TGA.

In some embodiments, the DNA molecule comprises a nucleic acid sequence with at least 74%, at least 78%, at least 85%, at least 89%, at least 92%, at least 95%, at least 97%, or at least 99% homology or identity to SEQ ID NO: 34, or any value and range therebetween. Each possibility represents a separate embodiment of the invention. In some embodiments, the DNA molecule comprises a nucleic acid sequence with 74% to 95%, 78% to 98%, 80% to 99%, or 75% to 100% homology or identity to SEQ ID NO: 34. Each possibility represents a separate embodiment of the invention.

In some embodiments, the DNA molecule comprises or consists of the nucleic acid sequence:

(SEQ ID NO: 35)
ATGCTATGTGCTCCAGCACGCACACGATTACTTCCATCAATTTCT
CTCTTACCTTCCCAACATAACATCTTCCGCCGCCTGAACTGTCTC
ATCCACCGTCGCAACCACCACCAAACGCCGATCACGATGTCTGCT
CAACAACAAATCGTGGAACACGTAGTGCTCTTCAAAGTAAAACCG
GATGTTGATTCTAGTAAAGTTGCTGCAATGGTCAACGGACTCAAC
GGATTGACCTCACTCGATCTTACTCTCCACCTCTCCGCCGGACAG
CTCCTCCGGTCACGGTCATCATCGCTGACCTTCACTCACATGCTT
CACAGTCGTTACAGATCAAAGGACGATCTCCGGGAGTACGCTGCT
CATCCTGACCACGTGCGAGTCGTGACGGAGAATATAAAACCGGTT
ATTGATGATATCATGGCTGTTGATTGGATATCTAACGATGCCAGT
GTATCGCCTAAGCCAGGGTCGGCGATGAGAGTAACATTTTTGAAA
TTAAAGGAGAATTTGGGGGAAAATGAGAAATCTAGGGTTTTGGAA
GTGATTGGAGGAATCAAAAATCAGTTTAAATCAATTGAGGAGTTA
AGTGTTGGTGAGAATTTTTCTCATGATAGAGCCAAGGGGTATACG
ATTGCTTCAATTGCTGTGTTACCCGGGCCTTCCGAGCTGGAGGCA
TTGGATTCGAATACTGAGCTGGTGAAGTTGGAAAAGGAGAAAGTG
AAGGACTTACTGGAGAGCGTTGTGGTTGTTGATTATGTGATTCCA
TCTCTGCAATCGGCTAGTCTTTAA.

In some embodiments, the DNA molecule comprises a nucleic acid sequence with at least 69%, at least 75%, at least 85%, at least 95%, or at least 99% homology or identity to SEQ ID NO: 35, or any value and range therebetween. Each possibility represents a separate embodiment of the invention. In some embodiments, the DNA molecule comprises a nucleic acid sequence with 69% to 95%, 70% to 100%, 80% to 99%, or 68% to 100% homology or identity to SEQ ID NO: 35. Each possibility represents a separate embodiment of the invention.

In some embodiments, the DNA molecule comprises or consists of the nucleic acid sequence:

(SEQ ID NO: 36)
ATGGCAGTTGCTCAACTTTCTTCCTCCCTCTGTATCTCCACACCC
GCTAGAATCTCTACTGGTTCTGGGTTTTCGTCATCAGGTTTGCCT
CGGATTGGGACAACGTTTGTATGCGGTTCAGGTTCGCCTCTTGTG
ATATCTGGAACATATCATCAGAAGGCTCGAGTACATAAGCCTGCA
GCATTATCTGTGAGATGTGAACAAAGTAGTAAGGATGGAAATGGT
TTAAATGTGTGGCTTGGTCGAACAGCAATGGTTGGCTTTGCAGTG
GCAATTAGTGTTGAAGTATCAACTGGGAAGGGGCTTCTTGAGAAC
TTTGGGCTCACATCACCCTTGCCAACAGTGGCCTTGGCACTGACT
GCACTTGGGGGCGTTCTTACAGCACTTTTCATCTTCCAGTCTGCT
TCTGAGAGTTGA.

In some embodiments, the DNA molecule comprises a nucleic acid sequence with at least 73%, at least 75%, at least 85%, at least 95%, or at least 99% homology or identity to SEQ ID NO: 36, or any value and range therebetween. Each possibility represents a separate embodiment of the invention. In some embodiments, the DNA molecule comprises a nucleic acid sequence with 73% to 95%, 73% to 100%, 80% to 99%, or 80% to 100% homology or identity to SEQ ID NO: 36. Each possibility represents a separate embodiment of the invention.

In some embodiments, the DNA molecule comprises or consists of the nucleic acid

(SEQ ID NO: 37)
ATGATTGAACACATAGTCCTCCTCAAATTTAAATCCGACGTCGAC
TCTACCAAAGTCGAGTCCATGATTAACGAACTCAACGGATTGGCT
TCACTCGATGTTGCACTCGACGTGAGTGCCGGTAAAATCCTGCGA
GTGAGTAGTACATCATCCTCTTCTCTCACTTTCACCCACCTCTTT
CGCTGTTGTTTCAGATCAGCCGATGATCAGCAAGTCTTCTCTACT
CATCCTGACCATCTACGAGTGGCCATTGAAGTTCGACCCGTAATT
GAAGATATGGTAGTTGTTGACTTGGTATCCAAAACTACAATTGAC
TCACCAAACCCAGGATCTGCAATGAAAGTTAGGATATTTAAGTTG
AAAGACGATCTGATCGAAGATAGTAAGTTAGTAGTGATGGAAGGA
ATTAAAAATGAGTTAAAAGCAGTTGAACATATTAGGTTTGGTGAC
AACATTAATGTTATGGCAAAGGGATACTCGATTGCTATGATTGCT
TTTTTTCCTGATTTGGAATCTTCGGTTGCAGGTGCAGAAATTGTT
AAGGATTATATAGAGAGCGAGCTGGTGGTGGATTTTGTGTTTCCA
CCACCAAACGTTACAAGTCATTCATGA.

In some embodiments, the DNA molecule comprises a nucleic acid sequence with at least 69%, at least 78%, at least 85%, at least 89%, at least 92%, at least 95%, at least 97%, or at least 99% homology or identity to SEQ ID NO: 37, or any value and range therebetween. Each possibility represents a separate embodiment of the invention. In some embodiments, the DNA molecule comprises a nucleic acid sequence with 69% to 95%, 70% to 98%, 71% to 99%, or 69% to 100% homology or identity to SEQ ID NO: 37. Each possibility represents a separate embodiment of the invention.

In some embodiments, the DNA molecule comprises or consists of the nucleic acid sequence:

(SEQ ID NO: 38)
ATGGCGGAGTTCACACATTTAGTGGTGGTTAAGTTCAAAGAAGAG
GTGGTTGTAGAGGATATTATGAAAGGGTTGGAGAAACTTGCATCT
CAACTTGATAGTGTCAAGTCCTTTGTTTGGGGAAAGGATATTGAA
AGCATGGAGATGTTAAGGCAAGGATTCACCCATGCAATCATGATG
ACATTTGGTTCTAAAGAAGATTTTACTGCATTTCAATCCCACCCA
AACCATGTTGAATTCTCGGCTACGTTTTCAGCAGCAATCGAAAAG
ATCGTTCTTCTTGATTTCCCAGTTGTTGCAGTCAAGACTGCAACT
GCTTGA.

In some embodiments, the DNA molecule comprises a nucleic acid sequence with at least 88%, at least 90%, at least 92%, at least 95%, at least 96%, at least 97%, at least 98%, or at least 99% homology or identity to SEQ ID NO: 38, or any value and range therebetween. Each possibility represents a separate embodiment of the invention. In some embodiments, the DNA molecule comprises a nucleic acid sequence with 88% to 95%, 88% to 98%, 89% to 99%, or 88% to 100% homology or identity to SEQ ID NO: 38. Each possibility represents a separate embodiment of the invention.

In some embodiments, the DNA molecule comprises or consists of the nucleic acid

(SEQ ID NO: 47)
ATGGAGTTATCACTCTCATCATCTTCTTCTTCATCCCTTCCCCAA
CTTCATACTCATCCTTCATCATCATCATCTTCTTCACATTACATA
AAAAAATCACCTTTTTTTATTAATAAATTCAATAATCACACCAAA
TGCAAATTCCACAATTCCTCTGCTCTGAGAACTAATTTCTTCTAC
ACTACCATAACTAAAACCTCATCATCAAGATTCGTTCTAAACAAA
AACCCAAACCAATTTTCCGTCAAGGCTTGCAGTCAAGTTGGTTCT
GCTGGATCCGATCCAGCATTGAATAAAGTTGCAGACTTTAAAGAT
GCATTTTGGAGGTTTCTAAGGCCCCATACTATTCGTGGGACAGCA
TTAGGATCAGTGTCTTTAGTAACGAGAGCACTACTTGAAAACCCA
AACTTGATTCGGTGGTCACTTTTGCTCAAGGCATTTTCAGGTCTT
GTTGCTTTGATATGTGGGAATGGTTATATAGTCGGGATCAATCAG
ATCTATGATATCGGTATTGATAAGGTGAACAAACCATATTTACCT
ATTGCTGCGGGAGATCTTTCTGTCCAGTCAGCATGGTTTTTGGTG
TTAGCATTTGCAATGGTAGGCGTTATTATTGTTGGGATGAACTTC
GGCCCATTCATCACCTCCCTTTATTCTCTCGGTCTTTTCTTGGGC
ACCATCTATTCCGTTCCACCACTTCGAATGAAGAGATTTCCTGTT
GTTGCATTTCTTATCATCGCCACGGTGAGAGGTTTTCTTCTAAAT
TTTGGTGTGTATTATGCGGTTAGAGCAGCTCTGGGACTAACATTC
CAATGGAGCTCAGCAGTGGCTTTTATCACAACCTTCGTTACATTA
TTTGCTTTAGTCATTGCCATTACTAAAGATCTTCCTGATGTAGAG
GGTGACCGAAAGTTTCAAATTTCTACTTTTGCAACAAAACTTGGA
GTAAGAAACATTGCATTATTAGGGTCAGGACTTCTGCTGATCAAT
TATATTGGGTCTATCGTTGCAGCACTTTACATGCCTCAGGCTTTC
AGGAGCAGCTTGATGATACCATTACATACCATATTAGCTTCCTGT
TTGATTTACCAGGCATGGATACTTGAGCGTGCGAATTACACCCAG
GAGGCGATAGCTGGGTACTACCGATTTGTATGGAATCTGTTTTAT
TCAGAGTACATCATATTTCCTTTCATCTGA.

In some embodiments, the DNA molecule comprises a nucleic acid sequence with at least 75%, at least 79%, at least 85%, at least 95%, or at least 99% homology or identity to SEQ ID NO: 47, or any value and range therebetween. Each possibility represents a separate embodiment of the invention. In some embodiments, the DNA molecule comprises a nucleic acid sequence with 75% to 100%, 80% to 100%, 90% to 100%, or 95% to 100% homology or identity to SEQ ID NO: 47. Each possibility represents a separate embodiment of the invention.

In some embodiments, the DNA molecule comprises or consists of the nucleic acid sequence:

(SEQ ID NO: 48)
ATGGCTACTATGGCTTCTTCTTTGCTGAATCCTCTTTCTTGTTCCATTA
AACCCAACTCAAACAGACTACCATTACCAACACCCATTTCTCTATCTCG
TTCTTGTAGAAGGCTAACAATCAAAGCAACGGAGACAGATGCAAATGAA
GTGAAGCCAAAGGCGCCAGAGAAAGCACCAGCTGCAAGTGGATCTGGTT
TTAATCAAATTCTTGGGATTAAAGGGGCTAAACAAGAAACTAATAAATG
GAAGATCCGTGTTCAACTTACAAAGCCGGTTACTTGGCCTCCATTAATT
TGGGGAGTCGTATGTGGAGCTGCTGCTTCTGGTAACTTCCAATGGACTG
TGGAAGATGTTGCTAAATCAATTGTTTGCATGTTGATGTCTGGCCCATT
TCTAACCGGTTACACACAGACGATCAATGATTGGTATGATAGAGACATT
GATGCTATTAATGAACCTTACCGTCCAATTCCTTCCGGAGCCATATCTG
AAAATGAGGTCATTACTCAAATTTGGGTACTTCTTTTAGGAGGCATCGG
ATTGGCTGGTATATTAGACGTGTGGGCAGGGCATAAGTCCCCTACAATA
TTCTATCTTGCTTTGGGTGGATCATTGTTATCTTATATCTACTCAGCTC
CACCTTTAAAGCTCAAACAGAATGGATGGATTGGCAACTTTGCATTAGG
AGCAAGCTATATTAGCTTACCATGGTGGGCTGGTCAAGCATTGTTCGGA
ACTCTTACACCTGATATAGTAGTTCTCACACTTTTGTACAGCATAGCTG
GGCTTGGTATTGCTATAGTAAATGACTTTAAAAGTGTTGAAGGAGACAG
GAAAATGGGGCTTCAGTCCCTTCCCGTGGCTTTTGGTGAAGAGACAGCT
AAATGGATATGTGTTGGTGCCATTGACATAACTCAACTCTCTATTGCAG
GTTACCTTTTAGGATCTGGTAAACCATATTACGCCTTAGCACTCGTTGG
GTTGATTGTTCCACAAATCTTTTTTCAGTTCAAGTACTTTCTTAAAGAT
CCAGTTAAATATGATGTCAAGTATCAGGCTAGTGCTCAACCATTTCTCA
TTCTTGGTCTTCTGGTGACTGCCTTAGCTACTAGTCACTGA.

In some embodiments, the DNA molecule comprises a nucleic acid sequence with at least 80%, at least 85%, at least 90%, at least 95%, at least 97%, or at least 99% homology or identity to SEQ ID NO: 48, or any value and range therebetween. Each possibility represents a separate embodiment of the invention. In some embodiments, the DNA molecule comprises a nucleic acid sequence with 80% to 100%, 85% to 100%, 90% to 100%, or 95% to 100% homology or identity to SEQ ID NO: 48. Each possibility represents a separate embodiment of the invention.

In some embodiments, the DNA molecule comprises or consists of the nucleic acid

(SEQ ID NO: 49)
ATGAAGTCTTTGATTATTGGGTCTTTTTCTAATAAGGTTTCTTGTTATT
CCCCATCATTACCAGATTCATCTTCTTCACTTATACCAACAGGTTGTTA
TCATGTATCACTAAGAACATTTCAGCGTAACCGAGCCATTCAAGCTCAA
TCAAGTCTTGTGAGATGCAATATTGGCAAATTCAATGAAACATTACTAC
TTTCGCGGAAACGAAGTACAAAACATGTTGCATGTGCGGTTTCTGAACA
ACCCATTGAACCAGATGCTACAAACCCTCAAAGTTCATTACCAAATGCT
TTGGATGCTTTCTATAGGTTTTCAAGACCTCATACAGTTATAGGAACTG
CATTGAGCATAGTTTCGGTTTCACTCCTAGCGGTTCAAAAGCTTTCGGA
TTTTTCTCCACTATTCTTCATTGGCGTTTTCGAGGCTATTGTTGCTGCC
TTCTTTATGAACATATACATTGTTGGCTTGAACCAGCTATCCGATATTG
AAATAGACAAGGTTAACAAGCCGTACCTTCCATTGGCATCTGGAGAATA
TTCAGTTCAAACTGGTATTATCATTGTATCATCATTTGCAGTCATGAGT
TTCTGGCTTGGATGGATCGTGGGCTCATGGCCTTTATTTTGGGCACTTT
TCATAAGTTTTCTTCTAGGGACCGCATATTCAATCAATATACCGATGTT
GAGATGGAAGCGCTTTGCTCTTGTGGCAGCAATGTGTATTCTAGCTGTA
AGAGCTATTATAGTTCAAGTTGCATTTTATTTGCACATTCAGACTTTTG
TGTATGGAAGACTCGCCGTGTTCCCAAAACCCGTGATATTTGCAACCGG
ATTTATGAGTTTCTTCTCTGTTGTTATAGCATTGTTCAAGGACATACCC
GACATTGTTGGAGACAAGATTTTTGGCATTCAATCATTTACTGTCCGTA
TGGGTCAAAAACGGGTGTTTTGGATTTGCATCTTATTACTTGAAATAGC
TTATGGTGTTGCTATTCTAGTTGGGGCATCATCTCCCTTCCTTTGGAGC
CGATACATAACGGTATTGGGTCATGCGATTCTTGGTCTGATTCTCTGGG
GTCGTGCCAAGTCAACGGATCTGGAGAGCAAATCAGCAATAACCTCATT
TTACATGTTCATATGGCAGTTGTTCTATGCCGAGTATTTGCTCATACCG
CTCGTGAGATGA.

In some embodiments, the DNA molecule comprises a nucleic acid sequence with at least 75%, at least 79%, at least 85%, at least 95%, or at least 99% homology or identity to SEQ ID NO: 49, or any value and range therebetween. Each possibility represents a separate embodiment of the invention. In some embodiments, the DNA molecule comprises a nucleic acid sequence with 75% to 100%, 80% to 100%, 90% to 100%, or 95% to 100% homology or identity to SEQ ID NO: 49. Each possibility represents a separate embodiment of the invention.

In some embodiments, the DNA molecule comprises or consists of the nucleic acid

(SEQ ID NO: 50)
ATGGAGTTATCACTCTCATCATCTTCTTCTTCATCCCTTCCCCAACTTC
ATACTCATCCTTCATCATCATCATCTTCTTCACATTACATAAAAAAATC
ACCTTTTTTTATTAATAAATTCAATAATCACACCAAATGCAAATTCCAC
AATTCCTCTGCTCTGAGAACTAATTTCTTCTACACTACCATAACTAAAA
CCTCATCATCAAGATTCGTTCTAAACAAAAACCCAAACCAATTTTCCGT
CAAGGCTTGCAGTCAAGTTGGTTCTGCTGGATCCGATCCAGCATTGAAT
AAAGTTGCAGACTTTAAAGATGCATTTTGGAGGTTTCTAAGGCCCCATA
CTATTCGTGGGACAGCATTAGGATCAGTGTCTTTAGTAACGAGAGCACT
ACTTGAAAACCCAAACTTGATTCGGTGGTCACTTTTGCTCAAGGCATTT
TCAGGTCTTGTTGCTTTGATATGTGGGAATGGTTATATAGTCGGGATCA
ATCAGATCTATGATATCGGTATTGATAAGGTGAACAAACCATATTTACC
TATTGCTGCGGGAGATCTTTCTGTCCAGTCAGCATGGTTTTTGGTGTTA
GCATTTGCAATGGTAGGCGTTATTATTGTTGGGATGAACTTCGGCCCAT
TCATCACCTCCCTTTATTCTCTCGGTCTTTTCTTGGGCACCATCTATTC
CGTTCCACCACTTCGAATGAAGAGATTTCCTGTTGTTGCATTTCTTATC
ATCGCCACGGTGAGAGGTTTTCTTCTAAATTTTGGTGTGTATTATGCGG
TTAGAGCAGCTCTGGGACTAACATTCCAATGGAGCTCAGCAGTGGCTTT
TATCACAACCTTCGTTACATTATTTGCTTTAGTCATTGCCATTACTAAA
GATCTTCCTGATGTAGAGGGTGACCGAAAGTTTCAAATTTCTACTTTTG
CAACAAAACTTGGAGTAAGAAACATTGCATTATTAGGGTCAGGACTTCT
GCTGATCAATTATATTGGGTCTATCGTTGCAGCACTTTACATGCCTCAG
GCTTTCAGGAGCAGCTTGATGATACCATTACATACCATATTAGCTTCCT
GTTTGATTTACCAGGCATGGATACTTGAGCGTGCGAATTACACCCAGCG
ATCACAGTACTTTGACATGTCATCTTGCAGGAGGCGATAG.

In some embodiments, the DNA molecule comprises a nucleic acid sequence with at least 91%, at least 92%, at least 95%, at least 97%, or at least 99% homology or identity to SEQ ID NO: 50, or any value and range therebetween. Each possibility represents a separate embodiment of the invention. In some embodiments, the DNA molecule comprises a nucleic acid sequence with 91% to 100%, 93% to 100%, 95% to 100%, or 97% to 100% homology or identity to SEQ ID NO: 50. Each possibility represents a separate embodiment of the invention.

In some embodiments, the DNA molecule comprises or consists of the nucleic acid

(SEQ ID NO: 51)
ATGGAGTTATCACTCTCATCATCTTCTTCTTCATCCCTTCCCCAACTTC
ATACTCATCCTTCATCATCATCATCTTCTTCACATTACATAAAAAAATC
ACCTTTTTTTATTAATAAATTCAATAATCACACCAAATGCAAATTCCAC
AATTCCTCTGCTCTGAGAACTAATTTCTTCTACACTACCATAACTAAAA
CCTCATCATCAAGATTCGTTCTAAACAAAAACCCAAACCAATTTTCCGT
CAAGGCTTGCAGTCAAGTTGGTTCTGCTGGATCCGATCCAGCATTGAAT
AAAGTTGCAGACTTTAAAGATGCATTTTGGAGGTTTCTAAGGCCCCATA
CTATTCGTGGGACAGCATTAGGATCAGTGTCTTTAGTAACGAGAGCACT
ACTTGAAAACCCAAACTTGATTCGGTGGTCACTTTTGCTCAAGGCATTT
TCAGGTCTTGTTGCTTTGATATGTGGGAATGGTTATATAGTCGGGATCA
ATCAGATCTATGATATCGGTATTGATAAGGTGAACAAACCATATTTACC
TATTGCTGCGGGAGATCTTTCTGTCCAGTCAGCATGGTTTTTGGTGTTA
GCATTTGCAATGGTAGGCGTTATTATTGTTGGGATGAACTTCGGCCCAT
TCATCACCTCCCTTTATTCTCTCGGTCTTTTCTTGGGCACCATCTATTC
CGTTCCACCACTTCGAATGAAGAGATTTCCTGTTGTTGCATTTCTTATC
ATCGCCACGGTGAGAGGTTTTCTTCTAAATTTTGGTGTGTATTATGCGG
TTAGAGCAGCTCTGGGACTAACATTCCAATGGAGCTCAGCAGTGGCTTT
TATCACAACCTTCGTTACATTATTTGCTTTAGTCATTGCCATTACTAAA
GATCTTCCTGATGTAGAGGGTGACCGAAAGTTTCAAATTTCTACTTTTG
CAACAAAACTTGGAGTAAGAAACATTGCATTATTAGGGTCAGGACTTCT
GCTGATCAATTATATTGGGTCTATCGTTGCAGCACTTTACATGCCTCAG
GTGAAAACCACTTCGATAGACCATTACAGACCATACAGCTTCCTGGTTG
ATTTACCAGGTCAAAATGGGATTACTTTAGCAGCTTGA.

In some embodiments, the DNA molecule comprises a nucleic acid sequence with at least 91%, at least 92%, at least 95%, at least 97%, or at least 99% homology or identity to SEQ ID NO: 51, or any value and range therebetween. Each possibility represents a separate embodiment of the invention. In some embodiments, the DNA molecule comprises a nucleic acid sequence with 91% to 100%, 93% to 100%, 95% to 100%, or 97% to 100% homology or identity to SEQ ID NO: 51. Each possibility represents a separate embodiment of the invention.

In some embodiments, the DNA molecule comprises or consists of the nucleic acid

(SEQ ID NO: 52)
ATGGCTACTATGGCTTCTTCTTTGCTGAATCCTCTTTCTTGTTCCATTA
AACCCAACTCAAACAGACTACCATTACCATTACCAATACCCATTTCTCT
ATCTCGTTCTTGTAGAAGGCTAACAATCAAAGCAACGGAGACAGATGCA
AATGAAGTGAAGCCAAAGGCGCCAGAGAAAGCACCAGCTGCAAGTGGAT
CTGGTTTTAATCAAATTCTTGGGATTAAAGGGGCTAAACAAGAAACTAA
TAAATGGAAGATCCGTGTTCAACTTACAAAGCCGGTTACTTGGCCTCCA
TTAATTTGGGGAGTCGTATGTGGAGCTGCTGCTTCTGGTAACTTCCAAT
GGACTGTGGAAGATGTTGCTAAATCAATTGTTTGCATGTTGATGTCTGG
CCCATTTCTAACCGGTTACACACAGACGATCAATGATTGGTATGATAGA
GACATTGATGCTATTAATGAACCTTACCGTCCAATTCCTTCCGGAGCCA
TATCTGAAAATGAGGTCATTACTCAAATTTGGGTACTTCTTTTAGGAGG
CATCGGATTGGCTGGTATATTAGACGTGTGGGCAGGGCATAAGTCCCCT
ACAATATTCTATCTTGCTTTGGGTGGATCATTGTTATCTTATATCTACT
CAGCTCCACCTTTAAAGCTCAAACAGAATGGATGGATTGGCAACTTTGC
ATTAGGAGCAAGCTATATTAGCTTACCATGGTGGGCTGGTCAAGCATTG
TTCGGAACTCTTACACCTGATATAGTAGTTCTCACACTTTTGTACAGCA
TAGCTGGGCTTGGTATTGCTATAGTAAATGACTTTAAAAGTGTTGAAGG
AGACAGGAAAATGGGGCTTCAGTCCCTTCCCGTGGCTTTTGGTGAAGAG
ACAGCTAAATGGATATGTGTTGGTGCCATTGACATAACTCAACTCTCTA
TTGCAGGTTACCTTTTAGGATCTGGTAAACCATATTACGCCTTAGCACT
CGTTGGGTTGATTGTTCCACAAATCTTTTTTCAGTTCAAGTACTTTCTT
AAAGATCCAGTTAAATATGATGTCAAGTATCAGGCTAGTGCTCAACCAT
TTCTCATTCTTGGTCTTCTGGTGACTGCCTTAGCTACTAGTCACTGA.

In some embodiments, the DNA molecule comprises a nucleic acid sequence with at least 90%, at least 92%, at least 95%, at least 97%, or at least 99% homology or identity to SEQ ID NO: 52, or any value and range therebetween. Each possibility represents a separate embodiment of the invention. In some embodiments, the polynucleotide comprises a nucleic acid sequence with 90% to 100%, 92% to 100%, 95% to 100%, or 97% to 100% homology or identity to SEQ ID NO: 52. Each possibility represents a separate embodiment of the invention.

In some embodiments, the DNA molecule comprises or consists of the nucleic acid

(SEQ ID NO: 53)
ATGGCATCTCTAGCTATTGGTTCACTTGGTAGCCCAAGCTCACGTCAGT
GTTCTAGCCCCGTTGCATCATCTTCTTCATTTGCGATAGGGTCACAAAT
AGCTTCAAAGTTTCTTCGGATATCAAAATTTGATAAGACTAAGAACAGC
CCCTTAACATTGCAACAAAAGCATATAAACAAAAGCATAGATCAAAGCT
TCTTTGAGCCGCTTCCATTGCACAAAATAAACAAAGACAAGTTTAAGTT
GTATGCAACATCTACAAACAATCCTCAGTTTGATGCAACTCATGATTTG
AAGACTCCGGAAGTATCCATTATCAACTTTGTGGACGCTCTTTATAGGT
TAATAAGGCCGTATACAGCAGTTGTAACGATCGTAAGTGTAGTCGCGAT
GTCCCTTCTTACAGTTAATAGCCTTTCAGATTTTTCCCCATTGTTCTTC
ATCAAAGTGGTACAGGCTCTTATTGGAGGCATATTCATGCAAATGTATG
TTAGTGGTTTCAATCAAATTTGTGATATAGAACTCGACAAGGTTAACAA
ACAGTCTCTTCCATTAGCGGCTGGAGAACTATCTATGAAAACTGCGATC
GTCATCGCATCACTATCAGCTATCATGAGCTTATCGATTGGTTGGTTTG
TTGGCTCCCCACCATTATTGTGGTGTCTTGTTTGGTGGTTTATTGTTGG
GACTGCATATTCGGCCAACGTGCTGCCTTATTTGCGATGGAAAAGGTTT
CCTTTCACAGCAGCATTTTGCGCCATGACGTCTCGGGCACTAGTTCTTC
CTATTGGATATTACTTGCATATGCAGAATTCCATCCCGGGAGTATCTGC
ATTACTTTCAAGGCCAATATTATTTGCAGTCGCAATGCTCAGTGCATTT
TCTTTATCAGCGATGTTCTTTAAGGACATCCCTGATATTAAGGGAGATA
GGATGCATGGAATCAAGTCTCTAGCAATTAAACTGGGTGAAAAACGGGT
GTATTGGATTTCCATTTCGATTATTGAAATTGCTTATATTGCTGCTGCA
TTTATTGGAGCAACTTCACCCATAAGCTGGAGCAAGTATGTAACGATTA
TCGGTCATCTTGGAATGGGATTACTACTTTGGGTACGAGCCAGATCAGT
AGATCCGACGAACACGGTAGCCGTTCAATCGATGTATATGTTCCTTATT
AAGCTAGTATATGCAGAATACGGACTTATCTCGCTTGTACGCTGA.

In some embodiments, the DNA molecule comprises a nucleic acid sequence with at least 77%, at least 79%, at least 85%, at least 95%, or at least 99% homology or identity to SEQ ID NO: 53, or any value and range therebetween. Each possibility represents a separate embodiment of the invention. In some embodiments, the DNA molecule comprises a nucleic acid sequence with 77% to 100%, 85% to 100%, 90% to 100%, or 95% to 100% homology or identity to SEQ ID NO: 53. Each possibility represents a separate embodiment of the invention.

In some embodiments, the DNA molecule comprises or consists of the nucleic acid sequence:

(SEQ ID NO: 54)
ATGAAGTCTTTGATTATTGGGTCTTTTTCTAATAAGGTTTCTTGTTATT
CCCCATCATTACCAGATTCATCTTCTTCACTTATACCAACAGGTTGTTA
TCATGTATCACTAAGAACATTTCAGCGTAACCGAGCCATTCAAGCTCAA
TCAAGTCTTGTGAGATGCAATATTGGCAAATTCAATGAAACATTACTAC
TTTCGCGGAAACGAAGTACAAAACATGTTGCATGTGCGGTTTCTGAACA
ACCCATTGAACCAGATGCTACAAACCCTCAAAGTTCATTACCAAATGCT
TTGGATGCTTTCTATAGGTTTTCAAGACCTCATACAGTTATAGGAACTG
CATTGAGCATAGTTTCGGTTTCACTCCTAGCGGTTCAAAAGCTTTCGGA
TTTTTCTCCACTATTCTTCATTGGCGTTTTCGAGGCTATTGTTGCTGCC
TTCTTTATGAACATATACATTGTTGGCTTGAACCAGCTATCCGATATTG
AAATAGACAAGGTTAACAAGCCGTACCTTCCATTGGCATCTGGAGAATA
TTCAGTTCAAACTGGTATTATCATTGTATCATCATTTGCAGTCATGAGT
TTCTGGCTTGGATGGATCGTGGGCTCATGGCCTTTATTTTGGGCACTTT
TCATAAGTTTTCTTCTAGGGACCGCATATTCAATCAATATACCGATGTT
GAGATGGAAGCGCTTTGCTCTTGTGGCAGCAATGTGTATTCTAGCTGTA
AGAGCTATTATAGTTCAAGTTGCATTTTATTTGCACATTCAGACTTTTG
TGTATGGAAGACTCGCCGTGTTCCCAAAACCCGTGATATTTGCAACCGG
ATTTATGAGTTTCTTCTCTGTTGTTATAGCATTGTTCAAGGACATACCC
GACATTGTTGGAGACAAGATTTTTGGCATTCAATCATTTACTGTCCGTA
TGGGTCAAAAACGGGTGTTTTGGATTTGCATCTTATTACTTGAAATAGC
TTATGGTGTTGCTATTCTAGTTGGGGCATCATCTCCCTTCCTTTGGAGC
CGATACATAACGGTATTGGGTCATGCGATTCTTGGTCTGATTCTCTGGG
GTCGTGCCAAGTCAACGGATCTGGAGAGCAAATCAGCAATAACCTCATT
TTACATGTTCATATGGCAGTTGTTCTATGCCGAGTATTTGCTCATACCG
CTCGTGAGATGA.

In some embodiments, the DNA molecule comprises a nucleic acid sequence with at least 89%, at least 90%, at least 92%, at least 95%, at least 96%, at least 97%, at least 98%, or at least 99% homology or identity to SEQ ID NO: 54, or any value and range therebetween. Each possibility represents a separate embodiment of the invention. In some embodiments, the DNA molecule comprises a nucleic acid sequence with 89% to 100%, 92% to 100%, 94% to 100%, or 97% to 100% homology or identity to SEQ ID NO: 54. Each possibility represents a separate embodiment of the invention.

In some embodiments, the DNA molecule comprises or consists of the nucleic acid sequence:

(SEQ ID NO: 55)
ATGTTGATTCACCATGAACATTTTTTGACAACCGGATTTGAAAGTTCAA
ACGATCGAGCTGCTTATTCAATAAACTTTTCGAAACAACATCACTTACA
CATGGCGTCTATAGCTACTGGTTCACTTTGTAGGCCAACCTCACATCAA
TTTTCTATCCCCGTTGCATCATCTTCTTCATTTGCGACAGGATCACAAT
TCGCTTCAAAGTTTCTTCATATATCAATATCTGCTAAAAAAAGCTCATT
GACATTGCAACAAAGGCATATTCATAAAAACATAGATCAAAGCTTCTTA
AAGCCGCTTGCACTTCAAAAATTGAACAAAGACAAGTTTAAGTTGAATG
GAACATCTCCAGACAATCCTCAGTTTGATGCAACTCATGATTTGAAGAC
TCAAATAGAATCCACTATCAACTTTGTGGACGTTCTTTATAGGTTGTTA
AGGCCGTATGCATTACTTCAAATGGGTTTATGTGTAGTCACGATGAGTC
TTCTTACCGTTGAAAGCCTTTCAGATTTTTCCCCATTGTTCTTCGTCAA
AGTGGCACAGGCTCTTATTGGAGGCATATTCATGCAAATGTATGTTAAT
GGTTTTAATCAGATTTGTGATATAGAACTCGACAAGGTTAACAAACCGT
CTCTTCCGTTAGCATCTGGGGAACTATCTAAGACAACTACTATAGTCGT
CTCTTCACTATCAGCTATTACGAGCTTATCGATTGGTTGGTTTGTTGGC
TCCCCACCATTGTTGTGGAGTCTTGTTGTGTGGTTTATTGCTGGGACTA
CATATTCGGCTAATCTGCCATATTTGCGATGGAAAAGGTTTCCTTTCAC
AAATATGTTTTGCAACTTGACGATGGCACTAGTTGTTCCTATTGGAACT
TACTTGCATATGGAGAATTCCATCCACGGAGTATCCACATTACTTTCAA
GGCCACTATTATTTACAGTTGCAATGTGCACTGTGTTTCCTGTTTCGAT
AATACTCTTTAAGGACATCCCTGATATTAAGGGAGACCGGATGCATGGA
ATGAAGTCTCTAGCAATTATACTGGGTGAAAAACGGACGTATTGGATAT
GCATTTGGATTCTTGAAATCACTTATATTGCTGCTGCTTTTTTCGGAGC
AACTTCACCCATCAGCTGGAGCAAATATGTAACGATTATTAGTCATCTA
GGAATGGGGTTCTTACTTTGGCTACGATCCAAATCAGTAGATGTGAAGA
ACACAGTAGCCGTTCAATCTATGTATATGTTCCTTTGGAAGCTACTCTA
TGCAGAATATGGCCTTATCTTGCTTGTACGCTGA.

In some embodiments, the DNA molecule comprises a nucleic acid sequence with at least 76%, at least 80%, at least 85%, at least 90%, at least 95%, at least 97%, at least 98%, or at least 99% homology or identity to SEQ ID NO: 55, or any value and range therebetween. Each possibility represents a separate embodiment of the invention. In some embodiments, the DNA molecule comprises a nucleic acid sequence with 76% to 100%, 83% to 100%, 90% to 100%, or 95% to 100% homology or identity to SEQ ID NO: 55. Each possibility represents a separate embodiment of the invention.

In some embodiments, the DNA molecule comprises or consists of the nucleic acid sequence:

(SEQ ID NO: 56)
ATGTTTATTCACCATGAACAGTTTTTGACAACCGGATTTGAAAGTTCAA
ACGATCGAGCTGCCTATTCAATAAACTTTTTGAAACAACATCACTTACA
CATGGTGTCTATAGCTACTGGTTCACTTTGTAGGCCAACCTCACATCGA
TTCTCTATCCCCGTTGCATCATCTTCTTCATTTGCGACAGGATCACAAT
TCGCTTCAATATCTGCTAAAAAAAGCTCATTGACATTGAAACAAAGGCA
TACTCATAAAAACATAGATCAAAGCTTCTTCAAGCCGCTTGCACTTCAA
AAAATGAACAAAGGCAAGTTTAAGTTGAATGCAACATCTCCAGACAATT
CTCAGTTGGATGCAACTCATGATTTGAAGACTCAAATAGAATCCATTAT
CAACTTTGTGGACGTTCTTTATAGGTTGATAAGGCCGTATGTAGTACTT
GGAATGGGTGTAACTATAGTCACGATGTGTCTTCTTACCGTTGATAGCC
TTTCAGATTTTTCCCCATTGTTCTTCGTCAAAGTGGCACAGGCTCTTAT
TGGAAGCATATTCATGGCAATGTATGTTAATAGTTTTAATGAGATTTGT
GATATAGAACTCGACAAGGTTAACAAACCGTCTCTTCCGTTAGCGTCTG
GGGAACTATCTATGACAACTGCTATTGTCGTCTCTTCACTATCAGCTAT
CATGAGCTTATCGATTGGTTGGTTTGTTGGCTCCCCACCATTGTTGTGG
AGTCTTGTTGTGTGGTTTATTCTTGGGACTGCATATTCGGCTAATCTGC
CATATTTGCGATGGAAAAGGTTTCCTTTAACAACACTGTCTTCCGCCCT
GACGATGGGGGCACTAGTTATTCCTATTGGAAATTACATGCATATGGAG
AATTCCATCCGCGGAGTAACCACATTACTTTCAAGGCCACTATTATTTG
CAGTTGCAATGTGCGCTGCGTTTCATGTTTCGACGATACTCTTTAAGGA
CATCCCTGATATTAAGGGAGACCGGATGCATGGAATGAAGTCTCTAGCA
ATTAAACTGGGTGAAAAACGGATGTATTGGATATGCATTTGGATTCTTG
AAATCGCTTATATTGCTGCTGCTTTTTTCGGAGCAACTTCACCCATCAG
CTGGAGCAAATATGTAACGATTATTAGTCATCTAGGAATGGGGTTCTTA
CTTTGGCTACGATCCAAATCAGTAGATGTGAAGAACACAGTAGCCGTTC
AATCTATGTATATGTTCCTTTGGAAGCTATTCTATGTAGAACATGGTCT
TATCTTGCTTGTACGTTGA.

In some embodiments, the DNA molecule comprises a nucleic acid sequence with at least 75%, at least 80%, at least 85%, at least 90%, at least 95%, at least 97%, at least 98%, or at least 99% homology or identity to SEQ ID NO: 56, or any value and range therebetween. Each possibility represents a separate embodiment of the invention. In some embodiments, the DNA molecule comprises a nucleic acid sequence with 75% to 100%, 80% to 100%, 90% to 100%, or 95% to 100% homology or identity to SEQ ID NO: 56. Each possibility represents a separate embodiment of the invention.

In some embodiments, the DNA molecule comprises or consists of the nucleic acid sequence:

(SEQ ID NO: 57)
ATGGCGTCTATAGCTACTGGTTCACTTTGTAGGCCAACCTCACATCGAT
TTTCTATCCACGTTGCATCATCTTCTTCATTTGCGACAGGATCACAGTT
TGCTTCAAAGATTCTTCAGATATCAATATCTGCTAAAAAAAGCTCATTG
ACATTGCAACAAAGGCATATTCATAAAAACATAGATCAAAGCTTCTTCA
AGCCGCTTGCACTTCAAAAAATGAACAAAGACAAGTTTAAGTTGAATGC
AACATCTCCAGACAATCCACAGTTTGATGCAACTCGTGATTTGAAGACT
CAAATAGAATCCATTATCAAGTTTGTGGACGTTCTTTATAGGTTGTTAA
GGCCGTACGCAATACTTGAAATGGGTTTAAGTGTAGTCACGATGAGTCT
TCTTACCGTTGAAAGCCTTTCAGATTTTTCCCCGTTGTTCTTCGTCAAA
GTGGCACAAGCTCTTATTGGAGGCATATTCATGCAAATGTATGTTAATG
GTTTTAATCAGATTTGTGATATAGAACTCGACAAGGTTAACAAACCGTC
TCTTCCGTTAGCGTCTGGGGAACTATCTACGACAACTACTATAGTCGTC
TCTTCACTATCAGCTATTATGAGCTTATCGATTGGTTGGTTTGTTGGCT
CCCCACCATTGTTGTGGAGTCTTGTTGTGTGGTTTATTGTTGGGACAAC
ATATTCGACTAATCTGCCATATTTGCGATGGAAAAGGTTTCCTTTCACA
GCAATGTTTTGCAACCTGACGAGGGCACTAGTTGTTCCTATTGGAACTT
ACTTGCATATGAAGAATTCCATCCACGAAGTATCCACATTACTTTCAAG
GCCACTGTTATTTGCAGTTGCAATGTGCACTGTGTTTCCTATTTCGATA
ATACTCTTTAAGGACATCCCTGATATTAAGGGAGACCGGATGCATGGAA
TGAAGTCTCTAGCAATTATACTGGGTGAAGAACGGACGTATTGGATATG
CATTTGGATTCTTGAAATCGCTTATATTGCTGCTGCTTTTTTCGGAGCA
ACTTCACCCATCAGCTGGAGCAAATATGTAATGATTATTAGTCATCTAG
GAATGGGGTTCTTACTTTGGCTACGATCCAAATCAGTAGATGTGAAGAA
CACAGTAGCCGTTCAATCTATGTATATGTTCCTTTGGAAGCTACTCTAT
GCAGAATATGGCCTTATTTTGCTTGTACGCTGA.

In some embodiments, the DNA molecule comprises a nucleic acid sequence with at least 76%, at least 80%, at least 85%, at least 90%, at least 95%, at least 97%, at least 98%, or at least 99% homology or identity to SEQ ID NO: 57, or any value and range therebetween. Each possibility represents a separate embodiment of the invention. In some embodiments, the DNA molecule comprises a nucleic acid sequence with 76% to 100%, 85% to 100%, 90% to 100%, or 96% to 100% homology or identity to SEQ ID NO: 57. Each possibility represents a separate embodiment of the invention.

In some embodiments, the DNA molecule comprises or consists of the nucleic acid sequence:

(SEQ ID NO: 58)
ATGGCATCTCTAGCTATTGGTTCACTTGGTAGCCCAAGCTCACGTCAGT
GTTCTAGCCCCGTTGCATCATCTTCTTCATTTGCGATAGGGTCACAAAT
AGCTTCAAAGTTTCTTCGGATATCAAAATTTGATAAGACTAAGAACAGC
CCCTTAGCATTGCAACAAAAGCATATAAACAAAAGCATAGATCAAAGCT
TCTTTGAGCCGCTTCCATTGCACAAAATAAACAAAGACAAGTTTAAGTT
GTATGCAACATCTACAAACAATCCTCAGTTTGATGCAACTCATGATTTG
AAGACTCCGGAAGTATCCATTATCAACTTTGTGGACGCTCTTTATAGGT
TAATAAGGCCGTATACAGCAGTTGTAACGATCGTAAGTGTAGTCGCGAT
GTCCCTTCTTACAGTTAATAGCCTTTCAGATTTTTCCCCATTGTTCTTC
ATCAAAGTGGTACAGGCTCTTATTGGAGGCATATTCATGCAAATGTATG
TTAGTGGTTTCAATCAAATTTGTGATATAGAACTCGACAAGGTTAACAA
ACAGTCTCTTCCATTAGCGGCTGGAGAACTATCTATGAAAACTGCGATC
GTCATCGCATCACTATCAGCTATCATGAGCTTATCGATTGGTTGGTTTG
TTGGCTCCCCACCATTATTGTGGTGTCTTGTTTGGTGGTTTATTGTTGG
GACTGCATATTCGGCCAACGTGCTGCCTTATTTGCGATGGAAAAGGTTT
CCTTTCACAGCAGCATTTTGCGCCATGACGTCTCGGGCACTAGTTCTTC
CTATTGGATATTACTTGCATATGCAGAATTCCATCCCGGGAGTATCTGC
ATTACTTTCAAGGCCAATATTATTTGCAGTCGCAATGCTCAGTGCATTT
TCTTTATCAGCGATGTTCTTTAAGGACATCCCTGATATTAAGGGAGATA
GGATGCATGGAATCAAGTCTCTAGCAATTAAACTGGGTGAAAAACGGGT
GTATTGGATTTCCATTTCGATTATTGAAATTGCTTATATTGCTGCTGCA
TTTATTGGAGCAACTTCACCCATAAGCTGGAGCAAGTATGTAACGATTA
TCGGTCATCTTGGAATGGGATTACTACTTTGGGTACGAGCCAGATCAGT
AGATCCGACGAACACGGTAGCCGTTCAATCGATGTATATGTTCCTTATT
AAGCTAGTATATGCAGAATACGGACTTATCTCGCTTGTACGCTGA.

In some embodiments, the DNA molecule comprises a nucleic acid sequence with at least 77%, at least 79%, at least 85%, at least 95%, or at least 99% homology or identity to SEQ ID NO: 58, or any value and range therebetween. Each possibility represents a separate embodiment of the invention. In some embodiments, the DNA molecule comprises a nucleic acid sequence with 77% to 100%, 85% to 100%, 90% to 100%, or 95% to 100% homology or identity to SEQ ID NO: 58. Each possibility represents a separate embodiment of the invention.

In some embodiments, the DNA molecule comprises or consists of the nucleic acid

(SEQ ID NO: 71)
ATGGGGCTAAACATTTGCACTAGATTTATACCTTGTTTGGTAGTGGTTC
TCATGTTTTTGTTCACTTCAACATATTCAGCTACACCAGAAGACAAATT
CCTTCAATGCATATCTCAAAAATTAAATATCACAAACTCAGATGAAGTG
TTCACTCAATCAAACACACGATATTCATCTGTTCTTGAGTCAACAATAG
TTAACCTTAGATTTGCCACTTCTACAACGCCAAAACCATTTGCTATAAT
CACACCTTTGTCATATTCACATGTACAATCTGCTGTAGTTTGTGCTAAA
AAAGCCGGAATCCGAATTAGAATCAGAAGTGGTGGCCATGACTATGTGG
GCCTTTCATATACTTCATCTGATAATGTCCCTTTTGTTGTTCTTGACCT
TAAACAGCTGCAGAATGTTACGGTCGAGTATAGTAAGAAAACGGCTTGG
GTTGAATCTGGTGCAACCATCGGTCAACTGTATTATTGGGTGTCTCAGA
AAAGTAAAAATCTAGGATTCCCGGGTGGGACCTGCGCAACTATAGGGGT
CGGAGGGCACCTAAGTGGTGGGGGTTTTGGTACTTTGGTAAGAAAGTAT
GGTCTATCGGCTGATAACGTTATTGATGCTAAGATAGTTGATGTCAATG
GTAGACTTCTTGATAGAAAGTCTATGGGGGAAGATTTGTTTTGGGCAAT
TAGAGGAGGCGGTGGAGGAAGTTTCGGTGTTGTAGTAGCTTGGATGGTC
AATCTTGTTCATGTTCCTGAAAAAGTTACAGCTTTTACTATTGTCAGGA
CTTTGGAACAAGGTGGTTCGGATCTTTTCAACAAGTGGCAGCACGTTGG
GCCCAAATTAACCAAAGATTTGTTCATTAGTGTTATAATACAGCCCATT
TCTGTTTGGAATGGAAACGGAACAGTTCAAGTTATATTCAACTCGATGT
ATCTTGGGACGGTTGATAAGCTCATGAAGACCGTCAACAGTAGCTTTCC
GGAGTTGGGGTTACAAGCAAAAGACTGCACTGAGATGAGTTGGATTCAG
TCAGTACTTTATTTTGCGGGTTACCCTATAGAAGGAAGTATGGATGTTC
TTAAAGATAGGAAACCCCAGACCAGAAGATACTTTAATAATAAATCAGA
TCACGTGAAAGAACCGATACCCAAAGAAAGATTAGAAGATTTATGGAAA
TGGTGTATGGAAGGTGATTTTCCGATTCTTCTAATGGACCCACTCGGTG
GAAAGATGAACGAGATTGACACAACAAGAATTCCGTACCCTTATAGAAA
TGGTTATTCGTATATGATACAATACGTTGAGACCTGGGAAAACATTGGG
GACTCAGAAAAGCGTATAAGTTGGATGAGACAGATGTATGAGAATATGA
CACCGTATGTGTCGAAGAATCCAAGGTCAGCTTATGTGAATTATAGGGA
TTTGGATTTAGGTAAAAACGATAACGCTAAAAACACGAGTTACTTGGAA
GCCATGAAATGGGGAAGCAAGTACTTTGGTGACAATTTCAAGAGGTTGG
CTATGGTGAAAGGTGTAGTTGATCCAGACAATTTCTTCTTTCATGAACA
AAGCATCCCACCTCTGAAAGTGTGA.

In some embodiments, the DNA molecule comprises a nucleic acid sequence with at least 68%, at least 75%, at least 85%, at least 95%, or at least 99% homology or identity to SEQ ID NO: 71, or any value and range therebetween. Each possibility represents a separate embodiment of the invention. In some embodiments, the DNA molecule comprises a nucleic acid sequence with 68% to 95%, 75% to 100%, 72% to 99%, or 68% to 100% homology or identity to SEQ ID NO: 71. Each possibility represents a separate embodiment of the invention.

In some embodiments, the DNA molecule comprises or consists of the nucleic acid

(SEQ ID NO: 72)
ATGGGGTGTAATCTCTTGCAAAAACTTACTATTTTTGTTTTCTTTATCA
TGTCTATTTCCATACCTTCTTTCGCTTACGAACACGAGCACGAGCATGA
GCACGAACACGAAAATGATCAAGATCGAGTACAGGATGAAAAGGAACCT
ACGGATGTCTTCACTTCGTGTTTAACTCGGTTCGGTGTTCATAATTTTA
CAACTCATTCCAAGTCGAATAATGATAATTCGGTTTACTATGAGCTTCT
TAATTTTTCAATTCAAAATCTTAGATTTACGGGTTTATCGATGCCTAAA
CCGGTTGTTATCGTGTTCCCGGAGACGAAAGAACAGTTAGCAAAAACCG
TGGTTTGTGCTCGAGAATCGTCGCTAGAAATTCGGGTTCGGTGTGGTGG
TCATAGCTATGAAGGGACATCATCCGTCTCCACGGACGGACGTCCATTT
GTGGTGATTGATATGACGAGATTAGACAATGTTTCGGTGGACGTGAACT
CGGGAACCGCATGGGTTGAAGCTGGCGCGACACTTGGTCAAATGTACTG
CGCGATAGCAGAGTCGAGCACGGTCCATGGTTTCTCGGCAGGGTCATGC
CCCACTGTCGGAACAGGTGGTCATATTTCGGGTGGTGGGTTTGGGTTAT
TGTCGCGAAAATACGGGCTGGCTGCGGATAATGTAGTCGATGCGGTTTT
AGTAACCGCAGATGGTGAATTACTGAACCGCGACACGATGGGTGAGGAT
GTTTTTTGGGCGATTAGAGGTGGTGGTGGCGGGGTTTGGGGAATTGTGT
ACGCTTTTAATGTTAAATTATCAAGCGTACCAAAAACAGTCACTAATTT
CGTCGTGTCTAGGCCAGGCACGAAGGGACAAGTGACTGATTTGGTATAT
AAATGGCAGCATGTTGCGCCTAAATTGCCCGACGACTTCTACTTATCCT
CTTTCGTTGGTGCGGGTTTGCCTGAACGAAAAAATAAACCGGGTTTATC
GGCTACGTTCAAAGGTTTTTATTTGGGATCGAAAAGCAAAGCTTTATCG
ATCATGAACCAAACTTTCCCCGAGCTAAAAGTCATGGAAAACGACTGTA
AAGAAACAAGTTGGATTGAGTCTATTCTTTTCTTCTCGGGTTATGGAGA
TGAAAGCTCGGTTTCTGACTTGAAAAATCGCTTCTTACAAGATAAATTG
TATTACAAGGCCAAATCGGACTATGTTCGGAAACCTATTCCAAGATTCG
GTCTAACTACGGCACTAGAAATACTCGAGAAACAACCAAAAGGGTATGT
GATCTTGGACCCATATGGTGGCGCAATGCAAACGATAAGTAGTGACTCG
ATCCCGTTCCCTCATAGGAAAGGTAATATTTTCACTATTCAATATCTAG
TGGAATGGAAAGAACCGGATAACGATAAAACGAATGATTACTTAGCGTG
GATACGAGACTTTCATGGCTCGATGACGCCCTATGTGGCACAAGACCCA
CGAGCCGCATACATTAACTACATGGATGTTGATATTGGAGTCATGAATT
GGATCAAAACTAGAGTGGACTCAGATGATGCAGTTGAGATGGGTCGAGA
ATGGGGGGAGAAGTACTTTTATAAGAATTACGATCGGCTAGTGAGAGCG
AAGACACAAATCGATCCGTACAATGTTTTTAGGCATCAACAAAGCATCC
CTCCAATGTCTTTGGAGAACAAGAATCGCAGGGGAAGTATATCTAGTGA
GTAG.

In some embodiments, the DNA molecule comprises a nucleic acid sequence with at least 71%, at least 77%, at least 85%, at least 93%, at least 97%, or at least 99% homology or identity to SEQ ID NO: 72, or any value and range therebetween. Each possibility represents a separate embodiment of the invention. In some embodiments, the DNA molecule comprises a nucleic acid sequence with 71% to 95%, 75% to 98%, 80% to 99%, or 71% to 100% homology or identity to SEQ ID NO: 72. Each possibility represents a separate embodiment of the invention.

In some embodiments, the DNA molecule comprises or consists of the nucleic acid

(SEQ ID NO: 73)
ATGAAAACATCATCAAATATGCTTTCCGTATTACTCATTCTATTCTTTA
TCACATGCTCAAAAGCAGCTCTGGATCCTGATTCCGTCTATCAATCATT
TCTCCAATGTTTACCGTTATACTCACCGGAGTCCGCGGAGGAACTCTCC
AAGGTCGTATACAGCTCCACCTTGAACACCACAACATACGAAACCGTAC
CGAACGATTTAACACCACCGCGACACCCAAACCGTCGGTTATCATAACC
CAACCGAATCTCAAGTCCAAGCGGCCGTCCTATGCGCGAAAAAAACCGG
TCTCCAAGAGTACATAAAAAACGAGTCCAAATTAAAATTCGTAGCGGCG
GACACGACTACGAAGGAATATCGTATATTTCATCCGAACCTGATTTTAT
CGTACTTGACATGTTTAACTTTCGGTCGATAAATGTTAATGTAGCGGAC
GAAACCGCGGTTGTGGGCGCCGGCGCGCAGTTGGGCGAGCTTTATTATA
GGATTTACGAAAAAAGTAAAACTCTCGGGTTCCCCGCGGGAGTTTGTCA
GACGGTTGGCGTGGGAGGTCATCTGAGCGGCGGTGGTTACGGAACTATG
CTGCGAAAATACGGGTTGTCAGTTGATCATGTGATTGATGCGAAAATTG
TTGATGTGAATGGTCAGGTTTTGGATCGGAAATCGATGGGTGAGGATCT
ATTTTGGGCGATACGAGGTGGCGGTGGCGGTAGTTTTGGTGTGATTTTG
TCGTATACTGTGAAGTTGGTTTCGGTTCCCGAGGTTAACACGGTCTTTC
GCGTGCTGAAAACGACGTCGGAAAATGCTTCTGAACTGATTTATAAGTG
GCAGTCGATTATGCCGGATATTGATAACGATTTGTTTATCAGAGTTTTG
TTACAACCGGTTACGGTGAATAAACAGAAAGTTGGTCGGGCTACGTTTA
TAGCGCATTTTTTAGGTGATTCTGATAGATTGGTGGCGTTGATGAGTAA
AAACTTCCCGGAATTGGGTTTAAAGAAAGAGGATTGTATCGAGGTGAGT
TGGATAGAATCGGTACTTTATTGGGCTAACTTTGATTTGAATACGACGA
AGCCAGAGATTCTTCTAGATCGACATTCCGACAGTGTGAGCTATGGTAA
ACGAAAGTCGGACTATGTGCAAACCCCGATTCCTGAATCCGGGTTGGAA
TCGATTTTTGAAAAGTTAGTCGAATTGGGTAAAATCGGGTTGGTTTTTA
ACTCGTATGGCGGGAGAATGTCGGAGGTTGCGGCTGACGCAACACCATT
CCCTCACCGAGCTGGGAACATTTTCAAGATTCAGTATTCGGTTAATTGG
AATGATGCGGACCCTGAACTAGAAGCGAATTACTTAAATCAAAGTAGGG
TTATGTACGACTTCATGACACCATTTGTATCGAAGAATCCGAGAGCTGC
ATTCTTGAATTATCGGGATCTCGATATTGGAGTAATGACTCCTGGCAAG
AACAGTTATAGTGAAGGTGAAGTTTATGGTGAGAAATACTTCATGGGAA
ATTTCGAAAGATTGGTGAAGATAAAAACCGCGGTTGATCCCGATAATTT
CTTTAGAAATGAACAAAGTATTCCGACTCGGGCCGCGAAAAATTCAGGC
AAGTCAAGAAAGATGATGAAGTAA.

In some embodiments, the DNA molecule comprises a nucleic acid sequence with at least 69%, at least 75%, at least 85%, at least 95%, or at least 99% homology or identity to SEQ ID NO: 73, or any value and range therebetween. Each possibility represents a separate embodiment of the invention. In some embodiments, the DNA molecule comprises a nucleic acid sequence with 69% to 95%, 75% to 100%, 72% to 99%, or 69% to 100% homology or identity to SEQ ID NO: 73. Each possibility represents a separate embodiment of the invention.

In some embodiments, the DNA molecule comprises or consists of the nucleic acid sequence:

(SEQ ID NO: 74)
ATGGGGCTAAACATTTGCACTAGATTTATACCTTGTTTGGTAGTGGTTC
TCATGTTTTTGTTCACTTCAACATATTCAGCTACACCAGAAGACAAATT
CCTTCAATGCATATCTCAAAAATTAAATATCACAAACTCAGATGAAGTG
TTCACTCAATCAAACACACGATATTCATCTGTTCTTGAGTCAACAATAG
TTAACCTTAGATTTGCCACTTCTACAACGCCAAAACCATTTGCTATAAT
CACACCTTTGTCATATTCACATGTACAATCTGCTGTAGTTTGTGCTAAA
AAAGCCGGAATCCGAATTAGAATCAGAAGTGGTGGCCATGACTATGTGG
GCCTTTCATATACTTCATCTGATAATGTCCCTTTTGTTGTTCTTGACCT
TAAACAGCTGCAGAATGTTACGGTCGAGTATAGTAAGAAAACGGCTTGG
GTTGAATCTGGTGCAACCATCGGTCAACTGTATTATTGGGTGTCTCAGA
AAAGTAAAAATCTAGGATTCCCGGGTGGGACCTGCGCAACTATAGGGGT
CGGAGGGCACCTAAGTGGTGGGGGTTTTGGTACTTTGGTAAGAAAGTAT
GGTCTATCGGCTGATAACGTTATTGATGCTAAGATAGTTGATGTCAATG
GTAGACTTCTTGATAGAAAGTCTATGGGGGAAGATTTGTTTTGGGCAAT
TAGAGGAGGCGGTGGAGGAAGTTTCGGTGTTGTAGTAGCTTGGATGGTC
AATCTTGTTCATGTTCCTGAAAAAGTTACAGCTTTTACTATTGTCAGGA
CTTTGGAACAAGGTGGTTCGGATCTTTTCAACAAGTGGCAGCACGTTGG
GCCCAAATTAACCAAAGATTTGTTCATTAGTGTTATAATACAGCCCATT
TCTGTTTGGAATGGAAACGGAACAGTTCAAGTTATATTCAACTCGATGT
ATCTTGGGACGGTTGATAAGCTCATGAAGACCGTCAACAGTAGCTTTCC
GGAGTTGGGGTTACAAGCAAAAGACTGCACTGAGATGAGTTGGATTCAG
TCAGTACTTTATTTTGCGGGTTACCCTATAGAAGGAAGTATGGATGTTC
TTAAAGATAGGAAACCCCAGACCAGAAGATACTTTAATAATAAATCAGA
TCACGTGAAAGAACCGATACCCAAAGAAAGATTAGAAGATTTATGGAAA
TGGTGTATGGAAGGTGATTTTCCGATTCTTCTAATGGACCCACTCGGTG
GAAAGATGAACGAGATTGACACAACAAGAATTCCGTACCCTTATAGAAA
TGGTTATTCGTATATGATACAATACGTTGAGACCTGGGAAAACATTGGG
GACTCAGAAAAGCGTATAAGTTGGATGAGACAGATGTATGAGAATATGA
CACCGTATGTGTCGAAGAATCCAAGGTCAGCTTATGTGAATTATAGGGA
TTTGGATTTAGGTAAAAACGATAACGCTAAAAACACGAGTTACTTGGAA
GCCATGAAATGGGGAAGCAAGTACTTTGGTGACAATTTCAAGAGGTTGG
CTATGGTGAAAGGTGTAGTTGATCCAGACAATTTCTTCTTTCATGAACA
AAGCATCCCACCTCTGAAAGTGTGA.

In some embodiments, the DNA molecule comprises a nucleic acid sequence with at least 79%, at least 85%, at least 92%, at least 97%, or at least 99% homology or identity to SEQ ID NO: 74, or any value and range therebetween. Each possibility represents a separate embodiment of the invention. In some embodiments, the DNA molecule comprises a nucleic acid sequence with 79% to 98%, 80% to 99%, 82% to 99%, or 79% to 100% homology or identity to SEQ ID NO: 74. Each possibility represents a separate embodiment of the invention.

In some embodiments, the DNA molecule comprises or consists of the nucleic acid

(SEQ ID NO: 75)
ATGGACCAATATGTCATAACTAAATTTATATCATATCTTCTGGCGGTTTT
TATGGCTTTATTCTGTTCAGATCCAACGGCTGATAAATTTCTTCAATGCT
TCACTAAAGATTCAAATGCAACAGATTCAAACTTTGTGTTCACCCAAGAA
AACACACAATATTCATCTGTTCTTGAGTCAACTATCATAAACCTTAGATT
TGCAACCTCCATAACTCCAAAACCAATAGCTGTAATCACACCATTATCAT
ATTCCCATGTACAATCAGCAATACTTTGTTCCAAAAAAATCGGATATCGA
ATTAGAATCAGAAGTGGTGGGCATGACTATGCAGGAGTTTCATACACTTC
ATATGATCATGATCATACCCCTTTTGTTGTTCTTGATCTTAAAGAGCTGA
GGACGATAACAATCGATTCGGGTGAGAACACTTCATGGGTTGAATCTGGT
GCAACTGTTGGTGAACTGTATTATTGGGTGTCCCAAAAAAGTCGAAATCT
TGGGTTCCCAGCTGGGATTTGTCCAACTGTTGGGGTAGGTGGTCATTTAA
GTGGAGGTGGGGTTGGTACTATGGTAAGAAAGTATGGTCTAGCGGCTGAT
AATGTAATCGATGCTAGGATTATTGATGTAAATGGGCGAATTCTTGATAG
GAAATCGATGGGGGAAGATTTGTTTTGGGCGATTAGAGGTGGTGGGGGAG
CTAGTTTTGGTGTTATAGTAGCTTGGAAGGTAAATCTTGTTTATGTTCCT
GAAAAAAGTTTCGGTTTTTAG.

In some embodiments, the DNA molecule comprises a nucleic acid sequence with at least 82%, at least 87%, at least 92%, at least 96%, or at least 99% homology or identity to SEQ ID NO: 75, or any value and range therebetween. Each possibility represents a separate embodiment of the invention. In some embodiments, the DNA molecule comprises a nucleic acid sequence with 82% to 98%, 83% to 99%, 85% to 99%, or 82% to 100% homology or identity to SEQ ID NO: 75. Each possibility represents a separate embodiment of the invention.

In some embodiments, the DNA molecule comprises or consists of the nucleic acid

(SEQ ID NO: 76)
ATGGAGTTGTATATTAGCACTAGATTTATACTATGTTTTCTAGTGGTTCT
TATGCTTATGTTCTCTTCAACATATTCAGATCCACTAGAAGATAAATTTC
TTCGATGTCTATCTCAAAATTCAAATGCCACAAATTCAGACAATGTGTTC
ACTCAAGAAAACACACAGTATTCATCTGTTCTTGAGTCAACTATCATAAA
CCTTAGATTTGCAACCTCTACAACTCCGAAACCGTTAGCTATAATCACAC
CGTTGTCATGTTCCCATGTACAATCTGCTGTACTTTGTGCCAAAAAAGTC
GGAATCCGAATTAGAATCAGAAGTGGTGGCCATGACTATGCAGGCCTTTC
ATACACTTCATCTGAGAATGCCCCTTTTGTTGTTCTTGATCTTAAACAGC
TGCAGAATGTTACGGTCGAGTCTAGTAAGAAAACGGCTTGGGTTGAATCT
GGTGCAACCATCGGTCAATTGTATTATTGGGTGTCTCAAAAAAGTAAAAA
TCTAGGATTCCCAGCTGGGACCTGCGCGACTATAGGGGTCGGAGGGCACC
TAAGTGGTGGGGGTTTCGGTACTTTGGTAAGAAAGTATGGTCTATCGGCT
GATAACGTCATCGATGCTAAGATAGTTGATGTCAATGGTAGACTTCTTGA
TAGAAAGTCTATGGGGGAAGATTTGTTTTGGGCAATTAGAGGAGGCGGTG
GAGGAAGTTTCGGTGTTGTAGTAGCTTGGAAGGTCAATCTTGTTCATGTT
CCCGAAAAAGTTACGGCTTTTACTATTGTCAGGACTTTGGAACAAGGTGG
TTCGGATATTTTCAACAAATGGCAGCACATTGGGCACAAATTAACTAAAG
ATTTGTTCATTAGAGTTATAATACAGCCTATTTCTGTTTCGAATGGAAAC
AGAACAGTTCAAGTTATATTCAACTCGATGTATCTGGGGACGGTTGATAA
GCTCATGAAGACCGTCAACAGTAGCTTCCCGGAGTTGGGCTTACAAGAAA
AAGACTGCACTGAGATGAGTTGGATTCAGTCAGTACTTTATTTTGCGGGT
TACCCAATAGAAGGAAGTATGGATGTTCTTAAAGATAGGAAACCCGACAC
CCGAAATTACTTTGATAATAAATCAGATCACGTGAAAGAACCGATACCCA
AAGAAAGATTAGAAGATCTATGGAAATGGTGTATGGAAGTTGATTTTCCG
ATTCTTATAATGGAGCCACTCGGTGGAAAGATGAACGAGATTGACACAAC
AAGAATTCCATACCCTTATAGAAAAGGTTATTCGTATATGATACAATATG
TTGAGGCTTGGGATAACATTGGGGACTCGGAAAAACATATAAGTTGGTTG
AGACAGATGTATGAGAATATGACACCATATGTGTCGAAGAATCCAAGGTC
AGCTTATGTGAATTACCGGGATTTGGATTTAGGTAAAAACGATAACGCTA
AAAACACGAGTTACTTGGAAGCCATGAAATGGGGAAGCAAGTACTTTGGT
GACAATTTCAAGAGGTTGGCTATGGTGAAAGGTGTAGTTGATCCAGACAA
TTTCTTCTTTCATGAACAAAGCATCCCACCTCTGAAAGTGTGA.

In some embodiments, the DNA molecule comprises a nucleic acid sequence with at least 80%, at least 87%, at least 93%, at least 97%, or at least 99% homology or identity to SEQ ID NO: 76, or any value and range therebetween. Each possibility represents a separate embodiment of the invention. In some embodiments, the DNA molecule comprises a nucleic acid sequence with 80% to 98%, 81% to 99%, 85% to 99%, or 80% to 100% homology or identity to SEQ ID NO: 76. Each possibility represents a separate embodiment of the invention.

In some embodiments, the DNA molecule comprises or consists of the nucleic acid sequence:

(SEQ ID NO: 77)
ATGGGGCTAAACATTTGCACTAGATTTATACCTTGTTTGGTAGTGGTTCT
CATGTTTTTGTTCACTTCAACATATTCAGCTACACCAGAAGACAAATTCC
TTCAATGCATATCTCAAAAATTAAATATCACAAACTCAGATGAAGTGTTC
ACTCAATCAAACACACGATATTCATCTGTTCTTGAGTCAACAATAGTTAA
CCTTAGATTTGCCACTTCTACAACGCCAAAACCATTTGCTATAATCACAC
CTTTGTCATATTCACATGTACAATCTGCTGTAGTTTGTGCTAAAAAAGCC
GGAATCCGAATTAGAATCAGAAGTGGTGGCCATGACTATGTGGGCCTTTC
ATATACTTCATCTGATAATGTCCCTTTTGTTGTTCTTGACCTTAAACAGC
TGCAGAATGTTACGGTCGAGTATAGTAAGAAAACGGCTTGGGTTGAATCT
GGTGCAACCATCGGTCAACTGTATTATTGGGTGTCTCAGAAAAGTAAAAA
TCTAGGATTCCCGGGTGGGACCTGCGCAACTATAGGGGTCGGAGGGCACC
TAAGTGGTGGGGGTTTTGGTACTTTGGTAAGAAAGTATGGTCTATCGGCT
GATAACGTTATTGATGCTAAGATAGTTGATGTCAATGGTAGACTTCTTGA
TAGAAAGTCTATGGGGGAAGATTTGTTTTGGGCAATTAGAGGAGGCGGTG
GAGGAAGTTTCGGTGTTGTAGTAGCTTGGATGGTCAATCTTGTTCATGTT
CCTGAAAAAGTTACAGCTTTTACTATTGTCAGGACTTTGGAACAAGGTGG
TTCGGATCTTTTCAACAAGTGGCAGCACGTTGGGCCCAAATTAACCAAAG
ATTTGTTCATTAGTGTTATAATACAGCCCATTTCTGTTTGGAATGGAAAC
GGAACAGTTCAAGTTATATTCAACTCGATGTATCTTGGGACGGTTGATAA
GCTCATGAAGACCGTCAACAGTAGCTTTCCGGAGTTGGGGTTACAAGCAA
AAGACTGCACTGAGATGAGTTGGATTCAGTCAGTACTTTATTTTGCGGGT
TACCCTATAGAAGGAAGTATGGATGTTCTTAAAGATAGGAAACCCCAGAC
CAGAAGATACTTTAATAATAAATCAGATCACGTGAAAGAACCGATACCCA
AAGAAAGATTAGAAGATTTATGGAAATGGTGTATGGAAGGTGATTTTCCG
ATTCTTCTAATGGACCCACTCGGTGGAAAGATGAACGAGATTGACACAAC
AAGAATTCCGTACCCTTATAGAAATGGTTATTCGTATATGATACAATACG
TTGAGACCTGGGAAAACATTGGGGACTCAGAAAAGCGTATAAGTTGGATG
AGACAGATGTATGAGAATATGACACCGTATGTGTCGAAGAATCCAAGGTC
AGCTTATGTGAATTATAGGGATTTGGATTTAGGTAAAAACGATAACGCTA
AAAACACGAGTTACTTGGAAGCCATGAAATGGGGAAGCAAGTACTTTGGT
GACAATTTCAAGAGGTTGGCTATGGTGAAAGGTGTAGTTGATCCAGACAA
TTTCTTCTTTCATGAACAAAGCATCCCACCTCTGAAAGTGTGA.

In some embodiments, the DNA molecule comprises a nucleic acid sequence with at least 79, at least 85%, at least 95%, or at least 99% homology or identity to SEQ ID NO: 77, or any value and range therebetween. Each possibility represents a separate embodiment of the invention. In some embodiments, the DNA molecule comprises a nucleic acid sequence with 79% to 95%, 82% to 97%, 81% to 98%, or 79% to 100% homology or identity to SEQ ID NO: 77. Each possibility represents a separate embodiment of the invention.

In some embodiments, the DNA molecule comprises or consists of the nucleic acid

(SEQ ID NO: 78)
ATGGGGGAAGATTTGTTTTGGGCAATTAGAGGAGGCGGTGGAGGAAGTTT
CGGTGTTGTAGTAGCTTGGATGGTCAATCTTGTTCATGTTCCTGAAAAAG
TTACAGCTTTTACTATTGTCAGGACTTTGGAACAAGGTGGTTCGGATCTT
TTCAACAAGTGGCAGCACGTTGGGCCCAAATTAACCAAAGATTTGTTCAT
TAGTGTTATAATACAGCCCATTTCTGTTTGGAATGGAAACGGAACAGTTC
AAGTTATATTCAACTCGATGTATCTTGGGACGGTTGATAAGCTCATGAAG
ACCGTCAACAGTAGCTTTCCGGAGTTGGGGTTACAAGCAAAAGACTGCAC
TGAGATGAGTTGGATTCAGTCAGTACTTTATTTTGCGGGTTACCCTATAG
AAGGAAGTATGGATGTTCTTAAAGATAGGAAACCCCAGACCAGAAGATAC
TTTAATAATAAATCAGATCACGTGAAAGAACCGATACCCAAAGAAAGATT
AGAAGATTTATGGAAATGGTGTATGGAAGGTGATTTTCCGATTCTTCTAA
TGGACCCACTCGGTGGAAAGATGAACGAGATTGACACAACAAGAATTCCG
TACCCTTATAGAAATGGTTATTCGTATATGATACAATACGTTGAGACCTG
GGAAAACATTGGGGACTCAGAAAAGCGTATAAGTTGGATGAGACAGATGT
ATGAGAATATGACACCGTATGTGTCGAAGAATCCAAGGTCAGCTTATGTG
AATTATAGGGATTTGGATTTAGGTAAAAACGATAACGCTAAAAACACGAG
TTACTTGGAAGCCATGAAATGGGGAAGCAAGTACTTTGGTGACAATTTCA
AGAGGTTGGCTATGGTGAAAGGTGTAGTTGATCCAGACAATTTCTTCTTT
CATGAACAAAGCATCCCACCTCTGAAAGTGTGA.

In some embodiments, the DNA molecule comprises a nucleic acid sequence with at least 80%, at least 85%, at least 89%, at least 92%, at least 95%, at least 97%, or at least 99% homology or identity to SEQ ID NO: 78, or any value and range therebetween. Each possibility represents a separate embodiment of the invention. In some embodiments, the DNA molecule comprises a nucleic acid sequence with 80% to 95%, 85% to 98%, 89% to 99%, or 80% to 100% homology or identity to SEQ ID NO: 78. Each possibility represents a separate embodiment of the invention.

In some embodiments, the DNA molecule comprises or consists of the nucleic acid sequence:

(SEQ ID NO: 79)
ATGGAGTTGAAGTTGTTTACATGTAAACTCGTAACAATTATTCTAGCTCT
GTCCCTCAGTTTTTTCACATCAACAAGCTCTAGTGACTTTCTTGATTGCA
TCTCTCAAAAAAACTTATCAAATATTATTTTCACTCCTAATGACACTTCA
TACTCAACTATTCTCCAATTTACCATCCCAAATCTTAGATTTAACACGCC
TAAAACCACAAAACCATTAGCAATAATCACACCTACAACGTATTCTCACG
TACAATCTACTATAATATGCAGCGTGCAATTCAAGCACCATGTTCGCATC
CGAAGTGGTGGTCATGACTACGAAGGTCTTTCGTATACTTCTTTCAATAA
CACCCCTTTTATACTTCTTGATCTCAACCAACTTCGGTCAGTAACGGTTG
ATTTAGATAGTAATACCACATGGGTCGAATCTGGTGCCACTCTAGGTGAA
CTTTTGTATTGGGTGTCTCGAAAAAGTAATATTCTTGGGATCCCAACCGG
CGAGTGTACATCGGTGGGCGTTGGGGGACAATTAAGTGGAGGAGGGTTTG
GAAATATGGCTAGAAAATATGGATTATTTTCGGATAATGCGGTTGACGCA
CTTATCATTGATGTAAATGGACGAATACTGGATAGAGATTCCATGGGTGA
AGATTTGTTTTGGGCAATTAGAGGAGGTGGGGGTGGAAATTTTGGAGTTG
TATTATCTTGGAAGATTAATCTAGTTTATGTTCCACCTAAAGTTACGGTT
TTTACTGTTTCTAAGATGTTAGATGAAAATGGTACCAAGATTGTTCACAA
GTGGCAATATATTGCGCATAATATAACGCAAGATTTGTTCATTAATCTTA
TAGTAAGTCCGGTTACCGTGTCAAATACAACGATTCTAGCAGTAACAATT
AACTCGTTGTTTTTGGGGATGAAAAACGAGCTTGTAGCAACAATGGATGT
AATATTTCCGGAATTAGGGTTACAAGAAAAGGATTGCATCGAAATGAGTT
GGATAGAATCGGTGGTTTACCATTCGGTTTATTTAAGAGGACAAAGTGTT
GATGCTCTAATAGAAAGAAGACCATGGCCTAAAAGTTACAACAAGTATAA
ATCAGATTATGTGAAGAAACCTATGTCAGAGAAAGCGCTTGAAAAACTGT
GGAAATGGTGTTTGGAAGAGAATTTGATTCTGGCGATCGAGCCACATGGT
GGAAAGATGAGCGAGATCGATGAGAGTTCGACTCCGTATCCGCATAGAAA
AGGGAATTTGTACATCATACAATATGTCATGCAATGGGATGAAGGGTATA
ACACAACTCAAAAGCATGTTGCTTCCATAAGAAGGGTATATAAGAAAATG
GCACCTTTTGTGTCCAAGAACCCTAGGGAAGCTTATGTGAACTTTAGAGA
TTTGGATTTGGGTACTAATGGTAATGCATGTGGTACAAGTGGTGCAAGCT
ATGTGCAAGCATTGAGATGGGGAAAAAAGTATTTTAAGGGAAATTTTAAG
AGGTTGGCAATAGTGAAAGGTAGAGTTGACCCAACTAATTTCTTCTGTAA
TGAACAAAGCATCCCACCTTATTCGTATTAG.

In some embodiments, the DNA molecule comprises a nucleic acid sequence with at least 79, at least 85%, at least 95%, or at least 99% homology or identity to SEQ ID NO:79, or any value and range therebetween. Each possibility represents a separate embodiment of the invention. In some embodiments, the DNA molecule comprises a nucleic acid sequence with 79% to 95%, 82% to 97%, 81% to 98%, or 79% to 100% homology or identity to SEQ ID NO: 79. Each possibility represents a separate embodiment of the invention.

In some embodiments, the DNA molecule comprises the nucleic acid sequence:

(SEQ ID NO: 89)
ATGACCAACTCGGAACTTGTTTTCATCCCATCTCCGGGAGCCGGCCACCT
ACCACCTACGGTGGAGCTAGCAAAGCTCCTCCTCCACCGCGAACCACAGC
TTTCGGTTACCATCATCATCATGAACCTCCCTCATGAAACAAAACCCACT
ACTGAAACTCGAATGTCCACTCCTCGTCTACGCTTTATTGACATACCTAA
AGACGAGTCAACAAAAGATCTTATCTCACGCCACACATTCATATCCGCCT
TCCTTGAACACCAAAAGCCACATGTTCGAAACATTGTCCGTTCAATCACC
GAGTCTGACTCGGTTCGGTTAGTTGGGTTCGTCGTAGACATGTTTTGTAT
TGCCATGATGGACGTCGCAAACGAGCTGGGTGCTCCAACTTATCTTTATT
TCACCTCCTCTGCCGCTTCACTTGGCCTCATGTTTTGCCTACAGGCCAAA
CGAGACGACGAGGAGTTTGATGTGACCGAGTTGAAGGACAAAGATTCGGA
ACTCTCCATTCCGTGTTACACCAACCCACTCCCAGCTAAGTTGTTACCTT
CGGTACTATTTGATAAGAGAGGTGGGTCAAAAACATTTATTGACCTCGCT
AGAAAGTATCGCGAGTCGAGGGGTATAGTTGTAAATACTTTTCAAGAACT
CGAAAGCTATGCTATTGAGTATCTTGCAAGTAGTAATGCTAACGTCCCAC
CGGTGTTTCCGGTGGGGGCGATACTAAACCAAGAAAAAAAGGTAAATGAT
GATAAGACGGAGGAGATTATGACATGGTTAAACGAGCAACCGGAGAGTTC
GGTGGTGTTTCTATGCTTCGGGAGCATGGGAAGCTTCGGTGAGGATCAAA
TTAAGGAAATAGCGCTTGCTATCGAAGAAAGCGGACAAAGGTTTTTGTGG
TCACTACGTCGTCCCCCTTCGAACGAAAATAAGTACCCGAAAGAATACGA
AAATTTTGGAGAGGTTCTTCCGGAAGGTTTCCTTGAACGAACATCGAGTG
TAGGGAAAGTGATAGGATGGGCCCCACAAATGGCAGTGTTGTCCCATTCT
TCAGTTGGTGGGTTTGTGTCACATTGCGGATGGAACTCGACACTCGAGAG
CATATGGTGTGGTGTACCGGTAGCTGCGTGGCCATTATATGCAGAACAAC
AACTTAATGCTTTTAAACTAGTGGTGGAGTTGGGCTTAGCGGTCGAGATT
AAGATTGATTATAGGAGTGAGAACGAGATTATTTTGACATCGAAAGAAAT
CGAGAGTGGGATTAGGAGGTTGATGAATGATGAAGAGTTGAGGATGAAAG
TGAAAGAGATGAAGGGGAATAGTAGGTTTGCAGTTTCAGAGGGTGGATCT
TCTTACGTATCCATTAGGCGTTTTATCGACCTTGTGATGACTAAGGAGTA
A.

In some embodiments, the DNA molecule comprises a nucleic acid sequence with at least 77%, at least 79%, at least 85%, at least 95%, or at least 99% homology or identity to SEQ ID NO: 89, or any value and range therebetween. Each possibility represents a separate embodiment of the invention. In some embodiments, the DNA molecule comprises a nucleic acid sequence with 77% to 95%, 78% to 100%, 79% to 99%, or 77% to 100% homology or identity to SEQ ID NO: 89. Each possibility represents a separate embodiment of the invention.

In some embodiments, the DNA molecule comprises or consists of the nucleic acid sequence:

(SEQ ID NO: 90)
ATGCCGACCTCAGAACTTGTTTTCATCCCATCCCCCGGTGTCGGCCACCT
GTCGCCTACCATCGAACTCGTCAATCAACTCCTCCACCGCGACCAGCGCC
TGTCTGTCACAATCATCGTCATGAAGTTCTCTCTTGAATCAAAACACGAT
ACAGAAACTCCTACATCCACTCCTCGATTACGCTTCATTGATATCCCTTA
TGACGAGTCCGCTATGGCTCTCATTAACCCGAACACGTTCCTCTCCGCTT
TCGTCGAGCACAACAAACCTCATGTTCGAAACATTGTTCGTGACATTTCC
GAGTCTAACTCGGTTCGGCTCGCGGGGTTTGTTGTGGACATGTTTTGTGT
AGCTATGACGGATGTAGTGAACGAGTTTGAAATTCCAACCTATATTTATT
TTACCTCGACCGCGAACTTACTCGGACTCATGTTTTACCTTCAGGCCAAG
CGTGACGACGAGGGTTTTGATGTCACCGTGTTGAAAGACTCAGAATCAGA
GTTTTTGTCTGTTCCGAGTTATGTCAACCCGGTTCCAGCTAAGGTTTTAC
CTGATGCAGTTTTGGATAAGAATGGTGGGTCTCAAATGTGTCTGGATCTT
GCAAAAGGGTTTCGTGAGTCGAAGGGCATAATAGTAAATACATTTCAAGA
ACTCGAAAGGCGTGGAATCGAGCACCTTTTAAGTAGTAACATGAACCTCC
CACCTGTGTTTCCTGTGGGGCCTATATTGAACTTGAGAAATGCGCCAAAC
GATGGTAAAACGGCCGATATCATGACATGGTTAAATGACCATCCAGAGAA
CTCGGTTGTGTTCTTGTGTTTCGGAAGTATGGGAAGCTTCGAGAAAGAAC
AAGTGAAGGAGATAGCGATTGCCATCGAACAGAGTGGGCAACGGTTTCTA
TGGTCACTCCGTCGTCCAACATCGCTAGAAAAGTTTGAGTTTCCAAAGGA
TTACGAGAACCCGGAGGAGGTTTTGCCAAAGGGATTTCTTGAAAGGACAA
AAGGTGTGGGAAAGGTTATCGGGTGGGCCCCACAAATGGCGGTGTTGTCT
CACCCGTCAGTGGGAGGGTTCGTGTCCCACTGTGGGTGGAACTCCACATT
GGAGAGCATATGGTGTGGGGTCCCAATAGCGGCTTGGCCACTATATGCGG
AACAAAAAATTAATGCTTTTCAATTGGTGGTAGAGATGGGAATGGCAGCT
GAGATTAGGATCGACTATCGGACTAATACGAGACCGGGTGGTGGTAAAGA
GATGATGGTAATGGCTGAAGAGATTGAGAGTGGTATTAGGAAGTTGATGA
GCGATGATGAGATGAGAAAGAAAGTGAAAGGTATGAAGGATAAAAGTAGG
GCTGCTGTTCTTGAAGGTGGATCATCTCACACATCAATTGGGATTTTAAT
TGAGAATTTGGTGAGTATAACGATCTAG.

In some embodiments, the DNA molecule comprises a nucleic acid sequence with at least 76%, at least 77%, at least 85%, at least 93%, at least 97%, or at least 99% homology or identity to SEQ ID NO: 99, or any value and range therebetween. Each possibility represents a separate embodiment of the invention. In some embodiments, the DNA molecule comprises a nucleic acid sequence with 76% to 95%, 77% to 98%, 80% to 99%, or 76% to 100% homology or identity to SEQ ID NO: 90. Each possibility represents a separate embodiment of the invention.

In some embodiments, the DNA molecule comprises or consists of the nucleic acid

(SEQ ID NO: 91)
ATGGTGGGTCTCAAATGTTTTTGGATCTTGCAAAAAGGTTTTCGTGAGTC
GAAGGGCATAATAGTAAATACATTTCAAGAACTCGAAAGGCGTGGAATCG
AGCACCTTTTAAGTAGTAACATGGACCTCCCACCTGTGTTTCCTGTGGGG
CCGATATTGAACTTGAGAAATGCGCGAAACGATGGTAAAATGGCCGATAT
CATGACATGGTTAAATGACCAGCCAGAGAACTCGGTTGTGTTCTTGTGTT
TCGGAAGTAGGGGAAGCTTCAAGGAGGAACAAGTGAAGGAGATAGCAATT
GCCATCGAACAAAGTGGGCAACGGTTTCTATGGTCACTCCGTCGTCCAAC
ATCGATAGAAACGTTTGAGTTTCCAAAGTATTACGAGAACCCGGAGGAGG
TTTTGCCAAAGGGATTTCTTGAAAGGACAAAAAGTGTGGGAAAGGTTATC
GGGTGGGCCCCACAAATGGCGGTATTGTCTCACCCGTCAGTGGGAGGGTT
CGTGTCCCACTGTGGGTGGAACTCCACATTGGAGAGCATATGGTGTGGGG
TCCCAATAGCGGCTTGGCCACTATATGCGGAACAACAAACTAATGCTTTT
CAATTGGTGGTCGAGATGGGAATGGCAGCAGAGATTAGGATCGACTATCG
GACTAATACACCACTGGTTGGTGGTAAAGACATGATGGTAACGGCTGAAG
AGATTGAGAGAGGTATTAGGAAGTTGATGAGCGATGATGAGATGCGAAAG
AAAGTGAAAGACATGAAGGATAAGAGTAGAGGTGCAGTTTTAGAGGGTGG
GTCATCTCATACATCAATTGGGAATTTAATTGATGTTTTGGTGAGTATAA
CGATCTAG.

In some embodiments, the DNA molecule comprises a nucleic acid sequence with at least 78%, at least 80%, at least 85%, at least 95%, or at least 99% homology or identity to SEQ ID NO: 91, or any value and range therebetween. Each possibility represents a separate embodiment of the invention. In some embodiments, the DNA molecule comprises a nucleic acid sequence with 79% to 95%, 78% to 100%, 80% to 99%, or 79% to 100% homology or identity to SEQ ID NO: 91. Each possibility represents a separate embodiment of the invention.

In some embodiments, the DNA molecule comprises or consists of the nucleic acid sequence:

(SEQ ID NO: 92)
ATGGCGACCAACAACCTCCATTTCCTTCTAATTCCCCATATAGGTCCAGG
CCACACTATTCCCATGATAGATATGGCTAAACTTCTTGCAAAACAACCAA
ATGTAATGGTTACAATAGCTACAACACCTCTTAATATCACCCGTTACGGG
CACACTCTCGCAGACGCCATCAACTCGTTTCGCTTCTTTGAGGTTCCATT
TCCGGCAGTTGAGGCTGGATTACCTGAAGGATGTGAAAGCACGGATAAAA
TCCCAAGTATGGATCTAGTACCGAACTTTTTAACCGCGATTGGTATGCTA
GAACAAAAGCTAGAAGAGCATTTTCACTTGCTAGAGCCTCGTCCGAATTG
TATTATTTCTGATAAGTACATGTCGTGGACGGGTGATTTTGCTGATAAGT
ATCGGATCCCTAGAATTATGTTTGATGGAATGAGCTGTTTTAACGAGTTA
TGTTACAACAATTTGTATGAAAACAAGGTGTTTGAAGGGATGCATGAAAC
AGAACCATTTGTTGTCCCTGGTTTACCCGATAAAATTGAGCTAACACGAA
AACAGCTCCCACCTGAGTTTAACCCGAGCTCGATTGATACAAGTGAGTTT
CGTCAGCGGGCTAGGGACGCTGAGGTGAGGGCTTATGGAGTTGTGATCAA
TAGTTTTGAGGAGTTGGAACAAGAATATGTTAATGAGTATAAGAAGTTAA
GAAAGGGTAAGGTTTGGTGTATCGGCCCGCTGTCACTGTGCAATAGTGAC
AATTCGGATAAAGCCCAAAGAGGAAATATAGCGTCAGTCGATGAAGAAAA
ATGTTTAAAATGGCTTGATTCTCATGAAGCCGACTCAGTAGTTTACGCTT
GTTTTGGTAGCCTTGTTCGGGTCAACACCCCACAACTAATTGAGCTTGGT
TTAGGCCTAGAAGCATCAAATCGCCCGTTCATTTGGGTGGTTAGATCGGT
TCATAGAGAAAAAGAGGTCGAGGAATGGCTAGTGGAAAGTGGTTTTGAGG
AGAGAATTAAAGATAGAGGTTTAATAATCCGAGGTTGGGCCCCACAAGTA
CTTATCTTGTCTCACCCTTCTATTGGAGGGTTTTTAACGCATTGCGGTTG
GAACTCGACCCTAGAATCAGTCTGTGCAGGTGTTCCAATGATCACATGGC
CTCAATTTGCAGAGCAATTTATCAACGAGAAGCTAATAGTGCAAGTGTTG
GGGATTGGTGTGGGTGTTGGAGTTGATTCTGTTGTCCATGTGGGCGAAGA
AGATAGATCTGGGGTGAAAGTGAAGAGGGAGAGTGTTACGAAGGCTATTG
AGAAAGTCATGGATGACGAGATTGATGGAAATGAGAGACGGAGGAGATCG
AAAGAGTTTGGAAAGATAGCTAATAACGCGATTAAAGAGGGAGGGTCTTC
ATACCTTAACTTGACTCTGCTAATTCAGGACATAATGCGTTATGCAAATG
CAGATGCTTCAAGCTAA.

In some embodiments, the DNA molecule comprises a nucleic acid sequence with at least 87%, at least 92%, at least 97%, or at least 99% homology or identity to SEQ ID NO: 92, or any value and range therebetween. Each possibility represents a separate embodiment of the invention. In some embodiments, the DNA molecule comprises a nucleic acid sequence with 87% to 100%, 88% to 99%, 89% to 99%, or 87% to 100% homology or identity to SEQ ID NO: 92. Each possibility represents a separate embodiment of the invention.

In some embodiments, the DNA molecule comprises or consists of the nucleic acid sequence:

(SEQ ID NO: 93)
ATGGAAAAAACACCTCATATAGCCATTGTACCAAGTCCAGGAATGGGCCA
CTTGATCCCTTTAGTTGAGTTTGCTAAAAAACTAAAAAATCACCACAACA
TACATGCAACTTTCATCATCCCAAATGATGGACCTTTATCTATTTCTCAA
AAGGTTTTTCTTGATTCACTTCCTAATGGTTTAAACTATCTCATTCTACC
TCCGGTAAATTTTGATGATTTACCACAAGATACCCAAATCGAAACTCGAA
TTAGTCTAATGGTAACACGGTCTCTTGATTCGCTACGTGAAGTGTTTAAG
TCATTAGTTGTGGAAAAAAATATGGTTGCTTTGTTTATTGATCTTTTTGG
GACAGATGCATTTGATGTTGCTATTGAATTTGGTGTTTCACCTTATGTGT
TCTTTCCATCAACTGCTATGGCTTTATCTTTGTTTCTATATTTGCCTAAA
CTTGATCAGATGGTTTCATGTGAGTATAGGGAGCTTCCTGAACCGGTTCA
AATTCCAGGTTGTATACCGGTTCGTGGACAAGACTTGGTTGACCCGGTTC
AAGATAGAAAGAATGATGCATACAAATGGGTGCTTCATAATGCAAAGAAG
TATTCAATGGCTAAGGGTATAGCGGTAAATAGCTTCAAGGAGTTAGAAGG
TGGAGCTTTGAATGCTTTGCTAGAAGATGAACCGGGTAAGCCAAAAGTTT
ATCCGGTCGGACCGTTAGTACAAACCGGTTTTAGTTGTGATGTTGATTCG
ATAGAGTGCTTGAAGTGGTTAGATGGTCAGCCATGTGGTTCTGTTTTGTA
TATATCTTTTGGAAGCGGTGGGACCCTTTCATCCAGTCAACTTAATGAGT
TAGCTATGGGTTTGGAGTTGAGTGAACAACGGTTCATATGGGTGGTTAGA
AGCCCGAACGATCAACCAAACGCCACGTACTTTGATTCTCATGGTCACAA
AGACCCTCTTGGTTTTTTGCCCAAAGGGTTCTTGGAAAGAACCAAAGGAA
TTGGGTTTGTGATCCCTTCTTGGGCTCCACAAGCCCAGATCCTGAGTCAC
AGTGCCACAGGTGGATTTTTAACCCACTGTGGTTGGAACTCAATTCTCGA
GACTGTAGTCCATGGTGTGCCGGTGATTGCTTGGCCACTTTATGCCGAGC
AAAAGATGAATGCAGTGTCTTTAACCGAGGGTATAAAAATGGCGTTAAGA
CCCACGGTTGGTGAAAATGGGATTGTGGGTCGCTTAGAGGTTGCGAGAGT
TGTGAAGAGTTTACTGGAAGGAGAAGAAGGGAAGGCGATTAGGAGTCGAG
TTCGTGATCTCAAGGATGCTGCTGCTAATGTTCTTAGTAAAGATGGGTCT
TCTACAAAAACTTTAGATCAATTGGCTGTACAGTTGAAAAAACAAGAATT
AAGCTAG.

In some embodiments, the DNA molecule comprises a nucleic acid sequence with at least 87%, at least 92%, at least 97%, or at least 99% homology or identity to SEQ ID NO: 93, or any value and range therebetween. Each possibility represents a separate embodiment of the invention. In some embodiments, the DNA molecule comprises a nucleic acid sequence with 87% to 100%, 88% to 99%, 89% to 99%, or 87% to 100% homology or identity to SEQ ID NO: 93. Each possibility represents a separate embodiment of the invention.

In some embodiments, the DNA molecule comprises or consists of the nucleic acid sequence:

(SEQ ID NO: 94)
ATGACTCAAAAGCAAATGCAAATGCAACCTCACTTTCTCTTAGTAACATA
TCCCGCACAAGGTCATATTAACCCGTCTCTCCAGTTCGCTGAACGTCTCA
TTCGGTTGGGTGTCAAAGTCACCTTCACAACAACTGTCTCTGCTTACCGC
CGAATGAGTAAAGCGGGCAACATCTCAGAGTTTTTAAATTTTGCTGCTTT
TTCAGACGGCTTTGATGACGGTTTCAACTTCGAAACAGACGATCATGGTC
TCTTCTTAACTCAATTGAGAAGCAGGGGAAAAGATAGCTTGAAAGAAACA
ATTCTTTCAAATGCTAAAAATGGAACTCCAATTAGTTGTTTGGTTTACAC
ACTCCTACTCCCTTGGGCTCCTGAGGTGGCACGTGGCCTAAACGTGCCCT
CAGCCTTTCTTTGGATTCAACCAGCTTCTGTTTTACGACTTTACTATTAC
TACTTCAATGGGTACAATGAACTCATCGGTGACGATTGTAATGAACCTTC
ATGGTCCATTCAATTACCAGGGTTACCATTGCTCAAAAGTCATGACCTTC
CCTCCTTTTGTCTCCCTTCAAATCCTTACAGTAATGTACTGGCTCTAGTC
AAAGAGCATTTAGATATGCTGGATCTGGAAGAGAAGCCTAAAATACTTGT
GAATAGTTTTGATGAGTTGGAGAGGGAGGCGTTGAATGAAATTAATGGAA
AACTAAAAATGGTCGCCGTAGGGCCTTTGATTCCATCAGCTTTTTTGGAT
GGACAAGATGCATCTGACAAATCTTTTAGGGGAGATTTGTTTGAAACATC
CAAAGATTATTTGGAATGGATGAATACAAAGCCTGAAGGGTCCATTGTTT
ACATATCTTTTGGTAGTCTTTTAGTGTTCTCAAAGATACAAAAGGAGGCA
ATGGCACATGCTTTGTTAGAGTGCGGGAGGCCGTTCTTGTGGGTGATAAG
AGATGGAGAACAAGGAGAACAACTAAGTTGTATTGAGAAATTGGAACAAT
TAGGTTTGATAGTCCCATGGTGTAGTCAACTAGAGGTATTATCACACCCT
TCTTTAGGTTGTTTTGTGACACATTGTGGTTGGAACTCGACTTTAGAGAG
TATAGTTTGTGGAGTTCCTGTGGTGGCATTTCCTCAATGGACAGATCAGA
CGACAAATGCAAAGCTTCTAGAAGACGTATGGGGAACAGGGGTGAGAGTG
ACAACTAATGAAGACGGGGTTGTTGAAAGCGAGGAGATAAGAAGGTGCAT
CGAAATGGTAATGGGAGGCCGTGATAGTGAATCAACAATGAGAAAGAATG
CTAAGAAGTGGAAGGATGTGGGAAGAGAGGCTATGAAAGAAACAGGATCT
TCTTATATGAATCTCAAGGCTTTTATTAAAGAAGTGAATGATGGTGAATC
AACCATCAAAACTGAAATTGTTTCAACTATATGA.

In some embodiments, the DNA molecule comprises a nucleic acid sequence with at least 80%, at least 87%, at least 93%, at least 97%, or at least 99% homology or identity to SEQ ID NO: 94, or any value and range therebetween. Each possibility represents a separate embodiment of the invention. In some embodiments, the DNA molecule comprises a nucleic acid sequence with 80% to 98%, 81% to 99%, 85% to 99%, or 80% to 100% homology or identity to SEQ ID NO: 94. Each possibility represents a separate embodiment of the invention.

In some embodiments, the DNA molecule comprises or consists of the nucleic acid

(SEQ ID NO: 95)
ATGACTAAAATACAACAGCAACCTCACTTTCTCTTAGTAACATATCCCGC
ACAAGGTCATATTAACCCGTCTCTCCGGTTCGCCGAACGACTCATTCGGT
TGGGTGTCAAAGTCACCTTCACAATAACTGTCTCTGCTTACCGCCGAATG
AGTAAAGCGGGCCACATCTCAGAGTTTTTAAATTTTGCTGTTTTTTCAGA
CGGCTTTGATGACGGTTTCAACTCCAAAACAGACGATTATGGTCTCTTCT
TAACTCAATTCAGAAGCAGGGGAAAAGATAGCTTGAAAGAAACAATTCTT
TCAAATGCTAAAAACGGAACTCCAGTTAGTTGTTTGGTTTACACACTCCT
ACTCCCTTGGGCTCCTGAGGTGGCACGTGGCCTAAACGTGCCCTCAGCCT
TTCTTTGGATTCAACCAGCTTCTGTTTTACGACTTTACTATTACTACTTC
AATGGGTACAATGAACTCATCGGCGACGATTGTAACGAACCTTCATGGTC
CATTCAATTACCAGGGTTACCATTGCTCAAAAGTCGTGACCTTCCCTCCT
TTTGTCTCCCTTCAAATCCTTACGCTGATGTACTGACTTTAGTCAAAGAG
CATTTAGATGTGTTGGATTTGGAAGAGAAGCCTAAAATACTTGTGAATAG
TTTTGATGAGTTGGAGAGGGAGGCGTTGAATGAAATTGATGGGAAACTAA
AAATGGTTGCCGTAGGGCCTTTGATTCCATCAGCTTTTTTTGGATGGACA
GGATGCATCTGA.

In some embodiments, the DNA molecule comprises a nucleic acid sequence with at least 77, at least 85%, at least 95%, or at least 99% homology or identity to SEQ ID NO: 95, or any value and range therebetween. Each possibility represents a separate embodiment of the invention. In some embodiments, the DNA molecule comprises a nucleic acid sequence with 77% to 95%, 82% to 97%, 81% to 98%, or 77% to 100% homology or identity to SEQ ID NO: 95. Each possibility represents a separate embodiment of the invention.

In some embodiments, the DNA molecule comprises or consists of the nucleic acid

(SEQ ID NO: 96)
ATGGGTTCATGGCGGAATTCAAGAACAACGTCTACAAAGTTTTTATGGTT
GATTTTACCGTTGATGGTGGTGACGGTGATTATAGGGGTAAAAAAGTCAA
ATTATGGGTCGAAGTATAATTATCCTTGGGTTTGGAGTTCAGTGATTAAT
TCTTATTCTTCTTCTGCGGTTAAAGAAGATGTAACGGTGGTGGCTGAAGG
TCCTGTTGAATCATTTGGGTTGCGGTCAACGGTGGTCAACGGTGGTGGTG
TGGTGGCGGAAGGGCCGTCGGAAGATTTTGGTTTTAATTCTTCTTATCCA
CCGTTGGCTATGGAAGATGAAATGGATGTTGAGCTACCTGCTATTGCCAA
GGAAGATGACTTGAACGCGACGTTGAGTGGACCCGACCTTTTTGTGTCTG
CAAATCAAACTGGCGGACTTCATGTTGATATTGGAATCAACAGTAAGTAT
ACCAGTTTGGATAAGCTTGAAGCCCGCTTAGGTCAGGTTCGAGCTGCAAT
AAAAGAAGCCGAATCAGGAAATAGAACTTACGATCCGGATTATGTACCAG
AGGGTCCTATGTACTGGCATGCAGCCTCATTTCACAGGAGTTATTTGGAG
ATGGAAAAGCAATTTAAGGTGTTTGTATATGAAGAAGGAGAACCACCAAT
ATTTCATAACGGTCCTTGCAAAAACATATATGCAATGGAAGGTAACTTTA
TCTACCATATGGAAACAACCAAGTTTAGGACAAAAAACCCCGAAAAAGCT
CACACGTTTTTTCTCCCAATGAGTGCTGCAATGATGGTGAGGTTTATCTT
TGAGCGTGATCCAAATGTTGACCATTGGCGTCCTATGAAGCAAACAATTA
AAGATTATGTTGATCTTGTGGGTGGTAAGTACCCATTTTGGAATCGAAGC
TTAGGAGCCGATCACTTTACTGTTGCGTGCCACGATTGGGTGAGTAAAGT
CTTTTATCCCATCATTTTCATGCTTTTACTAGTATTTATCTTCAGAATGT
CGACTGGATGCTGA.

In some embodiments, the DNA molecule comprises a nucleic acid sequence with at least 82%, at least 85%, at least 89%, at least 92%, at least 95%, at least 97%, or at least 99% homology or identity to SEQ ID NO: 96, or any value and range therebetween. Each possibility represents a separate embodiment of the invention. In some embodiments, the DNA molecule comprises a nucleic acid sequence with 82% to 95%, 83% to 98%, 82% to 99%, or 82% to 100% homology or identity to SEQ ID NO: 96. Each possibility represents a separate embodiment of the invention.

In some embodiments, the DNA molecule comprises or consists of the nucleic acid

(SEQ ID NO: 97)
ATGTCAACCGTTGAGGTTGCAAAGTTACTTGTGAATCGAGATCATCGTCT
CTTCATAACATTCCTTATCATTCAGCCTCCTAGCTCGGGTTCTGGCTCAG
CTATCACCACCTACATCGAATCATTAGCTGAGAAAGCTATGGACCGCATA
TCCTTCATTGAGCTACCTCAAGATAAAATCCCACCACCACGTTACCCGAA
ATCCCTGCCAACTGCAGAATCGAAAGCTCATCCCCTTATTTTCATGATTG
AGTTCATTAAGTGTCACTGCAAATATGTTAGAAACATTGTATCTGACATG
ATAAGTCAACCGAGTTCGGGTCGGGTAGCTGGGTTGGTAATCGACATGCT
TTGTTTCAGCATGATGGATGTCGCTAATGAGTTCAACATTCCAACCTATG
TATTTGTCACTTCTAATGCTGCTTTTCTTGGATTTTATTTATATGTCCAG
ATACTCTCTAATGATCAGAACCAAGACGTTGTTGAGCTGAGCAAATCTGA
TACCGAGATATCGGTTCCAGGTTTTGTAAAGCCGGTGCCAACGAAAGTCT
TCTGGACTGTTGTCCGCACTAAAGAAGGACTGGACTTTGTTTTGTCATCT
GCCCAGAAACTTAGACAAGCCAAAGCAATCATGGTTAATACCTTCTTGGA
GTTGGAAACACACGCAATCAAGTCGCTGTCTGATGACACCAGCATCCCGC
CTGTGTATCCAGTGGGACCGATACTCAATTTAGAAGGTGGTGCTGGCAAA
ACGTTCGACAATGACATTAGCAGGTGGTTGGACAGTCAACCGCCTTCCTC
GGTGGTGTTCTTGTGCTTTGGAAGCCACGGATGTTTTGATGAGATCCAAG
TGAAGGAGATAGCACATGCTTTAGAGCAGAGTGGCCACCGTTTCTTGTGG
TCCCTACGTCGACCTCCATCAGATCAAACATTAAAAGTTCCCGGTGATTA
CGAGGATCCAGGAGTGGTATTACCGGAAGGATTTCTTGAGCGAACTGCTG
GACGTGGGAAAGTAATTGGGTGGGCCCCGCAGGTGATGGTGCTGGCTCAC
CGTGCAGTTGGAGGCTTCGTGTCCCACTGTGGGTGGAACTCGTTGTTGGA
GAGTTTGTGGTTCGGCGTACCAACGGCAACATGGCCGATCTATGCTGAGC
AGCAGATGAATGCGTTTGAAATGGTGGTGGAGCTGGGACTGGCTGTGGAG
ATAACATTGGATTATAGGAATGATATGGATATGTTCATTGTCACCGCACA
GGAGATAGAAAGTGGTATAAGAAAGGTGATGGAGGATAATGAGGTAAGAA
CAAAAGTGAAAGAGAGAAGTGAGAAGAGTAGAGCAGCAGTGGCGGAGGGG
GGGTCATCGTATGCATCTGTTGGTCATCTTATTAAAGAATTTACAGGAAA
CATCTCCTAA.

In some embodiments, the DNA molecule comprises a nucleic acid sequence with at least 79, at least 85%, at least 95%, or at least 99% homology or identity to SEQ ID NO: 97, or any value and range therebetween. Each possibility represents a separate embodiment of the invention. In some embodiments, the DNA molecule comprises a nucleic acid sequence with 79% to 95%, 82% to 97%, 81% to 98%, or 79% to 100% homology or identity to SEQ ID NO: 97. Each possibility represents a separate embodiment of the invention.

In some embodiments, the DNA molecule comprises or consists of the nucleic acid

(SEQ ID NO: 98)
ATGTCATCATTCATCAACTTTGTTGAATCCACAACACAACTTCAACCACA
ATTCGAACAACTCATCCAAACACTTCTTCCCATAACTGCGATAATATCGG
ATGGTTTTTTGATGTGGACACAAGATTCCGCCGAAAAATTCAATATCCCA
CGTCTGGTTTTTTATGGGACAAACATATTTTTCATGACTATGTGTAACAT
TATGGCACAATTTAAGCCACATGCGGCTGTTAATTCTGATGATGAGGCGT
TTGATGTACCCGGTTTCACCAGGTTTAAGTTGACGGCTAATGATTTTGAG
CCGCCTTTTAATGAGGTTGAACCGAAAGGTTCAATGTTGGATTTTTTATT
GGAGCAACAAAAGGCTATGGTTAGGAGCCATGGGTTGGTGGTTAATAGTT
TTTATGAGATTGAACATGAGTTTAATGTTTATTGGAATCAGAACTATGGA
CCTAAAGCTTGGTTAATGGGACCATTTTGTGTAGCTAAGCCATATGCATC
AAACGTCATGGATTCCGAGATATCGACTAAGGTGGTGAAAAAATCAGCAT
GGATCCAGTGGCTTGACAGGAAGCTTGCAGCGAACGAGCCAGTGTTATAC
ATCTCATTTGGAACACAGGCAGAGGCGTCTATGGAGCACTTACACGAGGT
CGCTATTGGTTTGGAACGATCAAATGTAAGCTTCATTTGGGTGGTAAAAG
CGAAGCAGATGCAATTAATTGGAGCAGGGTTTGAAGAGAGGGTGAAGGGG
AGAGGAAAAGTGGTGACAGAATGGGTGGATCAGATGGAAATCTTGAAACA
TGAAATTGTAAGCGGGTTTTTAAGTCATTGTGGGTGGAACTCACTGCTAG
AGAGTATGTGTGTGGGTGTGCCGGTGCTTGCAATGCCGTTGATGGCGGAT
CAACTCTTAAATGCAAGGTTGGTTGTGGAGGAGATTGGGATGGGGCTACG
GTTGTGGCCGAGGGGTATGGTGGCACGTGGGATAGTTGGGGCGGAGGAAG
TCGAGAAAATGGTGGTGGAGTTGATGGAAGGGGAAGGTGGGAGAAGGGTG
CGGAAAAGGGTCATCGAGGTTAGAGAAATGGCATATGGTGCGATGAAGGA
AGGAGGGTCATCATCGAGGACATTAGACTCGTTGATTGATCATGTTTGTG
AAGCCTTTCATAAGACGGTTTAA.

In some embodiments, the DNA molecule comprises a nucleic acid sequence with at least 78, at least 85%, at least 95%, or at least 99% homology or identity to SEQ ID NO: 98, or any value and range therebetween. Each possibility represents a separate embodiment of the invention. In some embodiments, the DNA molecule comprises a nucleic acid sequence with 78% to 95%, 82% to 97%, 81% to 98%, or 78% to 100% homology or identity to SEQ ID NO: 98. Each possibility represents a separate embodiment of the invention.

In some embodiments, the DNA molecule comprises or consists of the nucleic acid

(SEQ ID NO: 99)
ATGGGGAGCTTGAAGAAAGGTGCACATATACTAATATTCCCATTC
CCAGCACAAGGTCATATGCTCCCACTCCTAGACCTAACTCACCAC
CTAGCCACCAATGGGTTAACCATAACCATATTAGTCACACCCAAA
AACCTACCAATCTTGAACCCACTTTTATCTTCATCTCCAAACATC
CAACCACTAGTCTTCCCTTTCCCACCTCACCCAAGACTTCCACCA
CATGTTGAAAATGTTAAAGACATAGGTAACCATGCAAATGTCCCA
ATCACAAACTCACTAGCCAAATTACAAGACCAAATAATCCAGTGG
TTTAACTCCCACCATAACCCTCCTGTTGCCATCATCTCAGATTTC
TTTCTTGGATGGACCCAACACCTTGCAAACAAACTTGGTATCCCT
CGTGTCGGGTTTTTTTCTTCTGGTGCTTACTTGACTGCTGTTCTT
GATTATGTTTGTCATAATATTAAAACTGTTAGGTCTCAAGAGGAG
ACTGTTTTTCATGACTTGCCAAATTCTCCTTGTTTTAAATTCGAG
CATCTTCCGGGTTTGGCCCAGATTTATAAAGAGTCCGACCCGGAA
TGGGAATTGGTTCTTGATGGTCATATTGCGAATGGGTTAAGTTGG
GGTTGGATTGTGAATACTTTTGATGGGTTGGAGTCTCGGTATATG
GAGTATCTGACAAAGAAAATGGGTGTCGGACGGGTTTTTGGTGTC
GGGCCAGTTAATTTGTTAAACGGGTCGGATCCCATGACCCGTGGG
AAATCGGAATCCGGGTCTGATTCCGGTGTGTTGAACTGGCTCGAT
GGAAAACCCGATGGGTCGGTTTTGTATGTGTGTTTTGGAAGTCAA
AAGTTTCTTACTAATGACCAAATGGAGGGATTGTCAATTGGGCTT
GAACAAAGTGGGGTCCATTATGTTTGGGTTGTGAAAGACGAACAA
GGTGATGCAATTAGGTCCGGGTCGGGTAGAGGACTAGTGGTAACG
GGTTGGGCCCCGCAAGTTTCAATATTGGGTCATGGAGCGGTGGGT
GGGTTTTTGAGTCATTGCGGGTGGAACTCTGTTTTGGAAGCAATT
GTAAATGGAGTTATGATATTGGCTTGGCCAATGGAGGCTGATCAA
TTTGTTAATGCTAAGTTGTTAGTGGATGACCATGGTATAGGGGTG
TGGGTTTGTGAGGGGCCGAATACGGTTCCTGATTCAACCGAGTTG
GCTCGTAAAATTGGTGAGTCAATGAGTACGGATAAGAGTGAGAAG
GTAAAGGCGAAAGAAATGAAAAACAAAGCAAATGAAGCAGTTAAA
GAAGGTGGGAGCTCATCAATGGAATTAAGCAGGCTTGTTAAGGAG
CTGTCTAACTTTGAGACAAATGGGCCATGA.

In some embodiments, the DNA molecule comprises a nucleic acid sequence with at least 82, at least 85%, at least 95%, or at least 99% homology or identity to SEQ ID NO: 99, or any value and range therebetween. Each possibility represents a separate embodiment of the invention. In some embodiments, the DNA molecule comprises a nucleic acid sequence with 82% to 95%, 82% to 97%, 83% to 98%, or 82% to 100% homology or identity to SEQ ID NO: 99. Each possibility represents a separate embodiment of the invention.

In some embodiments, the DNA molecule comprises or consists of the nucleic acid

(SEQ ID NO: 100)
ATGGATACCCAAACACAAGTCAAGAAACAAAAACTTGAAACCATG
GAACATAAAACATCATCCGCCGAAATCTTCGTGCTACCATTTTTT
GGTACGGGTCATATAAACCCAGCAATGGAGCTTTGCCGGAACATT
TCATCACATAATTACAAAACTACCCTCATCATCCCTTCACATCTT
TCTTCATCTATTCCTTCTCCCTTTTCTTCAACTTTACTTCATGTT
GCTGAGATCCCTTTCACTGCTTCTGACCCGGAACCCGGATCCGGA
AGAGGGAACCCACTTGATGCCCAGAACAAGCAAATGGGTGAAGGG
ATTAAGGCGTTTATGTCTGCAAGATCTGACGGATCAAAACTACCC
ACGTGTGTTGTTATTGATGTCATGATGAACTGGAGTAAAGAGATA
TTTGTTGATTACCAGATTCCTATTGTCTCTTTTTTTACTTCTGGA
GCTACTAATACTGCTATGGGTTATGGTAGGTGGAAAGCTAAAATT
GGTGATCTGAAGCCCGGGGAGACCCGTGTGATCCCCGGACTTCCT
ACTGAAATGGCCGTTACTTTTGCGGATTTAAATCAAGGTCCTAGA
GGCCGTGGGCCTCGGCCGGATGGGTCAAGGCCTGACGGGCCAAGG
TCTGGACCACCTGGTGGGATGAGGTCCGGACCACCTCACGGGATG
AGGGGTGGGGGACGAGGTGGGCGGGGCGGTGGACGACCCGGCCCG
GATGCGAAACCACGTTGGGTAGATGAAGTGGACGGGTCGGTAGCT
TTGCTTATCAACACGTGTGACAATCTCGAGCGTGTGTTTATTGAT
TACATTGCTGAAGAAACCAAGATTCCCGTTTATGGTGTTGGCCCG
TTGCTGCCCGAAAAGTATTGGAAGTCAGCGGGTTCGTTGCTTCGT
GATCATGAAATGAGGTCTAACCATAAAGCGAATTACTCGGAAGAT
GAGGTGTTTCAATGGCTAGAATCGAAACCAGTTGGGTCGGTTATT
TACATATCGTTTGGGAGTGAAGTTGGCCCGACTATAGACGAGTAT
AAAGAGTTAGCTGGATCATTGGAAGGATCGAATCAGAATTTCATT
TGGGTGATCCAGCCCGGTTCGGGGATAACGGGCATGCCAAGATCG
TTTTTGGGCCCGGTTAATACGGATAGTGAGGAAGAAGAGGAAGGG
TATTATCCTGAGGGATTAGATGTTAAAGTTGGGAACAGGGGTTTG
ATCATCACTGGATGGGCTCCACAGTTGTTGATTTTGAGCCACCCA
TCTACAGGCGGGTTCTTATCACATTGTGGGTGGAATTCAACTGTT
GAGGCGATTGGGCGAGGTGTTCCGATATTGGGTTGGCCCTTGAGG
GGTGATCAGTTTGATAATGCGAAACTTGTGGCGAATCATTTGAAA
ATTGGGTTTGCGATGTCAAGTGTGGCGAGTGAAGGCGGACGACCT
GGGAAGTTCAACAAGGAGACTATAACAGCAGGGATTGAGAAACTA
ATGAATGATGAAGATGTGCATAAACAGGCAAAGAAACTTAGTAAA
GAATTTGAGAGTGGGTTTCCAGTGAGTTCAGTTAAAGCATTGGGT
GCTTTCGTGGAGTCTATTAGCCAGAAAGCAACCTAA.

In some embodiments, the DNA molecule comprises a nucleic acid sequence with at least 74, at least 80%, at least 85%, at least 87%, at least 93%, or at least 99% homology or identity to SEQ ID NO: 100, or any value and range therebetween. Each possibility represents a separate embodiment of the invention. In some embodiments, the DNA molecule comprises a nucleic acid sequence with 74% to 95%, 75% to 97%, 76% to 98%, or 74% to 100% homology or identity to SEQ ID NO: 100. Each possibility represents a separate embodiment of the invention.

In some embodiments, the DNA molecule comprises or consists of the nucleic acid

(SEQ ID NO: 101)
ATGTCACTCGTGACTAATAACCCACATTTACTAGTCTACCCATTA
CCTACCTCCGGCCATATCATTCCGTTACTCGACCTGACCGACCTT
CTTCTCCGCCGTGGCCTCACCATCACCGTCGTGATATCCACCACA
GACCTTACGCTTCTCGACACTCTCCTATCCTCACACCCCACGTCT
CTACACAAACTTTACTTCCCCGACCCCGAAATCGGCCCATCTTCT
CATCCCGTTATTGCCAGAATAATTGCCACCCAAAAACTATTTGAT
CCAATTGTTAAATGGTTTGAATCGCACCCTTCGCCTCCAGTCGCC
ATCATTTCCGACTTCTTTCTTGGGTGGACTAATGAACTTGCATCA
CGTTTAGGTATTCGACGTGTGGTGTTTTCACCTTCGGGAGCTCTT
GGTCATTCCATTTTACAAAGTTTGTGGCGTGACGTGGCGGAGATC
AATGCAAAAAATGTTGATGGAAATGGAAACTACTCGATTTCTTTT
ACCGATATACCAAACTCGCCCGAATTTCATTGGTGGCAGTTGTCA
CAACTTTTGCGTGTTCATAGGGAGGGAGATCCGGACTTCGAATTT
TTTAGGAATGGAATGTTGGCTAATACGAAAAGTTGGGGTATTGTT
TACAACACATTTGAAAGGATTGAAAAGGTTTACATTGACCATGTG
AAGAAACAAATAGGTCATGATCGGGTATGGGCAATAGGCCCATTA
CTTCCCGAAGAACATGGCCCAGTTGGTAGCACCGCACGTGGTGGG
TCCAGTGTAGTGCCACCTCATGACCTTCTCACGTGGTTGGACAAA
AAGCCCCATGACTCGGTCGTATATATATGTTTTGGGAGTCGATTG
ACGTTAAGTGAGAAGCAAATGAGTGCATTAGCAAGTGCACTCGAG
CTCAGTAACGTTGATTTTATTTTGTGTGTGAAGGCAAGTGGTTCG
AGCTTCATTCCTAGTGGGTTCGAAGATCGAGTGGTGGGTCGGGGG
TTCGTAATCAAAGGTTGGGCCCCACAGTTGGCGATATTGAGACAT
CGGGCTGTGGGGTCGTTTGTGACTCATTGTGGGTGGAACTCAACA
TTGGAAGGTGTTTCATCAGGAGTGATGATGTTGACGTGGCCAATG
GGTGCAGACCAATATGCAAATGCTAAGCTATTGGTCGACCAGTTA
GGTGTTGGGAAACGAGTTTGTGAAGGTGGACCCGAGAGTGTTCCT
GATTCAACTGAGTTGGCTCGGTTGTTGGAAGAGTCACTGAGTGGT
GATACATCCGAGCGAGTTAAAGTCAAGGAGCTAAGTCGGGAAGCT
AACACAGCTGTGAAAGAAGGAACTTCAATAAGAGATTTRGAACAT
GTTCGTTAACCTTTTATCCGAGCTCTAA.

In some embodiments, the DNA molecule comprises a nucleic acid sequence with at least 80, at least 85%, at least 95%, or at least 99% homology or identity to SEQ ID NO: 101, or any value and range therebetween. Each possibility represents a separate embodiment of the invention. In some embodiments, the DNA molecule comprises a nucleic acid sequence with 80% to 95%, 82% to 97%, 81% to 98%, or 80% to 100% homology or identity to SEQ ID NO: 101. Each possibility represents a separate embodiment of the invention.

In some embodiments, the DNA molecule comprises or consists of the nucleic acid

(SEQ ID NO: 115)
ATGGCAACCCAAGTCAAAACCGAGGAGAAGCATTTGAAGGTAGAA
ATCATAAACAAAACCTATGTGAAACCTGAAACACCACTAGGAAGA
AAAGAGTGTCAATTGGTCACATTTGATCTTCCTTATATAGCCTTC
TACTACAACCAAAAGTTGATCATCTATAAAGGTGGTGTCGAGGAG
TTCGAGGATACCGTCGAGAAACTGAAAGACGGGTTAAAGGTAGTT
TTGGGAGAGTTTCATCAATTGGCTGGAAAATTAGACAAAGATGAT
GACGGGGTGTTTAAGGTAGTGTACGATGATGACATGGATGGGGTG
GAGGTGCTTTCTGCGGTCGCGGAAGACACTGCGACCGCAGATTTG
ATGGACGAAGAAGGGACCATCAAGCTTAAGGAGTTGGTCCCTTAT
AATAGTGTTTTGAACATAGAGGGGCTTCATCGTCCGCTTTTATCG
ATTCAGATAACAAAACTAAAAGATGGGCTTGTACTGGGCTGTGCG
TTCAACCACGCGATTTTAGACGGTACATCCACCTGGCACTTCATG
AGCTCCTGGGCCCAAATTTGCTCCGGATCCAAATCCATTTCAGCG
GCGCCTTTCCTTGACCGTACCCAAGCGCGTAACACGCGCGTGAAA
CTCGATCTCACCCCTCCCGCCCAAACTAACGGCAATTCAAACGGC
GACACTAACGGTGATGCGAGCGCCACGAAGCCACCAGCACCGGCA
CCGTTAAGAGAAAAAATCTTCAAATTCTCAGAGTCAGCAATCGAC
AAAATCAAAGCAAAAATCAATGCGAATCCACCGGAAGGATCAACC
AAGCCATTCTCCACATTTCAATCGCTCTCCACACACATATGGCAC
GCAGTTACACGCGCTCGCAATCTAAAACCGGAAGACTACACCGTT
TTCACTGTTTTCGCCGATTGCCGGAAACGTGTCGATCCTCCGATG
CCGGATAGCTATTTCGGAAACCTAATTCAAGCGATCTTCACCGTC
ACCGCTGCCGGATTATTGCAGGCGAATCCACCGGAATTCGCGGCG
TCAATGATACAAAAAGCGATTGATATGCACGATGCGAAAGCAATT
GAAGCGCGTAACAAAGAATGGGAAAGTAATCCGATTATATTTCAA
TACAAAGACGCCGGAGTTAATTGTGTTGCGGTTGGGAGTTCACCT
AGGTTTAAGGTTTATGATGTGGATTTCGGGTTTGGTAAACCCGAA
AGTGTTCGGAGCGGGGCGAATAACCGGTTTGATGGTATGGTTTAT
TTGTATCAGGGAAAAAGTGGTGGAAGGAGTATTGATGTGGAGATT
AGTTTGGATGCAAGTGCAATGGGAAATCTTGAAAAGGATAAGGAA
TTTCTTATCCAAGAATAA.

In some embodiments, the DNA molecule comprises a nucleic acid sequence with at least 84%, at least 87%, at least 90%, at least 95%, or at least 99% homology or identity to SEQ ID NO: 115, or any value and range therebetween. Each possibility represents a separate embodiment of the invention. In some embodiments, the DNA molecule comprises a nucleic acid sequence with 84% to 100%, 88% to 100%, 90% to 100%, or 95% to 100% homology or identity to SEQ ID NO: 115. Each possibility represents a separate embodiment of the invention.

In some embodiments, the DNA molecule comprises or consists of the nucleic acid sequence:

(SEQ ID NO: 116)
ATGGCTTCTCTTCCTCTCTTAACTGTTCTTGAACAATCCCATGTA
TCACCACCGCCAGCCACCGTAGTCGATAAATCGTTGTCGCTAACC
TTTTTCGATTTCCTGTGGCTAACTCAACCTCCAATTCACAATCTT
TTCTTTTACGAGTTTTCAATCGACGAAACTCAGTTCGTGGAAACT
ATCGTTCCTAGTCTTAAAAACTCGTTATCAATCACTCTTCAACAT
TTTTACCCGTTCGCCGGTAACCTTATCTTATTTCCTGATAACAAA
AGGCCTGAAATTCGTTACGTTGAAGGTGATTATGTCATGGTTACA
TTTGCAAAATCTAGCCTTGACTTCAATGAACTAGTAGGAAACCAT
CCTAGAGATTGTGACCAGTTTTATGATCTTATTCCTCCATTAGGT
GAAAGTGTGAAAACTTCTGAATTTCGAAAAATCCCACTCTTTTCG
GTCCAGGTGACGTTTTTTCCACAAAAAGGCGTATCGATTGGTATG
ACGAATCATCATAGTCTTGGCGATGCTAGCACTCGGTTTTGTTTC
TTGAACGCGTGGACATCGATTTCTAGATCTAGTTCAGATGAGTCA
TTTCTAGCAAACGGAACTAAACCGTTTTACGATAGAGTGATAAGT
AACCCGAAACTAGATCAAAGTTATCTAAAATTTTCCAAGATCGAT
ACTCTTTACGAGAAGTATCAACCTTTAAGCCTCTCTAGACCATCT
AATAAACTTCGTGGCACGTTTATCTTGACGCGAAAAATCCTAAAC
GAGTTGAAAAAAAGTGTGTCAATTAAACTACCAACTTTATCATAT
GTATCATCTTTTACGGTTGCATGTGGTTATATTTGGAGTTGCATA
GCGAAATCACGAAACGATGATCTACAACTATTCGGGTTCACTATT
GATTGTAGGGCACGTTTGGATCCACCGGTTCCATCAACTTATTTT
GGGAATTGTGTCGGGGGTTGTATGGCGATGGCAAAAACAACGTTG
TTAACCGAAGACGATGGATTTATAACGGCTGCTAAATTGCTTGGA
GAAAGTTTACACAAGACGTTGACCGAATCGGGTGGAATCGTGAAA
GATATAGAAGTGTTTGAAGATTTGTTTAAGGATGGATTACCAACA
ACTATGATAGGAGTTGCGGGAACACCAAAGCTTAAGTTTTATGAG
ACGGATTTCGGGTGGGGGAACCCGAAAAAGGTGGAAACGATTTCG
ATTGATTATAACATGTCGATTTCTATGAACGCTTGTAGAGAATCG
AAGGATGATTTGGAGATTGGTGTTTGCCTTATGAATACTGAAATG
GAAGCTTTTGTTCGTTTATTTGATGAAGGATTAGAATCATACGTT
TAG.

In some embodiments, the DNA molecule comprises a nucleic acid sequence with at least 77%, at least 85%, at least 93%, at least 97%, or at least 99% homology or identity to SEQ ID NO: 116, or any value and range therebetween. Each possibility represents a separate embodiment of the invention. In some embodiments, the DNA molecule comprises a nucleic acid sequence with 77% to 100%, 80% to 100%, 85% to 100%, or 93% to 100% homology or identity to SEQ ID NO: 116. Each possibility represents a separate embodiment of the invention.

In some embodiments, the DNA molecule comprises or consists of the nucleic acid sequence:

(SEQ ID NO: 117)
ATGGGAAGTGAAAATGTTCACAAAATAATGAAAATCAACATCACT
AAATCATCATTTGTACAACCCTCAAAGCCTACAGTACTACCCACT
AACCACATATGGACTTCTAACTTAGATTTAGTTGTGGGTAGAATT
CATATTTTAACCGTTTACTTTTACCGTCCAAATGGTGCTTCGAAT
TTTTTTGATCCAATTGTTATGAAAAAAGCTTTAGCTGATGTGCTT
GTTTCTTTTTATCCGATGGCCGGAAGAATAAGTAAAGATGATAAT
GGTAGAGTTGTAATTAATTGTAATGATGAAGGTGTTTTGTTTGTT
GAAGCTGAGTCAGATTCCACGTTGGATGACTTCGGTGAGTTTACA
CCGTCTCCGGAGCTCCGACAACTTACCCCGACGATTGATTACTCC
GGTGACATTTCAACGTACCCGCTATTTTTTGCACAGGTAACGCAT
TTCAAGTGTGGAGGAGTTGGTTTTGGTTGTGGTGTGTTTCATACA
CTTGCAGATGGTCTATCCTCTATACATTTCATCAACACATGGTCG
GACATGGCTCGTGGTCTCTCGATAGCCATCCCGCCATTCACTGAC
CGGACCCTTCTTCGTGCACGTGAACCACCCACTCCCACTTTTGAC
CACGTAGAGTACCACCTCCCTCCGTCCATGAAAACTACCTCACAA
ACCAACAAATCCAGAAAGCCTTCCACGGCCATGTTAAAGCTTACG
CTTGATCAACTAAATGCTCTCAAAGCTGCTGCTAAGAATGAAGGC
GGCAACACCAATTATAGCACGTACGAGATCCTGGCGGCTCATTTA
TGGCGGTGTGCCTGCAAGGCTCGAGGACTCCCTGATGACCAACTA
ACCAAATTGTACGTGGCAACAGATGGACGGTCCAGATTGAGCCCT
CAACTCCCACCAGGCTATCTAGGCAATGTTGTGTTCACCGCCACC
CCAGTTGCCAAATCAGCTGACCTCACGACTCAACCATTGTCTAAT
GCAGCATCTTTGATCCGAACCACATTGACAAAAATGGATAACGAC
TATTTGAGATCTGCCATTGATTACCTTGAGGTGCAGCCAGATCTA
TCTGCTTTAATTCGTGGTCCTAGTTACTTTGCTAGCCCGAATTTG
AACATAAACACGTGGACCCGGTTGCCAGTACATGATGCGGATTTC
GGGTGGGGTCGGCCTGTTTTCATGGGACCAGCAGTGATATTGTAT
GAGGGCACCATCTATGTTCTACCAAGCCCAAACAATGATAGGAGT
ATGTCATTGGCAGTCTGTTTAGATGCAGATGAACAACCATCGTTT
GAGAAGTTCCTGTATGACTTTTAA.

In some embodiments, the DNA molecule comprises a nucleic acid sequence with at least 87%, at least 90%, at least 93%, at least 95%, or at least 99% homology or identity to SEQ ID NO: 117, or any value and range therebetween. Each possibility represents a separate embodiment of the invention. In some embodiments, the DNA molecule comprises a nucleic acid sequence with 87% to 100%, 90% to 100%, 93% to 100%, or 95% to 100% homology or identity to SEQ ID NO: 117. Each possibility represents a separate embodiment of the invention.

In some embodiments, the DNA molecule comprises or consists of the nucleic acid sequence:

(SEQ ID NO: 118)
ATGCCTTCATCATCATCATCGCCTTCTTCAACAGCTGATTCAGTT
ACCATAATCTCAAAATGCACAGTCTACCCACATATGAAAAACTCA
ACACCAGAATCCTTGCAGCTCTCTGTTTCTGATCTCCCAATGCTT
TCATGTCAATACATACAAAAAGGTGTCTTACTTTCTCAACCGCCA
CCCAATCACACCAACAATATCATTTCCCACTTAAAACTCTCTCTC
TCTAAAACCCTCTCTCACTTCCCACCTCTCGCCGGCCGTCTTTCG
ACCGACTCTCACGGCCACGTCTCTATCATCTGCAACGATTCCGGC
GTCGAATTCGTTCACTCCACCGCTAACCACCTCCACACCCACCAA
ATCTTACCCCTCAATTCCGACGTTCACCCATGTTTTAAAACCTTT
TTTGCTTTTGATAAAACTCTGAGTTACGCCGGCCACCACCAACCA
ATCGCCGCCGTGCAAGTCACGGAGCTTGCTGATGGACTCTTTATT
GGGTGTACGGTAAATCATGCTGTCGTTGACGGGACTTCTTTTTGG
AACTTTTTTAATACTTTTGCTGAGATCACAAAAGGGTGTCAGAAA
GTAACGAACTTGCCGGATTTTAGCCGGGAAAATGTTTTCATTTCT
CCGGTTGTTTTGCCTCTTCCCTCCGGCGGCCCGTCGGCGACGTTC
TCAGGTGATGAGCCGTTGAGGGAAAGGATCATTCATTTCAGTAGA
GACGCGATTCTGAAGATGAAATTCAGAGCTAATAATCCTTTATGG
CGGCAACCACAAAATTCGGATCTGGATGATACAGAGATTTACGGG
AAAGTGTGTAACGACATTAACGGCAAAGTTAACGGGGCGTTTAAA
CCCAAAAGTGAAATTTCGTCCTTCCAGTCTTTATGTGGTCAGTTA
TGGCGTGCGGTTACACGCGCGCGTAAATTCAACGACCCTATAAAA
ACGACGACGTTTCGAATGGCGGTGAATTGTAGGCATAGGCTAGAC
CCAAAGGTCGACAAACTTTATTTCGGGAACTTGATCCAAAGCATC
CCGACCGTTGCTTCAGTTGGGGAGTTGTTATCACATGATTTGTCG
TGGGCAGCCAATGAGCTTCACCAAAATGTGGTGGCGCATGATAAT
GCTACCGTGCGCAGGGGTGTTAAGGATTGGGAGAATAATCCAAAG
TTGTTTCCTTTGGGGAATTTTGATGGTGCTATGATCACAATGGGA
AGTTCTCCTAGGTTTCCAATGTATAATAACGATTTCGGGTGGGGC
CGCCCAATGGCGGTTCGTAGTGGTAAAGCTAATAAGTTTGATGGA
AAGATTTCGGCTTTTCCGGGACGTGATGGTGATGGTAGTGTCGAT
CTTGAGGTTGTTTTAGCTCCCGAAACCATGGCATGTCTTGAACGT
GACCATGAATTTATGCAATATGTATCTTAA.

In some embodiments, the DNA molecule comprises a nucleic acid sequence with at least 82%, at least 90%, at least 97%, or at least 99% homology or identity to SEQ ID NO: 118, or any value and range therebetween. Each possibility represents a separate embodiment of the invention. In some embodiments, the DNA molecule comprises a nucleic acid sequence with 82% to 100%, 85% to 100%, 90% to 100%, or 95% to 100% homology or identity to SEQ ID NO: 118. Each possibility represents a separate embodiment of the invention.

In some embodiments, the DNA molecule comprises or consists of the nucleic acid sequence:

(SEQ ID NO: 119)
ATGAAGTGGTTTTTCATAACCCATAAAGCAACCCAGCGTTGCCTT
AATTCTAAACAATTTCATCTTCACGGAGGTTCGAATTTCGTTTCC
GGTAATAGATGTTTTCTTGCATCACACTCAATGGAGCGGCCAAAA
TTCATGTTGATACCATATTATCCCTACCAAATTCGGTCCTTAAAT
TCGAGTCACCGATATAGTAGTACGTCACCCAGCGGATCCCCTCAC
AGTTTTCTGAATGGTACTAAGAATGAAAACTATACGAAGAAGGTA
GATCTTGAAATAATTTCAAGAGAAATCATCAAACCAGCTTCTCCA
ACTCCACATCATTTAAGAAACTTCAACTTATCACTTTTGGACCAA
ATAGTATTTGATTGCTACACCCCTGTAATCCTCTTTATTCCAAAT
AGTAATAAGGCTACTGTTACGGATGTCATGATCAAAAGATTGAAA
CATCTCAAGGAGACTTTATCTCGAATTCTAAGTCAATTTTATCCC
TTTGCGGGAGAAGTTAAGGACAGATTGCATATCGAATGCAATGAC
AAGGGAGTCAATTACATCGAGGCTCAAATCAATGAGACATTGGAA
GAATTTCTATGTCATCCAGATAACGAAAAGGCGAGGGAGCTTATG
CCCGAAAGCCCTCATGTTCAAGAATCTGCAATAGGAAACTATGCT
ATGGGTATTCAGATAAACATTTTCAGTTGCGGAGGGATTGGACTT
TCCATGAGCATGGCACACAAGATCATGGACTTCTACACATATACG
ATCTTCATGAAAGCATGGGCTGCAGCTGTTCGAGGTTCACCAGAT
ACAATTATTTCACCAAGTTTTGTGGCTTCTGAGGTCTTTCCTAAT
GATCCCAGCCAAGAAGATTCAATTCCTATCGAGTTAAAGTCTAGT
AATTTGCTTAGCACAAAAAGATTTGAGTTTGATCCTACTGCGTTG
GCTCTCCTAAAGGGACAAGTTGTCGCCAGCGGATCACCTCCCCAA
CGAGGACCAAGTCGTATGGAGGCGACAACAGCCGTTATTTGGAAG
GCCGCTGCAAAAGCTGCATCGACTGTCAGAAGATTCGATCCAAAG
TCACCTCATGCGCTGGCGTTACCAGTAAATATACGTAAAAGGGCA
TCACCTGCTCTCCCAGACAATTCCATAGGAAACATAGTTATGCGA
GGTATAGCAATTTGTTTTCCTGAGAGCCAACCGGACTTGCCAACT
CTTATGGGTAAAGTGAGAGAATCAATAGCGAAACTTAACTCAGAT
TACATTGAGTCCCTGAAAGGTGAAAAGGGGCATGAGACAGTTAAT
AAGATGTTGAAGGAGTTGAAGCTTCGGACGAATATGACAAAGGTA
GGAGGGAAATTCGTTGCTAGTTGCATATTTAATAGTGGAATATAT
GAGTTGGATTTCGGGTGGGGAAAACCGATATGGTTCTATGTTGTG
AATCCAGGAAGCGATAGTTGTGTGGTTTTGACTGATACGCTGAAG
GGTGGTGGTGTTGAAGCCACAATTACACTACCACCAGATGAAATG
GAGATATTCGAACGTGATCATGAGCTTCTATCCTATACTACCATC
AACCCTAGTCCACTGCGATTTCTTGACCATTGA.

In some embodiments, the DNA molecule comprises a nucleic acid sequence with at least 74%, at least 80%, at least 85%, or at least 95% homology or identity to SEQ ID NO: 119, or any value and range therebetween. Each possibility represents a separate embodiment of the invention. In some embodiments, the DNA molecule comprises a nucleic acid sequence with 74% to 100%, 80% to 100%, 87% to 100%, or 95% to 100% homology or identity to SEQ ID NO: 119. Each possibility represents a separate embodiment of the invention.

In some embodiments, the DNA molecule comprises or consists of the nucleic acid

(SEQ ID NO: 120)
ATGGAGGTGCCTGACCAATTCCACCTAAACATTCTTGAACAATGC
CACGTTTCACCATCACCAAATTCCATCATACCTTCATTTTCACTA
CCCTTAACATTCTTAGACATCCCATGGCTTTTTTACCCTTCAAAT
CAAACCCTTTTTTTCTTCCCAGAACCACCACCCAAAACCACCATC
ATCACCACCCTTAAACAATCACTCTCTCTTACCCTCCACCACTTC
CACCCTCTCGCCGGAAACCTCTCACTTCCATCACCTCCGGCGGAA
CCCCACATTGTTTACACCAAAAATGACTCAATTGCACTCACAATT
GCTCAAACAAACACCAACATCCACCATCTTTCTTGCAATCACCCA
AGAAGTGTAAAAAATCTTTACTCTCTTTTACCCAAACTCCCATCT
CCATCCATGTCACGTGAAACTCACGTGGGCCTTGTTATCCCCCTT
CTTACCATCCAAATTACGGTTTTTGCTGATTTGGGGTATTCGATC
GGAGTCACTATGCAACATGCAGCAGTTGATGAACGGACATTTGAT
CAGTTTATGAAATGTTGGGCGTCTGTTTGTACATCTTTGTTGAAA
AATGACTCACTTTTTACATTCAAGTCTACACCTTGGTACGATAGG
AGCGTAATTATCGACCCCAAATCGCTGAAAACAACGTTTTTAAAG
CAATGGTGGAACCGATCTAATTCTCTCAATGAGTCACATGATCAA
GAAAATGATGATCATGATCTTGTTCTAGCAACTTTTGTTTTGAGT
TCATTAGATATTAACATGATCAAGAATCATATTCTTGCAAAATGC
AAGATGATAAATGAGGATCCACCACTACATTTATCTCCTTATGTT
AGTGCATGTGCTTATTTATGGAAATGTTTAATCAAAATTCAAGAA
ACCCATGATTCTATTAAGGGTGGTCCTCTCTATTTAGGGTTTAAT
GCCGGTGGGATTACTCGATTAGGGTACGACATACCTTCAACTTAT
TTTGGGAATTGTATAGCTTTTGGGAGATGCAAGGCATTTGAGAGT
GAATTATTGGGTGATAATGGTATTGTTTTCGCGGCAAAATCGATT
GGAAAAGAGATCAAGAGGCTTGATAAGGATGTTTTAGGAGGTGCT
AATAAGTGGATTAGTGATTGGGATGAATTAACCATTAGGCTTCTT
GGTTCACCAAAAGTTGATTCATATGGTATGGATTTTGGATGGGGT
AAAGTTGAGAAGGTTGAAAAAATATCAAGTATTTCAAATCACGGT
AGGGTTAATGTAATTTCTTTGAGTGGATGTAAGGATTTTAAAGGT
GGAATAGAGATAGGGGTTGTTCTTTCTGTGGCTAAAATGAATGTT
TTCACTTCCCTCTTTCATGGAGGTTTAATGGAGTTTGCATATTGA.

In some embodiments, the DNA molecule comprises a nucleic acid sequence with at least 79%, at least 87%, at least 93%, at least 97%, or at least 99% homology or identity to SEQ ID NO: 120, or any value and range therebetween. Each possibility represents a separate embodiment of the invention. In some embodiments, the DNA molecule comprises a nucleic acid sequence with 79% to 100%, 85% to 100%, 90% to 100%, or 95% to 100% homology or identity to SEQ ID NO: 120. Each possibility represents a separate embodiment of the invention.

In some embodiments, the DNA molecule comprises or consists of the nucleic acid

(SEQ ID NO: 121)
ATGAAAAATAAGAACCCGACTAGTGTGATCAGAGAGGCTTTAGCT
AAGGTATTGGTGTTTTATTATCCGTTTGCTGGCCGGCTCAAGGAA
GGGCCGGCCAGGAAACTGATGGTGGATTGTTCTGGTGAAGGTGTG
TTGTTTATTGAGGCAGAAGCTGATGTCACGTTGAAACAATTTGGT
GACGCACTTCAACCGCCATTTCCTTGTTTAGAAGAGCTTCTTTAC
GATGTTCCTGGATCTACTGGTATTCTAGATACACCATTATTGCTG
ATTCAGGTGACACGATTGTTATGTGGAGGTTTTATCTTTGCTCTA
CGACTCAACCACACCATGAGCGACGCAGCAGGTCTCGTTCAATTC
ATGACAGGGCTTGGTGAAATGGCACAAGGTGCATCAAGGCCATCA
ACGTTGCCTGTATGGCAAAGGGAGTTGCTTTTTGCAAGGGACCCA
CCACGCGTGACTTGTACTCATCACGAGTATACTGAAGTGGAAGAC
ACCAATGGTACAATCATTCCGCTAGATGACATGGCACATAAATCA
TTTTTCTTTGGACCTTCTGAGATATCAGCGTTGCGAAGGTTCGTT
CCATCATACCTAAAAAAGTGTTCTACTTTTGAGGTCTTAACCGCT
TGCCTATGGCGTTGTCGTACAATTGCACTCCAGCCAGATCCCGAA
GAAGAGATGCGCATGATATGCATTGTTAATGCGCGTGGAAAGTTT
AATCCTCCCCTATTACCCAAAGGATATTATGGAAATGGTTTCGCT
ATACCAGTGGCCATTTCAACAGCTGGAGACCTATCTAGCAAACCA
TTAGGTCACGCATTGGAACTTGTAATGAAAGCCAAATCCAATGTC
ACTGAGGAGTATATGAGATCAGTAGCCGACTTAATGGTAATCAAG
GGACGACCCCACTATACGGTTGTCCGAAGCTACCTTGTATCGGAT
GTGACTCACGCTGGATTTGATGTTGTTGATTTCGGGTGGGGGAAA
GCGTCCTATGGAGGACCTGCAAAAGGGGGAGTAGGTGCTATTCCC
GGAGTTGTTACTTTCTTTATACCTTTTACAAACCATAAAGGCGAG
TCTGGAATTGTGCTACCTATATGTTTGCCGAGTGCAGCCATGGAT
AAGTTTGTTGAAGAGTTAAATAAGATGTTGGTCCCAGACAACAAC
GAACAAGTACTCCGAGAACACAAGTTACTAGTTCTCGCTAGATTG
TAA.

In some embodiments, the DNA molecule comprises a nucleic acid sequence with at least 82, at least 85%, at least 95%, or at least 99% homology or identity to SEQ ID NO: 121, or any value and range therebetween. Each possibility represents a separate embodiment of the invention. In some embodiments, the DNA molecule comprises a nucleic acid sequence with 82% to 100%, 85% to 100%, 90% to 100%, or 95% to 100% homology or identity to SEQ ID NO: 121. Each possibility represents a separate embodiment of the invention.

In some embodiments, the DNA molecule comprises or consists of the nucleic acid

(SEQ ID NO: 122)
ATGGCACAAATCGACACTCCATTGACATTCAAAGTCCGGAGACAT
GCACCGGAGCTGATCGCTCCAGCGAAACCTACGCCACGAGAACTA
AAACCTCTATCCGACATTGATGATCAAGAAGGCCTTAGGTTTCAT
ATCCCAGTGATTCAATTCTATCGTAGCGATCCAAAGATGAAAAAT
AAGAACCCGGCTAGTGTGATCAGAGAGGCTTTAGCTAAGGTGTTG
GTGTTTTACTATCCGTTTGCTGGCCGGCTCAAGGAAGGGCCTGCC
AGGAAACTGATGGTAGATTGCTCTGGTGAAGGTGTGTTGTTTATT
GAGGCGGAAGCTGATGTCACGTTGAAACAATTTGGTGACGCCCTT
CAACCGCCGTTTCCTTGTTTGGAAGAGCTTCTTTACGATGTTCCT
GGATCTACTGGCGTTCTAGATACACCGTTATTGCTGATTCAGGTG
ACACGATTGTTATGTGGAGGTTTTATCTTTGCTCTACGACTCAAT
CACACCATGAGCGACGCACCAGGTCTCGTTCAATTCATGACAGGG
CTCGGTGAAATGGCACAAGGTGCATCAAGGCCATCTACGTTGCCT
GTATGGCAAAGGGAGTTGCTTTTAGCAAGGGACCCACCACGCGTG
ACATGTACTCATCACGAGTATACTGAAGTGGAAGACACCAAGGGT
ACAATCATTCCGCTAGATGACATGGCACATAAATCATTTTTCTTT
GGACCTTCTGAGATATCAGCATTGCGAAGGTTCGTTCCATCATAC
CTAAAAAAGTGTTCTACTTTTGAGGTCTTAACCGCTTGCCTATGG
CGTTGTCGTACAATTGCACTCCAGCCAGATCCCGAAGAAGAGATG
CGCATAATATGCATTGTTAATGCGCGCGGAAAGTTTAATCCACCC
CTTCCTAAAGGTTATTATGGAAATGGTTTTGCTTTCCCAGTGGCC
ATTTCAACAGCTGGAGATCTATCCAGCAAACCATTAGGTCATGCA
TTGGAACTTGTAATGAAAGCCAAATCCGATGTCACTGAGGAGTAT
ATGAGATCAATAGCCGACTTAATGGTAATCAAGGGACGTCCCCAC
TTTACGGTTGTCAGAAGCTACCTTGTCTCGGATGTGACTCACGCT
GGATTTGATGTTGTTGATTTCGGGTGGGGGAAAGCGGCCTATGGA
GGACCCGCTAAAGGGGGAGTAGGTGCTATCCCAGGTGTTGCTAGT
TTCTATATACCTTTTACAAACCATAAAGGCGAGTCTGGAATTGTG
CTACCTATATGTTTGCCGAGTGCGGCCATGGATAAGTTTGTTGAA
GAGTTAAATAAGATGTTGGTCCCAGACAACAACGAACAAGTACTC
CGAGAACACAAGTTACTAGTTCTTGCTAGATTGTAA.

In some embodiments, the DNA molecule comprises a nucleic acid sequence with at least 83%, at least 85%, at least 89%, at least 92%, at least 95%, at least 97%, or at least 99% homology or identity to SEQ ID NO: 122, or any value and range therebetween. Each possibility represents a separate embodiment of the invention. In some embodiments, the DNA molecule comprises a nucleic acid sequence with 83% to 100%, 88% to 100%, 92% to 100%, or 95% to 100% homology or identity to SEQ ID NO: 122. Each possibility represents a separate embodiment of the invention.

In some embodiments, the DNA molecule comprises or consists of the nucleic acid

(SEQ ID NO: 123)
ATGGAAATACAAGTAATAAACTACTCATCAAAGCTAGTAAAACCC
TTGACACCAACACCCACCGCAAATCGTTACTATAACATTTCTTTC
ACCGATGAGCTCGTCCCAACCATTTACGTCCCACTCATTCTCTAC
TACGCAACACCGAAAAACCCAAATGGTGATCACTTTGAAAACATT
TGTGACCGTCTGGAGGAGTCGTTATCGAAAACGTTAAGTGATTTT
TACCCACTGGCCGCGAGATTCATTCGTAAACTCTCCTTAATTGAT
TGTAACGATCAAGGGGTTTTGTTTGTCCTAGGCAATGTAAATATC
CGACTTTCGGATGTTACAGGCCTAGGACTGACGTTTAAAACCAGT
GTTTTAAATGATTTTCTCCCGTGTGAGATTGGAGGAGCGGATGAA
GTCGATGATCCTATGCTTTGTGTCAAAGTCACCACTTTTGAGTGT
GGTGGTTTTGCAATTGGTATGTGTTTTTCGCATAGGCTTTCGGAT
ATGGGTACCATGTGTAACTTTATTAACAATTGGGCTGCTAGAACT
ATTGGTGAATATGATAATGAAAAACATACTCCTATTTTTAATTCG
CCGTTGTACTTCCCGCAACGAGGATTACCTGAACTTGACCTAAAA
GTACCTAGGTCAAGTATTGGTGTGAAAAATGCAGCACGCATGTTT
CACTTTAATGGGAAGGCAATATCATCCATGAGAGAAGTTTTTGGA
GTTGATGAAAATGGGTCTCGTAGACTCTCAAAGGTTCAACTTGTT
GTAGCCTTGTTGTGGAAGGCCTTTGTTCGCATAGATGATGTGAAC
GATGGCCAATCTAAGGCGTCTTTTCTGATCCAACCAGTTGGGTTG
AGGGACAAAGTTGTCCCTCCATTACCATCAAACTCATTTGGGAAT
TTTTGGGGTCTAGCGACTTCCCAACTTGGTCCTGGTGAGGGTCAC
AAAATCGGTTTCCAAGAATATTTTTACATTTTGCGTGAATCTATT
AAGAAAAGAGCTAGGGATTGCGCTAAAATATTGACACACGGTGAA
GAAGGATATGGGGTTGTAATCGATCCATATCTTGAGTCGAATCAA
AAGATAGCTGATAATGGTACAAACTTTTACTTGTTCACTTGTTGG
TGCAAGTTTTCGTTCTACGAAGCTGATTTTGGTTGTGGTAAGCCG
ATTTGGGCTAGCACCGGAAAGTTTCCGGTTCAAAATTTGGTGATC
ATGATGGATGATAATGAGGGTGATGGTGTAGAAGCGTGGGTTCAT
TTAGACGATAAACGCATGAATGAGTTAGAACAAGATCCTGATGTT
AAACTCTACGCATGCAATTTAGCTTAA.

In some embodiments, the DNA molecule comprises a nucleic acid sequence with at least 77, at least 85%, at least 95%, or at least 99% homology or identity to SEQ ID NO: 123, or any value and range therebetween. Each possibility represents a separate embodiment of the invention. In some embodiments, the DNA molecule comprises a nucleic acid sequence with 77% to 100%, 82% to 100%, 87% to 100%, or 95% to 100% homology or identity to SEQ ID NO: 123. Each possibility represents a separate embodiment of the invention.

In some embodiments, the DNA molecule comprises or consists of the nucleic acid

(SEQ ID NO: 124)
ATGAAATTAGCAGTGAAGGAATCAGTGATAGTAAAACCATCCAAA
ACGACACCGTGTCAGCAAATATGGACATCAAATCTTGATTTAGTG
GTGGGTCGGATCCATATATTAACCGTTTACCTTTACAGACCAAAT
GGGTCTTCAAATTTCTTTGATTCCATGGTTTTAAAGAAGGCTCTA
GCCGACGTTTTAGTTTCTTTTTTTCCGGTGGCCGGACGGTTGGAT
AAAGACGGTGACGGCAGAGTTGTAATAGATTGTAACGGTGAGGGT
GTTTTGTTTGTGGAAGCTGAAGCTGATTGTTGCATTGATGATTTT
GGTGAGATTACTCCGTCGCCGGAGTTACGACGGTTGGTGCCGACG
GTGGATTATTCCGGTGATATGTCTTCTTATCCGTTATTTATTACG
CAGGTTACACGGTTCAAGTGTGGGGGAGTTTCGTTAGGCTGTGGA
CTACACCATACGTTATCGGATGGACTCTCAGCACTTCACTTCATC
AACACATGGTCTGATGTAGCTAGAGGCCTATCGGTGGCAATCCCA
CCGTTCATTGACCGCTCCCTTCTTCGAGCTCGTGATCCACCATCC
CCTGTGTTTGACCACATCGAATACCACCCACCACCGTCACTGATC
ACTCCGTTGCAAAACCAAAAGAACGCGTCACATTCGAGGTCTGCT
TCAACTTTAATCCTACGGCTCACACTCCATCAAATAAACAATCTT
AAATCAAAGGCTAAAGGCGATGGGAGCATGTACCATAGCACGTAC
GAGATCCTAGCTGCTCATCTATGGCGATGTGCGTGCAAAGCACGT
GGACTAGCAAACGATCAACCAACCAAATTGTATGTGGCCACCGAT
GGACGGTCAAGATTGATTCCTCCACTCCCTCCGGGCTACCTTGGG
AATGTCGTTTTCACGGCTACTCCTGTCGCTAAATCGGGAGATTTC
GAATCTGAATCCTTGGCAGAGACAGCAAGGAGGATTCGCAGTGAG
TTGGGTAAAATGAACGATGAGTATCTTAGATCAGCTATTGACTAC
TTAGAGTCGGTATCTGATATTTCGACCCTTGTTAGAGGGCCGACT
TACTTTGCGAGTCCAAATCTGAATGTAAACAGTTGGACTCGGTTA
CCAATATACGAATCTGACTTCGGTTGGGGTCGACCTATTTTCATG
GGACCCGCAAGTATACTTTACGAGGGTACGATTTACATCATACCG
AGCCCTAGTGGTGATCGGAGTGTGTCTCTGGCCGTGTGCTTGGAC
CCTGATCACATGGCTTTGTTTAAAGAATGCTTGTACGTTTTTTAG.

In some embodiments, the DNA molecule comprises a nucleic acid sequence with at least 84, at least 89%, at least 95%, or at least 99% homology or identity to SEQ ID NO: 124, or any value and range therebetween. Each possibility represents a separate embodiment of the invention. In some embodiments, the DNA molecule comprises a nucleic acid sequence with 84% to 100%, 88% to 100%, 93% to 100%, or 97% to 100% homology or identity to SEQ ID NO: 124. Each possibility represents a separate embodiment of the invention.

In some embodiments, the DNA molecule comprises or consists of the nucleic acid

(SEQ ID NO: 125)
ATGAAGCTAGCAGTGAAGGAATCAGTGATAGTAAAACCATCCAAA
ACGACACCGTGTCAGCAAATACGGACATCAAATCTTGATTTAGTG
GCGGGTCGGATCCATATATTAGTCGTTTTCTTTTACAGACCAAAT
GGGTCTTCGAATTTCTTTGATTCCTTGGTTTTAAAGAAGGCTCTC
GCCGACGTTTTAGTTCCTTTTTTTCCGGTGGCCGGACGGTTCAGT
GAAGACGGTGACGGCAGAGTTGTAATTGATTGTAACGGTGAGGGT
GTTTTGTTTGTGGAATCTGAAGCTGATTGTTGCATTGATGATTTT
GGTGAGATTACTCTGTCGCCGGAGTTACAACAGTTGGTGCCGACG
GTGGATTATTCCGGTGATATGTCTTCTTATCCGTTATTTATTGCG
CAGGTCACACGGTTCAAGTGTGGGGGAGTTTCGTTAGGTTGGGGA
CTACACCATACATTATTGGATGGACTCTCAGCACTTCACTTCGTC
AACACATGGGGTGATGTAGCTAGAGGCCTATCGGTGGCAATCCAA
CCGTTCATTGACCGCTCCCTTCTTCGAGCTCGTGATCCACCGACC
CCTGTGTTTGACCACATCGAATACCACCCACCACCGTCACTGATC
ACTCCATTGCAAAACCAAAAGAACGCATCACATTCGAGGTCTGCT
TCAACTTTAATCCTACAGCTCACACCCGATCAAATAAAGAATCTT
AAATCAAAGGCTAAAGGCGATGGGAGCATGTACCATAGCACATAC
GAGATCCTAGCTGCTCATCTATGGCGATGTGCGTGCAAAGCGCGT
GGACTAGCAAACGATCAACCAACCAAATTGTATGTGGCCGCCAAT
GGACGGTCAAGATTGATTCCTCCACTCCCTCCGGGCTACCTTGGG
AATGTCGTTTTCAACGCTACTCATGTCGCTAAATCGGGGGATTTT
GAATCTGAATCCTTGGCAGAGACTGCAAGGAGGATTCACTGTGAG
TTGGGTAAAATGAACGATGAGTATTTTAGATCAGCTATCGACTAC
TTAGAGTCGGTAGATGATATTTCAACCCTTGTCAAAGGGCCGACT
TACTTTGCGAGTCCAAATCTGAATGTATACAGTTGGATTGGGATA
CCAATATATGCATGTGACTTCGGATGGGGTCAACCTATTTTCATG
AGACCCGCAAGTTTCCTTTACGATGGTTCCATTTACATCATACCG
AGCCCTAGTGGTGATCGGAGTGTGTTGTTGGCCGTGTGCTTGGAC
CCTGATCACATGGATTTGTTTAAAGAATGCTTGTACGCTTTTTAG.

In some embodiments, the DNA molecule comprises a nucleic acid sequence with at least 82, at least 85%, at least 95%, or at least 99% homology or identity to SEQ ID NO: 125, or any value and range therebetween. Each possibility represents a separate embodiment of the invention. In some embodiments, the DNA molecule comprises a nucleic acid sequence with 82% to 100%, 85% to 100%, 90% to 100%, or 95% to 100% homology or identity to SEQ ID NO: 125. Each possibility represents a separate embodiment of the invention.

In some embodiments, the DNA molecule comprises or consists of the nucleic acid

(SEQ ID NO: 126)
ATGGTGATGATTAGCAAGCTTTTACGATTAGGTAGAAGAAAACTT
CACACAATTGTATCAAGAGATACCATTAGACCTTCTTCTCCAACT
CCCTCTCATTCCAAAACATATAATCTCTCCTTGCTCGATCAAATA
GCTGTAAATTCATACGTGCCGATTGTTGCTTTTTACCCAAGCTCA
AATGTTTGTCGAAGTTCCGATGATAAGACGCTGGAGTTGAAGAAC
TCATTATCGAAAATATTAACTCATTACTATCCGTTTGCCGGTAGA
ATGAAGAAGAATCGCCCTACCGTCGTTGATTGCAATGATGAAGGG
GTTGAGTTCGTTGAAGCACGTAATACCAACTCGTTATCAGATTTC
CTCCAACAATCGGAGCACGAAGATCTAGATCAACTCTTTCCAGAT
GATTGTGTATGGTTCAAACAAAACCTTAAAGGTTCTATTAATGAC
GCAAATAATAGTAGCGTATGTCCATTGAGCATTCAAGTCAACCAT
TTCGCGTGTGGAGGTGTAGCAGTTGCAACTTCGTTACGCCACAAG
ATTGGAGACGGAAGCAGTGCGTTAAATTTCATTAAACACTGGGCT
GCAGTTACGTCACACTCTCGAGCAGGGAATCATCAAATTGATGCG
ACATCACCCATCATTAATCCCCATTTCATTTCTTACCCAACTAGA
ACTTTTAAATTGCCAGATAGGTCACCATACATACCACCTAGTGAT
GTTGTGTCAAAAAGTTTTGTTTTCCCCAACACAAATATAAAGGAC
CTCCAAGCCAAGGTGGTAACCATGACCATGGGCTCTAGACAACCT
ATCGTGAACCCTACCCGAGCTGATGTCGTATCATGGCTTCTACAT
AAGTGTGTAGTAGCAGCAGCTACCAAAAGGATATCGGGAAATTTT
AAAGAAAGTTGCGTGATCTCGCCATTAAATCTGAGAAACAAGTTA
GAAGAGCCATTGCCTGAAACAAGCATAGGAAATATTTTCTATCTG
ATAACCTTTCCAATAAGCAATAATCATGGCGATCTCATGCCCGAT
GACTTCATTAGCCAACTCAGGCTAGGAATACGTAAGTTTCAAAAT
ATACGAAATTTGGAAACTGCATTACGAACCGTTGAAGAGATGATA
TCTGAAACTTTTATCTTGGGTACGGCAGAAAGCATGGATACTAGT
TATGTATATTCGAGCATCCGTGGGTTTCCGATGTATGATATTGAT
TTTGGGTGGGGGAAGCCCGTAAAAGTAACCGTTGGGGGAGCCCTT
AAGAACTTAAGTATTCTGATGGACACTCCTGATGTCAATGGCATC
GAAGCACTAGTGTCTTTGGATAAACAAGACATGAAGATACTTCTA
AACGACCCTGAGTTGTTGGCCTTTTGCTTGTAA.

In some embodiments, the DNA molecule comprises a nucleic acid sequence with at least 72, at least 80%, at least 85%, at least 87%, at least 93%, or at least 99% homology or identity to SEQ ID NO: 126, or any value and range therebetween. Each possibility represents a separate embodiment of the invention. In some embodiments, the DNA molecule comprises a nucleic acid sequence with 72% to 100%, 79% to 100%, 86% to 100%, or 91% to 100% homology or identity to SEQ ID NO: 126. Each possibility represents a separate embodiment of the invention.

In some embodiments, the DNA molecule comprises or consists of the nucleic acid sequence:

(SEQ ID NO: 127)
ATGAGTACTAGTGACAAAATGAAGATAACAATAAGAGAATCATCAATGAT
AAAACCATCCAAACCGACGCCGGATCAACGGATATGGAACTCAAATCTTG
ATTTGGTAGTGGGTCGGATCCATATCTTGACCCTTTACTTTTTTAGGCCA
AATGGGTCTTCGGATTTCTTTGATTCTGAGGTTTTAAAGCAATCACTTGC
CGACGTTCTTGTTTCTTTTTTTCCGATGGCCGGACGATTGGGATTAGACG
GCGATGGCAGAGTTGAAATTAATTGCAACGGTGAAGGTGTTTTGTTTGTT
GAAGCTGAAGCGGATTGTAGTATTGATGATTTTGGTGAGATTACTCCGTC
GCCGGAGCTACGGCGGTTGGCGCCAACAGTGGATTATTCCGGCGATATCT
CATCTTATCCACTCGTTATTACCCAGGTAACACATTTCAAATGTGGTGGA
GTTTCTCTTGGGTGTGGACTACACCATACATTATCCGATGGACTTTCATC
TCTTCACTTCATCAACACATGGTCCGATGTTACCCGAGGCTTACCCGTTG
CGATCCCGCCATTCGTAGATCGTACGGTTCTTCGTGCTAGGGACCCGCCA
ACCGTGGTCTTTGATCACGTGGAATACCACACTCCTCCTTCCATGACCTC
AAGTTTGGACAAAGACAAACCTCAATCCGAAGATGTTCATGTTTCCACTT
CCATGCTACGGCTCACACTCGATCAAATAAATGCACTAAAAGCAAAAGGC
AAAGGTGACGGAATTGTGTACCATAGCACATATGAAATCCTAGCTGCTCA
TTTATGGCGATGTGCGTGTAAAGCACGTGGGCTCCTGAATGATCAAATGA
CTAAATTGTATGTAGCTACCGATGGACGGTCCAGATTGATTCCCCCACTC
CCACCGGGGTACTTAGGCAATGTGGTCTTCACCGCCACACCAATTGCCAA
ATCCGGCGAGCTCCAACAGGAACCACTAGCTACCACTGCAAGAAAAATTC
ATACAGAGTTGGCCAAAATGGATGACAAGTACCTCAGGTCGGCCCTCGAC
TACTTAGAGTCACAACAGGACTTGTCAGCACTAATTCGAGGGCCAGCCTA
TTTTGCGTGCCCTAACCTCAACATCAATAGTTGGACTCGCCTTCCAATAT
ATGATGCGGACTTTGGGTGGGGTCGGCCCATATTTATGGGACCCGCCAGC
ATACTTTACGAGGGCACGATTTACATTATTCCGAGCCCTAGTGGTGACCG
AAGTGTGTCGTTGGCTGTGTGCTTAGACCCCTCTCATATGCCTCTCTTCC
AAAAGTACTTGTATGAACTTTAA.

In some embodiments, the DNA molecule comprises a nucleic acid sequence with at least 79, at least 85%, at least 95%, or at least 99% homology or identity to SEQ ID NO: 127, or any value and range therebetween. Each possibility represents a separate embodiment of the invention. In some embodiments, the DNA molecule comprises a nucleic acid sequence with 79% to 100%, 85% to 100%, 90% to 100%, or 95% to 100% homology or identity to SEQ ID NO: 127. Each possibility represents a separate embodiment of the invention.

In some embodiments, the DNA molecule comprises or consists of the nucleic acid sequence:

(SEQ ID NO: 128)
ATGGTGAATGTTGAGATCATTTCTAATGAATACATAAAACCATCCTCCCC
AACACCACCACATCTTAAAATATACAATCTTTCCATCTTAGATCAACTCA
TTCCTGCCCCCTATGCACCTATCATACTATATTATCCGAATCAAGATCAC
ATTAACGATTTTGAGGTTCACGAACGGTTGAAACTACTAAAAGATTCGTT
ATCGAAAACGCTAACTCGTTTTTACCCATTAGCCGGAACCATCAAAGGCG
ATCTTTCCATTGATTGTAACGATATTGGTGCTTACTTTGCAGTAGCTCAT
GTAAATACTCGCCTTGATGTGTTCCTGAACCATCCTGATCTTGACCTAAT
AAACTGTTTTCTTCCACGTGGGCCTTACTTGAATGGTTCTAGTGAAGGAA
GTTGTGTGAGTAATGTTCAAGTGAACATTTTTGAGTGTTGTGGGATTGCA
ATTAGTTTATGCATTTCTCACAAGATTCTTGATGGTGCTGCGTTGAGTAC
TTTTCTTAAAGCATGGGCAGGGACAAGTTACGGGTCGAAAGAAGTAGTGT
ATCCAAACATGAGTGCACCATCTTTATTTCCTGCTAAAGATTTGTGGCTT
AAAGATTCATCAATGGTCATGTTTGGGTCTTTGTTTAAGATGGGTAAGTG
TAGTACTAAAAGATTTGTTTTTGATTCATCAAAATTATCCTTCCTCAAAG
CTAAGGCATCGCTAAATGGGCTAAAAGACCCAACCCGCGTAGAGGTGGTG
TCTGCTTTACTATGGAAGTGTATCATGGCTGCATCTGAAGAAAACACTGG
TTCTTGGAAGCCATCTCTGTTAAGCCATGTAGTTAACCTTCGCAAAAGGT
TGGTTTCAACTTTATCAGAAGACTCAATTGGGAACTTAATTTGGTTAGCA
AGCGCAGAATGTAGAACCAACGCTCAATCCCGATTGAGTGATCTTGTTGA
AAAGGTACGTGATAGTGTGTCGAAAATCAATAGTGAGTTTGTGAAGAAAA
TACAAGGCGATAAAGGGACAAAAGTGATGGAAGAGTCTCTCAAGAGTATG
AAAGATTGTGCGGATTATATCGGGTTTACGAGTTGGTGTAAGATGGGGTT
TTACGATGTGGATTTTGGTTGGGGAAAGCCTGTATGGGTTTGTGGTAGCG
TTTGTGAAGGTAGCCCGGTGTTCATGAATTTTGTCATATTAATGGACACA
AAATATGGTGATGGAATAGAAGCATGGGTGAGCTTGGATGAACACGAAAT
GCATATCTTAAAGCATAATCCCGAGCTCTTGGAATATGCATCAATCGATC
CAAGTCCTCTGCAAATGAATAAGTGA.

In some embodiments, the DNA molecule comprises a nucleic acid sequence with at least 82, at least 85%, at least 95%, or at least 99% homology or identity to SEQ ID NO: 128, or any value and range therebetween. Each possibility represents a separate embodiment of the invention. In some embodiments, the DNA molecule comprises a nucleic acid sequence with 82% to 100%, 88% to 100%, 93% to 100%, or 97% to 100% homology or identity to SEQ ID NO: 128. Each possibility represents a separate embodiment of the invention.

In some embodiments, the DNA molecule comprises or consists of the nucleic acid

(SEQ ID NO: 129)
ATGGGAACTATTTATCAATCTCCCATGATCAAATCTTCTACTCCCAAAAT
AATTGAAGACCTCAAAGTTATCATCCATGACACATTCACAATCTTCCCAC
CTCACGAAACCGAAAAGCGGTCCATGTTCTTATCGAACATTGACCAAGTT
CTTACTTTCAACGTTGAAACGGTCCATTTTTTTGCAGCCAACCCTGACTT
TCCGCCACAAGTAGTGGCGGAAAAGCTCAAGTTGGCTCTAAGTAAGGCGC
TGGTGCCATATGATTTTTTGGCAGGGAGGTTGAAGTTGAACCATGAGTCG
CAACGGTTTGAGTTTGATTGTAATGGTGCTGGGGCTCGGTTCGTGGTGGG
TTCGAGTGAGTTTGAGTTGGGTGAGATTGGTGACTTGGTGTATCCAAACC
CTGGGTTTAGACAATTGGTTCAAAAGAGTTATGATAACTTGGAGTTACAT
GAAAAGCCACTATGCATTTTACAGCTGACATCCTTCAAGTGTGGAGGATT
TGCACTTGGTGTAGCAACAAATCATGCCACTTTTGATGGCTTAAGTTTCA
AAACATTTCTTCAAAATCTTGGTTCTTTGGCTGCTGATCAACCACTTGCC
GTCGATCCCTGCAACGATCGCCACCTATTGGCAGCACGATCACCACCAAA
AGTCCAATTTGACCACCCTGAACTCCTCAAAATCCCAACAGGAACAGACA
TCCCAAACCCAACAGTCTTTGACTGCCCAGAAAGTCAACTTGACTTCAAG
ATTTTCAACTTGACCTCAGATGACATAGCCCACTTAAAAACGAAAGCCAA
AGATGGGCCTGGGTCAACCAATGCAAAAATCACTGGATTCAATGTGGTTG
CAGCCCATGTATGGCGGTGCAAAGCGTTGTCCTCAGGGTCAGAATATGAC
CCCGAGAGAGTGTCAACCGTGTTATATGCTGTTGACATTCGGTCAAGATT
GAACTTACCATTATCATTAGCTGGCAATGCAGTTCTTAGTGCATACGCCT
CGGCCAAATGCAAAGAGATTGAAGAAGGCCCGTTGTCAAGACTAGTGGAA
ATGGTGACCGAAGGTACTAACAGAATGACTGGTGAGTATGCAAGATCGGT
GATCGATTGGGGAGAGGTGAATAAAGGGTTTCCAAATGGGGAGTTTCTGA
TATCGTCATGGTGGCGATTGGGGTTTGCTGACGTGGAATATCCGTGGGGT
AAACCTAGGTATAGTTGTCCCGTGGTTTATCATAGGAAAGATATAATATT
ACTCTTTCCGGATATTGTTGGTGCCGATAACAACAATGAAGTGAATGTGT
TGGTGGCTTTGCCTGGCAAAGAAATGGAGAAATTTGAGACTTTATTTCAT
AAGTTTTTGGCATGA.

In some embodiments, the DNA molecule comprises a nucleic acid sequence with at least 87, at least 91%, at least 95%, or at least 99% homology or identity to SEQ ID NO: 129, or any value and range therebetween. Each possibility represents a separate embodiment of the invention. In some embodiments, the DNA molecule comprises a nucleic acid sequence with 87% to 100%, 90% to 100%, 94% to 100%, or 97% to 100% homology or identity to SEQ ID NO: 129. Each possibility represents a separate embodiment of the invention.

In some embodiments, the DNA molecule comprises a plurality of nucleic acid sequences. In some embodiments, the polynucleotide comprises a plurality of types of polynucleotides.

As used herein, the term “plurality” comprises any integer equal to or greater than 2.

In some embodiments, plurality of nucleic acid sequences encode proteins of different enzymatic functions or families as described herein. In some embodiments, plurality of nucleic acid sequences encode at least two proteins of the same enzymatic function or family as described herein. In some embodiments, plurality of nucleic acid sequences encode a plurality of proteins of a plurality of different enzymatic functions or families as described herein.

In some embodiments, the DNA molecule encodes a protein characterized by acyl activating enzymatic (AAE) activity. In some embodiments, the DNA molecule encodes an AAE protein. In some embodiments, the AAE is an AAE derived from Helichrysum umbraculigerum. In some embodiments, the DNA molecule encoding a protein characterized by acyl activating enzymatic (AAE) activity comprises a nucleic acid sequence set forth in SEQ ID Nos.: 1-11.

As used herein, the terms “acyl activating enzyme” and “AAE” are interchangeable, and refer to any peptide, polypeptide, or a protein, capable of catalyzing the activation of a carboxylic acid. In some embodiments, AAE activity comprises forming or formation of a thioester bond. In some embodiments, AAE activity comprises coupling a carboxyl group to an amine group. In some embodiments, AAE activity comprises coupling a carboxyl group to an alcohol. In some embodiments, the AAE is an acid-thiol ligase.

In some embodiments, the DNA molecule encodes a protein characterized by polyketide synthesizing activity. In some embodiments, the DNA molecule encodes a protein being a polyketide synthase (PKS). In some embodiments, the PKS is a PKS derived from Helichrysum umbraculigerum. As used herein, the terms “polyketide synthase” and “PKS” encompasses any enzyme derived from H. umbraculigerum and having or characterized by being functional analog of the “olivetol synthase” or “OLS” of Cannabis sativa. In some embodiments, the DNA molecule encoding a protein characterized by polyketide synthesizing activity comprises a nucleic acid sequence set forth in SEQ ID Nos.: 23-26.

As used herein, the terms “polyketide synthase” and “PKS” are interchangeable, and refer to any peptide, polypeptide, or a protein, capable of catalyzing the elongation of a ketide or a polyketide chain. In some embodiments, PKS activity transacylation. In some embodiments, PKS activity comprises Claisen condensation. In some embodiments, PKS activity comprises reduction of β-keto group to a β-hydroxy group. In some embodiments, PKS activity comprises H₂O splitting, thereby obtaining, providing, or resulting in a α-β-unsaturated alkene. In some embodiments, PKS activity comprises reducing a α-β-double-bond to a single-bond. In some embodiments, PKS activity comprises hydrolyzing a polyketide chain or a completed polyketide chain from an acyl carrier protein domain of the PKS. In some embodiments, PKS activity comprises polymerizing and/or ligating a diketide substrate into a polyketide chain. In some embodiments, PKS activity comprises elongating a diketide to a polyketide chain. In some embodiments, PKS activity comprises elongating a polyketide chain.

In some embodiments, the DNA molecule encodes a protein characterized by polyketide cyclizing activity. In some embodiments, the DNA molecule encodes a protein being a polyketide cyclase (PKC). In some embodiments, the PKC is a PKC derived from Helichrysum umbraculigerum. As used herein, the terms “polyketide cyclase” and “PKC” encompasses any enzyme derived from H. umbraculigerum and having or characterized by being functional analog of the “olivetolic acid cyclase” or “OAC” of Cannabis sativa. In some embodiments, the DNA molecule encoding a protein characterized by polyketide cyclizing activity comprises a nucleic acid sequence set forth in SEQ ID Nos.: 31-38.

As used herein, the terms “polyketide cyclase” and “PKC” are interchangeable, and refer to any peptide, polypeptide, or a protein, capable of folding and/or cyclizing a polyketide. In some embodiments, PKC activity comprises an action of a cyclase subunit. In some embodiments, PKC activity comprises site-specific keto-reductase activity.

In some embodiments, the DNA molecule encodes a protein characterized by prenyl transferring activity. In some embodiments, the DNA molecule encodes a protein being a prenyltransferase (PT). In some embodiments, the PT is a PT derived from Helichrysum umbraculigerum. As used herein, the terms “prenyltransferase” and “PT” encompass any enzyme derived from H. umbraculigerum and having or characterized by being functional analog of the “geranylpyrophosphate: olivetolate geranyltransferase” or “GOT” of Cannabis sativa. In some embodiments, the GOT is GOT4 or CsGOT4. In some embodiments, the DNA molecule encoding a protein characterized by prenyl transferring activity comprises a nucleic acid sequence set forth in SEQ ID Nos.: 47-58.

As used herein, the terms “prenyltransferase” and “PT” are interchangeable, and refer to any peptide, polypeptide, or a protein, capable of transferring an allylic prenyl group to an acceptor molecule. In some embodiments, PT activity comprises cyclization. In some embodiments, PT activity comprises transferring an allylic prenyl group to an acceptor molecule.

In some embodiments, the DNA molecule encodes a protein characterized by cannabigerolic acid (CBGA) cyclization or cyclizing activity. In some embodiments, cycling activity comprises cyclization of CBGA to CBCA. In some embodiments, the polynucleotide encodes a protein capable of cyclizing or cyclization of CBGA to CBCA. In some embodiments, the DNA molecule encodes a protein characterized by being capable of synthesizing CBCA or being a CBCA synthase (CBCAS). In some embodiments, the CBCAS is a CBCAS derived from Helichrysum umbraculigerum. As used herein, the terms “CBCA synthase” and “CBCSA” encompass any enzyme derived from H. umbraculigerum and having or characterized by being a functional analog of the CBCA synthase of Cannabis sativa (e.g., CsCBCAS). In some embodiments, the DNA molecule encoding a protein characterized by CBGA cyclization or cyclizing activity comprises a nucleic acid sequence set forth in SEQ ID Nos.: 71-79.

In some embodiments, the polynucleotide encodes a protein characterized by catalytic activity of transfer a glucuronic acid component of UDP-glucuronic acid to a small hydrophobic molecule (e.g., a UGT). In some embodiments, the polynucleotide encodes a protein characterized by glycosyltransferase catalytic activity. In some embodiments, the polynucleotide encodes a protein characterized by being capable of transferring glucuronic acid component of UDP-glucuronic acid to a cannabinoid or a precursor thereof. In some embodiments, the polynucleotide encodes a protein characterized by having a catalytic activity of glycosylating a cannabinoid or a precursor thereof. In some embodiments, the polynucleotide encodes a UGT enzyme.

In some embodiments, the UGT is a UGT derived from Helichrysum umbraculigerum. As used herein, the term “UGT” encompass any enzyme derived from H. umbraculigerum and having or characterized by having an activity as described herein.

In some embodiments, the UGT protein is encoded by a DNA molecule comprising SEQ ID Nos.: 89-101.

In some embodiments, the DNA molecule encodes a protein characterized by being capable of acting on an acyl group. In some embodiments, the DNA molecule encodes a protein characterized by catalytic activity of transferring an acyl group from a donor molecule to an acceptor molecule. In some embodiments, the acceptor molecule is a hydrophobic molecule, a small molecule, or both. In some embodiments, the donor molecule comprises an acyl group, CoA, or both. In some embodiments, the DNA molecule encodes a protein characterized by acyltransferase catalytic activity. In some embodiments, the DNA molecule encodes a protein characterized by being capable of transferring an acyl group to a cannabinoid. In some embodiments, the DNA molecule encodes a protein characterized by having a catalytic activity of acylating a cannabinoid. In some embodiments, the acyltransferase (AT) is an alcohol acyltransferase (AAT). In some embodiments, the DNA molecule encodes an AT enzyme. In some embodiments, the polynucleotide encodes an AAT enzyme.

In some embodiments, the AAT is an AAT derived from Helichrysum umbraculigerum. As used herein, the term “AAT” encompass any enzyme derived from H. umbraculigerum and having or characterized by having an activity as described herein.

In some embodiments, the AAT protein is encoded by a DNA molecule comprising or consisting of SEQ ID Nos.: 115-129.

In some embodiments, the artificial vector comprises a plasmid. In some embodiments, the artificial vector comprises or is an agrobacterium comprising the artificial nucleic acid molecule. In some embodiments, the artificial vector is an expression vector. In some embodiments, the artificial vector is a plant expression vector. In some embodiments, the artificial vector is for use in expressing any one of: AAE, PKS, PKC, PT, or CBCAS encoding nucleic acid sequence as disclosed herein, or any combination thereof. In some embodiments, the artificial vector is further for the use in expressing UGT, AAT, or both. In some embodiments, the artificial vector is for use in heterologous expression of any one of: AAE, PKS, PKC, PT, or CBCAS encoding nucleic acid sequence as disclosed herein, or any combination thereof, in a cell, a tissue, or an organism. In some embodiments, the artificial vector is further for the use in heterologous expression of UGT, AAT, or both in a cell, in a tissue, or an organism. In some embodiments, the artificial vector is for use in producing or the production of an acyl-coenzyme A (acyl-CoA), a polyketide, a cannabinoid, e.g., CBGA, CBCA, any precursor thereof, or any combination thereof, in a cell, a tissue, or an organism. In some embodiments, the artificial vector is further used in producing or the production of a modified acyl-coenzyme A (acyl-CoA), a polyketide, a cannabinoid, e.g., CBGA, CBCA, any precursor thereof, or any combination thereof, in a cell, a tissue, or an organism, wherein the modified further comprises an acyl group, a glycan (e.g., glycosylated), or both.

Expressing a polynucleotide within a cell is well known to one skilled in the art. It can be carried out by, among many methods, transfection, viral infection, or direct alteration of the cell's genome. In some embodiments, the DNA molecule is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the DNA molecule as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the DNA molecule is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide encoding a protein comprising an amino acid sequence as described herein.

According to some embodiments, there is provided a protein encoded by: (a) the DNA molecule disclosed herein; (b) the artificial vector disclosed herein; or the plasmid or agrobacterium disclosed herein.

In some embodiments, the protein is an isolated protein.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

In some embodiments, the protein comprises or consists of the amino acid sequence:

(SEQ ID NO: 12)
MTSSKKFTVEVEPAIPAKDGKPSAGPVYRSIFAKDGFPAHIDGLDSCWDI
FRLSVEKYPNNRMLGTREFVNGKHGPYVWSTYKQVYDKVIKVGNAIRACG
VEPGGRCGIYGANCAEWIMSMEACNAHGLYCVPLYDTLGAGAIEFILCHA
EVTIAFVEEKKIPELLKTFPKAGEFLKTIVSFGKVTPEQREQAENFGLKI
HSWDEFLTLGDDKNFDLPLKEKTDICTIMYTSGTTGDPKGVLISNNSMAT
LIAGVNRLLDSAKESLNQHDVYLSFLPLAHIFDRVIEECFINHGASIGFW
RGDVKLLIEDIGELKPTIFCAVPRVLDRIYSGLQQKISAGGFIKRNLFNL
AYSYKLRNMKGGKTHSEASPLSDKIVFSKVKQGLGGNVRIILSGAAPLAP
HVEAYLKVVACSHVLQGYGLTETCAGSFVSLPNEMEMLGTVGPPVPVLDA
RLESVPEMNYDACSSKPQGEICIRGDVLFSGYYKREDLTKEVFVDGWFHT
GDIGEWQPDGSMKIIDRKKNIFKLSQGEYVAVENLENVYGNVSDIDTIWI
YGNSFEFCLVAVVNPNEPAIKRYAEANNISGDFDSLCENPKIKEYILGEL
ARIGKEKKLKGFEFVKAVHLDPVPFDMERDLLTPTFKKKRPQMLKYYQDV
IDNMYKTINKK.

In some embodiments, the protein comprises an amino acid sequence with at least 91%, at least 93%, at least 95%, or at least 97% homology or identity to SEQ ID NO: 12, or any value and range therebetween. Each possibility represents a separate embodiment of the invention. In some embodiments, the protein comprises an amino acid sequence with 91% to 97%, 92% to 99%, 93% to 98%, or 90% to 100% homology or identity to SEQ ID NO: 12. Each possibility represents a separate embodiment of the invention.

In some embodiments, the protein comprises or consists of the amino acid sequence:

(SEQ ID NO: 13)
MDALRKPNSANSSPLTPIGFLERAAVVFANSPSIVYNNLIYTWSDTFHRC
LRLASSISRLAIRKGDVVSVLAPNIPAIYELHFGITMTGAIINTINTRLD
ARTISILLCHSESKLVFVDYQLTRLIREAVSLMPDACVPPQLVLIVDDGH
NLSLLSDQFINTYEAMVETGDPGFNWVRPDSDWDPLTLNYTSGTTSSPKG
VVNSHRGSFIVAFDSLLEWHVPKQPIMLWTLPMFHANGWSFVWGMAAVGG
TNVCLRKFDATIIYDTIRNHHVTHMCGAPVVLNMLSEGKPLEHTVHIMTA
GAPPPAAVLLRTESLGFEVTHGFGMTETGGLVVSCSWKKEWNRLPVTEKA
RLKARQGVRTLGMTEVDIVDPESGVSVTRDGLTQGELVLRGGSIMLGYLK
DPETTNKSVKNGWFYTGDVAVMHPDGYLEIKDRSKDVIISGGENISSVEV
ESILYQHPAINEAAVVGRPDEFWGESPCAFVSLKDDNGKVAVPTADEIMK
FCKGKLPGYMVPKSVVFKKDLPKTSTGKIQKYVLRKLAKDLGFAVKSRI.

In some embodiments, the protein comprises an amino acid sequence with at least 83%, at least 85%, at least 90%, at least 95%, at least 97%, or at least 99% homology or identity to SEQ ID NO: 13, or any value and range therebetween. Each possibility represents a separate embodiment of the invention. In some embodiments, the protein comprises an amino acid sequence with 83% to 95%, 85% to 99%, 83% to 100%, or 84% to 97% homology or identity to SEQ ID NO: 13. Each possibility represents a separate embodiment of the invention.

In some embodiments, the protein comprises or consists of the amino acid sequence:

(SEQ ID NO: 14)
MTEEEKNKAESMGIKTYAWSDFLHLGSKNPSELQTPKATDICTIMYTSGT
SGDPKGVILTHENATTNIRGVDLFMEQFEDKMTVDDVYISFLPLAHILDR
MIEEYFFRSGASVGFYHGDINALKEDLAELKPTFLAGVPRVLEKIHEGVL
KGLEEVNPRRRKIFSILYNHKLKYMKAGYKHKYASPLADLLAFRKVKNRL
GGRIRLMVSGGAPLSTEIEEFMRVTSCAFVAQGYGLTETCGLATLGFPDE
MCMIGTVGSPFVYTELRLEEVSDMGYDPLANPPRGEICVKGKTPFAGYYK
NPELTNEVMKDGWFHTGDIGEMQPNGVLKIIDRKKHLIKLSQGEYIALEY
LEKVYCITPILEDIWVYGDSFKSSLVAVAVPNKENAEKWADQKGLKVSYS
ELCTLTQFRDYIQSELKSTAERNKLRGFEHIKAIIVEPRTFEGDQELLTA
TMKKRRNKLLNRYKEGIDNLYKNLAANKR.

In some embodiments, the protein comprises an amino acid sequence with at least 86%, at least 88%, at least 90%, at least 95%, or at least 99% homology or identity to SEQ ID NO: 14, or any value and range therebetween. Each possibility represents a separate embodiment of the invention. In some embodiments, the protein comprises an amino acid sequence with 86% to 93%, 86% to 95%, 88% to 97%, or 86% to 100% homology to SEQ ID NO: 14. Each possibility represents a separate embodiment of the invention.

In some embodiments, the protein comprises or consists of the amino acid sequence:

(SEQ ID NO: 15)
MVYKSLNSISISDIVNLGISPETATQLHQKLTEIIQIYGFDAPQTWTQIS
TRILHPDLPFCFHQMMYYGCYVDFGPDPPAWSPDPKDAKLTNIGSLLERR
GKEFLGPSYKDPISSYSALQEFSALNLEVFWKTILDEMNITFSVPPKRIL
VDDLSKESQLLHPGGRWLPGAYVNPARNCLSLSSKRRLSDIAVIWRDEGN
DDMPVNKMTFQQLRSEVWLVAYALDTLGVEKGSAIAIDMPMDVKSVVIYL
AIVLAGYVVVSIADSFAAGEISTRLVLSKAKAIFTQDLIIRGDRSHPLYS
RVVDAQSPLAIVIPTRGSSFSIKLRDGDISWHDFLERANTYRNVEFVAVE
RPVEAFSNILFSSGTTGEPKAIPWTLATPFKAGADAWCHMDVHKGDVVAW
PTNLGWMMGPWLIYASLLNGGSLALYNGSPLTSGFAKFVQDAKVTLLGVI
PSIVRAWRTNNSTAGFDWSTIRCFGSTGEASNTDECLWLMGRAHYKPVIE
YCGGTEIGGGFITGSLLQPQCLSAFSTPSLGCKLLILGEDGIPIPQNAPG
IGELALNPLMFGASSTLLNANHYDVYFKGMPSWNGKVLRRHGDVFERTSK
GYYRAHGRADDTMNLGGIKVSSVEIERVCNSIDDRILETAAIGVTPSGGG
PERLVIVVAFKDGSGSKPDLIKLKVTLNSALQKNLNPLFKVSDVVPFPSL
PRTATNKVMRRVLRQQLTQIGQNSKL.

In some embodiments, the protein comprises an amino acid sequence with at least 86%, at least 88%, at least 90%, at least 95%, or at least 99% homology or identity to SEQ ID NO: 15, or any value and range therebetween. Each possibility represents a separate embodiment of the invention. In some embodiments, the protein comprises an amino acid sequence with 86% to 93%, 86% to 95%, 88% to 97%, or 86% to 100% homology to SEQ ID NO: 15. Each possibility represents a separate embodiment of the invention.

In some embodiments, the protein comprises or consists of the amino acid sequence:

(SEQ ID NO: 16)
MGDSEGSSISTPTTEQVGFLSNIMEDKSYSAAVAIMVAIAVPLVLSSVFA
AKKKVKQRGVPVQVGGEPGFAMRNSRSNKLVDVPWEGARTMAALFEQSCK
KHSQLRFLGTRKLIERSFVSGSDGRKFEKLHLGEYQWETYGQIFERVCNF
ASGLIQLGHDPDTRIAIFSDTRAEWLIAFEGCFRQNITVVTIYASLGDDA
LIHSLNETKVSTLICDSKLLKKVAAVSSSLKTVENFIYFESDNTEALNEI
GDWKISSFSEVESLGQKSPVSARLPIKKDVAVIMYTSGSTGLPKGVMMTH
GNVVATAAAVMTVIPNIGTNDVYLAYLPLAHIFELAAETVMVTAGIPIGY
GSALTLTDTSNKIKKGTLGDASILKPTLMAAVPAILDRVRDGVLKKVEEK
GGLTTKIFNIAYKRRLLAVDGSWLGAWGLEKLLWDAIVFKKIRSVLGGDI
RFMLCGGAPLAADTQRFINVCVGAPIGQGYGLTETCAGAAFSEADDNSVG
RVGPPLPCVYIKLVSWDEGGYLTSDKPMPRGEVVVGGYSVTAGYFNNEEK
TNEVYKVDESGMRWFYTGDIGRFHPDGCLEIIDRKKDIVKLQHGEYISLG
KVEAALASSKYVENVMLHADPFHTYCVALVVPARQVIEQWAQDAGISYQD
FAELCDKKETVSEVQQSLTKVAKDAKLDKFETPAKIKLMPDPWTPESGLV
TAALKLKREQLKSKFKDDLDKLYG.

In some embodiments, the protein comprises an amino acid sequence with at least 89%, at least 92%, at least 94%, at least 97%, or at least 99% homology or identity to SEQ ID NO: 16, or any value and range therebetween. Each possibility represents a separate embodiment of the invention. In some embodiments, the protein comprises an amino acid sequence with 89% to 95%, 89% to 98%, 90% to 99%, or 89% to 100% homology to SEQ ID NO: 16. Each possibility represents a separate embodiment of the invention.

In some embodiments, the protein comprises or consists of the amino acid sequence:

(SEQ ID NO: 17)
MSVYTVKVEDSRAASGETPSAGPVYRCIYAKDALMELPPGYESPWDFFSE
SVKRNPKNPALGRRQVIDGKAGGYSWLSYQEAYNSALRIASAIRSRSVNP
GDRCGIYGPNCPEWIISMEACNSNGITYVPLYDTLGANAVEYIINHAEIS
LVFVQENKLSAILSCLPNCSSNLKTIVSFGKFSESQKNEAMEHGVDCFSW
EEFSSMGNLEDELPAKNKTDICTIMYTSGTTGEPKGVVLSNRAFMSEVLS
MHELLIETDKPGTEEDTYFSFLPLAHIFDQIMETYFIYSGASIGFWQGDI
RYLIEDLLVLQPTIFCGVPRVYDRIYTGIMAKISTGGAIRKALFDFAYNY
KLRNLEKGIQQDKSAPLLDKLVFDKIKQGFGGRVRLMLSGAAPLPKHVEE
FLRVTCCTVLSQGYGLTESCGGCFTSIANVYSMIGTVGVPMTTIEARLES
VPEMGYDALSSVPCGEICLRGNTLFSGYHKRDDLTDAVLVDGWFHTGDIG
EWQADGAMKIIDRKKNIFKLSQGEYVAVESIESTYSRCPLVTSIWVYGNS
FESFLVAVVVPDRVAVEEFAAKNNESGDYASLCKNPNVRKYVLEELNAEA
QCNKLRGFEMLKAVHLDPVPFDFERDLITPTFKLKRQQLLKYYKDCVEQL
YAEAKTSKK.

In some embodiments, the protein comprises an amino acid sequence with at least 93%, at least 94%, at least 95%, at least 97%, or at least 99% homology or identity to SEQ ID NO: 17, or any value and range therebetween. Each possibility represents a separate embodiment of the invention. In some embodiments, the protein comprises an amino acid sequence with 93% to 98%, 93% to 99%, 93% to 100%, or 95% to 100% homology to SEQ ID NO: 17. Each possibility represents a separate embodiment of the invention.

In some embodiments, the protein comprises or consists of the amino acid sequence:

(SEQ ID NO: 18)
METHGPRLLGAAYKDPITSYKQFQKFSVQHLEVYWSLVLEKLSIQFQERP
KCIVDTSDKSKHGGTWLPGSVLNIAECCILSTTETDEKVAIVWRDERCDN
LDVNKMTFKELRQQVMLVANALKLLFSKGDPIAIDMPMTVTAVILYLAIV
YSGFVVVSIADSFAAKEIATRLRVSNAKAIFTQDYIVRGGRRFPLYSRVI
EATQCRAIVVPAIGENVEVILRKQDISWGDFLSGAKQLPSPDYCSPVYQS
IDTLTNILFSSGTTGDPKAIPWTQISPMRCAADGWAHMDIQAGDVYCWPT
NLGWVMGPIVLYSSFLTGATLALYNGSPLGHGFGKFVQDAGVTILGTVPS
IVKSWKSTRCMEGLDWTKIKAFGSTGEASNVDDDLWLSSKAYYKPVLECC
GGTELASSYVQGNLLQPQAFGALSSASMGTGFVIFDDHGVPYPDDEPCVG
EVGLFPVYMGASDRLLNADHEKIYFKGMPSYKGMQLRRHGDIIKRTIGGY
LVVQGRADDTMNLGGIKTSSIEIERVCEQADGSIMETAAVSVAPATGGPE
LLAIFVVLKNGCNTQPQDLKMIFSKAIQKNLNPLFKVSFVKVVPEFPRTA
SNKLLRRVLRNQVKEELQTRSKI.

In some embodiments, the protein comprises an amino acid sequence with at least 84%, at least 87%, at least 91%, at least 95%, or at least 99% homology or identity to SEQ ID NO: 18, or any value and range therebetween. Each possibility represents a separate embodiment of the invention. In some embodiments, the protein comprises an amino acid sequence with 84% to 99%, 85% to 99%, 84% to 100%, or 90% to 100% homology to SEQ ID NO: 18. Each possibility represents a separate embodiment of the invention.

In some embodiments, the protein comprises or consists of the amino acid sequence:

(SEQ ID NO: 19)
MEITKSIQELGLQDLLNTGLTPNDAKSLQIEIKHIINSQTTNSNPVELWR
QITSAKLLKPSYPHSLHQLIYYAVYCNYDASIYGPPLYWFPSEIDSKRSN
LGNIMETHGPRLLGAAYKDPITSYKQFQKFSVQHLEVYWSLVLEKLSIQF
QERPKCIVDTSDKSKHGGTWLPGSVLNIAECCILSTSETDDKVAIVWRDE
RCDNLDVNKMTFKELRQQVMLVANALKLLFSKGDPIAIDMPMTVTAVILY
LAIVYSGFVVVSIADSFAAKEIATRLRVSNAKAIFTQDYIVRGGRRFPLY
SRVIEATQCRAIVVPAIGENVEVILRKQDISWGDFLSGAKQLPSPDYCSP
VYQSIDTLTNILFSSGTTGDPKAIPWTQISPMRCAADGWAHMDIQAGDVY
CWPTNLGWVMGPIVLYSSFLTGATLALYNGSPLGHGFGKFVQDAGVTILG
TVPSIVKSWKSTRCMEGLDWTKIKAFGSTGEASNVDDDLWLSSKAYYKPV
LECCGGTELASSYVQGNLLQPQAFGALSSASMGTGFVIFDDHGVPYPDDE
PCVGEVGLFPVYMGASDRLLNADHEKIYFKGMPSYKGMQLRRHGDIIKRT
IGGYLVVQGRADDTMNLGGIKTSSIEIERVCEQADGSIMETAAVSVAPAT
GGPELLAIFVVLKNGCNTQPQDLKMIFSKAIQKNLNPLFKVFS.

In some embodiments, the protein comprises an amino acid sequence with at least 82%, at least 87%, at least 91%, at least 95%, or at least 99% homology or identity to SEQ ID NO: 19, or any value and range therebetween. Each possibility represents a separate embodiment of the invention. In some embodiments, the protein comprises an amino acid sequence with 82% to 99%, 83% to 99%, 82% to 100%, or 85% to 100% homology to SEQ ID NO: 19. Each possibility represents a separate embodiment of the invention.

In some embodiments, the protein comprises or consists of the amino acid sequence:

(SEQ ID NO: 20)
MVYKSLNSISISDIVNLGISPETATQLHQKLTEIIQIYGFDAPQTWTQIS
TRILHPDLPFCFHQMMYYGCYVDFGPDPPAWSPDPKDAKLTNIGSLLERR
GKEFLGPSYKDPISSYSALQEFSALNLEVFWKTILDEMNITFSVPPKRIL
VDDLSKESQLLHPGGRWLPGAYVNPARNCLSLSSKRRLSDIAVIWRDEGN
DDMPVNKMTFQQLRSEVWLVAYALDTLGVEKGSAIAIDMPMDVKSVVIYL
AIVLAGYVVVSIADSFAAGEISTRLVLSKAKAIFTQDLIIRGDRSHPLYS
RVVDAQSPLAIVIPTRGSSFSIKLRDGDISWHDFLERANTYRNVEFVAVE
RPVEAFSNILFSSGTTGEPKAIPWTLATPFKAGADAWCHMDVHKGDVVAW
PTNLGWMMGPWLIYASLLNGGSLALYNGSPLTSGFAKFVQDAKVTLLGVI
PSIVRAWRTNNSTAGFDWSTIRCFGSTGEASNTDECLWLMGRAHYKPVIE
YCGGTEIGGGFITGSLLQPQCLSAFSTPSLGCKLLILGEDGIPIPQNAPG
IGELALNPLMFGASSTLLNANHYDVYFKGMPSWNGKVLRRHGDVFERTSK
GYYRAHGRADDTMNLGGIKVSSVEIERVCNSIDDRILETAAIGVTPSGGG
PERLVIVVAFKDGSGSKPDLIKLKVTLNSALQKNLNPLFKVSDVVPFPSL
PRTATNKVMRRVLRQQLTQIGQNSKL.

In some embodiments, the protein comprises an amino acid sequence with at least 86%, at least 88%, at least 90%, at least 95%, or at least 99% homology or identity to SEQ ID NO: 20, or any value and range therebetween. Each possibility represents a separate embodiment of the invention. In some embodiments, the protein comprises an amino acid sequence with 86% to 93%, 86% to 95%, 88% to 97%, or 86% to 100% homology to SEQ ID NO: 20. Each possibility represents a separate embodiment of the invention.

In some embodiments, the protein comprises or consists of the amino acid sequence:

(SEQ ID NO: 21)
MTFQQLRSEVWLVAYALDTLGVEKGSAIAIDMPMDVKSVVIYLAIVLAGY
VVVSIADSFAAGEISTRLVLSKAKAIFTQDLIIRGDRSHPLYSRVVDAQS
PLAIVIPTRGSSFSIKLRDGDISWHDFLERANTYRNVEFVAVERPVEAFS
NILFSSGTTGEPKAIPWTLATPFKAGADAWCHMDVHKGDVVAWPTNLGWM
MGPWLIYASLLNGGSLALYNGSPLTSGFAKFVQDAKVTLLGVIPSIVRAW
RTNNSTAGFDWSTIRCFGSTGEASNTDECLWLMGRAHYKPVIEYCGGTEI
GGGFITGSLLQPQCLSAFSTPSLGCKLLILGEDGIPIPQNAPGIGELALN
PLMFGASSTLLNANHYDVYFKGMPSWNGKVLRRHGDVFERTSKGYYRAHG
RADDTMNLGGIKVSSVEIERVCNSIDDRILETAAIGVTPSGGGPERLVIV
VAFKDGSGSKPDLIKLKVTLNSALQKNLNPLFKVSDVVPFPSLPRTATNK
VMRRVLRQQLTQIGQNSKL.

In some embodiments, the protein comprises an amino acid sequence with at least 89%, at least 92%, at least 94%, at least 97%, or at least 99% homology or identity to SEQ ID NO: 21, or any value and range therebetween. Each possibility represents a separate embodiment of the invention. In some embodiments, the protein comprises an amino acid sequence with 89% to 95%, 89% to 98%, 90% to 99%, or 89% to 100% homology to SEQ ID NO: 21. Each possibility represents a separate embodiment of the invention.

In some embodiments, the protein comprises or consists of the amino acid sequence:

(SEQ ID NO: 22)
MNITFSVPPKRILVDDLSKESQLLHPGGRWLPGAYVNPARNCLSLSSKRR
LSDIAVIWRDEGNDDMPVNKMTFQQLRSEVWLVAYALDTLGVEKGSAIAI
DMPMDVKSVVIYLAIVLAGYVVVSIADSFAAGEISTRLVLSKAKAIFTQD
LIIRGDRSHPLYSRVVDAQSPLAIVIPTRGSSFSIKLRDGDISWHDFLER
ANTYRNVEFVAVERPVEAFSNILFSSGTTGEPKAIPWTLATPFKAGADAW
CHMDVHKGDVVAWPTNLGWMMGPWLIYASLLNGGSLALYNGSPLTSGFAK
FVQDAKVTLLGVIPSIVRAWRTNNSTAGFDWSTIRCFGSTGEASNTDECL
WLMGRAHYKPVIEYCGGTEIGGGFITGSLLQPQCLSAFSTPSLGCKLLIL
GEDGIPIPQNAPGIGELALNPLMFGASSTLLNANHYDVYFKGMPSWNGKV
LRRHGDVFERTSKGYYRAHGRADDTMNLGGIKVSSVEIERVCNSIDDRIL
ETAAIGVTPSGGGPERLVIVVAFKDGSGSKPDLIKLKVTLNSALQKNLNP
LFKVSDVVPFPSLPRTATNKVMRRVLRQQLTQIGQNSKL.

In some embodiments, the protein comprises an amino acid sequence with at least 88%, at least 92%, at least 94%, at least 97%, or at least 99% homology or identity to SEQ ID NO: 22, or any value and range therebetween. Each possibility represents a separate embodiment of the invention. In some embodiments, the protein comprises an amino acid sequence with 88% to 95%, 89% to 98%, 90% to 99%, or 88% to 100% homology to SEQ ID NO: 22. Each possibility represents a separate embodiment of the invention.

In some embodiments, the protein comprises or consists of the amino acid sequence:

(SEQ ID NO: 27)
MASSINISKIREAQRAQGPASILAVGTANPSNCVYQADYPDYYFRITKSE
HMVDLKRKFKRMCDQSMIRKRYMQITEEYLKENPNICEYMAPSLDARQDV
VVVEVPKLGKEAATKAIKEWGQPKSKITHLIFCTTSGVDMPGADYQLTKL
LGLCPSVKRFMMYQQGCFAGGTVLRLAKDIAENNKGARVLVVCSEITAVI
FRGPNDTHLDSLIGQALFGDGASSVIVGSDPDLTTERPLFEIISAAQTIL
PDSEGAIDGHLREAGLTFHLLKDVPRLISKNIEKALTQAFSPLGISDWNS
IFWVTHPGGPAILDQVELKLGLKEEKMRTTRHVLSEYGNMSSACVFFVLD
EMRKRSAKGGARTTGEGLDWGVLFGFGPGLTVETVVLHSLPTTMSIAT.

In some embodiments, the protein comprises an amino acid sequence with at least 92%, at least 96%, at least 98%, or at least 99% homology or identity to SEQ ID NO: 27, or any value and range therebetween. Each possibility represents a separate embodiment of the invention. In some embodiments, the protein comprises an amino acid sequence with 92% to 100%, 95% to 100%, 96% to 100%, or 98% to 100% homology or identity to SEQ ID NO: 27. Each possibility represents a separate embodiment of the invention.

In some embodiments, the protein comprises or consists of the amino acid sequence:

(SEQ ID NO: 28)
MASSINISKIREAQRAQGPASILAVGTANPSNCVYQADYPDYYFRITKS
EHMVDLKEKFORMCDKSMIRKRHIHITEEFLKENPNLCEYMAPSLDTRQ
DVVVVEVPKLGKEAATKAIKEWGQPKSKITHLIFCTTSGVDMPGADYQL
TKLLGLHPSVKRFMMYQQGCFAGGTVLRLAKDLAENNKGARVLAVCSEI
TAVTFRGPNDTHIDSLVGQALFGDGAAAVIVGSDPDLTTERPLFEIISA
AQTILPNSEGAIDGHVREVGVTIHILKDVPVLISKNIEKALTQAFSPLG
ISDWNSIFWVVHPGGPAILDQVELKLGLKEEKMRTTRHVLSEYGNMSSA
CVFFVLDEMRKRSAKGGARTTGEGLDWGVLFGFGPGLTVETVVLHSLPT
TMSIAT.

In some embodiments, the protein comprises an amino acid sequence with at least 91%, at least 94%, at least 95%, or at least 97% homology or identity to SEQ ID NO: 28, or any value and range therebetween. Each possibility represents a separate embodiment of the invention. In some embodiments, the protein comprises an amino acid sequence with 91% to 100%, 94% to 100%, 97% to 100%, or 97% to 100% homology or identity to SEQ ID NO: 28. Each possibility represents a separate embodiment of the invention.

In some embodiments, the protein comprises or consists of the amino acid sequence:

(SEQ ID NO: 29)
MASSINISKIREAQRAQGPASILAVGTANPSNCVYQADYPNYYFRITKS
EHMVDLKRKFKRMCDQSMIRKRYMQITEEYLKENPNICEYMAPSLDARQ
DVVVVEVPKLGKEAATKAIKEWGQPKSKITHLIFCTTSGVDMPGADYQL
TKLLGLCPSVKRFMMYQQGCFAGGTVLRLAKDIAENNKGARVLVVCSEI
TAVIFRGPNDTHLDSLIGQALFGDGASSVIVGSDPDLTTERPLFEIISA
AQTILPDSEGAIDGHLREAGLTFHLLKDVPGLISKNIEKALTQAFSPLG
ISDWNSIFWVTHPGGPAILDQVELKLGLKEEKMRASRHVLSEYGNMSSA
CVFFILDEMRKKSDEDGAPTTGEGLDWGVLFGFGPGLTVETVVLHSLPT
TMSIAT.

In some embodiments, the protein comprises an amino acid sequence with at least 93%, at least 95%, or at least 97% homology or identity to SEQ ID NO: 29, or any value and range therebetween. Each possibility represents a separate embodiment of the invention. In some embodiments, the protein comprises an amino acid sequence with 93% to 100%, 94% to 100%, 96% to 100%, or 98% to 100% homology or identity to SEQ ID NO: 29. Each possibility represents a separate embodiment of the invention.

In some embodiments, the protein comprises or consists of the amino acid sequence:

(SEQ ID NO: 30)
MASSINISKIREAQRAQGPASILAVGTANPSNYEIQADFPDYYFRVTKS
EHMADMKGTFQRMCDKSMIRKRHMLITEEFLKENPNLCEYMAPSLDTRQ
DVVVVEVPKLGKEAATKAIKEWGQPKSKITHLIFCTTTGVDMPGADYQL
TKLLGLAPSVKRFMIYQQGCFAGGTVLRLAKDIAENNKGARVLAVCSEI
TAMSFRGPNDTHVDSLVGQALFGDGAAAVIVGSDPDLTTERPLFEIISA
AQTILPNSEGAIDGHVREVGLTIHILKDVPVLISKNIEKALTQAFSPLG
ISDWNSIFWIVHPGGPAILDQVELKVGLKKEKMATSRHVLSEYGNMSSA
CVFFIMDEMRKRSAKGGARTTGEGLDWGVLFGFGPGLTVETVVLHSLPT
TM.

In some embodiments, the protein comprises an amino acid sequence with at least 88%, at least 92%, at least 95%, or at least 97% homology or identity to SEQ ID NO: 30, or any value and range therebetween. Each possibility represents a separate embodiment of the invention. In some embodiments, the protein comprises an amino acid sequence with 88% to 100%, 91% to 100%, 93% to 100%, or 95% to 100% homology or identity to SEQ ID NO: 30. Each possibility represents a separate embodiment of the invention.

In some embodiments, the protein comprises or consists of the amino acid sequence:

(SEQ ID NO: 39)
MAEFTHLVVVKFKEEVVVEDIMKGLEKLVSQLDSVKSFVWGKDIESMEM
LRQGFTHAIMMTFGSKEDFTAFQSHPNHVEFSATFSAAIEKIVLLDFPV
VAVKTATA.

In some embodiments, the protein comprises an amino acid sequence with at least 86%, at least 91%, at least 95%, or at least 99% homology or identity to SEQ ID NO: 39, or any value and range therebetween. Each possibility represents a separate embodiment of the invention. In some embodiments, the protein comprises an amino acid sequence with 86% to 99%, 88% to 98%, 90% to 99%, or 89% to 100% homology or identity to SEQ ID NO: 39. Each possibility represents a separate embodiment of the invention.

In some embodiments, the protein comprises or consists of the amino acid sequence:

(SEQ ID NO: 40)
MSSLQNKFIEHIALIKIKPGVESTTLIDKLNGLSSIEVLLHFSAGELLG
SSHGFTHIVHCRVRSKDDLQIYLTHPIHLHLADDTLPLLDDVTVVDWFS
SNSDIVDPPKPGSAMRVTLLKLKHDSTESNKLVVIEGIKNQFKGIEDVI
VTTTFGENLFHEMHENFSIEIDKGYSIGSIAFVPGSADFQVLNSKVDNN
KLNDLTESEVVVDYVFPSAN.

In some embodiments, the protein comprises an amino acid sequence with at least 45%, at least 50%, at least 60%, at least 70%, at least 80%, at least 90%, at least 95%, or at least 99%, homology or identity to SEQ ID NO: 40, or any value and range therebetween. Each possibility represents a separate embodiment of the invention. In some embodiments, the protein comprises an amino acid sequence with 45% to 90%, 50% to 99%, 65% to 98%, or 55% to 100% homology or identity to SEQ ID NO: 40. Each possibility represents a separate embodiment of the invention.

In some embodiments, the protein comprises or consists of the amino acid sequence:

(SEQ ID NO: 41)
MSSEEQIVEHVVLFKVKPDADPSKVAAWVNGLNGLTSLQLALHLSAGQL
IRCRSSSLTFTHMLHSRYRSKEHLRQYTVHPEHVRVVTEGKSIIDDVMA
LDWMISNGAASSVCPKPGSAVRVGFYKLMESLGEIEKARVLEVMGGIEE
LSVGESFCDDRAKGYTIASTAVFPNGNPAADLDLYHSGDQLLLKEEVMK
DSIQSVVVVDYVIPSP.

In some embodiments, the protein comprises an amino acid sequence with at least 71%, at least 80%, at least 90%, or at least 99% homology or identity to SEQ ID NO: 41, or any value and range therebetween. Each possibility represents a separate embodiment of the invention. In some embodiments, the protein comprises an amino acid sequence with 71% to 97%, 75% to 99%, 80% to 98%, or 71% to 100% homology or identity to SEQ ID NO: 41. Each possibility represents a separate embodiment of the invention.

In some embodiments, the protein comprises or consists of the amino acid sequence:

(SEQ ID NO: 42)
MGEVKHILLAKFKDGISEQQIQHLITGYANLVNLVEPMKSFRWGKDVSI
ENLHQGFTHVFESTFETTEGIATYISHPAHVEFATGFLDQLEKVIVIDY
KPTSVDP.

In some embodiments, the protein comprises an amino acid sequence with at least 87%, at least 92%, at least 96%, or at least 97% homology or identity to SEQ ID NO: 42, or any value and range therebetween. Each possibility represents a separate embodiment of the invention. In some embodiments, the protein comprises an amino acid sequence with 87% to 97%, 88% to 99%, 90% to 98%, or 87% to 100% homology or identity to SEQ ID NO: 42. Each possibility represents a separate embodiment of the invention.

In some embodiments, the protein comprises or consists of the amino acid sequence:

(SEQ ID NO: 43)
MLCAPARTRLLPSISLLPSQHNIFRRLNCLIHRRNHHQTPITMSAQQQI
VEHVVLFKVKPDVDSSKVAAMVNGLNGLTSLDLTLHLSAGQLLRSRSSS
LTFTHMLHSRYRSKDDLREYAAHPDHVRVVTENIKPVIDDIMAVDWISN
DASVSPKPGSAMRVTFLKLKENLGENEKSRVLEVIGGIKNQFKSIEELS
VGENFSHDRAKGYTIASIAVLPGPSELEALDSNTELVKLEKEKVKDLLE
SVVVVDYVIPSLQSASL.

In some embodiments, the protein comprises an amino acid sequence with at least 85%, at least 88%, at least 90%, at least 92%, at least 95%, at least 97%, or at least 99% homology or identity to SEQ ID NO: 43, or any value and range therebetween. Each possibility represents a separate embodiment of the invention. In some embodiments, the protein comprises an amino acid sequence with 85% to 97%, 87% to 99%, 89% to 98%, or 85% to 100% homology or identity to SEQ ID NO: 43. Each possibility represents a separate embodiment of the invention.

In some embodiments, the protein comprises or consists of the amino acid sequence:

(SEQ ID NO: 44)
MAVAQLSSSLCISTPARISTGSGFSSSGLPRIGTTFVCGSGSPLVISGT
YHQKARVHKPAALSVRCEQSSKDGNGLNVWLGRTAMVGFAVAISVEVST
GKGLLENFGLTSPLPTVALALTALGGVLTALFIFQSASES.

In some embodiments, the protein comprises an amino acid sequence with at least 79%, at least 82%, at least 85%, at least 90%, at least 95%, at least 97%, or at least 99% homology or identity to SEQ ID NO: 44, or any value and range therebetween. Each possibility represents a separate embodiment of the invention. In some embodiments, the protein comprises an amino acid sequence with 79% to 95%, 79% to 99%, 80% to 98%, or 79% to 100% homology or identity to SEQ ID NO: 44. Each possibility represents a separate embodiment of the invention.

In some embodiments, the protein comprises or consists of the amino acid sequence:

(SEQ ID NO: 45)
MIEHIVLLKFKSDVDSTKVESMINELNGLASLDVALDVSAGKILRVSST
SSSSLTFTHLFRCCFRSADDQQVESTHPDHLRVAIEVRPVIEDMVVVDL
VSKTTIDSPNPGSAMKVRIFKLKDDLIEDSKLVVMEGIKNELKAVEHIR
FGDNINVMAKGYSIAMIAFFPDLESSVAGAEIVKDYIESELVVDFVFPP
PNVTSHS.

In some embodiments, the protein comprises an amino acid sequence with at least 50%, at least 60%, at least 70%, at least 80%, at least 90%, at least 95%, at least 97%, or at least 99% homology or identity to SEQ ID NO: 45, or any value and range therebetween. Each possibility represents a separate embodiment of the invention. In some embodiments, the protein comprises an amino acid sequence with 50% to 90%, 55% to 99%, 60% to 97%, or 50% to 100% homology or identity to SEQ ID NO: 45. Each possibility represents a separate embodiment of the invention.

In some embodiments, the protein comprises or consists of the amino acid sequence:

(SEQ ID NO: 46)
MAEFTHLVVVKFKEEVVVEDIMKGLEKLASQLDSVKSFVWGKDIESMEM
LRQGFTHAIMMTFGSKEDFTAFQSHPNHVEFSATFSAAIEKIVLLDFPV
VAVKTATA.

In some embodiments, the protein comprises an amino acid sequence with at least 87%, at least 93%, at least 95%, or at least 97% homology or identity to SEQ ID NO: 46, or any value and range therebetween. Each possibility represents a separate embodiment of the invention. In some embodiments, the protein comprises an amino acid sequence with 87% to 97%, 88% to 99%, 89% to 98%, or 87% to 100% homology or identity to SEQ ID NO: 46. Each possibility represents a separate embodiment of the invention.

In some embodiments, the protein comprises or consists of the amino acid sequence:

(SEQ ID NO: 59)
MELSLSSSSSSSLPQLHTHPSSSSSSSHYIKKSPFFINKFNNHTKCKFH
NSSALRTNFFYTTITKTSSSRFVLNKNPNQFSVKACSQVGSAGSDPALN
KVADFKDAFWRFLRPHTIRGTALGSVSLVTRALLENPNLIRWSLLLKAF
SGLVALICGNGYIVGINQIYDIGIDKVNKPYLPIAAGDLSVQSAWFLVL
AFAMVGVIIVGMNFGPFITSLYSLGLFLGTIYSVPPLRMKRFPVVAFLI
IATVRGFLLNFGVYYAVRAALGLTFQWSSAVAFITTFVTLFALVIAITK
DLPDVEGDRKFQISTFATKLGVRNIALLGSGLLLINYIGSIVAALYMPQ
AFRSSLMIPLHTILASCLIYQAWILERANYTQEAIAGYYRFVWNLFYSE
YIIFPFI.

In some embodiments, the protein comprises an amino acid sequence with at least 82%, at least 85%, at least 90%, or at least 99% homology or identity to SEQ ID NO: 59, or any value and range therebetween. Each possibility represents a separate embodiment of the invention. In some embodiments, the protein comprises an amino acid sequence with 82% to 99%, 85% to 98%, 84% to 99%, or 82% to 100% homology or identity to SEQ ID NO: 59. Each possibility represents a separate embodiment of the invention.

In some embodiments, the protein comprises or consists of the amino acid sequence:

(SEQ ID NO: 60)
MATMASSLLNPLSCSIKPNSNRLPLPTPISLSRSCRRLTIKATETDANE
VKPKAPEKAPAASGSGFNQILGIKGAKQETNKWKIRVQLTKPVTWPPLI
WGVVCGAAASGNFQWTVEDVAKSIVCMLMSGPFLTGYTQTINDWYDRDI
DAINEPYRPIPSGAISENEVITQIWVLLLGGIGLAGILDVWAGHKSPTI
FYLALGGSLLSYIYSAPPLKLKQNGWIGNFALGASYISLPWWAGQALFG
TLTPDIVVLTLLYSIAGLGIAIVNDFKSVEGDRKMGLQSLPVAFGEETA
KWICVGAIDITQLSIAGYLLGSGKPYYALALVGLIVPQIFFQFKYFLKD
PVKYDVKYQASAQPFLILGLLVTALATSH.

In some embodiments, the protein comprises an amino acid sequence with at least 92%, at least 93%, at least 95%, at least 97%, at least 98%, or at least 99% homology or identity to SEQ ID NO: 60, or any value and range therebetween. Each possibility represents a separate embodiment of the invention. In some embodiments, the protein comprises an amino acid sequence with 92% to 98%, 93% to 99%, 94% to 98%, or 92% to 100% homology or identity to SEQ ID NO: 60. Each possibility represents a separate embodiment of the invention.

In some embodiments, the protein comprises or consists of the amino acid sequence:

(SEQ ID NO: 61)
MKSLIIGSFSNKVSCYSPSLPDSSSSLIPTGCYHVSLRTFQRNRAIQAQ
SSLVRCNIGKFNETLLLSRKRSTKHVACAVSEQPIEPDATNPQSSLPNA
LDAFYRFSRPHTVIGTALSIVSVSLLAVQKLSDFSPLFFIGVFEAIVAA
FFMNIYIVGLNQLSDIEIDKVNKPYLPLASGEYSVQTGIIIVSSFAVMS
FWLGWIVGSWPLFWALFISFLLGTAYSINIPMLRWKRFALVAAMCILAV
RAIIVQVAFYLHIQTFVYGRLAVFPKPVIFATGFMSFFSVVIALFKDIP
DIVGDKIFGIQSFTVRMGQKRVFWICILLLEIAYGVAILVGASSPFLWS
RYITVLGHAILGLILWGRAKSTDLESKSAITSFYMFIWQLFYAEYLLIP
LVR.

In some embodiments, the protein comprises an amino acid sequence with at least 89%, at least 90%, at least 95%, or at least 99% homology or identity to SEQ ID NO: 61, or any value and range therebetween. Each possibility represents a separate embodiment of the invention. In some embodiments, the protein comprises an amino acid sequence with 89% to 97%, 89% to 99%, 90% to 98%, or 89% to 100% homology or identity to SEQ ID NO: 61. Each possibility represents a separate embodiment of the invention.

In some embodiments, the protein comprises or consists of the amino acid sequence:

(SEQ ID NO: 62)
MELSLSSSSSSSLPQLHTHPSSSSSSSHYIKKSPFFINKFNNHTKCKFH
NSSALRTNFFYTTITKTSSSRFVLNKNPNQFSVKACSQVGSAGSDPALN
KVADFKDAFWRFLRPHTIRGTALGSVSLVTRALLENPNLIRWSLLLKAF
SGLVALICGNGYIVGINQIYDIGIDKVNKPYLPIAAGDLSVQSAWFLVL
AFAMVGVIIVGMNFGPFITSLYSLGLFLGTIYSVPPLRMKRFPVVAFLI
IATVRGFLLNFGVYYAVRAALGLTFQWSSAVAFITTFVTLFALVIAITK
DLPDVEGDRKFQISTFATKLGVRNIALLGSGLLLINYIGSIVAALYMPQ
AFRSSLMIPLHTILASCLIYQAWILERANYTQRSQYFDMSSCRRR.

In some embodiments, the protein comprises an amino acid sequence with at least 81%, at least 85%, at least 90%, at least 95%, at least 97%, or at least 99% homology or identity to SEQ ID NO: 62, or any value and range therebetween. Each possibility represents a separate embodiment of the invention. In some embodiments, the protein comprises an amino acid sequence with 81% to 97%, 83% to 99%, 84% to 98%, or 81% to 100% homology or identity to SEQ ID NO: 62. Each possibility represents a separate embodiment of the invention.

In some embodiments, the protein comprises or consists of the amino acid sequence:

(SEQ ID NO: 63)
MELSLSSSSSSSLPQLHTHPSSSSSSSHYIKKSPFFINKFNNHTKCKFHN
SSALRTNFFYTTITKTSSSRFVLNKNPNQFSVKACSQVGSAGSDPALNKV
ADFKDAFWRFLRPHTIRGTALGSVSLVTRALLENPNLIRWSLLLKAFSGL
VALICGNGYIVGINQIYDIGIDKVNKPYLPIAAGDLSVQSAWFLVLAFAM
VGVIIVGMNFGPFITSLYSLGLFLGTIYSVPPLRMKRFPVVAFLIIATVR
GFLLNFGVYYAVRAALGLTFQWSSAVAFITTFVTLFALVIAITKDLPDVE
GDRKFQISTFATKLGVRNIALLGSGLLLINYIGSIVAALYMPQVKTTSID
HYRPYSFLVDLPGONGITLAA.

In some embodiments, the protein comprises an amino acid sequence with at least 81%, at least 85%, at least 90%, at least 95%, at least 97%, or at least 99% homology or identity to SEQ ID NO: 63, or any value and range therebetween. Each possibility represents a separate embodiment of the invention. In some embodiments, the protein comprises an amino acid sequence with 81% to 97%, 83% to 99%, 84% to 98%, or 81% to 100% homology or identity to SEQ ID NO: 63. Each possibility represents a separate embodiment of the invention.

In some embodiments, the protein comprises or consists of the amino acid sequence:

(SEQ ID NO: 64)
MATMASSLLNPLSCSIKPNSNRLPLPLPIPISLSRSCRRLTIKATETDAN
EVKPKAPEKAPAASGSGFNQILGIKGAKQETNKWKIRVOLTKPVTWPPLI
WGVVCGAAASGNFQWTVEDVAKSIVCMLMSGPFLTGYTQTINDWYDRDID
AINEPYRPIPSGAISENEVITQIWVLLLGGIGLAGILDVWAGHKSPTIFY
LALGGSLLSYIYSAPPLKLKQNGWIGNFALGASYISLPWWAGQALFGTLT
PDIVVLTLLYSIAGLGIAIVNDFKSVEGDRKMGLQSLPVAFGEETAKWIC
VGAIDITQLSIAGYLLGSGKPYYALALVGLIVPQIFFQFKYFLKDPVKYD
VKYQASAQPFLILGLLVTALATSH.

In some embodiments, the protein comprises an amino acid sequence with at least 92%, at least 93%, at least 95%, at least 97%, at least 98%, or at least 99% homology or identity to SEQ ID NO: 64, or any value and range therebetween. Each possibility represents a separate embodiment of the invention. In some embodiments, the protein comprises an amino acid sequence with 92% to 98%, 93% to 99%, 94% to 98%, or 92% to 100% homology or identity to SEQ ID NO: 64. Each possibility represents a separate embodiment of the invention.

In some embodiments, the protein comprises or consists of the amino acid sequence:

(SEQ ID NO: 65)
MASLAIGSLGSPSSRQCSSPVASSSSFAIGSQIASKFLRISKFDKTKNSP
LTLQQKHINKSIDQSFFEPLPLHKINKDKFKLYATSTNNPQFDATHDLKT
PEVSIINFVDALYRLIRPYTAVVTIVSVVAMSLLTVNSLSDFSPLFFIKV
VQALIGGIFMQMYVSGFNQICDIELDKVNKQSLPLAAGELSMKTAIVIAS
LSAIMSLSIGWFVGSPPLLWCLVWWFIVGTAYSANVLPYLRWKRFPFTAA
FCAMTSRALVLPIGYYLHMQNSIPGVSALLSRPILFAVAMLSAFSLSAMF
FKDIPDIKGDRMHGIKSLAIKLGEKRVYWISISIIEIAYIAAAFIGATSP
ISWSKYVTIIGHLGMGLLLWVRARSVDPTNTVAVQSMYMFLIKLVYAEYG
LISLVR.

In some embodiments, the protein comprises an amino acid sequence with at least 71%, at least 75%, at least 80%, at least 85%, at least 90%, at least 95%, at least 97%, or at least 99% homology or identity to SEQ ID NO: 65, or any value and range therebetween. Each possibility represents a separate embodiment of the invention. In some embodiments, the protein comprises an amino acid sequence with 71% to 90%, 75% to 99%, 73% to 97%, or 71% to 100% homology or identity to SEQ ID NO: 65. Each possibility represents a separate embodiment of the invention.

In some embodiments, the protein comprises or consists of the amino acid sequence:

(SEQ ID NO: 66)
MKSLIIGSFSNKVSCYSPSLPDSSSSLIPTGCYHVSLRTFQRNRAIQAQS
SLVRCNIGKFNETLLLSRKRSTKHVACAVSEQPIEPDATNPQSSLPNALD
AFYRFSRPHTVIGTALSIVSVSLLAVQKLSDFSPLFFIGVFEAIVAAFFM
NIYIVGLNQLSDIEIDKVNKPYLPLASGEYSVQTGIIIVSSFAVMSFWLG
WIVGSWPLFWALFISFLLGTAYSINIPMLRWKRFALVAAMCILAVRAIIV
QVAFYLHIQTFVYGRLAVFPKPVIFATGFMSFFSVVIALFKDIPDIVGDK
IFGIQSFTVRMGQKRVFWICILLLEIAYGVAILVGASSPFLWSRYITVLG
HAILGLILWGRAKSTDLESKSAITSFYMFIWQLFYAEYLLIPLVR.

In some embodiments, the protein comprises an amino acid sequence with at least 89%, at least 90%, at least 95%, or at least 99% homology or identity to SEQ ID NO: 66, or any value and range therebetween. Each possibility represents a separate embodiment of the invention. In some embodiments, the protein comprises an amino acid sequence with 89% to 97%, 89% to 99%, 90% to 98%, or 89% to 100% homology or identity to SEQ ID NO: 66. Each possibility represents a separate embodiment of the invention.

In some embodiments, the protein comprises or consists of the amino acid sequence:

(SEQ ID NO: 67)
MLIHHEHFLTTGFESSNDRAAYSINFSKQHHLHMASIATGSLCRPTSHQF
SIPVASSSSFATGSQFASKFLHISISAKKSSLTLQQRHIHKNIDQSFLKP
LALQKLNKDKFKLNGTSPDNPQFDATHDLKTQIESTINFVDVLYRLLRPY
ALLQMGLCVVTMSLLTVESLSDFSPLFFVKVAQALIGGIFMQMYVNGFNQ
ICDIELDKVNKPSLPLASGELSKTTTIVVSSLSAITSLSIGWFVGSPPLL
WSLVVWFIAGTTYSANLPYLRWKRFPFTNMFCNLTMALVVPIGTYLHMEN
SIHGVSTLLSRPLLFTVAMCTVFPVSIILFKDIPDIKGDRMHGMKSLAII
LGEKRTYWICIWILEITYIAAAFFGATSPISWSKYVTIISHLGMGFLLWL
RSKSVDVKNTVAVQSMYMFLWKLLYAEYGLILLVR.

In some embodiments, the protein comprises an amino acid sequence with at least 68%, at least 75%, at least 80%, at least 855, at least 90%, at least 95%, or at least 99% homology or identity to SEQ ID NO: 67, or any value and range therebetween. Each possibility represents a separate embodiment of the invention. In some embodiments, the protein comprises an amino acid sequence with 68% to 97%, 69% to 99%, 70% to 98%, or 68% to 100% homology or identity to SEQ ID NO: 67. Each possibility represents a separate embodiment of the invention.

In some embodiments, the protein comprises or consists of the amino acid sequence:

(SEQ ID NO: 68)
MFIHHEQFLTTGFESSNDRAAYSINFLKQHHLHMVSIATGSLCRPTSHRF
SIPVASSSSFATGSQFASISAKKSSLTLKQRHTHKNIDQSFFKPLALQKM
NKGKFKLNATSPDNSQLDATHDLKTQIESIINFVDVLYRLIRPYVVLGMG
VTIVTMCLLTVDSLSDFSPLFFVKVAQALIGSIFMAMYVNSFNEICDIEL
DKVNKPSLPLASGELSMTTAIVVSSLSAIMSLSIGWFVGSPPLLWSLVVW
FILGTAYSANLPYLRWKRFPLTTLSSALTMGALVIPIGNYMHMENSIRGV
TTLLSRPLLFAVAMCAAFHVSTILFKDIPDIKGDRMHGMKSLAIKLGEKR
MYWICIWILEIAYIAAAFFGATSPISWSKYVTIISHLGMGFLLWLRSKSV
DVKNTVAVQSMYMFLWKLFYVEHGLILLVR.

In some embodiments, the protein comprises an amino acid sequence with at least 66%, at least 75%, at least 80%, at least 855, at least 90%, at least 95%, or at least 99% homology or identity to SEQ ID NO: 68, or any value and range therebetween. Each possibility represents a separate embodiment of the invention. In some embodiments, the protein comprises an amino acid sequence with 66% to 97%, 67% to 99%, 70% to 98%, or 66% to 100% homology or identity to SEQ ID NO: 68. Each possibility represents a separate embodiment of the invention.

In some embodiments, the protein comprises or consists of the amino acid sequence:

(SEQ ID NO: 69)
MASIATGSLCRPTSHRFSIHVASSSSFATGSQFASKILQISISAKKSSLT
LQQRHIHKNIDQSFFKPLALQKMNKDKFKLNATSPDNPQFDATRDLKTQI
ESIIKFVDVLYRLLRPYAILEMGLSVVTMSLLTVESLSDFSPLFFVKVAQ
ALIGGIFMQMYVNGFNQICDIELDKVNKPSLPLASGELSTTTTIVVSSLS
AIMSLSIGWFVGSPPLLWSLVVWFIVGTTYSTNLPYLRWKRFPFTAMFCN
LTRALVVPIGTYLHMKNSIHEVSTLLSRPLLFAVAMCTVFPISIILFKDI
PDIKGDRMHGMKSLAIILGEERTYWICIWILEIAYIAAAFFGATSPISWS
KYVMIISHLGMGFLLWLRSKSVDVKNTVAVQSMYMFLWKLLYAEYGLILL
VR.

In some embodiments, the protein comprises an amino acid sequence with at least 68%, at least 75%, at least 80%, at least 855, at least 90%, at least 95%, or at least 99% homology or identity to SEQ ID NO: 69, or any value and range therebetween. Each possibility represents a separate embodiment of the invention. In some embodiments, the protein comprises an amino acid sequence with 68% to 97%, 69% to 99%, 70% to 98%, or 68% to 100% homology or identity to SEQ ID NO: 69. Each possibility represents a separate embodiment of the invention.

In some embodiments, the protein comprises or consists of the amino acid sequence:

(SEQ ID NO: 70)
MASLAIGSLGSPSSRQCSSPVASSSSFAIGSQIASKFLRISKFDKTKNSP
LALQQKHINKSIDQSFFEPLPLHKINKDKFKLYATSTNNPQFDATHDLKT
PEVSIINFVDALYRLIRPYTAVVTIVSVVAMSLLTVNSLSDFSPLFFIKV
VQALIGGIFMQMYVSGFNQICDIELDKVNKQSLPLAAGELSMKTAIVIAS
LSAIMSLSIGWFVGSPPLLWCLVWWFIVGTAYSANVLPYLRWKRFPFTAA
FCAMTSRALVLPIGYYLHMQNSIPGVSALLSRPILFAVAMLSAFSLSAMF
FKDIPDIKGDRMHGIKSLAIKLGEKRVYWISISIIEIAYIAAAFIGATSP
ISWSKYVTIIGHLGMGLLLWVRARSVDPTNTVAVQSMYMFLIKLVYAEYG
LISLVR.

In some embodiments, the protein comprises an amino acid sequence with at least 71%, at least 75%, at least 80%, at least 85%, at least 90%, at least 95%, at least 97%, or at least 99% homology or identity to SEQ ID NO: 70, or any value and range therebetween. Each possibility represents a separate embodiment of the invention. In some embodiments, the protein comprises an amino acid sequence with 71% to 100%, 80% to 100%, 90% to 100%, or 95% to 100% homology or identity to SEQ ID NO: 70. Each possibility represents a separate embodiment of the invention.

In some embodiments, the protein comprises or consists of the amino acid sequence:

(SEQ ID NO: 80)
MGLNICTRFIPCLVVVLMFLFTSTYSATPEDKFLQCISQKLNITNSDEVF
TQSNTRYSSVLESTIVNLRFATSTTPKPFAIITPLSYSHVQSAVVCAKKA
GIRIRIRSGGHDYVGLSYTSSDNVPFVVLDLKQLQNVTVEYSKKTAWVES
GATIGQLYYWVSQKSKNLGFPGGTCATIGVGGHLSGGGFGTLVRKYGLSA
DNVIDAKIVDVNGRLLDRKSMGEDLFWAIRGGGGGSFGVVVAWMVNLVHV
PEKVTAFTIVRTLEQGGSDLFNKWQHVGPKLTKDLFISVIIQPISVWNGN
GTVQVIFNSMYLGTVDKLMKTVNSSFPELGLQAKDCTEMSWIQSVLYFAG
YPIEGSIDVLKDRKPDTRNYFDNKSDHVKEPIPKERLEDLWKWCMEGDFP
ILLMDPLGGKMNEIDTTRIPYPYRNGYSYMIQYVETWENIGDSEKRISWM
RQMYENMTPYVSKNPRSAYVNYRDLDLGKNDNAKNTSYLEAMKWGSKYFG
DNFKRLAMVKGVVDPDNFFFHEQSIPPLKV.

In some embodiments, the protein comprises an amino acid sequence with at least 69%, at least 75%, at least 85%, at least 92%, at least 95%, or at least 99% homology or identity to SEQ ID NO: 80, or any value and range therebetween. Each possibility represents a separate embodiment of the invention. In some embodiments, the protein comprises an amino acid sequence with 69% to 99%, 70% to 98%, 75% to 99%, or 69% to 100% homology or identity to SEQ ID NO: 80. Each possibility represents a separate embodiment of the invention.

In some embodiments, the protein comprises or consists of the amino acid sequence:

(SEQ ID NO: 81)
MGCNLLQKLTIFVFFIMSISIPSFAYEHEHEHEHEHENDQDRVQDEKEPT
DVFTSCLTRFGVHNFTTHSKSNNDNSVYYELLNFSIQNLRFTGLSMPKPV
VIVFPETKEQLAKTVVCARESSLEIRVRCGGHSYEGTSSVSTDGRPFVVI
DMTRLDNVSVDVNSGTAWVEAGATLGQMYCAIAESSTVHGFSAGSCPTVG
TGGHISGGGFGLLSRKYGLAADNVVDAVLVTADGELLNRDTMGEDVFWAI
RGGGGGVWGIVYAFNVKLSSVPKTVTNFVVSRPGTKGQVTDLVYKWQHVA
PKLPDDFYLSSFVGAGLPERKNKPGLSATFKGFYLGSKSKALSIMNQTFP
ELKVMENDCKETSWIESILFFSGYGDESSVSDLKNRFLQDKLYYKAKSDY
VRKPIPRFGLTTALEILEKQPKGYVILDPYGGAMQTISSDSIPFPHRKGN
IFTIQYLVEWKEPDNDKTNDYLAWIRDFHGSMTPYVAQDPRAAYINYMDV
DIGVMNWIKTRVDSDDAVEMGREWGEKYFYKNYDRLVRAKTQIDPYNVFR
HQQSIPPMSLENKNRRGSISSE.

In some embodiments, the protein comprises an amino acid sequence with at least 81%, at least 85%, at least 90%, at least 95%, at least 97%, or at least 99% homology or identity to SEQ ID NO: 81, or any value and range therebetween. Each possibility represents a separate embodiment of the invention. In some embodiments, the protein comprises an amino acid sequence with 92% to 98%, 93% to 99%, 94% to 98%, or 92% to 100% homology or identity to SEQ ID NO: 81. Each possibility represents a separate embodiment of the invention.

In some embodiments, the protein comprises or consists of the amino acid sequence:

(SEQ ID NO: 82)
MKTSSNMLSVLLILFFITCSKAALDPDSVYQSFLQCLPLYSPESAEELSK
VVYSSTLNTTTYETVLQEYIKNERFNTTATPKPSVIITPTTESQVQAAVL
CAKKTGVQIKIRSGGHDYEGISYISSEPDFIVLDMFNFRSINVNVADETA
VVGAGAQLGELYYRIYEKSKTLGFPAGVCQTVGVGGHLSGGGYGTMLRKY
GLSVDHVIDAKIVDVNGQVLDRKSMGEDLFWAIRGGGGGSFGVILSYTVK
LVSVPEVNTVFRVLKTTSENASELIYKWQSIMPDIDNDLFIRVLLQPVTV
NKQKVGRATFIAHFLGDSDRLVALMSKNFPELGLKKEDCIEVSWIESVLY
WANFDLNTTKPEILLDRHSDSVSYGKRKSDYVQTPIPESGLESIFEKLVE
LGKIGLVFNSYGGRMSEVAADATPFPHRAGNIFKIQYSVNWNDADPELEA
NYLNQSRVMYDFMTPFVSKNPRAAFLNYRDLDIGVMTPGKNSYSEGEVYG
EKYFMGNFERLVKIKTAVDPDNFFRNEQSIPTRAAKNSGKSRKMMK.

In some embodiments, the protein comprises an amino acid sequence with at least 86%, at least 90%, at least 95%, or at least 99% homology or identity to SEQ ID NO: 82, or any value and range therebetween. Each possibility represents a separate embodiment of the invention. In some embodiments, the protein comprises an amino acid sequence with 86% to 97%, 87% to 99%, 88% to 98%, or 86% to 100% homology or identity to SEQ ID NO: 82. Each possibility represents a separate embodiment of the invention.

In some embodiments, the protein comprises or consists of the amino acid sequence:

(SEQ ID NO: 83)
MGLNICTRFIPCLVVVLMFLFTSTYSATPEDKFLQCISQKLNITNSDEVF
TQSNTRYSSVLESTIVNLRFATSTTPKPFAIITPLSYSHVQSAVVCAKKA
GIRIRIRSGGHDYVGLSYTSSDNVPFVVLDLKQLQNVTVEYSKKTAWVES
GATIGQLYYWVSQKSKNLGFPGGTCATIGVGGHLSGGGFGTLVRKYGLSA
DNVIDAKIVDVNGRLLDRKSMGEDLFWAIRGGGGGSFGVVVAWMVNLVHV
PEKVTAFTIVRTLEQGGSDLFNKWQHVGPKLTKDLFISVIIQPISVWNGN
GTVQVIFNSMYLGTVDKLMKTVNSSFPELGLQAKDCTEMSWIQSVLYFAG
YPIEGSMDVLKDRKPQTRRYFNNKSDHVKEPIPKERLEDLWKWCMEGDFP
ILLMDPLGGKMNEIDTTRIPYPYRNGYSYMIQYVETWENIGDSEKRISWM
RQMYENMTPYVSKNPRSAYVNYRDLDLGKNDNAKNTSYLEAMKWGSKYFG
DNFKRLAMVKGVVDPDNFFFHEQSIPPLKV.

In some embodiments, the protein comprises an amino acid sequence with at least 69%, at least 75%, at least 80%, at least 85%, at least 92%, at least 97%, or at least 99% homology or identity to SEQ ID NO: 83, or any value and range therebetween. Each possibility represents a separate embodiment of the invention. In some embodiments, the protein comprises an amino acid sequence with 69% to 97%, 70% to 99%, 75% to 98%, or 69% to 100% homology or identity to SEQ ID NO: 83. Each possibility represents a separate embodiment of the invention.

In some embodiments, the protein comprises or consists of the amino acid sequence:

(SEQ ID NO: 84)
MDQYVITKFISYLLAVFMALFCSDPTADKFLQCFTKDSNATDSNFVFTQE
NTQYSSVLESTIINLRFATSITPKPIAVITPLSYSHVQSAILCSKKIGYR
IRIRSGGHDYAGVSYTSYDHDHTPFVVLDLKELRTITIDSGENTSWVESG
ATVGELYYWVSQKSRNLGFPAGICPTVGVGGHLSGGGVGTMVRKYGLAAD
NVIDARIIDVNGRILDRKSMGEDLFWAIRGGGGASFGVIVAWKVNLVYVP
EKSFGF.

In some embodiments, the protein comprises an amino acid sequence with at least 84%, at least 87%, at least 90%, at least 95%, at least 97%, or at least 99% homology or identity to SEQ ID NO: 84, or any value and range therebetween. Each possibility represents a separate embodiment of the invention. In some embodiments, the protein comprises an amino acid sequence with 84% to 97%, 86% to 99%, 85% to 98%, or 84% to 100% homology or identity to SEQ ID NO: 84. Each possibility represents a separate embodiment of the invention.

In some embodiments, the protein comprises or consists of the amino acid sequence:

(SEQ ID NO: 85)
MELYISTRFILCFLVVLMLMFSSTYSDPLEDKFLRCLSQNSNATNSDNVF
TQENTQYSSVLESTIINLRFATSTTPKPLAIITPLSCSHVQSAVLCAKKV
GIRIRIRSGGHDYAGLSYTSSENAPFVVLDLKQLQNVTVESSKKTAWVES
GATIGQLYYWVSQKSKNLGFPAGTCATIGVGGHLSGGGFGTLVRKYGLSA
DNVIDAKIVDVNGRLLDRKSMGEDLFWAIRGGGGGSFGVVVAWKVNLVHV
PEKVTAFTIVRTLEQGGSDIFNKWQHIGHKLTKDLFIRVIIQPISVSNGN
RTVQVIFNSMYLGTVDKLMKTVNSSFPELGLQEKDCTEMSWIQSVLYFAG
YPIEGSMDVLKDRKPDTRNYFDNKSDHVKEPIPKERLEDLWKWCMEVDFP
ILIMEPLGGKMNEIDTTRIPYPYRKGYSYMIQYVEAWDNIGDSEKHISWL
RQMYENMTPYVSKNPRSAYVNYRDLDLGKNDNAKNTSYLEAMKWGSKYFG
DNFKRLAMVKGVVDPDNFFFHEQSIPPLKV.

In some embodiments, the protein comprises an amino acid sequence with at least 72%, at least 75%, at least 85%, at least 92%, at least 95%, or at least 99% homology or identity to SEQ ID NO: 85, or any value and range therebetween. Each possibility represents a separate embodiment of the invention. In some embodiments, the protein comprises an amino acid sequence with 72% to 99%, 74% to 98%, 78% to 99%, or 72% to 100% homology or identity to SEQ ID NO: 85. Each possibility represents a separate embodiment of the invention.

In some embodiments, the protein comprises or consists of the amino acid sequence:

(SEQ ID NO: 86)
MGLNICTRFIPCLVVVLMFLFTSTYSATPEDKFLQCISQKLNITNSDEVF
TQSNTRYSSVLESTIVNLRFATSTTPKPFAIITPLSYSHVQSAVVCAKKA
GIRIRIRSGGHDYVGLSYTSSDNVPFVVLDLKQLQNVTVEYSKKTAWVES
GATIGQLYYWVSQKSKNLGFPGGTCATIGVGGHLSGGGFGTLVRKYGLSA
DNVIDAKIVDVNGRLLDRKSMGEDLFWAIRGGGGGSFGVVVAWMVNLVHV
PEKVTAFTIVRTLEQGGSDLFNKWQHVGPKLTKDLFISVIIQPISVWNGN
GTVQVIFNSMYLGTVDKLMKTVNSSFPELGLQAKDCTEMSWIQSVLYFAG
YPIEGSMDVLKDRKPQTRRYFNNKSDHVKEPIPKERLEDLWKWCMEGDFP
ILLMDPLGGKMNEIDTTRIPYPYRNGYSYMIQYVETWENIGDSEKRISWM
RQMYENMTPYVSKNPRSAYVNYRDLDLGKNDNAKNTSYLEAMKWGSKYFG
DNFKRLAMVKGVVDPDNFFFHEQSIPPLKV.

In some embodiments, the protein comprises an amino acid sequence with at least 69%, at least 75%, at least 85%, at least 92%, at least 95%, or at least 99% homology or identity to SEQ ID NO: 86, or any value and range therebetween. Each possibility represents a separate embodiment of the invention. In some embodiments, the protein comprises an amino acid sequence with 69% to 99%, 70% to 98%, 75% to 99%, or 69% to 100% homology or identity to SEQ ID NO: 86. Each possibility represents a separate embodiment of the invention.

In some embodiments, the protein comprises or consists of the amino acid sequence:

(SEQ ID NO: 87)
MGEDLFWAIRGGGGGSFGVVVAWMVNLVHVPEKVTAFTIVRTLEQ
GGSDLFNKWQHVGPKLTKDLFISVIIQPISVWNGNGTVQVIFNSM
YLGTVDKLMKTVNSSFPELGLQAKDCTEMSWIQSVLYFAGYPIEG
SMDVLKDRKPQTRRYFNNKSDHVKEPIPKERLEDLWKWCMEGDFP
ILLMDPLGGKMNEIDTTRIPYPYRNGYSYMIQYVETWENIGDSEK
RISWMRQMYENMTPYVSKNPRSAYVNYRDLDLGKNDNAKNTSYLE
AMKWGSKYFGDNFKRLAMVKGVVDPDNFFFHEQSIPPLKV.

In some embodiments, the protein comprises an amino acid sequence with at least 71%, at least 75%, at least 85%, at least 92%, at least 95%, or at least 99% homology or identity to SEQ ID NO: 87, or any value and range therebetween. Each possibility represents a separate embodiment of the invention. In some embodiments, the protein comprises an amino acid sequence with 75% to 99%, 74% to 98%, 78% to 99%, or 71% to 100% homology or identity to SEQ ID NO: 87. Each possibility represents a separate embodiment of the invention.

In some embodiments, the protein comprises or consists of the amino acid sequence:

(SEQ ID NO: 88)
MELKLFTCKLVTIILALSLSFFTSTSSSDFLDCISQKNLSNIIFT
PNDTSYSTILQFTIPNLRFNTPKTTKPLAIITPTTYSHVQSTIIC
SVQFKHHVRIRSGGHDYEGLSYTSFNNTPFILLDLNQLRSVTVDL
DSNTTWVESGATLGELLYWVSRKSNILGIPTGECTSVGVGGQLSG
GGFGNMARKYGLFSDNAVDALIIDVNGRILDRDSMGEDLFWAIRG
GGGGNFGVVLSWKINLVYVPPKVTVFTVSKMLDENGTKIVHKWQY
IAHNITQDLFINLIVSPVTVSNTTILAVTINSLFLGMKNELVATM
DVIFPELGLQEKDCIEMSWIESVVYHSVYLRGQSVDALIERRPWP
KSYNKYKSDYVKKPMSEKALEKLWKWCLEENLILAIEPHGGKMSE
IDESSTPYPHRKGNLYIIQYVMQWDEGYNTTQKHVASIRRVYKKM
APFVSKNPREAYVNFRDLDLGTNGNACGTSGASYVQALRWGKKYF
KGNFKRLAIVKGRVDPTNFFCNEQSIPPYSY.

In some embodiments, the protein comprises an amino acid sequence with at least 74%, at least 79%, at least 85%, at least 92%, at least 95%, or at least 99% homology or identity to SEQ ID NO: 88, or any value and range therebetween. Each possibility represents a separate embodiment of the invention. In some embodiments, the protein comprises an amino acid sequence with 74% to 99%, 78% to 98%, 81% to 99%, or 74% to 100% homology or identity to SEQ ID NO: 88. Each possibility represents a separate embodiment of the invention.

In some embodiments, the protein comprises or consists of the amino acid sequence:

(SEQ ID NO: 102)
MTNSELVFIPSPGAGHLPPTVELAKLLLHREPQLSVTIIIMNLPH
ETKPTTETRMSTPRLRFIDIPKDESTKDLISRHTFISAFLEHQKP
HVRNIVRSITESDSVRLVGFVVDMFCIAMMDVANELGAPTYLYFT
SSAASLGLMFCLQAKRDDEEFDVTELKDKDSELSIPCYTNPLPAK
LLPSVLFDKRGGSKTFIDLARKYRESRGIVVNTFQELESYAIEYL
ASSNANVPPVFPVGAILNQEKKVNDDKTEEIMTWLNEQPESSVVF
LCFGSMGSFGEDQIKEIALAIEESGQRFLWSLRRPPSNENKYPKE
YENFGEVLPEGFLERTSSVGKVIGWAPQMAVLSHSSVGGFVSHCG
WNSTLESIWCGVPVAAWPLYAEQQLNAFKLVVELGLAVEIKIDYR
SENEIILTSKEIESGIRRLMNDEELRMKVKEMKGNSRFAVSEGGS
SYVSIRRFIDLVMTKE.

In some embodiments, the protein comprises an amino acid sequence with at least 75%, at least 85%, at least 92%, at least 95%, or at least 99% homology or identity to SEQ ID NO: 102, or any value and range therebetween. Each possibility represents a separate embodiment of the invention. In some embodiments, the protein comprises an amino acid sequence with 75% to 99%, 76% to 98%, or 75% to 100% homology or identity to SEQ ID NO: 102. Each possibility represents a separate embodiment of the invention.

In some embodiments, the protein comprises or consists of the amino acid sequence:

(SEQ ID NO: 103)
MPTSELVFIPSPGVGHLSPTIELVNQLLHRDQRLSVTIIVMKFSL
ESKHDTETPTSTPRLRFIDIPYDESAMALINPNTFLSAFVEHNKP
HVRNIVRDISESNSVRLAGFVVDMFCVAMTDVVNEFEIPTYIYFT
STANLLGLMFYLQAKRDDEGFDVTVLKDSESEFLSVPSYVNPVPA
KVLPDAVLDKNGGSQMCLDLAKGFRESKGIIVNTFQELERRGIEH
LLSSNMNLPPVFPVGPILNLRNAPNDGKTADIMTWLNDHPENSVV
FLCFGSMGSFEKEQVKEIAIAIEQSGQRFLWSLRRPTSLEKFEFP
KDYENPEEVLPKGFLERTKGVGKVIGWAPQMAVLSHPSVGGFVSH
CGWNSTLESIWCGVPIAAWPLYAEQKINAFQLVVEMGMAAEIRID
YRTNTRPGGGKEMMVMAEEIESGIRKLMSDDEMRKKVKGMKDKSR
AAVLEGGSSHTSIGILIENLVSITI.

In some embodiments, the protein comprises an amino acid sequence with at least 76%, at least 85%, at least 92%, at least 95%, or at least 99% homology or identity to SEQ ID NO: 103, or any value and range therebetween. Each possibility represents a separate embodiment of the invention. In some embodiments, the protein comprises an amino acid sequence with 76% to 99%, 80% to 98%, or 76% to 100% homology or identity to SEQ ID NO: 103. Each possibility represents a separate embodiment of the invention.

In some embodiments, the protein comprises or consists of the amino acid sequence:

(SEQ ID NO: 104)
MVGLKCFWILQKGFRESKGIIVNTFQELERRGIEHLLSSNMDLPP
VFPVGPILNLRNARNDGKMADIMTWLNDQPENSVVFLCFGSRGSF
KEEQVKEIAIAIEQSGQRFLWSLRRPTSIETFEFPKYYENPEEVL
PKGFLERTKSVGKVIGWAPQMAVLSHPSVGGFVSHCGWNSTLESI
WCGVPIAAWPLYAEQQTNAFQLVVEMGMAAEIRIDYRTNTPLVGG
KDMMVTAEEIERGIRKLMSDDEMRKKVKDMKDKSRGAVLEGGSSH
TSIGNLIDVLVSITI.

In some embodiments, the protein comprises an amino acid sequence with at least 77%, at least 85%, at least 92%, at least 95%, or at least 99% homology or identity to SEQ ID NO: 104, or any value and range therebetween. Each possibility represents a separate embodiment of the invention. In some embodiments, the protein comprises an amino acid sequence with 77% to 99%, 79% to 98%, or 77% to 100% homology or identity to SEQ ID NO: 104. Each possibility represents a separate embodiment of the invention.

In some embodiments, the protein comprises or consists of the amino acid sequence:

(SEQ ID NO: 105)
MATNNLHFLLIPHIGPGHTIPMIDMAKLLAKQPNVMVTIATTPLN
ITRYGHTLADAINSFRFFEVPFPAVEAGLPEGCESTDKIPSMDLV
PNFLTAIGMLEQKLEEHFHLLEPRPNCIISDKYMSWTGDFADKYR
IPRIMFDGMSCFNELCYNNLYENKVFEGMHETEPFVVPGLPDKIE
LTRKQLPPEFNPSSIDTSEFRQRARDAEVRAYGVVINSFEELEQE
YVNEYKKLRKGKVWCIGPLSLCNSDNSDKAQRGNIASVDEEKCLK
WLDSHEADSVVYACFGSLVRVNTPQLIELGLGLEASNRPFIWVVR
SVHREKEVEEWLVESGFEERIKDRGLIIRGWAPQVLILSHPSIGG
FLTHCGWNSTLESVCAGVPMITWPQFAEQFINEKLIVQVLGIGVG
VGVDSVVHVGEEDRSGVKVKRESVTKAIEKVMDDEIDGNERRRRS
KEFGKIANNAIKEGGSSYLNLTLLIQDIMRYANADASS.

In some embodiments, the protein comprises an amino acid sequence with at least 88%, at least 92%, at least 95%, or at least 99% homology or identity to SEQ ID NO: 105, or any value and range therebetween. Each possibility represents a separate embodiment of the invention. In some embodiments, the protein comprises an amino acid sequence with 88% to 100%, 90% to 100%, or 95% to 100% homology or identity to SEQ ID NO: 105. Each possibility represents a separate embodiment of the invention.

In some embodiments, the protein comprises or consists of the amino acid sequence:

(SEQ ID NO: 106)
MEKTPHIAIVPSPGMGHLIPLVEFAKKLKNHHNIHATFIIPNDGP
LSISQKVELDSLPNGLNYLILPPVNFDDLPQDTQIETRISLMVTR
SLDSLREVFKSLVVEKNMVALFIDLFGTDAFDVAIEFGVSPYVFF
PSTAMALSLFLYLPKLDQMVSCEYRELPEPVQIPGCIPVRGQDLV
DPVQDRKNDAYKWVLHNAKKYSMAKGIAVNSFKELEGGALNALLE
DEPGKPKVYPVGPLVQTGFSCDVDSIECLKWLDGQPCGSVLYISF
GSGGTLSSSQLNELAMGLELSEQRFIWVVRSPNDQPNATYFDSHG
HKDPLGFLPKGFLERTKGIGFVIPSWAPQAQILSHSATGGFLTHC
GWNSILETVVHGVPVIAWPLYAEQKMNAVSLTEGIKMALRPTVGE
NGIVGRLEVARVVKSLLEGEEGKAIRSRVRDLKDAAANVLSKDGS
STKTLDQLAVQLKKQELS.

In some embodiments, the protein comprises an amino acid sequence with at least 90%, at least 95%, or at least 99% homology or identity to SEQ ID NO: 106, or any value and range therebetween. Each possibility represents a separate embodiment of the invention. In some embodiments, the protein comprises an amino acid sequence with 90% to 100%, 93% to 100%, 95% to 100%, or 97% to 100% homology or identity to SEQ ID NO: 106. Each possibility represents a separate embodiment of the invention.

In some embodiments, the protein comprises or consists of the amino acid sequence:

(SEQ ID NO: 107)
MTQKQMQMQPHFLLVTYPAQGHINPSLQFAERLIRLGVKVTFTTT
VSAYRRMSKAGNISEFLNFAAFSDGFDDGFNFETDDHGLFLTQLR
SRGKDSLKETILSNAKNGTPISCLVYTLLLPWAPEVARGLNVPSA
FLWIQPASVLRLYYYYFNGYNELIGDDCNEPSWSIQLPGLPLLKS.

In some embodiments, the protein comprises an amino acid sequence with at least 77%, at least 85%, at least 92%, at least 95%, or at least 99% homology or identity to SEQ ID NO: 107, or any value and range therebetween. Each possibility represents a separate embodiment of the invention. In some embodiments, the protein comprises an amino acid sequence with 77% to 100%, 79% to 100%, 80% to 100%, or 90% to 100% homology or identity to SEQ ID NO: 107. Each possibility represents a separate embodiment of the invention.

In some embodiments, the protein comprises or consists of the amino acid sequence:

(SEQ ID NO: 108)
MTKIQQQPHFLLVTYPAQGHINPSLRFAERLIRLGVKVTFTITVS
AYRRMSKAGHISEFLNFAVFSDGFDDGFNSKTDDYGLFLTQFRSR
GKDSLKETILSNAKNGTPVSCLVYTLLLPWAPEVARGLNVPSAFL
WIQPASVLRLYYYYFNGYNELIGDDCNEPSWSIQLPGLPLLKSRD
LPSFCLPSNPYADVLTLVKEHLDVLDLEEKPKILVNSFDELEREA
LNEIDGKLKMVAVGPLIPSAFFGWTGCI.

In some embodiments, the protein comprises an amino acid sequence with at least 73%, at least 85%, at least 92%, at least 95%, or at least 99% homology or identity to SEQ ID NO: 108, or any value and range therebetween. Each possibility represents a separate embodiment of the invention. In some embodiments, the protein comprises an amino acid sequence with 73% to 100%, 77% to 100%, 85% to 100%, or 90% to 100% homology or identity to SEQ ID NO: 108. Each possibility represents a separate embodiment of the invention.

In some embodiments, the protein comprises or consists of the amino acid sequence:

(SEQ ID NO: 109)
MGSWRNSRTTSTKFLWLILPLMVVTVIIGVKKSNYGSKYNYPWVW
SSVINSYSSSAVKEDVTVVAEGPVESFGLRSTVVNGGGVVAEGPS
EDFGFNSSYPPLAMEDEMDVELPAIAKEDDLNATLSGPDLFVSAN
QTGGLHVDIGINSKYTSLDKLEARLGQVRAAIKEAESGNRTYDPD
YVPEGPMYWHAASFHRSYLEMEKQFKVFVYEEGEPPIFHNGPCKN
IYAMEGNFIYHMETTKFRTKNPEKAHTFFLPMSAAMMVRFIFERD
PNVDHWRPMKQTIKDYVDLVGGKYPFWNRSLGADHFTVACHDWVS
KVFYPIIFMLLLVFIFRMSTGC.

In some embodiments, the protein comprises an amino acid sequence with at least 81%, at least 90%, at least 95%, or at least 99% homology or identity to SEQ ID NO: 109, or any value and range therebetween. Each possibility represents a separate embodiment of the invention. In some embodiments, the protein comprises an amino acid sequence with 81% to 100%, 85% to 100%, 87% to 100%, or 91% to 100% homology or identity to SEQ ID NO: 109. Each possibility represents a separate embodiment of the invention.

In some embodiments, the protein comprises or consists of the amino acid sequence:

(SEQ ID NO: 110)
MSTVEVAKLLVNRDHRLFITFLIIQPPSSGSGSAITTYIESLAEK
AMDRISFIELPQDKIPPPRYPKSLPTAESKAHPLIFMIEFIKCHC
KYVRNIVSDMISQPSSGRVAGLVIDMLCFSMMDVANEFNIPTYVF
VTSNAAFLGFYLYVQILSNDQNQDVVELSKSDTEISVPGFVKPVP
TKVFWTVVRTKEGLDFVLSSAQKLRQAKAIMVNTFLELETHAIKS
LSDDTSIPPVYPVGPILNLEGGAGKTFDNDISRWLDSQPPSSVVF
LCFGSHGCFDEIQVKEIAHALEQSGHRFLWSLRRPPSDQTLKVPG
DYEDPGVVLPEGFLERTAGRGKVIGWAPQVMVLAHRAVGGFVSHC
GWNSLLESLWFGVPTATWPIYAEQQMNAFEMVVELGLAVEITLDY
RNDMDMFIVTAQEIESGIRKVMEDNEVRTKVKERSEKSRAAVAEG
GSSYASVGHLIKEFTGNIS.

In some embodiments, the protein comprises an amino acid sequence with at least 74%, at least 85%, at least 92%, at least 95%, or at least 99% homology or identity to SEQ ID NO: 110, or any value and range therebetween. Each possibility represents a separate embodiment of the invention. In some embodiments, the protein comprises an amino acid sequence with 74% to 100%, 79% to 100%, 85% to 100%, or 90% to 100% homology or identity to SEQ ID NO: 110. Each possibility represents a separate embodiment of the invention.

In some embodiments, the protein comprises or consists of the amino acid sequence:

(SEQ ID NO: 111)
MSSFINFVESTTQLQPQFEQLIQTLLPITAIISDGFLMWTQDSAE
KFNIPRLVFYGTNIFFMTMCNIMAQFKPHAAVNSDDEAFDVPGFT
RFKLTANDFEPPFNEVEPKGSMLDFLLEQQKAMVRSHGLVVNSFY
EIEHEFNVYWNQNYGPKAWLMGPFCVAKPYASNVMDSEISTKVVK
KSAWIQWLDRKLAANEPVLYISFGTQAEASMEHLHEVAIGLERSN
VSFIWVVKAKQMQLIGAGFEERVKGRGKVVTEWVDQMEILKHEIV
SGFLSHCGWNSLLESMCVGVPVLAMPLMADQLLNARLVVEEIGMG
LRLWPRGMVARGIVGAEEVEKMVVELMEGEGGRRVRKRVIEVREM
AYGAMKEGGSSSRTLDSLIDHVCEAFHKTV.

In some embodiments, the protein comprises an amino acid sequence with at least 76%, at least 85%, at least 92%, at least 95%, or at least 99% homology or identity to SEQ ID NO: 111, or any value and range therebetween. Each possibility represents a separate embodiment of the invention. In some embodiments, the protein comprises an amino acid sequence with 76% to 100%, 80% to 100%, 90% to 100%, or 95% to 100% homology or identity to SEQ ID NO: 111. Each possibility represents a separate embodiment of the invention.

In some embodiments, the protein comprises or consists of the amino acid sequence:

(SEQ ID NO: 112)
MGSLKKGAHILIFPFPAQGHMLPLLDLTHHLATNGLTITILVTPK
NLPILNPLLSSSPNIQPLVFPFPPHPRLPPHVENVKDIGNHANVP
ITNSLAKLQDQIIQWFNSHHNPPVAIISDFFLGWTQHLANKLGIP
RVGFFSSGAYLTAVLDYVCHNIKTVRSQEETVFHDLPNSPCFKFE
HLPGLAQIYKESDPEWELVLDGHIANGLSWGWIVNTFDGLESRYM
EYLTKKMGVGRVFGVGPVNLLNGSDPMTRGKSESGSDSGVLNWLD
GKPDGSVLYVCFGSQKFLTNDQMEGLSIGLEQSGVHYVWVVKDEQ
GDAIRSGSGRGLVVTGWAPQVSILGHGAVGGFLSHCGWNSVLEAI
VNGVMILAWPMEADQFVNAKLLVDDHGIGVWVCEGPNTVPDSTEL
ARKIGESMSTDKSEKVKAKEMKNKANEAVKEGGSSSMELSRLVKE
LSNFETNGP.

In some embodiments, the protein comprises an amino acid sequence with at least 81%, at least 92%, at least 95%, or at least 99% homology or identity to SEQ ID NO: 112, or any value and range therebetween. Each possibility represents a separate embodiment of the invention. In some embodiments, the protein comprises an amino acid sequence with 81% to 100%, 85% to 100%, 90% to 100%, or 93% to 100% homology or identity to SEQ ID NO: 112. Each possibility represents a separate embodiment of the invention.

In some embodiments, the protein comprises or consists of the amino acid sequence:

(SEQ ID NO: 113)
MDTQTQVKKQKLETMEHKTSSAEIFVLPFFGTGHINPAMELCRNI
SSHNYKTTLIIPSHLSSSIPSPFSSTLLHVAEIPFTASDPEPGSG
RGNPLDAQNKQMGEGIKAFMSARSDGSKLPTCVVIDVMMNWSKEI
FVDYQIPIVSFFTSGATNTAMGYGRWKAKIGDLKPGETRVIPGLP
TEMAVTFADLNQGPRGRGPRPDGSRPDGPRSGPPGGMRSGPPHGM
RGGGRGGRGGGRPGPDAKPRWVDEVDGSVALLINTCDNLERVFID
YIAEETKIPVYGVGPLLPEKYWKSAGSLLRDHEMRSNHKANYSED
EVFQWLESKPVGSVIYISFGSEVGPTIDEYKELAGSLEGSNQNFI
WVIQPGSGITGMPRSFLGPVNTDSEEEEEGYYPEGLDVKVGNRGL
IITGWAPQLLILSHPSTGGFLSHCGWNSTVEAIGRGVPILGWPLR
GDQFDNAKLVANHLKIGFAMSSVASEGGRPGKFNKETITAGIEKL
MNDEDVHKQAKKLSKEFESGFPVSSVKALGAFVESISQKAT.

In some embodiments, the protein comprises an amino acid sequence with at least 71%, at least 85%, at least 92%, at least 95%, or at least 99% homology or identity to SEQ ID NO: 113, or any value and range therebetween. Each possibility represents a separate embodiment of the invention. In some embodiments, the protein comprises an amino acid sequence with 71% to 100%, 77% to 100%, 85% to 100%, or 90% to 100% homology or identity to SEQ ID NO: 113. Each possibility represents a separate embodiment of the invention.

In some embodiments, the protein comprises or consists of the amino acid sequence:

(SEQ ID NO: 114)
MSLVTNNPHLLVYPLPTSGHIIPLLDLTDLLLRRGLTITVVISTT
DLTLLDTLLSSHPTSLHKLYFPDPEIGPSSHPVIARIIATQKLFD
PIVKWFESHPSPPVAIISDFFLGWTNELASRLGIRRVVFSPSGAL
GHSILQSLWRDVAEINAKNVDGNGNYSISFTDIPNSPEFHWWQLS
QLLRVHREGDPDFEFFRNGMLANTKSWGIVYNTFERIEKVYIDHV
KKQIGHDRVWAIGPLLPEEHGPVGSTARGGSSVVPPHDLLTWLDK
KPHDSVVYICFGSRLTLSEKQMSALASALELSNVDFILCVKASGS
SFIPSGFEDRVVGRGFVIKGWAPQLAILRHRAVGSFVTHCGWNST
LEGVSSGVMMLTWPMGADQYANAKLLVDQLGVGKRVCEGGPESVP
DSTELARLLEESLSGDTSERVKVKELSREANTAVKEGTSIRDLNM
FVNLLSEL.

In some embodiments, the protein comprises an amino acid sequence with at least 78%, at least 85%, at least 92%, at least 95%, or at least 99% homology or identity to SEQ ID NO: 114, or any value and range therebetween. Each possibility represents a separate embodiment of the invention. In some embodiments, the protein comprises an amino acid sequence with 78% to 100%, 85% to 100%, 90% to 100%, or 93% to 100% homology or identity to SEQ ID NO: 114. Each possibility represents a separate embodiment of the invention.

In some embodiments, the protein comprises or consists of the amino acid sequence:

(SEQ ID NO: 130)
MATQVKTEEKHLKVEIINKTYVKPETPLGRKECQLVTFDLPYIAF
YYNQKLIIYKGGVEEFEDTVEKLKDGLKVVLGEFHQLAGKLDKDD
DGVFKVVYDDDMDGVEVLSAVAEDTATADLMDEEGTIKLKELVPY
NSVLNIEGLHRPLLSIQITKLKDGLVLGCAFNHAILDGTSTWHFM
SSWAQICSGSKSISAAPFLDRTQARNTRVKLDLTPPAQTNGNSNG
DTNGDASATKPPAPAPLREKIFKFSESAIDKIKAKINANPPEGST
KPFSTFQSLSTHIWHAVTRARNLKPEDYTVFTVFADCRKRVDPPM
PDSYFGNLIQAIFTVTAAGLLQANPPEFAASMIQKAIDMHDAKAI
EARNKEWESNPIIFQYKDAGVNCVAVGSSPRFKVYDVDFGFGKPE
SVRSGANNRFDGMVYLYQGKSGGRSIDVEISLDASAMGNLEKDKE
FLIQE.

In some embodiments, the protein comprises an amino acid sequence with at least 87%, at least 92%, at least 95%, or at least 99% homology or identity to SEQ ID NO: 130, or any value and range therebetween. Each possibility represents a separate embodiment of the invention. In some embodiments, the protein comprises an amino acid sequence with 87% to 100%, 90% to 100%, or 95% to 100% homology or identity to SEQ ID NO: 130. Each possibility represents a separate embodiment of the invention.

In some embodiments, the protein comprises or consists of the amino acid sequence:

(SEQ ID NO: 131)
MASLPLLTVLEQSHVSPPPATVVDKSLSLTFFDFLWLTQPPIHNL
FFYEFSIDETQFVETIVPSLKNSLSITLQHFYPFAGNLILFPDNK
RPEIRYVEGDYVMVTFAKSSLDFNELVGNHPRDCDQFYDLIPPLG
ESVKTSEFRKIPLFSVQVTFFPQKGVSIGMTNHHSLGDASTRFCF
LNAWTSISRSSSDESFLANGTKPFYDRVISNPKLDQSYLKFSKID
TLYEKYQPLSLSRPSNKLRGTFILTRKILNELKKSVSIKLPTLSY
VSSFTVACGYIWSCIAKSRNDDLQLFGFTIDCRARLDPPVPSTYF
GNCVGGCMAMAKTTLLTEDDGFITAAKLLGESLHKTLTESGGIVK
DIEVFEDLFKDGLPTTMIGVAGTPKLKFYETDFGWGNPKKVETIS
IDYNMSISMNACRESKDDLEIGVCLMNTEMEAFVRLFDEGLESYV.

In some embodiments, the protein comprises an amino acid sequence with at least 72%, at least 80%, at least 89%, at least 95%, or at least 99% homology or identity to SEQ ID NO: 131, or any value and range therebetween. Each possibility represents a separate embodiment of the invention. In some embodiments, the protein comprises an amino acid sequence with 72% to 100%, 80% to 100%, or 90% to 100% homology or identity to SEQ ID NO: 131. Each possibility represents a separate embodiment of the invention.

In some embodiments, the protein comprises or consists of the amino acid sequence:

(SEQ ID NO: 132)
MGSENVHKIMKINITKSSFVQPSKPTVLPTNHIWTSNLDLVVGRI
HILTVYFYRPNGASNFFDPIVMKKALADVLVSFYPMAGRISKDDN
GRVVINCNDEGVLFVEAESDSTLDDFGEFTPSPELRQLTPTIDYS
GDISTYPLFFAQVTHFKCGGVGFGCGVFHTLADGLSSIHFINTWS
DMARGLSIAIPPFTDRTLLRAREPPTPTFDHVEYHLPPSMKTTSQ
TNKSRKPSTAMLKLTLDQLNALKAAAKNEGGNTNYSTYEILAAHL
WRCACKARGLPDDQLTKLYVATDGRSRLSPQLPPGYLGNVVFTAT
PVAKSADLTTQPLSNAASLIRTTLTKMDNDYLRSAIDYLEVQPDL
SALIRGPSYFASPNLNINTWTRLPVHDADFGWGRPVFMGPAVILY
EGTIYVLPSPNNDRSMSLAVCLDADEQPSFEKFLYDF.

In some embodiments, the protein comprises an amino acid sequence with at least 90%, at least 92%, at least 95%, at least 97%, or at least 99% homology or identity to SEQ ID NO: 132, or any value and range therebetween. Each possibility represents a separate embodiment of the invention. In some embodiments, the protein comprises an amino acid sequence with 90% to 100%, 95% to 100%, or 97% to 100% homology or identity to SEQ ID NO: 132. Each possibility represents a separate embodiment of the invention.

In some embodiments, the protein comprises or consists of the amino acid sequence:

(SEQ ID NO: 133)
MPSSSSSPSSTADSVTIISKCTVYPHMKNSTPESLQLSVSDLPML
SCQYIQKGVLLSQPPPNHTNNIISHLKLSLSKTLSHFPPLAGRLS
TDSHGHVSIICNDSGVEFVHSTANHLHTHQILPLNSDVHPCFKTF
FAFDKTLSYAGHHQPIAAVQVTELADGLFIGCTVNHAVVDGTSFW
NFFNTFAEITKGCQKVTNLPDFSRENVFISPVVLPLPSGGPSATF
SGDEPLRERIIHFSRDAILKMKFRANNPLWRQPQNSDLDDTEIYG
KVCNDINGKVNGAFKPKSEISSFQSLCGQLWRAVTRARKENDPIK
TTTFRMAVNCRHRLDPKVDKLYFGNLIQSIPTVASVGELLSHDLS
WAANELHQNVVAHDNATVRRGVKDWENNPKLFPLGNFDGAMITMG
SSPRFPMYNNDFGWGRPMAVRSGKANKFDGKISAFPGRDGDGSVD
LEVVLAPETMACLERDHEFMQYVS.

In some embodiments, the protein comprises an amino acid sequence with at least 86%, at least 92%, at least 95%, or at least 99% homology or identity to SEQ ID NO: 133, or any value and range therebetween. Each possibility represents a separate embodiment of the invention. In some embodiments, the protein comprises an amino acid sequence with 86% to 100%, 90% to 100%, or 95% to 100% homology or identity to SEQ ID NO: 133. Each possibility represents a separate embodiment of the invention.

In some embodiments, the protein comprises or consists of the amino acid sequence:

(SEQ ID NO: 134)
MKWFFITHKATQRCLNSKQFHLHGGSNFVSGNRCFLASHSMERPK
FMLIPYYPYQIRSLNSSHRYSSTSPSGSPHSFLNGTKNENYTKKV
DLEIISREIIKPASPTPHHLRNFNLSLLDQIVFDCYTPVILFIPN
SNKATVTDVMIKRLKHLKETLSRILSQFYPFAGEVKDRLHIECND
KGVNYIEAQINETLEEFLCHPDNEKARELMPESPHVQESAIGNYA
MGIQINIFSCGGIGLSMSMAHKIMDFYTYTIFMKAWAAAVRGSPD
TIISPSFVASEVFPNDPSQEDSIPIELKSSNLLSTKRFEFDPTAL
ALLKGQVVASGSPPQRGPSRMEATTAVIWKAAAKAASTVRRFDPK
SPHALALPVNIRKRASPALPDNSIGNIVMRGIAICFPESQPDLPT
LMGKVRESIAKLNSDYIESLKGEKGHETVNKMLKELKLRTNMTKV
GGKFVASCIFNSGIYELDFGWGKPIWFYVVNPGSDSCVVLTDTLK
GGGVEATITLPPDEMEIFERDHELLSYTTINPSPLRFLDH.

In some embodiments, the protein comprises an amino acid sequence with at least 59%, at least 65%, at least 75%, at least 85%, at least 90%, or at least 99% homology or identity to SEQ ID NO: 134, or any value and range therebetween. Each possibility represents a separate embodiment of the invention. In some embodiments, the protein comprises an amino acid sequence with 59% to 100%, 70% to 100%, 80% to 100%, 90% to 100%, 95% to 100%, or 97% to 100% homology or identity to SEQ ID NO: 134. Each possibility represents a separate embodiment of the invention.

In some embodiments, the protein comprises or consists of the amino acid sequence:

(SEQ ID NO: 135)
MEVPDQFHLNILEQCHVSPSPNSIIPSFSLPLTFLDIPWLFYPSN
QTLFFFPEPPPKTTIITTLKQSLSLTLHHFHPLAGNLSLPSPPAE
PHIVYTKNDSIALTIAQTNTNIHHLSCNHPRSVKNLYSLLPKLPS
PSMSRETHVGLVIPLLTIQITVFADLGYSIGVTMQHAAVDERTFD
QFMKCWASVCTSLLKNDSLFTFKSTPWYDRSVIIDPKSLKTTFLK
QWWNRSNSLNESHDQENDDHDLVLATFVLSSLDINMIKNHILAKC
KMINEDPPLHLSPYVSACAYLWKCLIKIQETHDSIKGGPLYLGFN
AGGITRLGYDIPSTYFGNCIAFGRCKAFESELLGDNGIVFAAKSI
GKEIKRLDKDVLGGANKWISDWDELTIRLLGSPKVDSYGMDFGWG
KVEKVEKISSISNHGRVNVISLSGCKDFKGGIEIGVVLSVAKMNV
FTSLFHGGLMEFAY.

In some embodiments, the protein comprises an amino acid sequence with at least 71%, at least 80%, at least 90%, at least 95%, or at least 99% homology or identity to SEQ ID NO: 135, or any value and range therebetween. Each possibility represents a separate embodiment of the invention. In some embodiments, the protein comprises an amino acid sequence with 71% to 100%, 80% to 100%, 87% to 100%, or 95% to 100% homology or identity to SEQ ID NO: 135. Each possibility represents a separate embodiment of the invention.

In some embodiments, the protein comprises or consists of the amino acid sequence:

(SEQ ID NO: 136)
MKNKNPTSVIREALAKVLVFYYPFAGRLKEGPARKLMVDCSGEGV
LFIEAEADVTLKQFGDALQPPFPCLEELLYDVPGSTGILDTPLLL
IQVTRLLCGGFIFALRLNHTMSDAAGLVQFMTGLGEMAQGASRPS
TLPVWQRELLFARDPPRVTCTHHEYTEVEDTNGTIIPLDDMAHKS
FFFGPSEISALRRFVPSYLKKCSTFEVLTACLWRCRTIALQPDPE
EEMRMICIVNARGKFNPPLLPKGYYGNGFAIPVAISTAGDLSSKP
LGHALELVMKAKSNVTEEYMRSVADLMVIKGRPHYTVVRSYLVSD
VTHAGFDVVDFGWGKASYGGPAKGGVGAIPGVVTFFIPFTNHKGE
SGIVLPICLPSAAMDKFVEELNKMLVPDNNEQVLREHKLLVLARL.

In some embodiments, the protein comprises an amino acid sequence with at least 88%, at least 92%, at least 95%, or at least 99% homology or identity to SEQ ID NO: 136, or any value and range therebetween. Each possibility represents a separate embodiment of the invention. In some embodiments, the protein comprises an amino acid sequence with 88% to 100%, 92% to 100%, 97% to 100%, or 99% to 100% homology or identity to SEQ ID NO: 136. Each possibility represents a separate embodiment of the invention.

In some embodiments, the protein comprises or consists of the amino acid sequence:

(SEQ ID NO: 137)
MAQIDTPLTFKVRRHAPELIAPAKPTPRELKPLSDIDDQEGLRFH
IPVIQFYRSDPKMKNKNPASVIREALAKVLVFYYPFAGRLKEGPA
RKLMVDCSGEGVLFIEAEADVTLKQFGDALQPPFPCLEELLYDVP
GSTGVLDTPLLLIQVTRLLCGGFIFALRLNHTMSDAPGLVQFMTG
LGEMAQGASRPSTLPVWQRELLLARDPPRVTCTHHEYTEVEDTKG
TIIPLDDMAHKSFFFGPSEISALRRFVPSYLKKCSTFEVLTACLW
RCRTIALQPDPEEEMRIICIVNARGKFNPPLPKGYYGNGFAFPVA
ISTAGDLSSKPLGHALELVMKAKSDVTEEYMRSIADLMVIKGRPH
FTVVRSYLVSDVTHAGFDVVDFGWGKAAYGGPAKGGVGAIPGVAS
FYIPFTNHKGESGIVLPICLPSAAMDKFVEELNKMLVPDNNEQVL
REHKLLVLARL.

In some embodiments, the protein comprises an amino acid sequence with at least 91%, at least 93%, at least 95%, or at least 99% homology or identity to SEQ ID NO: 137, or any value and range therebetween. Each possibility represents a separate embodiment of the invention. In some embodiments, the protein comprises an amino acid sequence with 91% to 100%, 93% to 100%, 95% to 100%, or 97% to 100% homology or identity to SEQ ID NO: 137. Each possibility represents a separate embodiment of the invention.

In some embodiments, the protein comprises or consists of the amino acid sequence:

(SEQ ID NO: 138)
MEIQVINYSSKLVKPLTPTPTANRYYNISFTDELVPTIYVPLILY
YATPKNPNGDHFENICDRLEESLSKTLSDFYPLAARFIRKLSLID
CNDQGVLFVLGNVNIRLSDVTGLGLTFKTSVLNDFLPCEIGGADE
VDDPMLCVKVTTFECGGFAIGMCFSHRLSDMGTMCNFINNWAART
IGEYDNEKHTPIFNSPLYFPQRGLPELDLKVPRSSIGVKNAARMF
HFNGKAISSMREVFGVDENGSRRLSKVQLVVALLWKAFVRIDDVN
DGQSKASFLIQPVGLRDKVVPPLPSNSFGNFWGLATSQLGPGEGH
KIGFQEYFYILRESIKKRARDCAKILTHGEEGYGVVIDPYLESNQ
KIADNGTNFYLFTCWCKFSFYEADFGCGKPIWASTGKFPVQNLVI
MMDDNEGDGVEAWVHLDDKRMNELEQDPDVKLYACNLA.

In some embodiments, the protein comprises an amino acid sequence with at least 73%, at least 85%, at least 92%, at least 95%, or at least 99% homology or identity to SEQ ID NO: 138, or any value and range therebetween. Each possibility represents a separate embodiment of the invention. In some embodiments, the protein comprises an amino acid sequence with 73% to 100%, 80% to 100%, 90% to 100%, or 95% to 100% homology or identity to SEQ ID NO: 138. Each possibility represents a separate embodiment of the invention.

In some embodiments, the protein comprises or consists of the amino acid sequence:

(SEQ ID NO: 139)
MKLAVKESVIVKPSKTTPCQQIWTSNLDLVVGRIHILTVYLYRPN
GSSNFFDSMVLKKALADVLVSFFPVAGRLDKDGDGRVVIDCNGEG
VLFVEAEADCCIDDFGEITPSPELRRLVPTVDYSGDMSSYPLFIT
QVTRFKCGGVSLGCGLHHTLSDGLSALHFINTWSDVARGLSVAIP
PFIDRSLLRARDPPSPVFDHIEYHPPPSLITPLQNQKNASHSRSA
STLILRLTLHQINNLKSKAKGDGSMYHSTYEILAAHLWRCACKAR
GLANDQPTKLYVATDGRSRLIPPLPPGYLGNVVFTATPVAKSGDF
ESESLAETARRIRSELGKMNDEYLRSAIDYLESVSDISTLVRGPT
YFASPNLNVNSWTRLPIYESDFGWGRPIFMGPASILYEGTIYIIP
SPSGDRSVSLAVCLDPDHMALFKECLYVF.

In some embodiments, the protein comprises an amino acid sequence with at least 83%, at least 88%, at least 92%, at least 95%, or at least 99% homology or identity to SEQ ID NO: 139, or any value and range therebetween. Each possibility represents a separate embodiment of the invention. In some embodiments, the protein comprises an amino acid sequence with 83% to 100%, 88% to 100%, 94% to 100%, or 97% to 100% homology or identity to SEQ ID NO: 139. Each possibility represents a separate embodiment of the invention.

In some embodiments, the protein comprises or consists of the amino acid sequence:

(SEQ ID NO: 140)
MKLAVKESVIVKPSKTTPCQQIRTSNLDLVAGRIHILVVFFYRPN
GSSNFFDSLVLKKALADVLVPFFPVAGRFSEDGDGRVVIDCNGEG
VLFVESEADCCIDDFGEITLSPELQQLVPTVDYSGDMSSYPLFIA
QVTRFKCGGVSLGWGLHHTLLDGLSALHFVNTWGDVARGLSVAIQ
PFIDRSLLRARDPPTPVFDHIEYHPPPSLITPLQNQKNASHSRSA
STLILQLTPDQIKNLKSKAKGDGSMYHSTYEILAAHLWRCACKAR
GLANDQPTKLYVAANGRSRLIPPLPPGYLGNVVFNATHVAKSGDF
ESESLAETARRIHCELGKMNDEYFRSAIDYLESVDDISTLVKGPT
YFASPNLNVYSWIGIPIYACDFGWGQPIFMRPASFLYDGSIYIIP
SPSGDRSVLLAVCLDPDHMDLFKECLYAF

In some embodiments, the protein comprises an amino acid sequence with at least 76%, at least 84%, at least 92%, or at least 99% homology or identity to SEQ ID NO: 140, or any value and range therebetween. Each possibility represents a separate embodiment of the invention. In some embodiments, the protein comprises an amino acid sequence with 76% to 100%, 83% to 100%, 90% to 100%, or 95% to 100% homology or identity to SEQ ID NO: 140. Each possibility represents a separate embodiment of the invention.

In some embodiments, the protein comprises or consists of the amino acid sequence:

(SEQ ID NO: 141)
MVMISKLLRLGRRKLHTIVSRDTIRPSSPTPSHSKTYNLSLLDQI
AVNSYVPIVAFYPSSNVCRSSDDKTLELKNSLSKILTHYYPFAGR
MKKNRPTVVDCNDEGVEFVEARNTNSLSDFLQQSEHEDLDQLFPD
DCVWFKQNLKGSINDANNSSVCPLSIQVNHFACGGVAVATSLRHK
IGDGSSALNFIKHWAAVTSHSRAGNHQIDATSPIINPHFISYPTR
TFKLPDRSPYIPPSDVVSKSFVFPNTNIKDLQAKVVTMTMGSRQP
IVNPTRADVVSWLLHKCVVAAATKRISGNFKESCVISPLNLRNKL
EEPLPETSIGNIFYLITFPISNNHGDLMPDDFISQLRLGIRKFQN
IRNLETALRTVEEMISETFILGTAESMDTSYVYSSIRGFPMYDID
FGWGKPVKVTVGGALKNLSILMDTPDVNGIEALVSLDKQDMKILL
NDPELLAFCL.

In some embodiments, the protein comprises an amino acid sequence with at least 60%, at least 70%, at least 80%, at least 90%, or at least 95% homology or identity to SEQ ID NO: 141, or any value and range therebetween. Each possibility represents a separate embodiment of the invention. In some embodiments, the protein comprises an amino acid sequence with 60% to 100%, 70% to 100%, 80% to 100%, or 90% to 100% homology or identity to SEQ ID NO: 141. Each possibility represents a separate embodiment of the invention.

In some embodiments, the protein comprises or consists of the amino acid sequence:

(SEQ ID NO: 142)
MSTSDKMKITIRESSMIKPSKPTPDQRIWNSNLDLVVGRIHILTL
YFFRPNGSSDFFDSEVLKQSLADVLVSFFPMAGRLGLDGDGRVEI
NCNGEGVLFVEAEADCSIDDFGEITPSPELRRLAPTVDYSGDISS
YPLVITQVTHFKCGGVSLGCGLHHTLSDGLSSLHFINTWSDVTRG
LPVAIPPFVDRTVLRARDPPTVVFDHVEYHTPPSMTSSLDKDKPQ
SEDVHVSTSMLRLTLDQINALKAKGKGDGIVYHSTYEILAAHLWR
CACKARGLLNDQMTKLYVATDGRSRLIPPLPPGYLGNVVFTATPI
AKSGELQQEPLATTARKIHTELAKMDDKYLRSALDYLESQQDLSA
LIRGPAYFACPNLNINSWTRLPIYDADFGWGRPIFMGPASILYEG
TIYIIPSPSGDRSVSLAVCLDPSHMPLFQKYLYEL.

In some embodiments, the protein comprises an amino acid sequence with at least 85%, at least 89%, at least 92%, at least 95%, or at least 99% homology or identity to SEQ ID NO: 142, or any value and range therebetween. Each possibility represents a separate embodiment of the invention. In some embodiments, the protein comprises an amino acid sequence with 85% to 100%, 90% to 100%, 93% to 100%, or 96% to 100% homology or identity to SEQ ID NO: 142. Each possibility represents a separate embodiment of the invention.

In some embodiments, the protein comprises or consists of the amino acid sequence:

(SEQ ID NO: 143)
MVNVEIISNEYIKPSSPTPPHLKIYNLSILDQLIPAPYAPIILYY
PNQDHINDFEVHERLKLLKDSLSKTLTRFYPLAGTIKGDLSIDCN
DIGAYFAVAHVNTRLDVFLNHPDLDLINCFLPRGPYLNGSSEGSC
VSNVQVNIFECCGIAISLCISHKILDGAALSTFLKAWAGTSYGSK
EVVYPNMSAPSLFPAKDLWLKDSSMVMFGSLFKMGKCSTKRFVFD
SSKLSFLKAKASLNGLKDPTRVEVVSALLWKCIMAASEENTGSWK
PSLLSHVVNLRKRLVSTLSEDSIGNLIWLASAECRTNAQSRLSDL
VEKVRDSVSKINSEFVKKIQGDKGTKVMEESLKSMKDCADYIGFT
SWCKMGFYDVDFGWGKPVWVCGSVCEGSPVFMNFVILMDTKYGDG
IEAWVSLDEHEMHILKHNPELLEYASIDPSPLQMNK.

In some embodiments, the protein comprises an amino acid sequence with at least 82%, at least 85%, at least 92%, at least 95%, or at least 99% homology or identity to SEQ ID NO: 143, or any value and range therebetween. Each possibility represents a separate embodiment of the invention. In some embodiments, the protein comprises an amino acid sequence with 82% to 100%, 85% to 100%, 90% to 100%, or 93% to 100% homology or identity to SEQ ID NO: 143. Each possibility represents a separate embodiment of the invention.

In some embodiments, the protein comprises or consists of the amino acid sequence:

(SEQ ID NO: 144)
MGTIYQSPMIKSSTPKIIEDLKVIIHDTFTIFPPHETEKRSMFLS
NIDQVLTENVETVHFFAANPDFPPQVVAEKLKLALSKALVPYDFL
AGRLKLNHESQRFEFDCNGAGARFVVGSSEFELGEIGDLVYPNPG
FRQLVQKSYDNLELHEKPLCILQLTSFKCGGFALGVATNHATFDG
LSFKTFLQNLGSLAADQPLAVDPCNDRHLLAARSPPKVQFDHPEL
LKIPTGTDIPNPTVFDCPESQLDFKIFNLTSDDIAHLKTKAKDGP
GSTNAKITGFNVVAAHVWRCKALSSGSEYDPERVSTVLYAVDIRS
RLNLPLSLAGNAVLSAYASAKCKEIEEGPLSRLVEMVTEGTNRMT
GEYARSVIDWGEVNKGFPNGEFLISSWWRLGFADVEYPWGKPRYS
CPVVYHRKDIILLFPDIVGADNNNEVNVLVALPGKEMEKFETLFH
KFLA.

In some embodiments, the protein comprises an amino acid sequence with at least 88%, at least 92%, at least 95%, or at least 99% homology or identity to SEQ ID NO: 144, or any value and range therebetween. Each possibility represents a separate embodiment of the invention. In some embodiments, the protein comprises an amino acid sequence with 88% to 100%, 90% to 100%, 93% to 100%, or 95% to 100% homology or identity to SEQ ID NO: 144. Each possibility represents a separate embodiment of the invention.

In some embodiments, a protein comprising an amino acid sequence set forth in SEQ ID Nos.: 12-22, is an AAE.

In some embodiments, a protein comprising an amino acid sequence set forth in SEQ ID Nos.: 27-30, is a PKS.

In some embodiments, a protein comprising an amino acid sequence set forth in SEQ ID Nos.: 39-46, is a PKC.

In some embodiments, a protein comprising an amino acid sequence set forth in SEQ ID Nos.: 59-70, is a PT.

In some embodiments, a protein comprising an amino acid sequence set forth in SEQ ID Nos.: 80-88, is a CBCAS.

In some embodiments, a protein comprising an amino acid sequence set forth in SEQ ID Nos.: 102-114, is a UGT.

In some embodiments, a protein comprising an amino acid sequence set forth in SEQ ID Nos.: 130-144, is a AAT.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. The degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percentage identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

In some embodiments, the protein comprises or is characterized by acyl activating enzymatic activity.

In some embodiments, an acyl is selected from: C1-C8 alkyl chain, and alpha-unsaturated phenylalkyl carboxylic acid.

In some embodiments, an acyl is a C1 alkyl chain. In some embodiments, an acyl is a C2 alkyl chain. In some embodiments, an acyl is a C3 alkyl chain. In some embodiments, an acyl is a C4 alkyl chain. In some embodiments, an acyl is a C5 alkyl chain. In some embodiments, an acyl is a C6 alkyl chain. In some embodiments, an acyl is a C7 alkyl chain. In some embodiments, an acyl is a C8 alkyl chain.

In some embodiments, a C1-C8 alkyl chain is hexanoic acid. In some embodiments, an acyl is hexanoic acid.

In some embodiments, an alpha-unsaturated phenylalkyl carboxylic acid comprises cinnamic acid or a derivative thereof.

In some embodiments, a cinnamic acid derivative comprises a hydroxylated derivative of cinnamic acid.

In some embodiments, a hydroxylated derivative of cinnamic acid comprises or is coumaric acid.

In some embodiments, the protein comprises or is characterized by polyketide synthesizing activity, as described herein. In some embodiments, the protein is characterized by having an activity of polymerizing a diketide substrate into a polyketide.

In some embodiments, a diketide substrate is obtained by coupling of an acyl CoA starting unit.

In some embodiments, an acyl CoA starting unit is selected from: acetyl COA, butyryl CoA, hexanoyl CoA, octanoyl CoA, cinnamoyl CoA, coumaroyl CoA, or any combination thereof.

In some embodiments, an acyl CoA is or comprises hexanoyl CoA, cinnamoyl CoA, or both.

In some embodiments, an acyl CoA is hexanoyl CoA.

In some embodiments, a polyketide comprises a tetraketide. In some embodiments, a polyketide comprises a linear polyketide. In some embodiments, a polyketide comprises a linear tetraketide.

In some embodiments, the protein comprises or is characterized by polyketide cyclization or cyclizing activity, as described herein. In some embodiments, the protein is characterized by having an activity of cyclizing a polyketide.

In some embodiments, polyketide cyclization comprises aldol cyclization, Claisen cyclization, or both.

In some embodiments, a polyketide comprises an acyl group, as described herein.

In some embodiments, the protein comprises or is characterized by prenyl transferring activity, as described herein. In some embodiments, the protein is characterized by being capable of transferring a prenyl group to a substrate molecule. In some embodiments, the protein is characterized by being capable of transferring an allylic prenyl group to an acceptor molecule. In some embodiments, the protein is a prenyl diphosphate synthase. In some embodiments, the protein is a trans-prenyltransferase. In some embodiments, the protein is a cis-prenyltransferase.

In some embodiments, the prenyl group is selected from: dimethylallyl diphosphate, geranyl diphosphate, farnesyl diphosphate, or geranylgeranyl diphosphate.

In some embodiments, the protein is characterized by being capable of synthesizing a compound represented by Formula I:

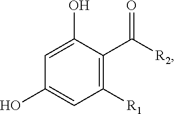

wherein: (i) R₁ is selected from: C1-C8 alkyl, an alpha-unsaturated phenylalkyl carboxylic acid, or an alpha saturated phenylalkyl carboxylic acid; and R₂ is OH; or (ii) R₁ is OH and R₂ is selected from: C1-C8 alkyl, an alpha-unsaturated phenylalkyl carboxylic acid, or an alpha saturated phenylalkyl carboxylic acid.

In some embodiments, the compound is represented by a formula selected from:

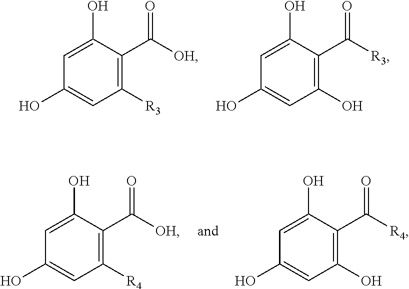

wherein R₃ is C1-C8 alkyl, and wherein R₄ is alpha-unsaturated phenylalkyl carboxylic acid.

In some embodiments, the compound is selected from the group:

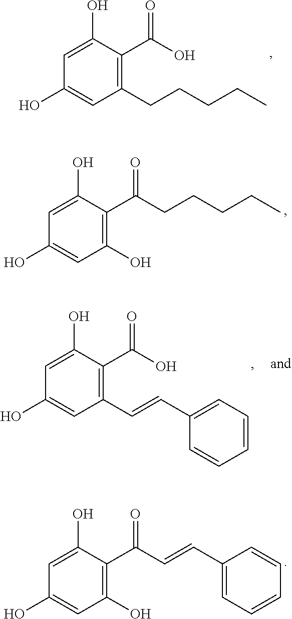 [SMILES: CCCCCc1cc(O)cc(O)c1C(=O)O ; CCCCCC(=O)c1c(O)cc(O)cc1O ; O=C(O)c1c(O)cc(O)cc1/C=C/c1ccccc1 ; O=C(/C=C/c1ccccc1)c1c(O)cc(O)cc1O]

In some embodiments, the compound is:

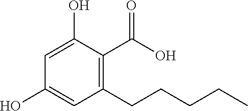 [SMILES: CCCCCc1cc(O)cc(O)c1C(=O)O]

In some embodiments, the protein is characterized by cannabigerolic acid (CBGA) cyclization or cyclizing activity. In some embodiments, cycling activity comprises cyclization of CBGA to CBCA. In some embodiments, the protein is characterized by being capable of cyclizing or cyclization of CBGA to CBCA. In some embodiments, the protein is characterized by being capable of synthesizing CBCA or being a CBCA synthase (CBCAS).

In some embodiments, the protein is characterized by being capable of transferring a glucuronic acid component of UDP-glucuronic acid to a cannabinoid or precursor thereof.

In some embodiments, the protein is characterized by being capable of transferring an acyl group from a donor molecule to the cannabinoid.

According to some embodiments, there is provided a transgenic cell comprising: (a) the DNA molecule disclosed herein; (b) the artificial nucleic acid molecule disclosed herein; (c) the plasmid or agrobacterium disclosed herein; (d) the protein disclosed herein; or any combination thereof.

In some embodiments, the cell further comprises a nucleic acid sequence encoding at least one enzyme related to cannabinoidogenesis derived from Cannabis sativa. In some embodiments, the at least one enzyme related to cannabinoidogenesis derived C. sativa is selected from: olivetol synthase (OLS), olivetolic acid cyclase (OAC), prenyltransferase 1 (PT1/GOT1), PT4/GOT4, or any combination thereof.

In some embodiments, the at least one enzyme related to cannabinoidogenesis derived C. sativa is selected from: OLS, OAC, or both.

As used herein, the term “transgenic cell” refers to any cell that has undergone human manipulation on the genomic or gene level. In some embodiments, the transgenic cell has had exogenous polynucleotide, such as the DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell is a unicellular organism, a cell of a multicellular organism, and a cell in a culture.

In some embodiments, a unicellular organism comprises a fungus or a bacterium.

In some embodiments, the fungus is a yeast cell.

In some embodiments, the cell is an insect cell. In some embodiments, the cell comprises an insect cell line.

Types of insect cell lines suitable for transformation and/or heterologous expression are common and would be apparent to one of ordinary skill in the art. Non-limiting examples of such insect cell lines include, but are not limited to, Sf-9 cells, SR+ Schneider cells, S2 cells, and others.

According to some embodiments, there is provided an extract derived from a transgenic cell disclosed herein, or any fraction thereof.

In some embodiments, the extract comprises the DNA molecule disclosed herein, a protein as disclosed herein, or any combination thereof.

According to some embodiments, there is provided a homogenate, lysate, extract, derived from a transgenic cell disclosed herein, any combination thereof, or any fraction thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar, or nonpolar solvent), liquid chromatography mass spectrometry, or others.

According to some embodiments, there is provided a transgenic plant, a transgenic plant tissue or a plant part. In some embodiments, there is provided a transgenic plant, or any portion, seed, tissue, or organ thereof, comprising at least one transgenic plant cell of the invention. In some embodiments, the transgenic plant, transgenic plant tissue or plant part, comprises: (a) the DNA molecule disclosed herein; (b) the artificial disclosed herein; (c) the plasmid or agrobacterium disclosed herein; (d) the protein of the invention; (e) the transgenic cell disclosed herein; or any combination thereof.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

In some embodiments, the transgenic plant, transgenic plant tissue, or plant part is or derived from a Cannabis sativa plant. In some embodiments, the transgenic plant is a C. sativa plant.

In some embodiments, the transgenic plant, transgenic plant tissue, or plant part is or derived from hemp. In some embodiments, C. sativa comprises or is hemp.

According to some embodiments, there is provided a composition comprising any one of the herein disclosed: (a) the DNA molecule of the invention; (b) artificial vector; (c) plasmid or agrobacterium; (d) protein of the invention; (e) transgenic cell; (f) extract; (g) transgenic plant tissue or plant part; and (h) any combination of (a) to (g), and an acceptable carrier.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Synthesis

According to some embodiments, there is provided a method for synthesizing a cannabinoid, a precursor thereof, or any combination thereof.

According to some embodiments, there is provided a method for synthesizing acyl coenzyme A (CoA), polyketide, a compound represented by Formula I, a compound represented by Formula II, a cannabinoid, or any combination thereof.

In some embodiments, the method further comprises glycosylating a compound represented by Formula I, a compound represented by Formula II, a cannabinoid, or any combination thereof. In some embodiments, the method further comprises transferring an acyl group to a compound represented by Formula I, a compound represented by Formula II, a cannabinoid, or any combination thereof.

As used herein, the term “cannabinoid” or “cannabinoids” refer to a heterogeneous family of molecules usually exhibiting pharmacological properties by interacting with specific receptors. To date, two membrane receptors for cannabinoids, both coupled to G protein and named CB1 and CB2 have been identified. While CB1 receptors are mainly expressed in the central and peripheral nervous system, CB2 receptors have been reported to be more abundantly detected in cells of the immune system.

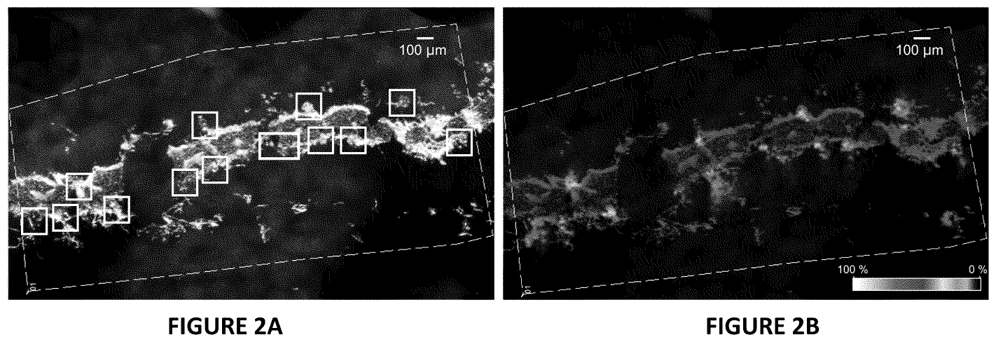

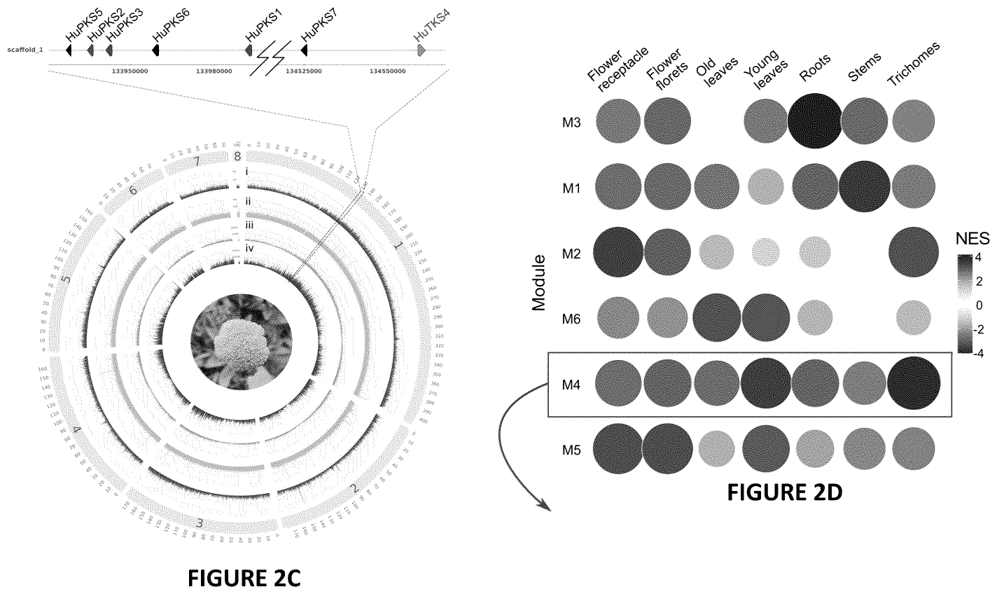

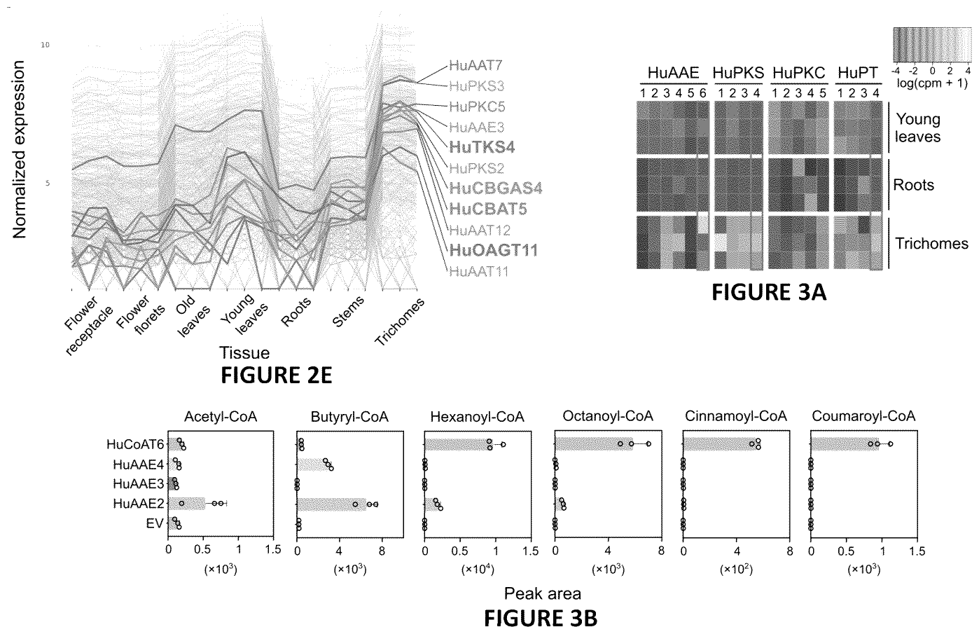

In some embodiments, the cannabinoid comprises any compound as presented in FIG. 2.

According to some embodiments, the method comprises the steps: (a) providing a transgenic cell or a cell transfected with the DNA molecule of the invention or the artificial nucleic acid molecule disclosed herein; and (b) culturing the transgenic cell the transfected cell from step (a) such that at least a first protein and a second protein encoded by DNA molecule or the artificial nucleic acid molecule are expressed, thereby synthesizing the cannabinoid, a precursor thereof, or any combination thereof.

In some embodiments, the precursor is selected from: acyl coenzyme A (CoA), a polyketide, a resorcinoid precursor, or any combination thereof.

In some embodiments, the resorcinoid precursor is olivetolic acid.

In some embodiments, the cannabinoid comprises or is CBGA, CBCA, or both.

According to some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell comprises the DNA molecule of the invention or a plurality thereof, as disclosed herein.

In some embodiments, the transgenic cell or the transfected cell comprises the artificial nucleic acid molecule or vector as disclosed herein.

In some embodiments, the cell is a transgenic cell, or a cell transfected with a DNA molecule as disclosed herein.

In some embodiments, the method further comprises a step preceding step (a), comprising introducing or transfecting the cell with the artificial nucleic acid molecule or vector, disclosed herein.

Method for introducing or transfecting a cell with an artificial nucleic acid molecule or vector are common and would be apparent to one of ordinary skill in the art.

In some embodiments, introducing or transfecting comprises transferring an artificial nucleic acid molecule or vector comprising the DNA molecule disclosed herein into a cell; or modifying the genome of a cell to include the polynucleotide disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

As used herein, the terms “transfecting” and “introducing” are interchangeable.

In some embodiments, the contacting is in a cell-free system.

Types of suitable cell-free systems for expression and/or synthesis utilizing any one of: the DNA molecule of the invention or a plurality thereof, as disclosed herein, and the protein of the invention, or a plurality thereof, would be apparent to one of ordinary skill in the art.

In some embodiments, the method further comprises a step preceding step (b), comprising separating the cultured transgenic cell or the cultured transfected cell from the medium.

Method for separating cell from a medium are common and may include, but not limited to, centrifugation, ultracentrifugation, or other, as would be apparent to a skilled artisan.

According to some embodiments, there is provided an extract of a transgenic cell, or a transfected cell obtained according to the herein disclosed method.

In some embodiments, the extract comprises a cannabinoid, a precursor thereof, or any combination thereof.

In some embodiments, the extract comprises CBGA, CBCA, or both.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof; or (c) any combination of (a) and (b), and an acceptable carrier, as described herein.

In some embodiments, a portion comprises a fraction or a plurality thereof.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### Materials

Unless otherwise stated, all the analytical metabolites were >95% pure. CBGA 1, CBCA 15, CBDA, acetic acid, propionic acid, butyric acid, pentanoic acid, hexanoic acid, heptanoic acid, octanoic acid, ±2-methyl butyric acid, phenylalanine, hexanoic-D₁₁ acid (D>98%), GPP, IPP, FPP, phloretin 98, naringenin 96, malonyl-CoA (≥90%), acetyl-CoA (≥93%), butyryl-CoA (≥90%), hexanoyl-CoA (≥85%), octanoyl-CoA, iso-valeryl CoA (≥90%), olivetol and sodium hexnoate were purchased from Sigma-Aldrich (Rehovot, Israel). Δ⁹-THCA was purchased from Silicol Scientific Equipment Ltd. (Or Yehuda, Israel). Acetic-D₃ acid (D>99%), propionic-D₅ acid (D>99%), butyric-D₅ acid (D>98%), pentanoic-D₉ acid (D>98%), heptanoic-D₅ acid (D>99%), octanoic-D₅ acid (D>99%), iso-butyric-D₇ acid (D>98%), ±2-methyl butyric-D₉ acid (D>99%), iso-valeric-D₉ acid (D>98%), iso-caproic-D₁₁ acid (D>98%) were purchased from C/D/N isotopes (Quebec, Canada). Phenylalanine-D₅ (D>98%) and phenylalanine-¹³C₉,¹⁵N₁ (¹³C,¹⁵N>99%) were synthesized by Cambridge Isotope Laboratories (Andover, MA). HeliCBGA 2 (NP009525, 90%) was purchased from Analyticon Discovery GmbH (Potsdam, Germany). APHA 3 was reported as an impurity (NP015136, 5%) in the heliCBGA analytical metabolite. OA 92 (>90%), VA (>90%) and iso-butyryl-CoA were purchased from Cayman Chemical (Ann Arbor, MI, USA). PCP 95, naringenin chalcone 97 and pinocembrin chalcone 100 were purchased from Wuhan ChemFaces Biochemical Co Ltd. (Hubei, China). Cinnamoyl-CoA and Coumaroyl-CoA were purchased from TransMIT GmbH (Hesse, Germany).

Seeds of H. umbraculigerum (Silverhill seeds, Cape Town, South Africa) were germinated, and grown in a greenhouse in a long-day photoperiod. Plants were propagated by cuttings.

### Feeding Experiments

All feeding solutions were prepared as aqua solutions of 0.5 mg ml⁻¹ of the precursor. The pH of the FA solutions was adjusted to 5.5-6.0. The phenylalanine feeding experiments were performed on leaves from young mother plants excised by cutting at the proximal side of the pedicel with scissors under water, leaving attached 1-2 cm of the pedicel. For the FA feeding experiments, 10 cm young cuttings were obtained from mother plants. The lower leaves were removed leaving 4-5 leaves on each stem, and the stem was peeled to increase the intake of the labeled solutions. Three to four leaves or the young cuttings were immersed in aqua solutions [DDW (control), unlabeled or labeled precursors, each group consisted of a minimum of three biological replicates]. All feeding experiments were performed in a controlled environment for 48-96 h under 25° C. and constant fluorescent illumination and humidity and the tubes were periodically refilled. Upon termination, the fresh leaves were rinsed with a small amount of water, dried gently, flash frozen and stored at −80° C. for extraction.

### LC-MS Chemical Analysis

Unless otherwise stated, 100 mg frozen powdered plant tissue were extracted with 300 μl ethanol, sonicated for 15 min, agitated for 30 min and centrifuged at 14,000 g for 10 min. The supernatant was filtered through a 0.22 μm syringe filter and analyzed in the obtained concentration. Detection was performed using both targeted and non-targeted approaches as described in Berman et al., “Parallel evolution of cannabinoid biosynthesis”; Nature Plants 9 817-831 (2023) using an ultrahigh-performance liquid chromatography-tandem quadrupole time-of-flight (UPLC-qTOF) system comprised of a UPLC (Waters Acquity) with a diode array detector connected either to a XEVO G2-S QTof (Waters) or to Synapt HDMS (Waters). The chromatographic separation was performed on a 100 mm×2.1 mm i.d. (internal diameter), 1.7 μm UPLC BEH C18 column (Waters Acquity). The mobile phase consisted of 0.1% formic acid in acetonitrile:water (5:95, v/v; phase A) and 0.1% formic acid in acetonitrile (phase B). Terpenophenols were analyzed using UPLC Method 1 as follows: Initial conditions were 40% B for 1 min, raised to 100% B until 23 min, held at 100% B for 3.8 min, decreased to 40% B until 27 min, and held at 40% B until 29 min for re-equilibration of the system. The flow rate was 0.3 ml min⁻¹, and the column temperature was kept at 35° C. Intermediates and glucosylated metabolites were analyzed using UPLC Method 2 as follows: Initial conditions were from 0% to 28% B over 22 min, raised to 100% B until 36 min, held at 100% B for 2 min, decreased to 0% B until 38.5 min, and held at 40% B until 40 min for re-equilibration of the system. The flow rate was 0.3 ml min⁻¹, and the column temperature was kept at 35° C. Electrospray ionization (ESI) was used in either positive or negative ionization modes at an m/z range of 50-1,000 Da. Masses were detected with the following settings: capillary 1 kV, source temperature 140° C., desolvation temperature 450° C., and desolvation gas flow 800 1 h⁻¹. Argon was used as the collision gas. The MS system was calibrated with sodium formate and Leu encephalin was used as the lock mass. Data acquisition for untargeted analysis was performed in negative ionization using the MSE mode. The collision energy was set to 4 eV for the low-energy function and to 15-50 eV ramp for the high-energy function. The R package Miso was run as previously described. Differential metabolites were selected if the fold change was greater or equal to 10 and the p-value was less than 0.05. MS/MS experiments were performed in positive or negative ionization modes according to the specific protonated or deprotonated masses with following settings: capillary spray of 1 kV; cone voltage of 30 eV; collision energy ramps were 10-45 eV for positive mode and 15-50 eV for negative mode.

### Absolute Quantification of CBGA 1

Fresh samples of leaves (dark and light), flowers, stems and roots were collected from a plant at the flowering stage. Florets and the receptacle of flowers were detached using a scalpel and analyzed separately. All tissues were flash frozen in liquid N₂ and ground into fine powder. To measure CBGA 1 content in a dry tissue, fresh leaves were flash frozen, ground and lyophilized. For the extraction, 100 mg of the frozen powders were accurately weighed in triplicates, extracted with 1 ml ethanol, and prepared as previously described. Samples were injected in several dilutions to fit into the linear range of the calibration curves. Injections were performed on a UPLC (Waters) connected to a Triple Quad detector (TQ-S, Waters) in multiple reaction monitoring (MRM) mode. The system was operated with a similar column and mobile phase as for UPLC-qTOF analysis as follows: Initial conditions were 57% B raised to 85% B until 4 min, raised to 100% B until 4.2 min, held at 100% B until 6 min, decreased to 67% B until 6.2 min, and held at 67% B until 7 min for re-equilibration of the system. The flow rate was 0.6 ml min⁻¹, and the column temperature was kept at 40° C. The instrument was operated in negative mode with a capillary voltage of 1.5 kV and a cone voltage of 40 V. Absolute quantification of CBGA 1 was performed by external calibration using two different transitions (359.3>191.2, 32 V for quantification; and 359.3>315.4, 21 V for qualification).

### Metabolite Purification for NMR Analysis

A total of 86 g of fresh leaves were flash frozen in liquid N₂ and ground into fine powder using an electrical grinder, extracted with 600 ml ethanol, sonicated for 20 min, and agitated for 30 min. The supernatant was filtered, evaporated using a rotary evaporator at 40° C. and lyophilized. The extract was reconstituted in 25 ml acetonitrile and used for either direct purification (following ten times dilution) or prefractionation via medium pressure liquid chromatography (MPLC). The Büchi Sepacore MPLC System was equipped with two C-605 pump modules, a C-620 control unit, C-660 fraction collector, C-640 UV photometer (Büchi Labortechnik AG, Switzerland), and a C18 manually packed column. The mobile phase consisted of acetonitrile:water (5:95, v/v; phase A) and acetonitrile (phase B), with the following multistep gradient method: initial conditions were 0% B for 10 min, raised to 99% B until 530 min, and slowly raised to 100% B until 660 min. The flow rate was 15 ml min⁻¹, the injection volume was 15 ml, and the wavelengths were: 210, 224, 270 and 350 nm. Fractions of 100 ml were collected throughout the run and analyzed by UPLC-qTOF to select specific metabolites for purification. The selected fractions were evaporated using a rotary evaporator at 40° C., lyophilized, reconstituted in ethanol or methanol (only for the fraction with Glc-OA 102 and Glc-DHSA 103), and filtered through a 0.22 μm syringe filter. Purification of metabolites was performed on either an Agilent 1290 Infinity II UPLC system (System 1, the general instrument setup was according to Jozwiak et al. 2020); or a UPLC system (Waters Acquity) equipped with a binary pump, an autosampler, a fraction manager and a diode array detector (System 2) with similar mobile phase as for the UPLC-qTOF. Triggering was performed using specific UV wavelengths according to the metabolite.

In System 1, method development was performed by acquisition of both MS and UV signals. MS spectra were acquired in negative full scan mode between m/z 50 and 1,700. HPLC columns were either XBridge (BEH C18, 250× 4.6 mm i.d., 5 μm; Waters) or Luna (C18, 250× 4.6 mm i.d., 5 μm; Phenomenex), and the conditions were adjusted and optimized for each metabolite. In this system, the eluent with the metabolites of interest were mixed with a makeup-flow of 1.8 ml min⁻¹ water and then trapped on solid phase extraction (SPE) cartridges (10×2 mm Hysphere resin GP cartridges). Each cartridge was loaded four times with the same metabolite, and 36-72 cartridges were used for trapping one metabolite, depending on the concentration of the sample injected. After collection, SPE cartridges were dried with a stream of N₂, and eluted with 150 μl methanol. Eluents containing the same metabolite were pooled, dried under a stream of N₂, and stored at −20° C. until NMR analysis. A UPLC BEH C18 column (100 mm×2.1 mm i.d., 1.7 μm; Waters) was used on System 2, apart from metabolites Glc-OA 102 and Glc-DHSA 103 which were fractionated on a Luna Phenyl-Hexyl column (150 mm×2 mm i.d., 3 μm; Phenomenex). The flow rate was 0.3 ml min⁻¹, and the column temperature was kept at 35° C. All other conditions were adjusted and optimized according to the sample. The eluent with the metabolite of interest was collected in 2 ml HPLC vials. Eluents containing the same metabolite were pooled, dried under a stream of N₂, lyophilized, and stored at −20° C. until NMR analysis.

### NMR Spectroscopy

Purified metabolites were resuspended in 300 μl of Methanol-d₄, dried under a stream of N₂, reconstituted in 70 μl Methanol-d₄ with 0.01% of 3-(trimethylsilyl) propionic-2,2,3,3-d₄ acid sodium salt (TMSP, used as an internal chemical shift reference for ¹H and ¹³C) and transferred into 1.7 mm micro-NMR test tubes for structure elucidation. NMR spectra were collected on a Bruker AVANCE NEO-600 NMR spectrometer equipped with a 5 mm TCI-xyz CryoProbe. All spectra were acquired at 298 K. The structures of the different metabolites were determined by one dimensional (1D) ¹H NMR spectra, as well as various two-dimensional (2D) NMR spectra: ¹H-¹H Correlation Spectroscopy (COSY), ¹H-¹H Total Correlation Spectroscopy (TOCSY), ¹H-¹H Rotating Frame Nuclear Overhauser Spectroscopy (ROESY), ¹H-¹³C Heteronuclear Single Quantum Coherence (HSQC), and ¹H-¹³C Heteronuclear Multiple Bond Correlation (HMBC) spectra.

One dimensional ¹H NMR spectra were collected using 16,384 data points and a recycling delay of 2.5 s. Two-dimensional COSY, TOCSY and ROESY spectra were acquired using 16,384-8,192 (t₂) by 400-512 (t₁) data points. 2D TOCSY spectra were acquired using isotropic mixing times of 100-300 ms. A T-ROESY experiment was used in this study, TOCSY-less ROESY that effectively suppresses TOCSY transfer in ROESY experiments. T-ROESY spectra were recorded using spin lock pulses of 100-400 ms. 2D HSQC and 2D HMBC spectra were collected using 4,096 (t₂) by 400-512 (t₁) data points. Multiplicity editing HSQC enables differentiating between methyl and methine groups that give rise to positive correlation, versus methylene groups that appear as negative peaks. HMBC delay for evolution of long-range couplings was set to observe long-range couplings of J_{H,C}=8 Hz. All data were processed and analyzed using TopSpin 4.1.1 software (Bruker).

### MALDI Imaging

For the peeling experiment, whole fresh leaves from a young plant were attached onto glass slides using double-sided tape with either the abaxial or adaxial surfaces, gently peeled above/below the midrib using duct tape and desiccated overnight under moderate vacuum. Images were taken using a digital camera. For localization of metabolites to individual trichomes, fresh leaves and flowers were sectioned, and matrix was sprayed as previously described. Sections were imaged with a Nikon DS-Ri2 microscope. MALDI imaging was performed using a 7 T Solarix FT-ICR (Fourier Transform Ion Cyclotron Resonance) mass spectrometer (Bruker Daltonics). The datasets were collected in positive ionization using lock mass calibration (DHB matrix peak: [3DHB+H-3H₂O]⁺, m/z 409.055408 Da) at a frequency of 1 kHz and a laser power of 40%, with 200 laser shots per pixel and 50, 15 or 25 μm pixel size for the peeled trichomes and for the sectioned leaves and flowers, respectively. Each mass spectrum was recorded in the range of m/z 150-3,000 in broadband mode with a Time Domain for Acquisition of 1M, providing an estimated resolving power of 115,000 at m/z 400. The spectra were normalized to root-mean-square intensity and MALDI images were plotted at theoretical m/z±0.005% with pixel interpolation on.

### Cryo-SEM, TEM, and Confocal Microscopy

For cryo scanning electron microscopy (cryo-SEM) analyses, frozen samples were attached to a holder either by mechanical clamping (leaves) or by a glue made of a concentrated PVP solution. The holder with the samples was then plunged frozen in liquid N₂, transferred to a BAF 60 freeze fracture device (Leica Microsystems, Vienna, Austria) using a VCT 100 Vacuum Cryo Transfer device (Leica) and was sublimed for 30 min at-95° C. Samples were transferred to an Ultra 55 cryo-SEM (Zeiss, Germany) using a VCT 100 shuttle and were and observed at −95° C. without coating using mostly mixed mode of InLens+SE detectors at 1-1.3 kV. For transmission electron microscopy (TEM) analysis, H. umbraculigerum leaves were fixed with 4% paraformaldehyde, 2% glutaraldehyde in 0.1 M cacodylate buffer containing 5 mM CaCl₂) (pH 7.4), then postfixed with 1% osmium tetroxide supplemented with 0.5% potassium hexacyanoferrate tryhidrate and potasssium dichromate in 0.1 M cacodylate (1 h), stained with 2% uranyl acetate in water (1 h), dehydrated in graded ethanol solutions and embedded in Agar 100 epoxy resin (Agar scientific Ltd., Stansted, UK). Ultrathin sections (70-90 nm) were viewed and photographed with a FEI Tecnai SPIRIT (FEI, Eidhoven, Netherlands) transmission electron microscope operated at 120 kV and equipped with an One View Gatan Camera. Confocal microscopy of trichomes was carried out on a Nikon eclipse A1 microscope. Transmitted light was used to image the trichomes since they lack fluorescence. Autofluorescence of chlorophyll (chloroplasts) was used as a contrast for better visualization of the trichomes. Far-red laser was used to detect autofluorescence of chlorophyll (excitation: 640 nm; emission: 663-738 nm).

### Trichome Enrichment

Trichomes were enriched following Bergau et al. guidelines with modifications. Briefly, young leaves were harvested and soaked in ice-cold, distilled water and then abraded using a BeadBeater machine (Biospec Products, Bartlesville, OK). The polycarbonate chamber was filled with 15 g of plant material and filled with half the volume with glass beads (0.5 mm diameter), XAD-4 resin (1 g/g plant material), and ethanol 80% to full volume. Leaves were beaten by 2-4 pulses of operation of 1 min each. This procedure was carried out at 4° C., and after each pulse the chamber was allowed to cool on ice. Following abrasion, the contents of the chamber were first filtered through a kitchen mesh strainer and then through a 100 μm nylon mesh to remove the plant material, glass beads, and XAD-4 resin. The residual plant material and beads were scraped from the mesh and rinsed twice with additional ethanol 80% that was also passed through the 100 μm mesh. The presence of enriched glandular trichome secretory cells was checked by visualization in an inverted optical microscope.

### Genome Assembly

High molecular weight DNA was extracted from young frozen leaves and sequenced in UC Davis Genome Center. Sequencing was done in a Pacbio Sequel II platform with ˜12-kilobase DNA SMRT bell library preparation according to the manufacturer's protocol. Three different SMRT 8M cells were used, yielding a total of 57.8 Gb of HiFi data (˜44× haploid coverage). In addition to Pacbio HiFi data, 200 M reads of PE 2×150 Illumina Hi-C data were obtained by the company Phase Genomics. Hifiasm software was used to integrate both Pacbio HiFi and HiC data to produce chromosome-scale and haplotype-resolved assemblies. Further scaffolding was performed with the Hi-C data, mapping the reads following Arima Genomics pipeline and the SALSA software. Visualizations of Hi-C heatmaps were performed with Juicer and quality metrics were obtained with Assemblathon 2 script. Finally, the assembly was softmasked for repetitive elements using EDTA with the −cds flag to incorporate CDS sequences from the transcriptomic data. Parameter details of each of the commands can be found in github.com/Luisitox/Helichrysum_paper.

### RNA Sequencing and Genome Annotation

RNA was extracted from seven tissues: young leaves, old leaves, florets and receptacles of flowers, stems, roots and trichomes (Berman et al., “Parallel evolution of cannabinoid biosynthesis”; Nature Plants 9 817-831 (2023)). RNA integrity was checked using a TapeStation instrument. Paired-end Illumina libraries were prepared for five of the tissues and sequenced on Illumina HiSeq 3000 instrument (PE 2×150, ˜40 M reads per sample) and processed following Freedman and Weeks guidelines. Briefly, random sequencing errors were corrected using Rcorrector and uncorrectable reads were removed. Adaptor and quality trimming were performed using TrimGalore! Ribosomal RNA was filtered by discarding reads mapping to SILVA_132_LSURef and SILVA_138_SSURef non-redundant databases using bowtie2. Fastq quality checks on each of the steps were performed using MultiQC. The remaining reads were pooled and used for genome-guided and genome-independent de novo transcriptome assembly using Trinity.

The Iso-Seq data was obtained from four of the tissues (Berman et al., “Parallel evolution of cannabinoid biosynthesis”; Nature Plants 9 817-831 (2023)) and processed with isoseq3. Fused and unspliced transcripts were removed, and only polyA-positive transcripts were kept for a unique set of high-quality isoforms. Iso-Seq and Trinity transcripts were aligned to the assembly using minimap2 and the BAM files were incorporated to the PASA pipeline to generate RNA-based gene model structures. In addition, de novo gene structures were obtained using the software braker2 and the BAM file alignments of long and short reads as extrinsic training evidence. Ab initio and RNA-based gene models were combined using EvidenceModeler followed by a final round of PASA pipeline. Gene functional annotation was performed for the predicted mature transcripts using TransDecoder, which takes into account HMMER hits against PFAM and BLASTP hits against UniProt databases for similarity retention criteria. Further annotation of protein-coding transcripts was performed by taking the best hit of BLASTP searches against other plant protein databases (Uniprot protein fasta files of sunflower id UP000215914_4232, Arabidopsis id UP000006548_3702, tomato id UP000004994_4081, rice id UP000059680_39947 and Cannabis NCBI id GCF_900626175.1_cs10). Signal peptides were predicted with SignalP, transmembrane domains were predicted with TMHMM, and GO and KEGG terms were obtained with Trinotate. The full script used for the functional annotation of the proteins can be found in github.com/Luisitox/Helichrysum_paper. BUSCO was used at multiple stages of the analysis to assess the completeness of the different versions of both the transcriptome and the genome.

### 3′ RNA Sequencing and Gene Co-Expression Network Analysis

UMI-based 3′ RNAseq of three replicates of the seven tissues was obtained similarly as described. Adaptor and quality trimming were performed using TrimGalore! in two steps, including PolyA trimming mode. Reads were mapped to the genome using STAR UMI-deduplicated using UMI-tools, and counts were obtained with featureCounts. Normalization was performed with the varianceStabilizingTransformation algorithm of DESeq2, and the CEMItools package was used for co-expression analysis (dissimilarity threshold of 0.6, pvalue of 0.1).

### Circos and Gene Cluster Plots

Gene and TEs density were calculated by intersecting the corresponding gff files with 0.1 Mb non-overlapping windows using bedtools makewindows and bedtools intersect. True-seq and Tran-seq coverage were calculated using bedtools genomecov in BedGraph format. The circus plot was made with the R circlize package, and the gene clusters plots were made with the gggenes package. The full R scripts can be found at github.com/Luisitox/Helichrysum_paper.

### Phylogenetic Analyses of Functionally Tested Enzymes

The selection of the proteins for each of the families analyzed in this study was based on functionally tested enzymes according to studies referenced in each Figure. The full list of IDs can be found in Berman et al., “Parallel evolution of cannabinoid biosynthesis”; Nature Plants 9 817-831 (2023). The Maximum Likelihood trees were constructed with 100 bootstrap tests based on a MUSCLE multiple alignment using the MEGA11 software. The evolutionary distances were computed using the JTTmatrix-based method.

### Orthology and Synteny Analyses

Proteomes were obtained from all available annotated Asteraceae genomes present in NCBI: GCA_003112345.1 (Artemisia annua), GCA_009363875.1 (Mikania micrantha), GCA_023376185.1 (Cichorium endivia), GCA_023525715.1 (Cichorium intybus), GCA_023525745.1 (Arctium lappa), GCA_023525975.1 (Smallanthus sonchifolius), GCA_024762085.1 (Ambrosia artemisiifolia), GCF_001531365.2 (Cynara cardunculus var. scolymus), GCF_002127325.2 (Helianthus annuus), GCF_002870075.4 (Lactuca sativa), GCF_010389155.1 (Erigeron canadensis) and Cannabis sativa GCA_900626175.1. Orthogroups and their phylogenetic relationship were inferred with Orthofinder. Genomic positions and putative function of all the genes belonging to the orthogroups of HuCoAT6 (OG0014461), HuOLS4 (OG0000313), and HuCBGAS4 (OG0002538) were determined using the corresponding GFF files and the plots were produced with the gggenomes package. Phylogenetic gene trees generated by Orthofinder were plotted with MEGA11.

### β-Glucosidase Assay for Preparation of DHSA 93

MPLC fractions (50 ml each) containing Glc-DHSA 103 were evaporated using a rotary evaporator at 40° C., lyophilized and reconstituted in 15 ml McIlvaine buffer (20 mM, pH 5.0). Reactions were performed in separate 20 ml vials incubated at 45° C. for 24 h. Each reaction consisted of 6 ml of McIlvaine buffer (pH 5.0), 3 ml of 0.1 mg ml⁻¹ of an almond β-glucosidase solution in Mcilvaine buffer (≥6 U mg⁻¹, Sigma Aldrich), and 1.5 ml of the fractions containing Glc-DHSA 103. The metabolites were extracted using 3 volumes of ethyl acetate: diethyl ether 1:1, evaporated using a rotary evaporator and reconstituted in 5 ml methanol. The products from the reaction contained a mixture of both glucosylated and non-glucosylated metabolites. DHSA 93 was therefore purified using System 2 and reconstituted in 100 μl methanol for the enzymatic assay. The purified DHSA 93 was analyzed via UPLC-qTOF to verify that the purified fraction did not contain Glc-DHSA 103.

AAE, PKS, PKC, UGT and AAT Expression in E. coli and Protein Purification

HuAAE1-6, HuUGT1-13 and HuAAT1-15 coding sequences from H. umbraculigerum and previously characterized sequences from rice (OsUGT) and stevia (SrUGT), were individually cloned into the pET28b vector digested with EcoRI using the ClonExpress II one step cloning kit (Vazyme, Germany). HuPKS1-4, HuPKC1-5, CsOLS and CsOAC were ligated into the pOPINF vector (digested with HindIII and KpnI) using the ClonExpress II one step cloning kit (Vazyme, Germany). Due to the high sequence similarity of the coding sequences, HuPKS2-4 were synthesized by the company Twist Biosciences. All constructs were expressed in E. coli BL21 (DE3) cells (a complete list of the primers can be found in Berman et al., “Parallel evolution of cannabinoid biosynthesis”; Nature Plants 9 817-831 (2023)). Bacterial starters were grown overnight in LB medium at 37° C., diluted in fresh LB 1:100, and re-incubated at 37° C. When cultures reached A600=0.6, protein expression was induced with 400 μM of isopropyl-1-thio-β-d-galactopyranoside (IPTG) overnight at 15° C. Bacterial cells were lysed by sonication in 50 mM Tris-HCl pH 8.0, 0.5 mM phenylmethylsulfonyl fluoride (PMSF, Sigma Aldrich) solution in isopropanol, 10% glycerol and protease inhibitor cocktail (Sigma Aldrich), and 1 mg ml⁻¹ lysozyme (Sigma Aldrich). The whole-cell extract was either kept for functional activity or used for protein purification. Purification of hexahistidine-tagged proteins was performed on Ni-NTA agarose beads (Adar Biotech). The proteins were eluted with 200 mM imidazole (Fluka) in buffer containing 50 mM NaH₂PO₄, pH 8.0. and 0.5 M NaCl. Protein concentration of the eluted fractions was measured with Pierce™ 660 nm protein assay reagent (Thermo Scientific).

### AAE Enzyme Assays

Recombinant AAE assays were performed in a 20 μl reaction mix that contained 0.1 μg recombinant AAE, 50 mM HEPES pH 9.0, 8 mM ATP, 10 mM MgCl₂, 0.5 mM CoA and 4 mM of the sodium salt of the respective acid (acetic, butyric, hexanoic, octanoic, cinnamic and coumaric acids) for 10 min at 40° C. Reactions were terminated with 2 μl of 1 M HCl and stored on ice until analysis. After centrifugation at 15 000 g for 5 min at 4° C., the samples were diluted 1:100 in water and analyzed on the TQ-S system in MRM mode using a similar column as previously described. The system was operated with an aqueous buffer pH 7.0 (10 mM Ammonium Acetate, 5 mM NH₄HCO₂, phase A) and acetonitrile (phase B). The flow rate was 0.3 ml min⁻¹, and the column temperature was kept at 25° C. Metabolites were analyzed using a 15 min multistep gradient method: initial conditions were 1% B raised to 35% B until 10.5 min, and then raised to 100% B until 11 min, held at 100% B for 1 min, decreased to 1% B until 12.5 min, and held at 1% B until 15 min for re-equilibration of the system. The instrument was operated in positive mode with a capillary voltage of 3.0 kV, and a cone voltage of 50 V. Metabolite identity was confirmed with authentic standards. Two different transitions were used for analysis of: acetyl-CoA (810.52>303.30, 27.0V; 810.52>428.25, 24.0V); butyryl-CoA (838.58>331.30, 28.0 V; 838.58>331.30, 25.0 V); hexanoyl-CoA (866.65>359.40, 28.0 V; 866.65>428.25, 26.0 V); octanoyl-CoA (894.65>387.55, 30.0 V; 894.65>428.25, 28.0 V); coumaroyl-CoA (914.59>407.37, 30.0 V; 914.59>428.25, 28.0 V); cinnamoyl-CoA (898.59>391.37, 30.0 V; 898.59>428.25, 28.0 V).

### PKS and PKC Enzyme Assays

Individual and coupled HuPKS and PKC (HuOACs or CsOAC) assays were carried out as described by Gagne et al. (2012) with some modifications. Enzyme assays were performed in 50 μL with 20 mM HEPES at pH 7.2, 5 mM DTT, 1.8 mM malonyl CoA and 0.6 mM of hexanoyl-CoA. HuPKSs (5 μg) and PKCs (10 μg), were added either individually or in combination. Reaction mixtures were incubated at 30° C. for 3 h. Reactions were stopped by extraction with 100 μL methanol, vortexing and centrifugation at 15 000 g for 10 min. The supernatant was filtered and analyzed with both UPLC-qTOF and triple-Quad systems. The column and mobile phase were as for the metabolic profiling. Initial conditions were 10% B raised to 70% until 6 min, raised to 100% B until 6.2 min, held at 100% B until 8 min, decreased to 10% B until 8.5 min, and held at 10% B until 11 min for re-equilibration of the system. The flow rate was 0.3 ml min⁻¹, and the column temperature was kept at 35° C. UPLC-qTOF was run in both polarities with MS or MS/MS modes using similar parameters as previously described. The TQ-S system was operated in MRM mode in both positive (for olivetol) and negative modes with a capillary voltage of 3.5 or 1.5 kV, respectively, and a cone voltage of 40 or 20 V, respectively. Two different transitions were used for analysis of: OA 92 (223.1>179.1, 15.0 V; 223.1>137.1, 20.0 V); PDAL (181.2>137.1, 10.0 V; 181.2>97.1, 20.0 V); HTAL (223.1>179.1, 10.0 V; 223.1>125.1, 10.0 V); PCP 95 (223.1>179.1, 20.0 V; 223.1>81.0, 25.0 V); olivetol (181.1>111.0, 10.0 V; 181.1>71.2, 10.0 V). Olivetol, OA 92 and PCP 95 identities were confirmed with authentic standards.

### PT Enzyme Assays

HuPT1-4 genes from H. umbraculigerum were separately cloned into pESC-TRP vector. Microsomal preparations from yeast cells transformed with pESC-TRP vectors were performed as described by Jozwiak et al. (2020). PT enzymatic assays were carried out as described previously for CsPT4⁸ with some modifications. The microsomes from yeasts expressing the HuPTs were resuspended in 3.3 ml buffer (10 mM Tris-HCl, 10 mM MgCl2, pH 8.0, 10% glycerol) and homogenized with a tissue grinder. The enzyme assays were performed in 50 μL with 2 μl of the respective membrane preparations dissolved in the reaction buffer (50 mM Tris-HCl, 10 mM MgCl2, pH 8.0), with 500 μM of the aromatic acceptor [OA 92, VA, DHSA 93, PCP 95, naringenin chalcone 97 or pinocembrin chalcone 100] and 500 μM of the isoprenoid (IPP, GPP or FPP). Samples were incubated for 1 h at 30° C. Kinetic assays were similarly performed with 1 mM of GPP and varying (0.5 μM-1.5 mM) concentrations of OA 92, with 15 min incubation at 30° C. Samples were extracted with 100 μl ethanol followed by vortexing and centrifugation. The supernatant was filtered and analyzed via UPLC-qTOF as for the terpenophenols (UPLC Method 1).

### UGT Enzyme Assays

The UGT enzyme assays were performed as described by Cai et al. (2021) with some modifications. UGT assays using different aromatic substrates were performed by mixing 1.5 μl of the UDP-Glc solution (80 mM, final concentration: 2.5 mM), 27.5 μl Tris buffer (100 mM, pH 8.0), 1 μl of each of the substrates (50 mM, final concentration: 1 mM) and 20 μl of the lysate enzyme solution. The reactions were incubated at 30° C. for 1 h. Reactions were stopped by extraction with 100 μl methanol, vortexing and centrifugation at 15,000 g for 10 min. The supernatant was filtered and analyzed via UPLC-qTOF using UPLC Method 2. The assay with the purified UGTs was performed by mixing 2 μl of the cannabinoid acceptors (OA 92, DHSA 93, CBGA 1, heliCBGA 2, CBDA, A9-THCA, CBCA 15, olivetol, CBG, CBD or A9-THC, PCP 95, naringenin chalcone 97 or pinocembrin chalcone 100) in the presence of 1.5 μl UDP-Glc 80 mM, 46.5 μl Tris buffer (100 mM, pH 8.0) and 1 μl of each enzyme. The metabolites were extracted and analyzed as previously described. Kinetic assays were performed with the purified enzymes (1.5 μg μl⁻¹) dissolved in 45 μl Tris buffer (100 mM, pH 8.0) and substrates were added using varying (0.5 μM-3 mM) and constant (1 mM) concentrations of OA 92 and UDP-Glc and the total reaction volume was 50 μl. To stop the reactions, 100 μl methanol was added to each tube, and the metabolites were extracted and analyzed as previously described.

### AAT Enzyme Assay

Recombinant AAT assays using different donor and acceptor substrates were performed by mixing 7 μl of the cannabinoid acceptors (OA 92, CBGA 1, or heliCBGA 2, 1 mg ml⁻¹) with 58 μl of a potassium phosphate buffer (100 mM, pH 7.4), 5 μl of the acyl-CoA donors (butyryl-CoA, hexanoyl-CoA, iso-valeryl-CoA, or acetyl-CoA, 10 mM) and 30 μl of the enzyme solutions. The reactions were incubated at 30° C. for 3 h. Samples were extracted with 100 μl ethanol followed by vortexing and centrifugation. The supernatant was filtered and used for UPLC-qTOF analysis using a similar column, mobile phase and MS parameters as previously described for terpenophenols. Initial conditions were 40% B for 1 min, raised to 100% B until 14 min, held at 100% B for 3.8 min, decreased to 40% B until 18 min, and held at 40% B until 20 min for re-equilibration of the system. The flow rate was 0.3 ml min⁻¹, and the column temperature was kept at 35° C.

The assay with the purified HuCBAT5 enzyme was performed by mixing 2 μl of the cannabinoid acceptors (OA 92, CBGA 1, heliCBGA 2, CBDA, A9-THCA or CBCA 15) with 2 μl of the acyl-CoA donors (butyryl-CoA, iso-butyryl-CoA, hexanoyl-CoA, iso-valeryl-CoA, or acetyl-CoA, 10 mM), 44 μl of a potassium phosphate buffer (100 mM, pH 7.4), and 2 μl of the purified HuCBAT5 enzyme solution. The reactions were incubated at 30° C. for 3 h. To stop the reactions, 50 μl ethanol was added to each tube and the acylated metabolites were extracted and analyzed via UPLC-qTOF as for the terpenophenols (UPLC Method 1) in both MS and MS/MS modes. Extracted ion chromatograms using the major products were selected from the LC-MS/MS analyses as follows: cannabinoid acceptors without CoAs: OA 92>179.107, CBGA 1, CBCA 15>191.107, heliCBGA 2>225.092, CBDA, A9-THCA>245.154; acylated cannabinoids: OA 92>179.107, CBGA 1>231.102, heliCBGA 2>265.086, CBDA>245.154, A9-THCA>245.154, CBCA 15>191.107).

Transient Expression of Selected Genes in N. benthamiana

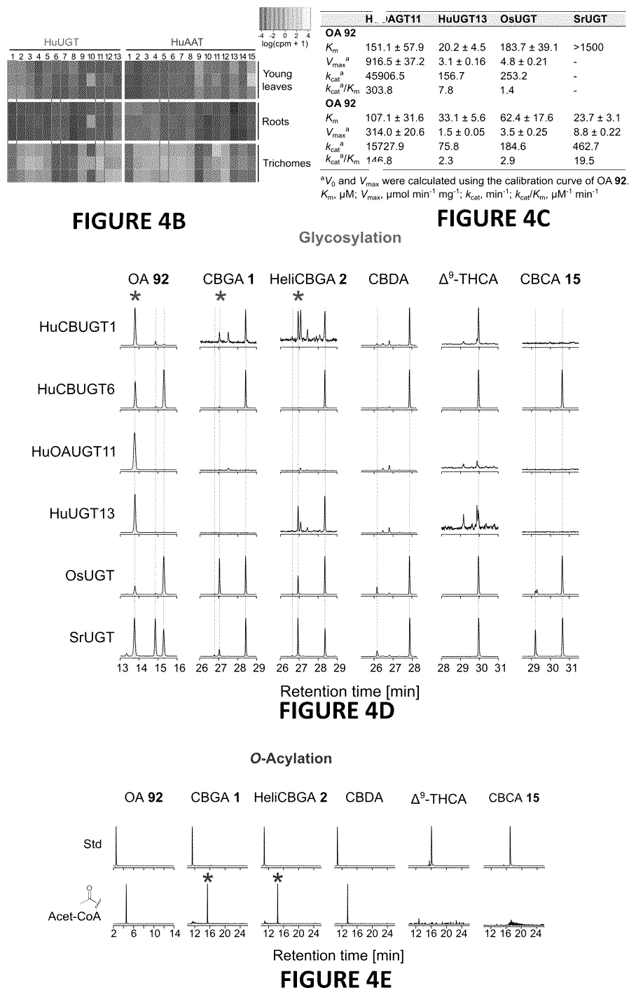

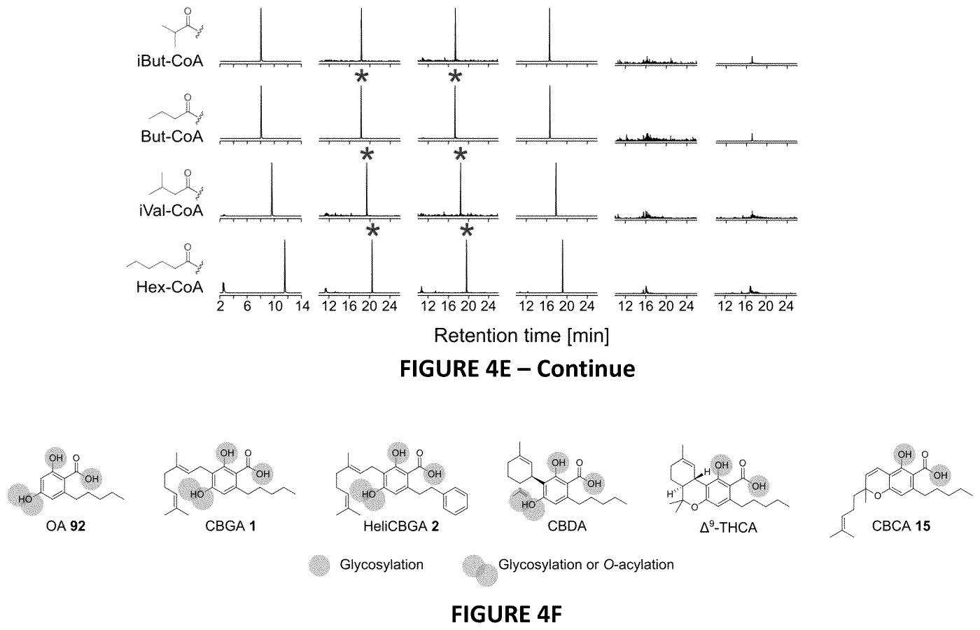

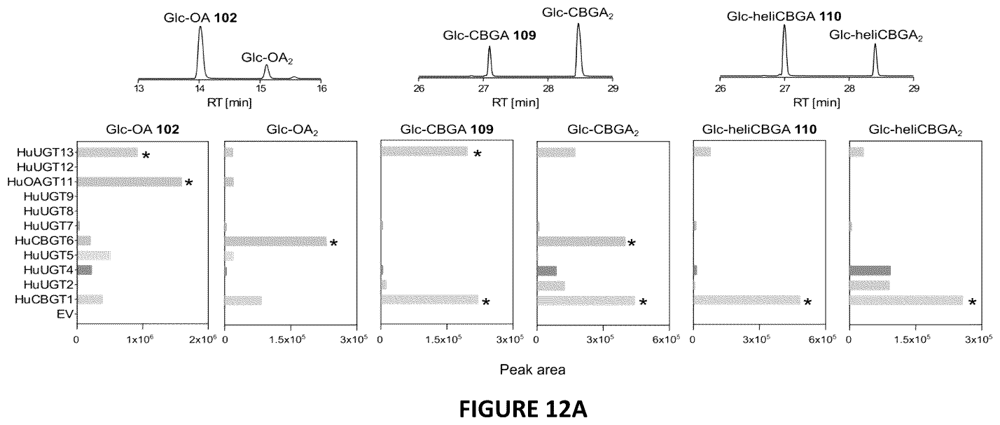

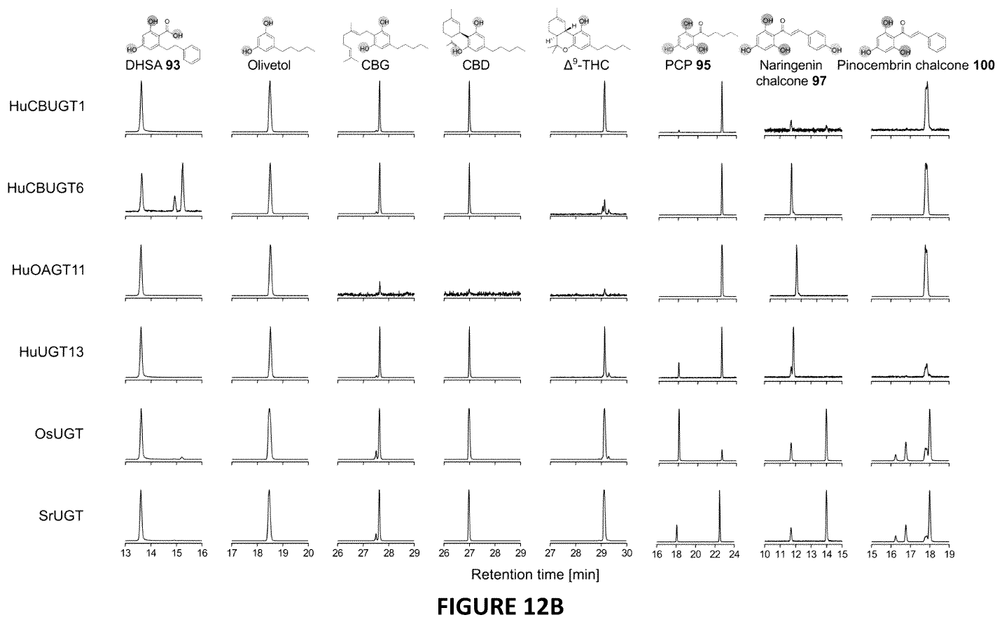

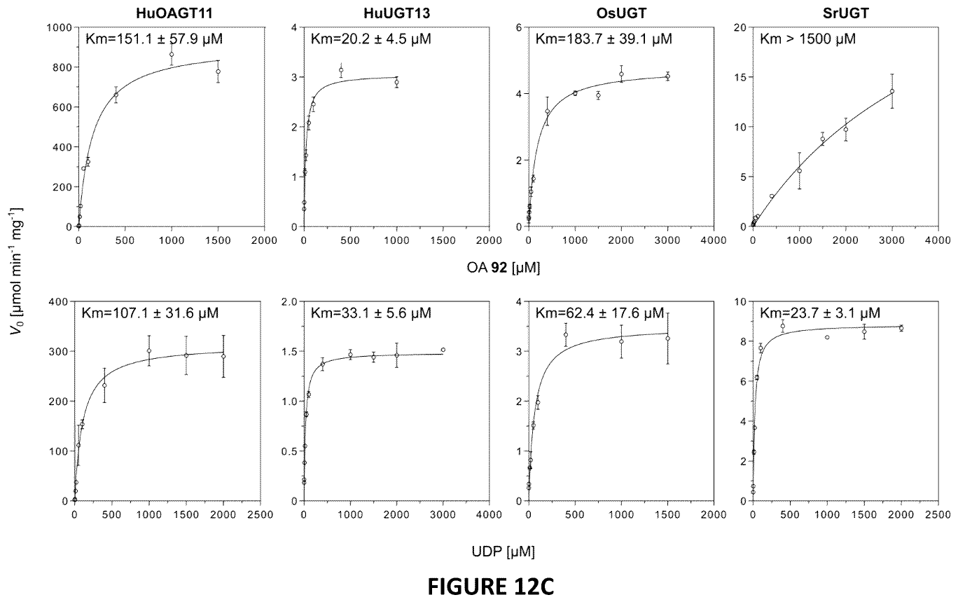

Overexpression constructs of GFP (as negative control), CsOLS and CsOAC were generated using GoldenBraid cloning as described by Jozwiak et al. 2020 to a final vector of pAlpha2-Ubq10-CCD-Ter10. HuCoAT6, HuTKS4, and HuCBGAS were amplified and cloned in pAlpha2-NPT II-Ubq10-CCD-Ter10 vector digested with Bsal using ClonExpress II One Step Cloning kit (Vazyme). The full list of oligonucleotides used for cloning can be found in Berman et al., “Parallel evolution of cannabinoid biosynthesis”; Nature Plants 9 817-831 (2023). All plasmids were sequenced and transformed into Agrobacterium tumefaciens strain GV3101 by electroporation. A. tumefaciens harboring the overexpression constructs were grown overnight at 28° C. in Luria-Bertani (LB) medium in the presence of kanamycin and gentamycin. Bacterial cells were collected by centrifugation, washed and resuspended in infiltration buffer (10 mM MES, 2 mM MgCl2, 2 mM Na3PO4, 0.5% glucose and 100 mM acetosyringone) to OD600=0.3. Equal volumes of A. tumefaciens suspension with different expression vectors were combined to obtain the desired gene combinations and incubated for 2 h at room temperature. The solutions were infiltrated into 4- or 5-week-old N. benthamiana leaves from the abaxial side using a 1-ml needleless syringe. Substrates (0.5 mM each) were infiltrated into the same leaf areas 2 days after initial infiltration, and leaves were collected for metabolite analysis after 24 h. Leaf samples were flash frozen and extracted as previously described with 300 μl methanol and analyzed on a similar UPLC system connected to an Orbitrap IQ-X Tribrid MS (Thermo Scientific, Bremen, Germany) using UPLC Method 2 in negative mode. The source parameters were: sheath gas flow rate, auxiliary gas flow rate and sweep gas flow rate: 45, 10 and 1 arbitrary units, respectively; vaporizer temperature: 300° C.; ion transfer tube temperature: 275° C.; spray voltage: 2.3 kV. The instrument was operated in full MS1 with data dependent MS/MS (MS-dd-MS2). Data acquisition in full MS1 mode was 60,000 resolution, the scan range 100-1000 m/z, normalized automatic gain control (AGC) target of 25% and a maximum injection time (IT) of 50 ms. Data acquisition in dd-MS2 mode was with 15,000 resolution, a normalized AGC target of 20%, maximum IT of 150 ms, isolation window of 1.5 m/z and normalized collision energy of 40. Identification of metabolites was performed using analytical standards and/or products from in vitro UGT enzyme assays (FIGS. 4D and 12B).

Heterologous Expression in S. cerevisiae

For the expression of HuCoAT6, HuTKS4, CsOAC and HuCBGAS in S. cerevisiae, the CDSs were amplified, and the purified amplicons were inserted into series of pESC (Amp^{R}) plasmids allowing simultaneous expression of two genes from one plasmid. HuCoAT6 and HuTKS4 were inserted using ClonExpress II One Step Cloning kit (Vazyme) into pESC-HIS plasmid linearized with SalI and SacI restriction enzymes, respectively. HuCBGAS and CsOAC were cloned in the same way into pESC-TRP plasmid linearized with SalI/SacI restriction enzymes, respectively. The full list of primers used for the cloning can be found in Berman et al., “Parallel evolution of cannabinoid biosynthesis”; Nature Plants 9 817-831 (2023). pESC constructs were transformed into S. cerevisiae WAT11 using Yeastmaker yeast transformation system (Clontech). The inventors transformed yeast cells with combinations of pESC vectors allowing expression of all the four genes at once. Transformed yeast were grown on SD minimal media supplemented with appropriate amino acids and 2% glucose. Colonies were screened and the presence of the transgene was confirmed by colony PCR. For induction of gene expression, transformed cells were grown in 2 ml minimal medium with 2% glucose and after 24 h transferred to a minimal medium with 2% galactose without additional supplementation or supplemented with GPP (0.21 mM) and either sodium hexanoate (1 mM) or OA 92 (0.2 mM), and grown for additional 24 h at 30° C. Cultures were transferred to 2 ml Eppendorf tube and centrifuged at 8,000 g for 1 min. The cell pellet was weighed, double the amount of glass beads (diameter 500 μm) and 500 μl of MeOH was added and lysed using a bead beater at 22 Hz for 6 min. Lysed cells were centrifuged at 14,000 r.p.m. for 5 min, clear supernatant was collected and dried using SpeedVac. Dry residues were dissolved in 100 μl of methanol, filtered through a 0.22 μm filter and analyzed on LC-MS as detailed for N. benthamiana samples.

### Example 1

### H. umbraculigerum Produces CBGA

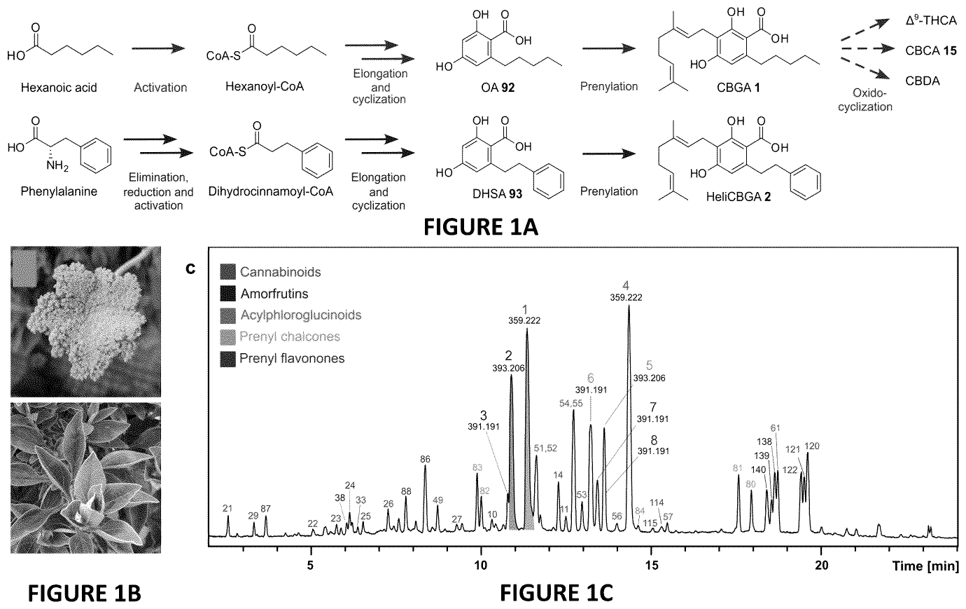

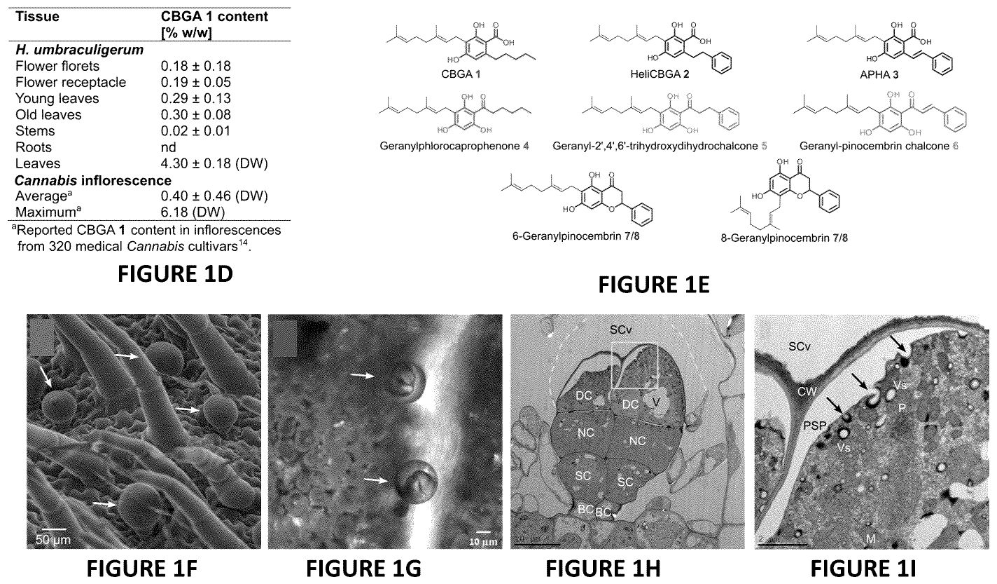

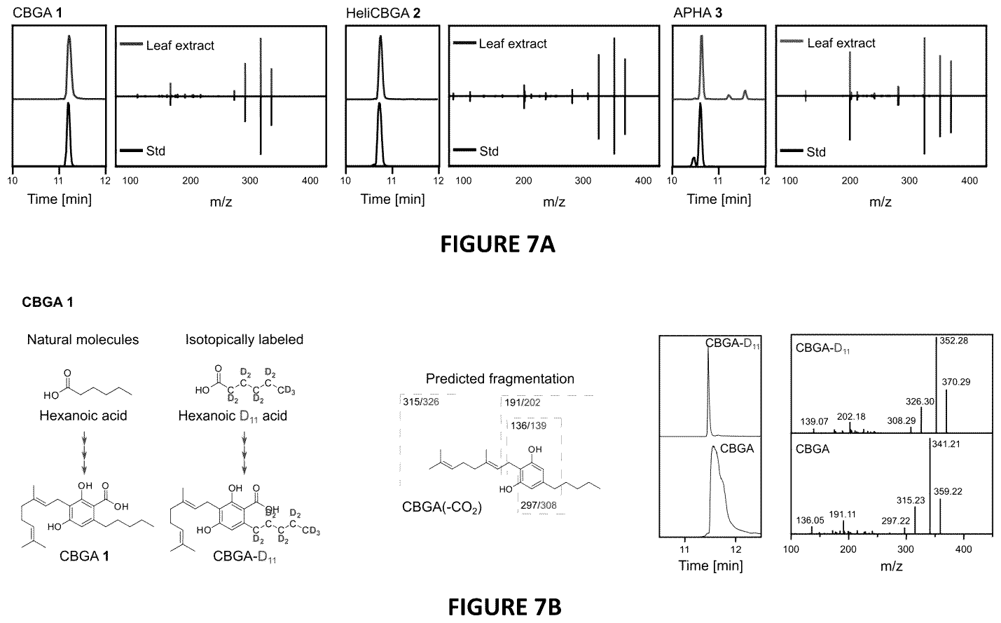

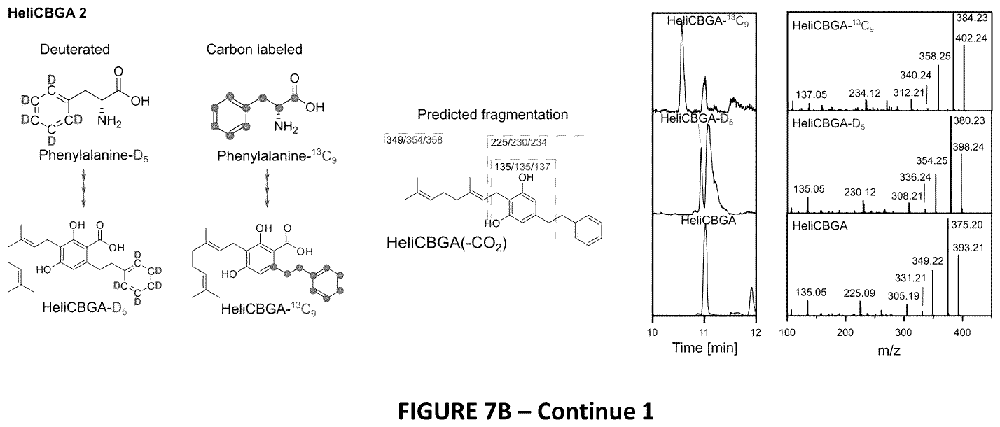

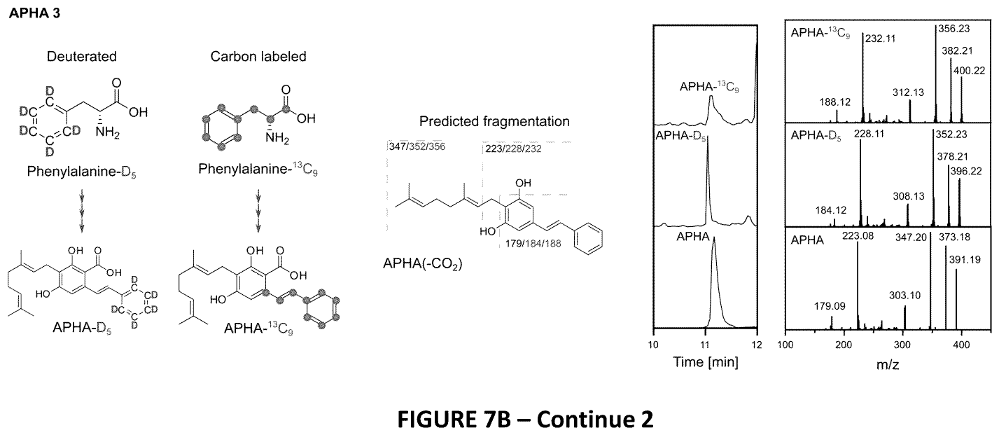

As two earlier reports regarding the presence of cannabinoids, specifically CBGA 1, in H. umbraculigerum were contradictory, the inventors decided to carry out comprehensive chemical profiling of cannabinoids in various H. umbraculigerum tissues. The inventors confirmed that CBGA 1 is a major component of H. umbraculigerum, accumulating up to 4.3% on a dry weight basis in leaves (FIGS. 1C-1D) comparable to the maximum typically measured concentrations in inflorescences of Cannabis chemotypes (FIG. 1D). CBGA 1, its phenethyl analog heliCBGA 2, and pre-amorphastilbol (APHA, 3), the stilbene form of heliCBGA 2, represent three of the major peaks in the total ion chromatogram of a fresh leaves ethanolic extract (FIGS. 1C-1E, and 7A).

The inventors predicted that CBGA 1 and heliCBGA 2 biosynthesis originates from hexanoic acid and phenylalanine, respectively (FIG. 1A). Therefore, the inventors fed H. umbraculigerum leaves with unlabeled and stable isotopically labeled hexanoic acid (hexanoic-D₁₁ acid) or phenylalanine (phenylalanine-D₅ or phenylalanine-¹³C₉) and compared the labeled versus non-labeled masses and their respective tandem mass spectrometry (MS/MS) spectra (FIG. 7B). Consequently, newly derived isotopologues were detected as co-eluting chromatographic peaks (unlabeled and labeled forms) with mass shifts and MS/MS fragmentation patterns corresponding with the isotopically-labeled parts of the molecule. These findings validated the existence of the alkyl and aralkyl cannabinoids in H. umbraculigerum and confirmed that their biosynthesis derives from the polyketide and phenylpropanoid pathways, respectively. Feeding experiments revealed the presence of some additional major prenyl-acyl-phloroglucinoids, prenylchalcones and prenylflavanones with similar chemical formulas as 1-3. Based on previously identified core structures and each metabolite MS/MS fragmentation spectra, the inventors assigned these peaks to the structures shown in FIG. 1E [see also Berman et al., “Parallel evolution of cannabinoid biosynthesis”; Nature Plants 9 817-831 (2023)].

### Example 2

### Cannabinoids Accumulate in Glandular Trichomes

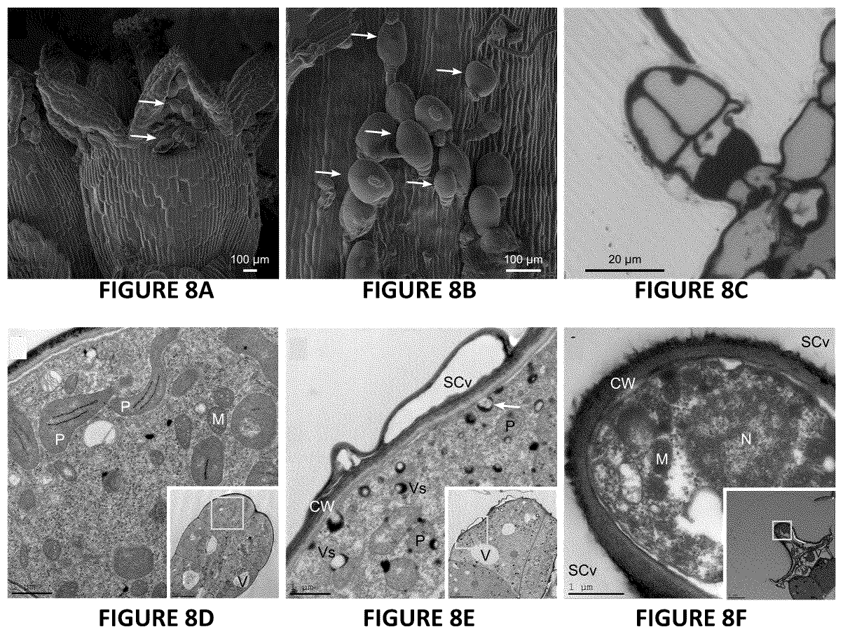

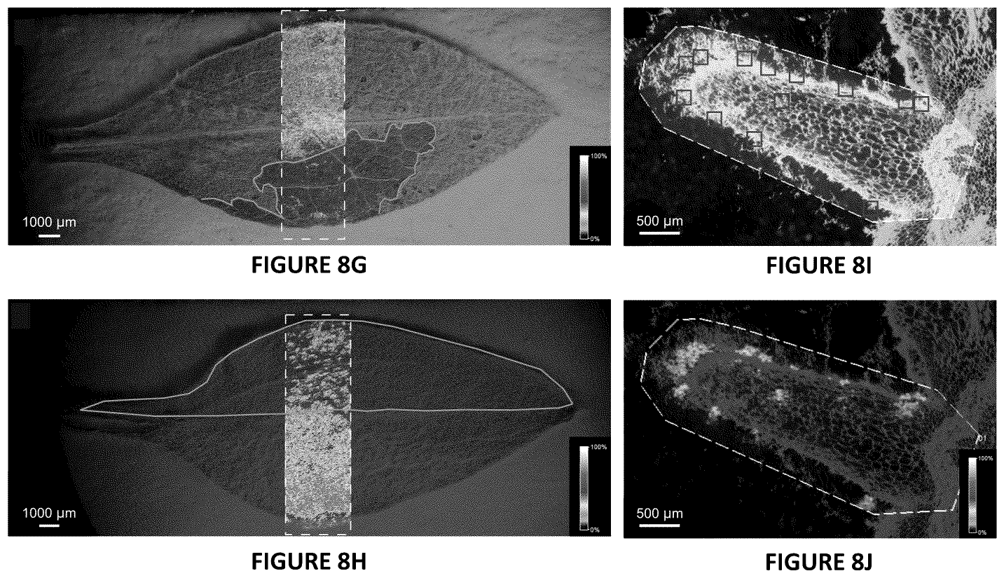

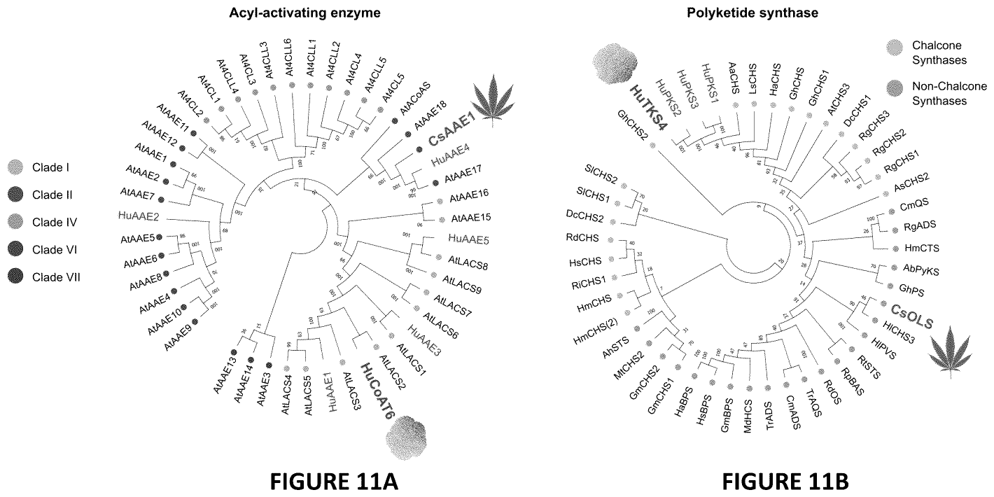

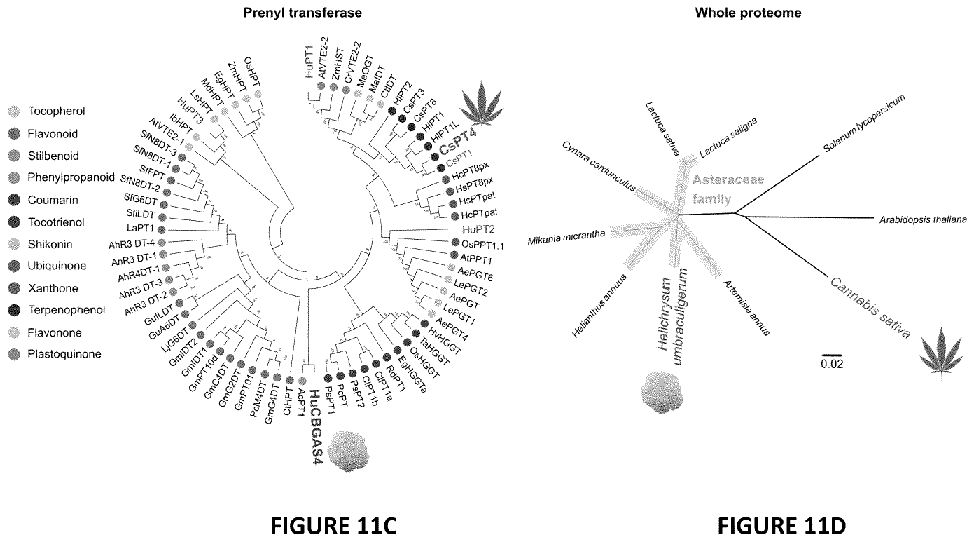

The inventors employed various high-resolution imaging technologies to examine if, like Cannabis, H. umbraculigerum develops and accumulates cannabinoids in glandular trichomes. The inventors found that in flowers, the involucral bracts of the capitula had numerous non-glandular and glandular trichomes. In individual florets, glandular trichomes were particularly abundant on the tips of the corolla lobe (FIGS. 8A-8B). In leaves, both the adaxial and abaxial surfaces were densely covered with both non-glandular and glandular trichomes (FIG. 1F). The glandular trichomes were slightly elevated from the epidermis and consisted of a biseriate stalk and a globose head (FIG. 8C). Two disk cells (DCs) were observed in the subcuticular space of the globose head (FIG. 1G). In Cannabis, cannabinoid biosynthesis takes place in these cells. The multicellular biseriate structure of the trichomes further consisted of two basal cells (BCs, not always observed), stalk cells (SCs), neck cells (NCs), and a secretory cavity (SCv) (FIG. 1H). DCs of trichomes at the secretion stage showed exudation of electron transparent secretions from plastids into vesicles, followed by exocytosis of their contents into the periplasmic space (PSP), where they accumulated prior to secretion into the SCv (FIGS. 11 and 2D-2F).

Next, the inventors applied matrix-assisted laser desorption/ionization-mass spectrometry imaging (MALDI-MSI) to spatially localize cannabinoids in H. umbraculigerum. The inventors first analyzed the abaxial and adaxial leaf surfaces following partial removal of trichomes (FIGS. 8G-8H imaging m/z [M+H]⁺=361.237 Da corresponding to CBGA 1 and geranylphlorocaprophenone 4). As shown, metabolites were detected in the intact parts, while areas with partially or fully removed trichomes showed less or no signals, respectively. The inventors further analyzed cross-sections of H. umbraculigerum leaves and flowers. The inventors sectioned leaves crosswise so that trichomes on the adaxial and abaxial parts were exposed on each side (FIG. 2A). In flowers, the inventors sectioned the receptacle, exposing trichomes on the outer surface of the involucral bracts (FIG. 81). As shown in FIG. 2B and FIG. 8J for the leaf and flower samples, respectively, CBGA 1 was found exclusively in glandular trichomes.

### Example 3

### H. umbraculigerum Produces Both Classical and Novel Cannabinoids

Cannabis produces various CBGA-type analogs with aliphatic chains of different lengths (one to seven carbons), derived from different linear short- and medium-chain fatty acids (FAs). The inventors observed in leaves of H. umbraculigerum several of these analogs, including cannabigerovarinic acid (CBGVA 9), cannabigerol butyric acid (CBGBA 10), cannabigerohexolic acid (CBGHA 11), and cannabigerophorolic acid (CBGPA 12), corresponding to three, four, six, and seven carbon-atom chains, respectively (Berman et al., “Parallel evolution of cannabinoid biosynthesis”; Nature Plants 9 817-831 (2023)). The inventors also observed two metabolites with similar masses and fragmentation patterns as CBGA 1 and CBGHA 11, which the inventors assigned as cannabinoids derived from branched FAs (13 and 14, respectively, Berman et al., “Parallel evolution of cannabinoid biosynthesis”; Nature Plants 9 817-831 (2023)). These branched cannabinoids have not been identified in Cannabis. The inventors also found small amounts of CBCA 15 and its aromatic analog helichromenic acid (heliCBCA 16) and their hydroxylated forms (17 and 18, respectively, Berman et al., “Parallel evolution of cannabinoid biosynthesis”; Nature Plants 9 817-831 (2023)), and the isoprenyl-forms of CBGA 1 and heliCBGA 2 according to MS/MS fragmentation (CBPA 19 and heliCBPA 20, respectively, Berman et al., “Parallel evolution of cannabinoid biosynthesis”; Nature Plants 9 817-831 (2023)). The inventors did not detect Δ⁹-THCA- or CBDA-type cannabinoids in any of the tissues.

Some additional peaks exerted MS/MS fragments and chemical formulas corresponding to one or two hydroxylations of the metabolites with five-carbon-atom chains, which were labeled following feeding with hexanoic-D₁₁ acid (21-33, Berman et al., “Parallel evolution of cannabinoid biosynthesis”; Nature Plants 9 817-831 (2023)). Interestingly, hydroxylated amorfrutins were observed with similar fragmentation patterns as the cannabinoids (with m/z difference of 33.984 Da), suggesting similar chemical structures and enzymes associated with their metabolism (34-46, Berman et al., “Parallel evolution of cannabinoid biosynthesis”; Nature Plants 9 817-831 (2023)). The inventors purified from this group metabolite 26 and identified by NMR spectroscopy a new tetrahydroxanthane-type cannabinoid (12-OH-cyclocannabigerolic acid 26). According to its MS/MS fragmentation pattern, the inventors also putatively identified cyclocannabigerolic acid (cycloCBGA 47) and analogous amorfrutin types [12-OH-heli-cyclocannabigerolic acid (12-OH-helicycloCBGA 39) and heli-cyclocannabigerolic acid (helicycloCBGA 48), respectively, Berman et al., “Parallel evolution of cannabinoid biosynthesis”; Nature Plants 9 817-831 (2023)].

According to the current feeding experiments, prenyl-acyl-phloroglucinoids, prenylchalcones, and prenylflavanones were derived from similar precursors as the cannabinoids and amorfrutins (49-91, Berman et al., “Parallel evolution of cannabinoid biosynthesis”; Nature Plants 9 817-831 (2023)). A summary of the identified metabolites 1-91 appears in Berman et al., “Parallel evolution of cannabinoid biosynthesis”; Nature Plants 9 817-831 (2023).

### Example 4

### Proposed Cannabinoid Biosynthetic Pathway in H. umbraculigerum

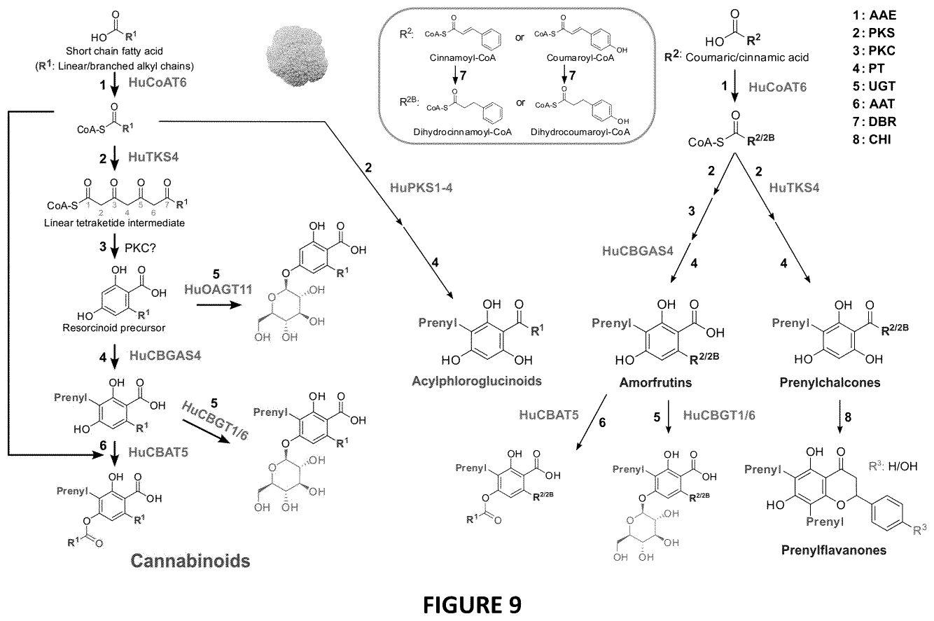

The inventors postulated that the core cannabinoid pathway leading to CBGA 1 in H. umbraculigerum consists of similar types of enzymes and reactions as in Cannabis (FIG. 9). These include: an acyl-activating enzyme (AAE) for the activation of hexanoic acid into hexanoyl-CoA; a type III polyketide synthase (PKS) and a polyketide cyclase (PKC) to produce olivetolic acid (OA 92) and a membrane-bound aromatic prenyl transferase (PT) for geranylation of OA 92 to CBGA 1. In addition to CBGA 1 and other cannabinoids, the inventors propose that all the identified terpenophenols are produced via five parallel pathways (Berman et al., “Parallel evolution of cannabinoid biosynthesis”; Nature Plants 9 817-831 (2023)). According to this scheme, cannabinoids and phloroglucinoids derive from a common linear or branched FA precursor activated via the same AAE enzyme. Amorfrutins and chalcones derive from cinnamic or coumaric acids, which originate from phenylalanine, and are also activated via an AAE enzyme (similar or different from the polyketide one). These activated intermediates can be further reduced by a double bond reductase (DBR) to form dihydro intermediates. The activated precursors are elongated using three malonyl CoAs by one or more PKS-type enzymes, and further cyclized by the PKS in a Claisen reaction to form the phloroglucinoid or chalcone backbone, or in an aldol reaction assisted by a PKC to form the cannabinoids and amorfrutins. The fifth pathway employs a chalcone isomerase (CHI) enzyme that cyclizes chalcones to flavanones. All these intermediates are further prenylated by one or more PTs to form the different types of terpenophenols. Although most of the molecules enclosed are monoprenyls, other prenyl types were also observed. The terpenophenols can be further cyclized by berberine bridge-like enzymes (BBE-like) to produce cyclized metabolites like CBCA 15, cyclocannabinoids and cycloamorfrutins (26, 47, 39 and 48), and also cyclophloroglucinoids previously identified by Pollastro et al. (2017). Additional functional groups and rearrangements include hydroxylation, double bond isomerization or reduction and others. In support of these five pathways, the inventors identified in H. umbraculigerum the primary intermediates (before prenylation) from all the corresponding metabolic routes (92-101, Berman et al., “Parallel evolution of cannabinoid biosynthesis”; Nature Plants 9 817-831 (2023)).

### Example 5

### Elucidation of the Core Cannabinoid Pathway

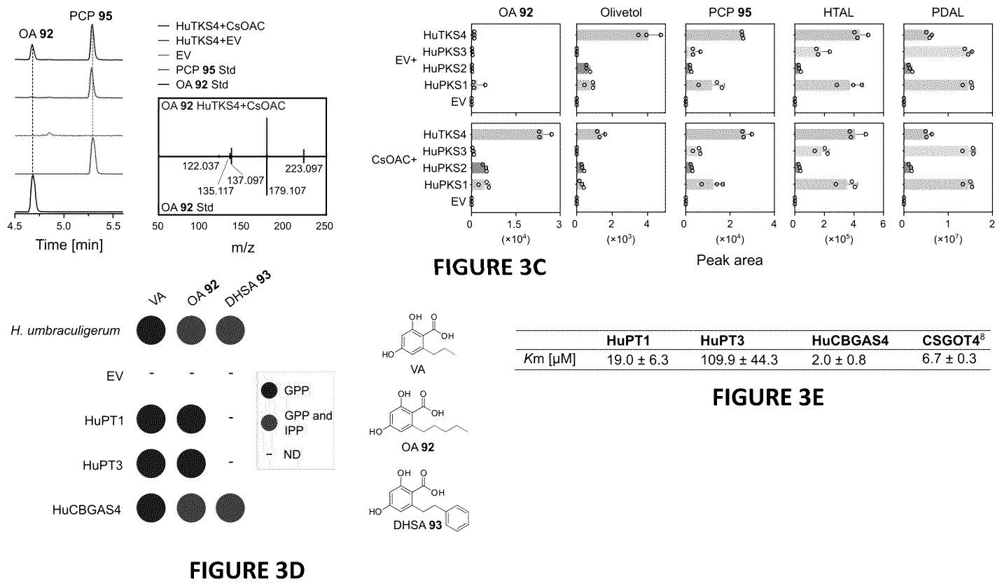

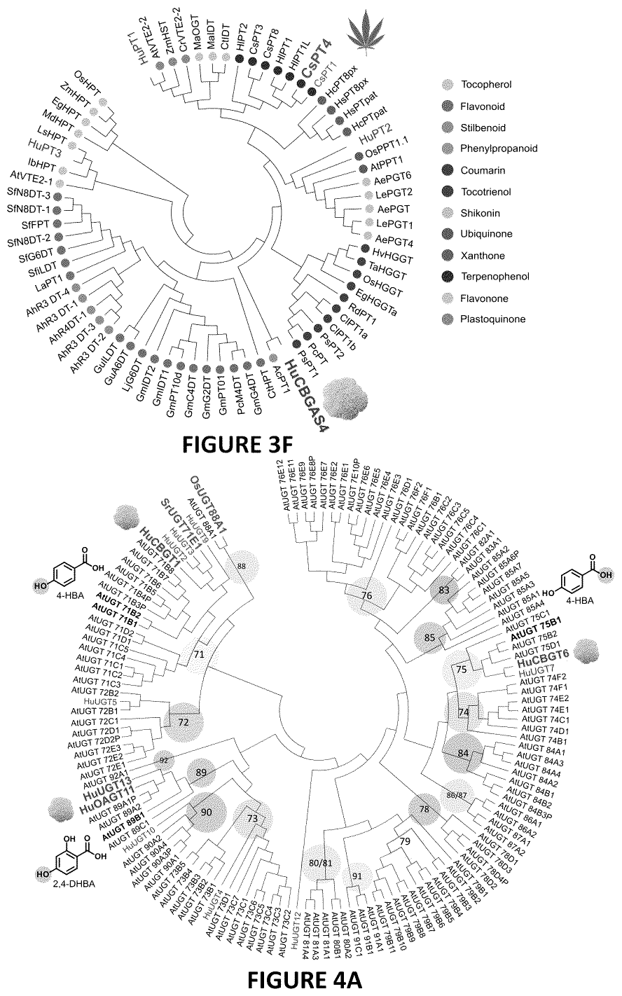

To identify the enzymes responsible for cannabinoid biosynthesis in H. umbraculigerum, the inventors obtained a haplotype resolved dual genome assembly using 44× Pacbio HiFi reads, and 200 M reads of Illumina HiC chromatin interaction data (haploid size of ˜1.3 Gb, Berman et al., “Parallel evolution of cannabinoid biosynthesis”; Nature Plants 9 817-831 (2023)). After scaffolding the N50 of the primary assembly was 174 Mb with eight scaffolds >10 Mb (FIG. 2C, and Berman et al., “Parallel evolution of cannabinoid biosynthesis”; Nature Plants 9 817-831 (2023)). The inventors also obtained RNAseq data using PacBio Iso-Seq, Illumina True-Seq, and Illumina UMI-aware 3′ Transeq of different tissues (Berman et al., “Parallel evolution of cannabinoid biosynthesis”; Nature Plants 9 817-831 (2023)). The genome was soft masked (Berman et al., “Parallel evolution of cannabinoid biosynthesis”; Nature Plants 9 817-831 (2023)) and gene models were obtained reaching BUSCO completeness values of 98.7% for the primary assembly and 99.3% for all transcripts, including those missing in the genome (Berman et al., “Parallel evolution of cannabinoid biosynthesis”; Nature Plants 9 817-831 (2023)). Based on FIG. 2B, the inventors expected that the biosynthetic genes would be highly expressed in trichomes. Weighted gene co-expression network analysis of H. umbraculigerum tissue transcriptomic data (Berman et al., “Parallel evolution of cannabinoid biosynthesis”; Nature Plants 9 817-831 (2023)) revealed a transcriptional module enriched in FA and terpenoid biosynthetic genes induced in trichomes and leaves (FIGS. 2D-2E, and Berman et al., “Parallel evolution of cannabinoid biosynthesis”; Nature Plants 9 817-831 (2023)). This module included two AAEs, three PKSs, one stress-related protein (potential PKC) and one PT (FIG. 2E and Berman et al., “Parallel evolution of cannabinoid biosynthesis”; Nature Plants 9 817-831 (2023)). Notably, three of these PKSs were also located in a tandem gene cluster consisting of seven enzymes of the same type (FIG. 2C). This region exhibited strong footprints of long terminal repeat (LTR) transposition activity, which might explain the observed patterns of gene duplication (Berman et al., “Parallel evolution of cannabinoid biosynthesis”; Nature Plants 9 817-831 (2023)). Overall, the inventors selected six HuAAEs, four HuPKSs, five HuPKCs and four HuPTs for further characterization (FIG. 3A and Berman et al., “Parallel evolution of cannabinoid biosynthesis”; Nature Plants 9 817-831 (2023)). The four selected PKSs showed subtle amino acid differences that would have been overlooked without the genomic sequence, and the inability to amplify the different variants from cDNA led us to produce the genes synthetically.

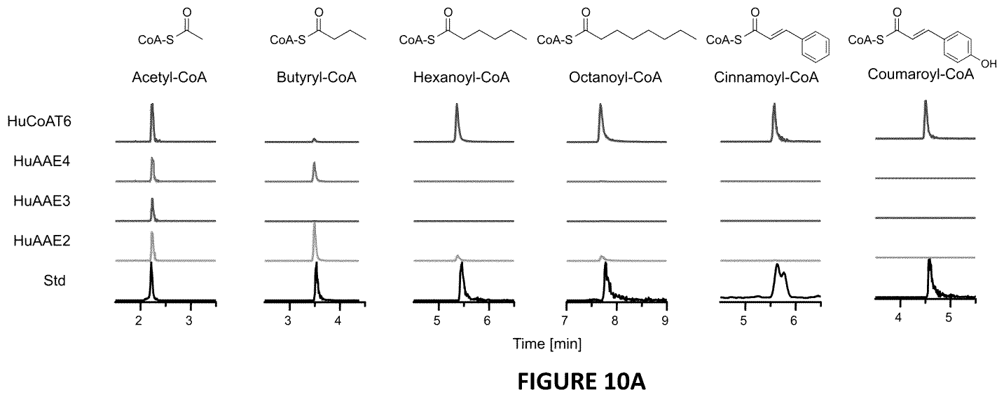

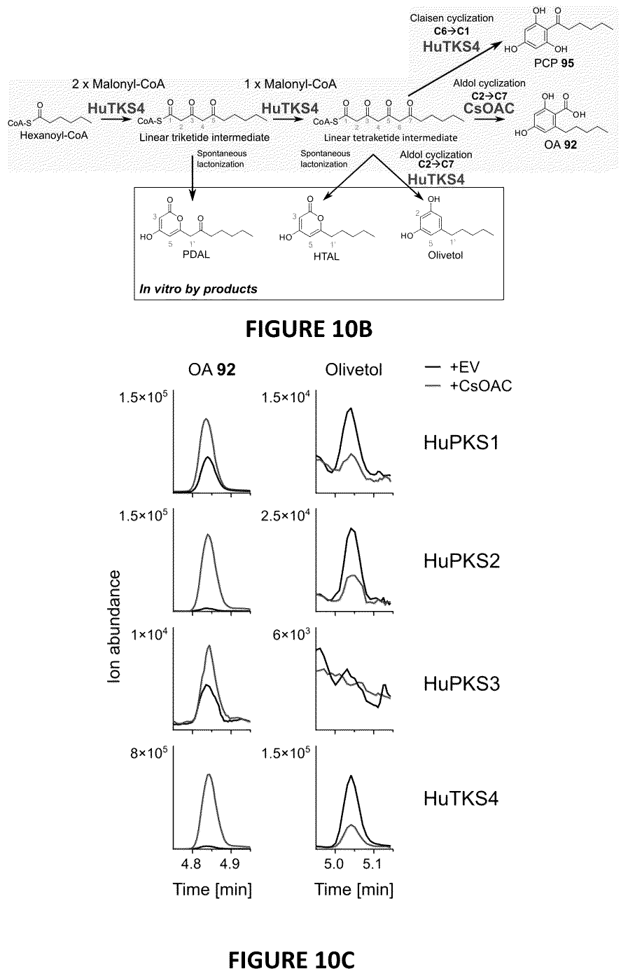

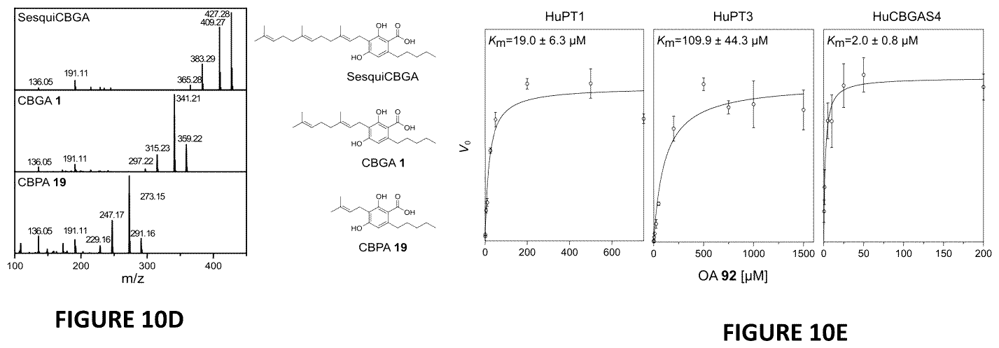

The first step in cannabinoid biosynthesis involves the formation of acyl-CoA thioesters by members of the AAE superfamily. As different acyl moieties are substrates for these enzymes, the inventors tested acetic, butyric, hexanoic, octanoic, cinnamic and coumaric acids. In vitro assays with purified recombinant proteins showed that HuAAE2 and HuAAE4 efficiently produced butyryl-CoA, and that HuAAE2 presented higher activity against acetic acid and formed acetyl-CoA (FIGS. 3B and 10A). HuAAE6 (HuCoAT6) was the only enzyme with activities towards both medium chain alkyl (e.g., hexanoic and octanoic acids) and aralkyl (e.g., cinnamic and coumaric acids) precursors required for the five types of terpenophenols observed in H. umbraculigerum. Interestingly, while HuAAE4 belongs to the same clade as the most active Cannabis enzyme, HuCoAT6 is located within the clade of long-chain acyl-CoA synthetases (LACS, FIG. 11A and Berman et al., “Parallel evolution of cannabinoid biosynthesis”; Nature Plants 9 817-831 (2023)).

In Cannabis, the next step is performed by a coupled enzymatic reaction involving a CsOLS and the accessory protein CsOAC, resulting in the condensation of hexanoyl-CoA with three molecules of malonyl-CoA to yield OA 92. In in vitro assays, derailment of the unstable intermediates occurs producing additional by-products not naturally identified in plant extracts [olivetol, pentyl acyl diacetic acid lactone (PDAL) and hexanoyl acyl triacetic acid lactone (HTAL), Berman et al., “Parallel evolution of cannabinoid biosynthesis”; Nature Plants 9 817-831 (2023)]. PDAL and HTAL are produced by spontaneous lactonization of the tri- and tetra-ketide unstable intermediates, whereas olivetol is produced by CsOLS in the absence of CsOAC in an aldol decarboxylation cyclization reaction resembling the production of resveratrol by a stilbene synthase (STS). When CsOAC is also present in the reaction, OA 92 is produced at the expense of olivetol. Here, the inventors cloned and expressed in E. coli HuPKS1-4, HuPKC1-5, CsOLS and CsOAC enzymes, and tested using hexanoyl-CoA and malonyl-CoA their ability to form OA 92 in coupled in vitro assays with all the possible combinations (Berman et al., “Parallel evolution of cannabinoid biosynthesis”; Nature Plants 9 817-831 (2023)). In the absence of PKCs, all the HuPKSs produced the PDAL and HTAL by-products, while HuPKS1, HuPKS2 and HuPKS4 produced also olivetol (FIGS. 3C and 10C). When the reactions were performed coupled to CsOAC, olivetol decreased and OA 92 increased, especially for HuPKS4 (HuTKS4) (FIG. 3C and Berman et al., “Parallel evolution of cannabinoid biosynthesis”; Nature Plants 9 817-831 (2023)). However, in all the reactions, considerably smaller amounts of olivetol and OA 92 were observed compared to HTAL and PDAL (FIG. 3C). Interestingly, regardless of CsOAC, all HuPKSs produced the phloroglucinoid precursor phlorocaprophenone 95 (PCP), present in H. umbraculigerum (Berman et al., “Parallel evolution of cannabinoid biosynthesis”; Nature Plants 9 817-831 (2023), and FIG. 3C). This suggested that the same HuPKS enzyme can carry out both the aldol and Claisen cyclization reactions. This phenomenon has been observed previously for CHS and STS enzymes producing different amounts of both naringenin and resveratrol, and PKSs producing different ratios of both resorcinolic-acid and phloroglucinoid products. Interestingly, the HuPKS protein sequences did not cluster with known resorcinolic-acid or phloroglucinoid producing PKSs such as CsOLS, Rhododendron dauricum orcinol synthase (RdOS) or Humulus lupulus valerophenone synthase (HIVPS) (FIG. 11B). None of the combinations including HuPKS1-HuPKS4 or CsOLS with the HuPKC enzymes (selected based on their expression profile and sequence homology to CsOAC) resulted in the formation of OA 92 (Berman et al., “Parallel evolution of cannabinoid biosynthesis”; Nature Plants 9 817-831 (2023)). This suggests that the cyclization and possibly stabilization of the tetraketide intermediate is mediated by a different type of enzyme than in Cannabis. This was previously suggested to occur in Rhododendron dauricum in the production of orselinic acid by RdOS and a yet to be identified PKC enzyme20. Alternatively, H. umbraculigerum may contain another CsOAC homolog that the inventors did not characterize in this study.

In the next step, OA 92 or OA-derivatives are prenylated by aromatic PTs to form CBGA 1 and its derivatives. The inventors expressed four enzymes in yeast and purified the microsomal fractions used for enzymatic assays (HuPT1-4, FIG. 3D). The inventors examined an array of aromatic substrates and either geranyl pyrophosphate (GPP) or isopentenyl pyrophosphate (IPP) as the isoprenoid donors. All the HuPTs geranylated OA 92 and divarinolic acid (VA) to yield CBGA 1 and CBGVA 9, respectively. HuPT4 geranylated also the aromatic dihydrostilbenic acid (DHSA 93) and was the only enzyme that isoprenylated OA 92 and DHSA 93 (FIG. 3D). HuPT4 was also active with farnesyl pyrophosphate (FPP) yielding sesquicannabigerolic acid (SesquiCBGA, FIG. 10D). Kinetic assays of the HuPTs with GPP and OA 92 revealed that HuPT4 (HuCBGAS4) exhibited a smaller Michaelis-Menten Km value than the reported one from Cannabis CsPT4 [FIGS. 3e, and 10E]. Interestingly, none of the HuPTs prenylated the phloroglucinoid or chalcone intermediates, and none of their sequences clustered with previously known terpenophenolic PTs (FIG. 3F).

To get more insight to the evolution of the pathway, the inventors searched for orthologous enzymes in Cannabis and in all other Asteraceae species with annotated genomes. To the best of inventors' knowledge, these species do not accumulate terpenophenols. Similarly, to the phylogenetic relationships observed for functionally tested enzymes (i.e., AAEs, PKSs and PTs, FIG. 11), the enzymes that enabled H. umbraculigerum to produce cannabinoids evolved independently in this lineage. Particularly for the PKS-type enzymes, multiple instances of gene duplication and subsequent specialization are likely to have occurred within this family. Interestingly, the PTs from Cannabis and ones from H. umbraculigerum did not cluster in the same orthogroup, suggesting that they are derived from evolutionary distant ancestors.

### Example 6

### Decorated Cannabinoids are Formed by UGT- and BAHD-Type Enzymes

Glycosylated cannabinoids have not been reported to occur naturally in planta. Here the inventors identified glucosylated OA (Glc-OA 102) and glucosylated DHSA (Glc-DHSA 103) as well as glucosylated C₃-C₆ alkyl-chain intermediates (104-108), glucosylated CBGA (Glc-CBGA 109) and heliCBGA (Glc-heliCBGA 110), and their isoprenylated forms (Glc-CBPA 111 and Glc-heliCBPA 112) (Berman et al., “Parallel evolution of cannabinoid biosynthesis”; Nature Plants 9 817-831 (2023)). All these metabolites exhibited neutral losses of 162.053 Da corresponding to hexose and similar fragments as the non-glucosylated compounds. Di-glucosylated metabolites were not identified in the extracts. In Arabidopsis thaliana uridine 5′-diphospho-glucuronosyltransferases (AtUGT89B1, AtUGT71B1, AtUGT75B1 and AtUGT71B2) catalyze the glycosylation of several hydroxybenzoic acids (HBA and DHBAs) which are structurally like OA 92 (FIG. 4A). The inventors selected thirteen gene candidates in H. umbraculigerum based on sequence similarity to these proteins and positive correlations between genes expression and the accumulation of glucosylated metabolites (HuUGT1-13, FIGS. 4A-4B, and Berman et al., “Parallel evolution of cannabinoid biosynthesis”; Nature Plants 9 817-831 (2023)).

Eleven of the thirteen UGTs from H. umbraculigerum were expressed in E. coli and examined for enzyme activity using OA 92, CBGA 1, and heliCBGA 2 in a reaction including uridine diphosphate glucose (UDP-Glc) as the sugar donor. Eight out of the eleven enzymes showed activity on the different substrates, including HuUGT1-2, HuUGT4-7, HuUGT11, and HuUGT13 (FIG. 12A). The production of Glc-OA 102 in the enzyme assays with UDP-Glc was supported by the NMR assignment of the glucose moiety. The inventors next purified the four most active enzymes (HuUGT1, HuUGT6, HuUGT11, and HuUGT13) and performed in vitro assays with an array of cannabinoid substrates, both natural and unnatural to H. umbraculigerum (FIGS. 4D, and 12B, Berman et al., “Parallel evolution of cannabinoid biosynthesis”; Nature Plants 9 817-831 (2023)). The inventors also included enzymes from stevia and rice (SrUGT and OsUGT, respectively) reported to possess cannabinoid glycosylation activity despite these plants not producing cannabinoids. All enzymes were active with varying substrate specificity and products. For example, HuUGT1 and HuUGT6 were most active on the cannabinoids (HuCBUGT1 and HuCBUGT6), while HuUGT11 (HuOAUGT11) and HuUGT13 were highly active on the cannabinoid intermediates while almost inactive on the prenylated metabolites. Di-glucosylation of acid metabolites was only observed in case of HuCBUGT6, while olivetol, cannabidiol (CBD) and cannabigerol (CBG) were di-glucosylated by different HuUGTs depending on the metabolite (Berman et al., “Parallel evolution of cannabinoid biosynthesis”; Nature Plants 9 817-831 (2023)). Interestingly, the UGTs from H. umbraculigerum also glucosylated the phloroglucinoid and flavonoid precursors naturally present in the plant (FIG. 12B), notwithstanding that, the glucosylated forms were not observed in the plant extracts. Kinetic assays of HuOAUGT11, HuUGT13, OsUGT, and SrUGT showed highly significant catalytic activity of HuOAUGT11 with OA 92 and UDP-Glc as compared to all other enzymes (FIGS. 4C and 12C). HuOAGT11 was also co-expressed with other cannabinoid-related enzymes (Berman et al., “Parallel evolution of cannabinoid biosynthesis”; Nature Plants 9 817-831 (2023)) and is therefore the most likely enzyme responsible for the large quantities of Glc-OA 102 and Glc-DHSA 103 produced in H. umbraculigerum (Berman et al., “Parallel evolution of cannabinoid biosynthesis”; Nature Plants 9 817-831 (2023)).

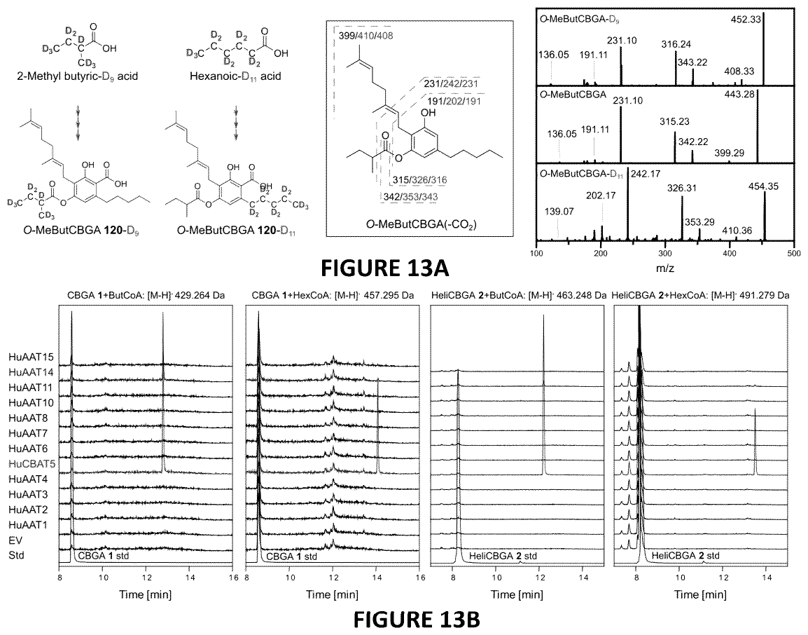

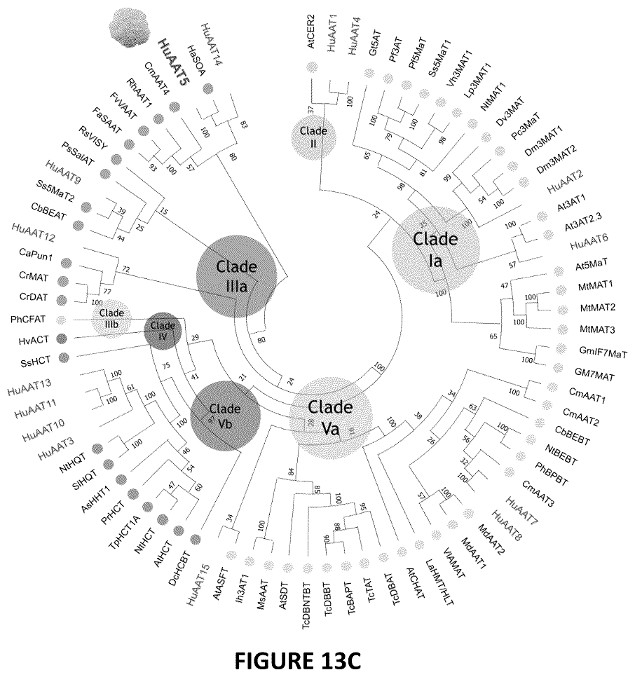

Previous reports identified in H. umbraculigerum isoprenylated O-acylated amorfrutins but not geranylated or alkyl-type ones which are also not found in Cannabis. Here the inventors identified a diverse group of O-acylated cannabinoids and amorfrutins including the O-acylated alkyl (113-130) and aralkyl (131-141) metabolites (Berman et al., “Parallel evolution of cannabinoid biosynthesis”; Nature Plants 9 817-831 (2023)). The inventors hypothesized that the acyl group is derived from short- or medium-chain FAs (FIG. 9) and verified this using a precursor isotope-labeling approach (FIG. 13A). Most of the alkyl cannabinoids in this group had five-carbon-atom tails (according to labeling with hexanoic-D₁₁ acid), and both alkyl and aralkyl metabolites comprised iso- or monoprenyls, and linear or branched short-chain O-acyl groups as shown by the specific labeling (Berman et al., “Parallel evolution of cannabinoid biosynthesis”; Nature Plants 9 817-831 (2023)). To confirm the identification of this group of metabolites, the inventors purified O-methylbutyryl-cannabigerolic acid (O-MeButCBGA 120) and O-methylbutyryl-helicannabigerolic acid (O-MeButheliCBGA 138) and confirmed their structure by NMR.

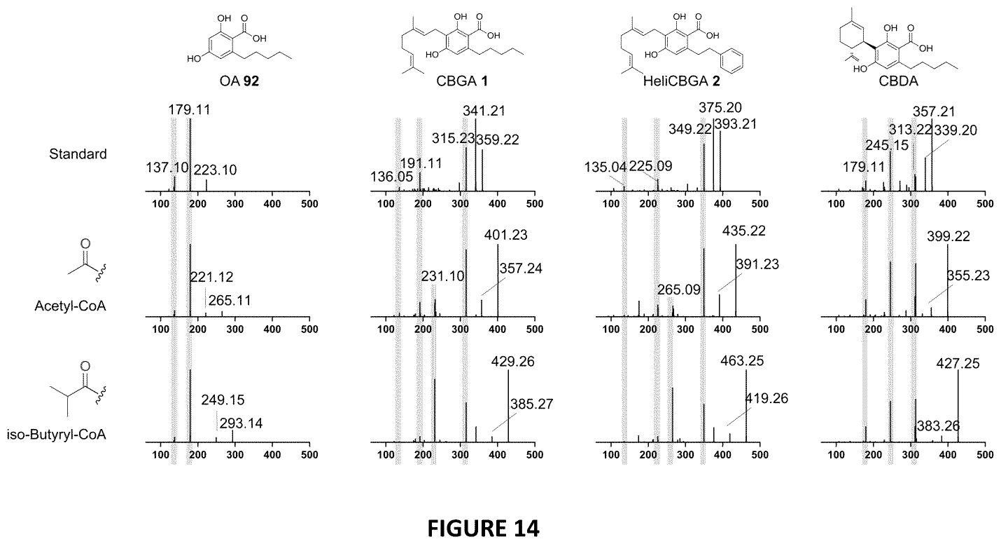

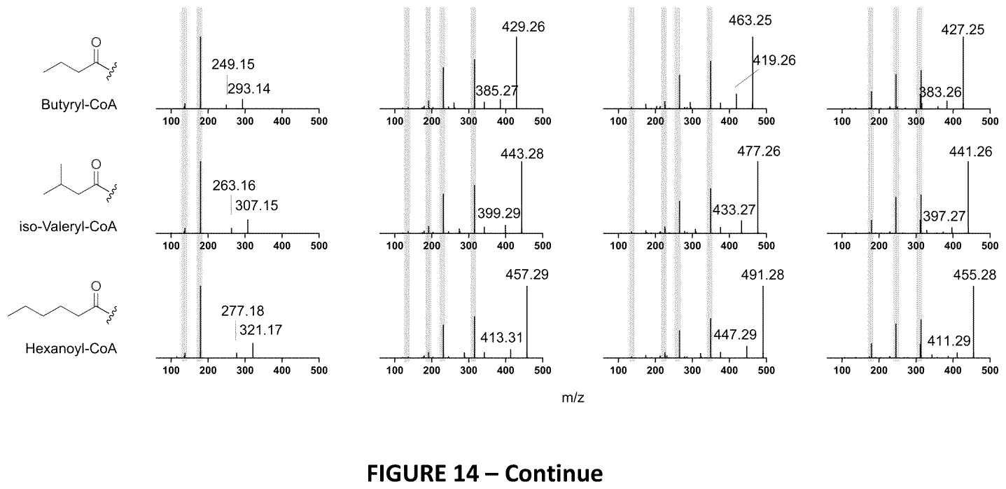

O-Acylation of specialized metabolites in plants is frequently catalyzed by BAHD-type alcohol acyl-transferase (AAT) enzymes. Therefore, the inventors selected fifteen H. umbraculigerum BAHD homologs, four of them co-expressed with other cannabinoid-related enzymes (FIGS. 2E, and 4B and Berman et al., “Parallel evolution of cannabinoid biosynthesis”; Nature Plants 9 817-831 (2023)). Twelve of the fifteen AATs were expressed in E. coli and examined for their activity with butyryl- and hexanoyl-CoA as acyl donors, and CBGA 1 and heliCBGA 2 as acceptors. Only HuAAT5 and HuAAT14 showed activity towards these substrates (FIG. 13B). Phylogenetic analysis showed that these two enzymes clustered in clade IIIa representing BAHDs of diverse catalytic functions (FIG. 13C). HuAAT5 (HuCBAT5) produced larger amounts of products and was therefore selected for in-detail characterization with an array of acyl donors and acceptors. It accepted all acyl donors tested and acylated OA 92, CBGA 1, heliCBGA 2, and CBDA, giving rise to a single O-acyl-cannabinoid from each pair of substrates (FIGS. 4E-4F, and 14 and Berman et al., “Parallel evolution of cannabinoid biosynthesis”; Nature Plants 9 817-831 (2023)). Many of the cannabinoids produced were naturally observed in the plant (marked with an asterisk in FIG. 4E). On the other hand, the enzyme was inactive on A9-THCA and CBCA 15. It is therefore likely that it only acylates the hydroxyl in C5. In addition, O-acyl esterification in H. umbraculigerum was only observed on prenylated cannabinoids and amorfrutins and not on their intermediates.

### Example 7

### In Vivo Reconstruction of the Core Cannabinoid Pathway in Heterologous Systems

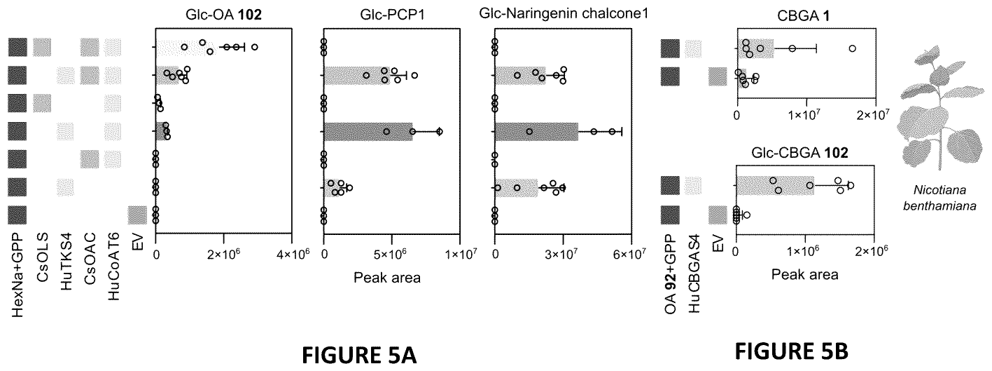

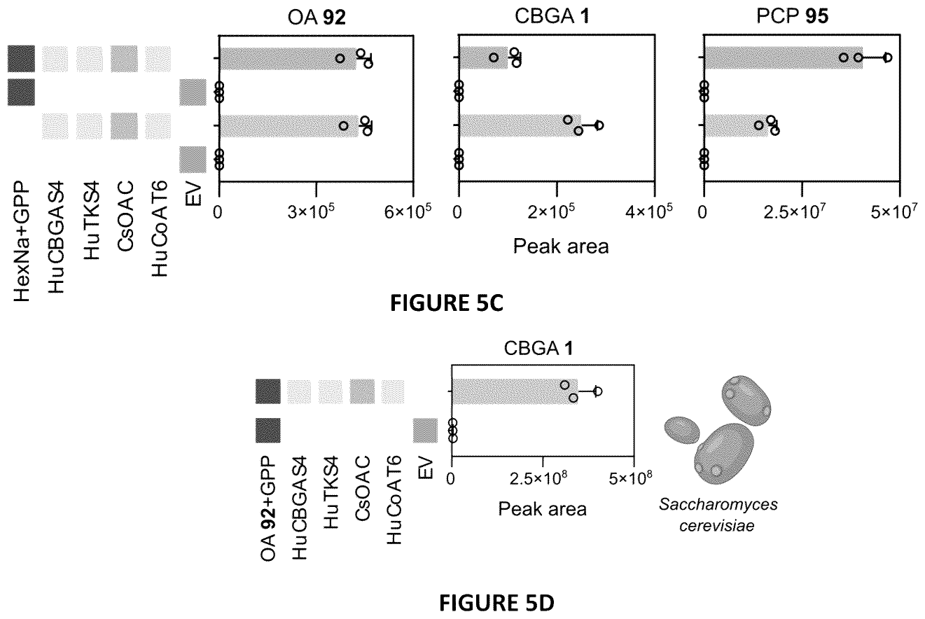

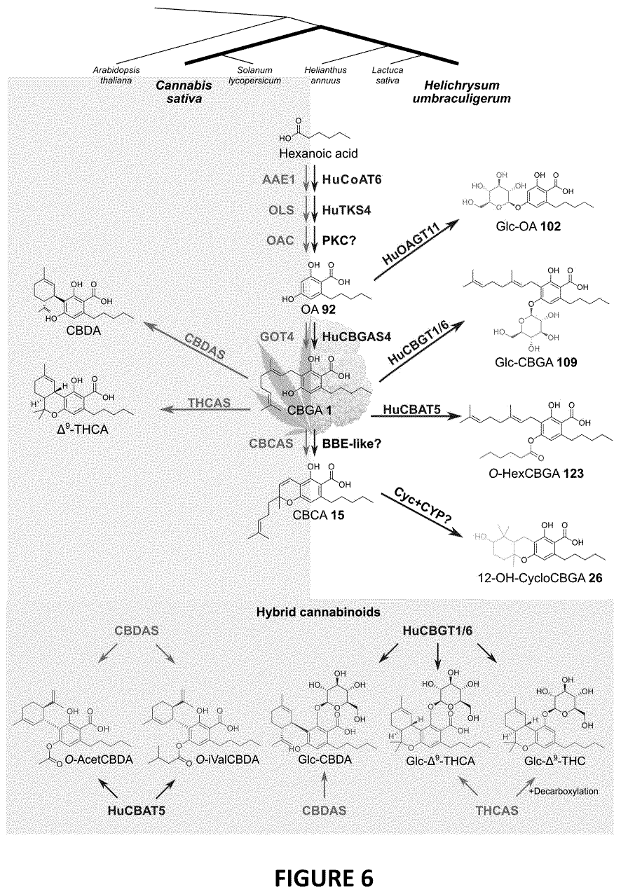

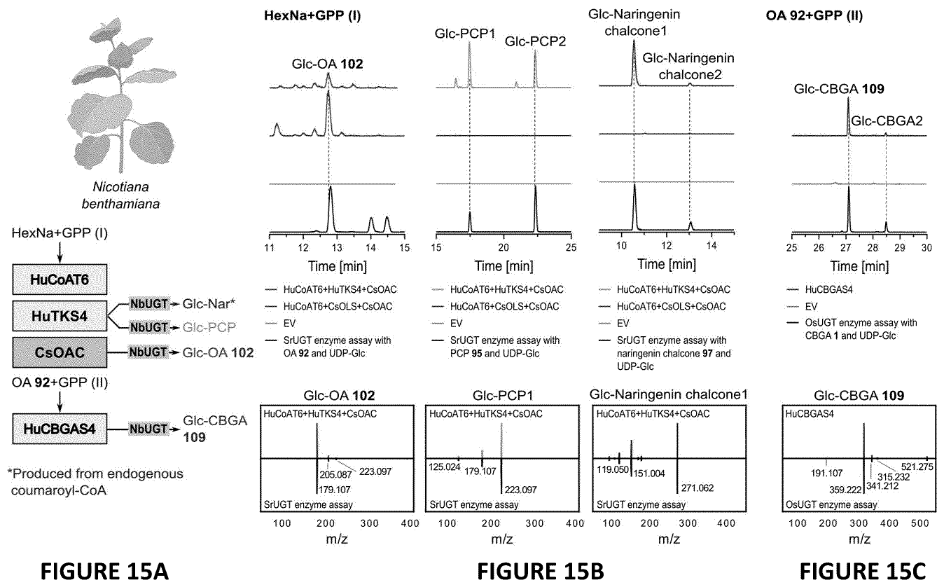

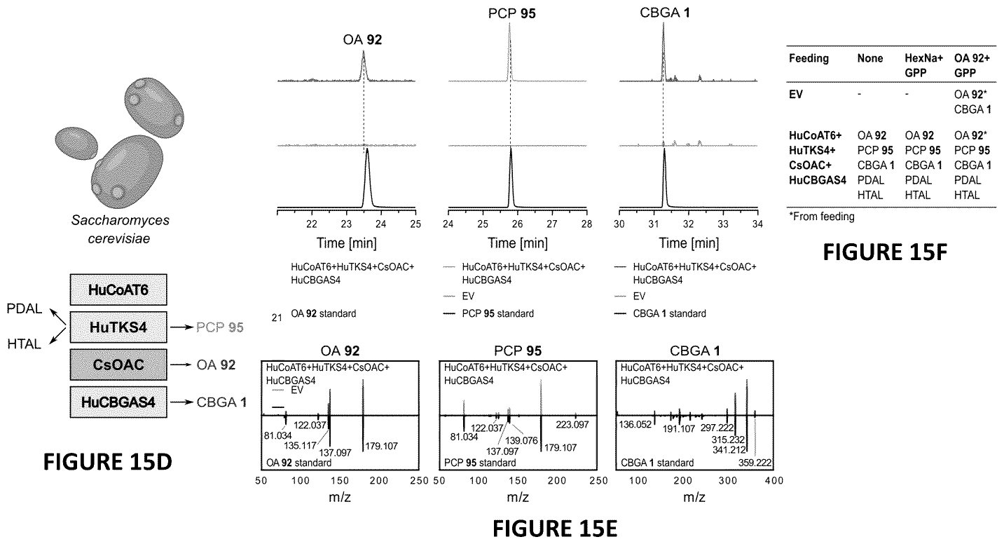

The inventors verified the in planta activity of the enzymes towards CBGA 1 by transiently co-expressing different combinations of HuCoAT6, HuTKS4, and HuCBGAS4, and the Cannabis CsOAC and CsOLS in N. benthamiana leaves. Following leaves infiltration with sodium hexanoate and GPP, the inventors observed the production of glycosylated forms of OA 92 (HuTKS4+CsOAC or CsOLS+CsOAC) and PCP 95 (only with HuTKS4, FIGS. 5A and 15A-15B). This was consistent with previous studies reporting OA 92 glycosylation by endogenous enzymes in this plant. Interestingly, the inventors also observed glycosylated products of naringenin chalcone 97 with HuTKS4, suggesting that this enzyme can accept aromatic substrates in addition to aliphatic types (FIGS. 5A and 15A-15B). However, the inventors did not observe CBGA 1 or its glycosylated forms with HuCBGAS4, likely due to the low availability of OA 92 and its rapid glycosylation in planta. When leaves expressing HuCBGAS4 were infiltrated with OA 92 and GPP, CBGA 1 and Glc-CBGA 109 were observed (FIGS. 5B, 15A, and 15C).

The inventors also reconstituted the cannabinoid pathway by expressing the HuCoAT6, HuTKS4, CsOAC and HuCBGAS4 genes in S. cerevisiae. The inventors observed the production of OA 92, CBGA 1 and PCP 95 without precursor feeding (FIGS. 5C, 15D, and 15E). Similarly, to the in vitro assays, the inventors also observed peaks of HTAL and PDAL which were not present in planta (FIG. 15F). When cells were supplemented with OA 92 and GPP, significantly larger amounts of CBGA 1 were produced (FIG. 5D).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. An isolated DNA molecule comprising at least a first nucleic acid sequence encoding a first protein and at least a second nucleic acid sequence encoding a second protein, wherein said first protein and said second protein are derived from Helichrysum umbraculigerum and belonging to an enzyme family selected from the group consisting of: acyl activating enzyme (AAE), polyketide synthase (PKS), polyketide cyclase (PKC), prenyltransferase (PT), and cannabichromenic acid synthase (CBCAS), and wherein said first protein and said second protein belong to different enzyme families.

2. The isolated DNA molecule of claim 1, further comprising at least a third nucleic acid sequence encoding a third protein derived from H. umbraculigerum and belonging to an enzyme family selected from the group consisting of: AAE, PKS, PKC, PT, and CBCAS, and wherein said first protein, said second protein, and said third protein, belong to different enzyme families, optionally further comprising at least a fourth nucleic acid sequence encoding a fourth protein derived from H. umbraculigerum and belonging to an enzyme family selected from the group consisting of: AAE, PKS, PKC, PT, and CBCAS, and wherein said first protein, said second protein, said third protein, and said fourth protein, belong to different enzyme families, and optionally further comprising at least a fifth nucleic acid sequence encoding a fifth protein derived from H. umbraculigerum and belonging to an enzyme family selected from the group consisting of: AAE, PKS, PKC, PT and CBCAS, and wherein said first protein, said second protein, said third protein, said fourth protein, and said fifth protein, belong to different enzyme families.

3.-4. (canceled)

5. The isolated DNA molecule of claim 1, further comprising a nucleic acid sequence encoding a protein derived from H. umbraculigerum and belonging to an enzyme family selected from the group consisting of: uridine diphosphate (UDP)-glycosyltransferase (UGT), alcohol acyltransferase (AAT), and both, and optionally wherein: (a) said UGT comprises an amino acid sequence with at least 90% homology to any one of: SEQ ID Nos.: 102-114; (b) said AAT comprises an amino acid sequence with at least 91% homology to any one of: SEQ ID Nos.: 130-144; or (c) both (a) and (b).

6. The isolated DNA molecule of claim 1, wherein:
a. said AAE is encoded by a nucleic acid sequence having at least 89% homology to any one of SEQ ID Nos.: 1-11, and any combination thereof;
b. said PKS is encoded by a nucleic acid sequence having at least 83% homology to any one of: SEQ ID Nos.: 23-26, and any combination thereof;
c. said PKC is encoded by a nucleic acid sequence having at least 88% homology to any one of: SEQ ID Nos.: 31-38, and any combination thereof;
d. said PT is encoded by a nucleic acid sequence having at least 91% homology to any one of: SEQ ID Nos.: 47-58, and any combination thereof;
e. said CBCAS is encoded by a nucleic acid sequence having at least 82% homology to any one of: SEQ ID Nos.: 71-79, and any combination thereof, or
f. any combination of (a) to (e).

7. The isolated DNA molecule of claim 5, wherein:
a. said UGT is encoded by a nucleic acid sequence having at least 87% homology to any one of: SEQ ID Nos.: 89-101, and any combination thereof;
b. said AAT is encoded by a nucleic acid sequence having at least 87% homology to any one of: SEQ ID Nos.: 115-129, and any combination thereof; or
c. both (a) and (b).

8. The isolated DNA molecule of claim 1, wherein:
a. said AAE comprises an amino acid sequence with at least 93% homology to any one of SEQ ID Nos.: 12-22;
b. said PKS comprises an amino acid sequence with at least 93% homology to any one of: SEQ ID Nos.: 27-30;
c. said PKC comprises an amino acid sequence with at least 87% homology to any SEQ ID Nos.: 39-46;
d. said PT comprises an amino acid sequence with at least 92% homology to any one of: SEQ ID Nos.: 59-70;
e. said CBCAS comprises an amino acid sequence with at least 86% homology to any one of: SEQ ID Nos.: 80-88; or
f. any combination of (a) to (e).

9. The isolated DNA molecule of claim 5, wherein:
a. said UGT consists of an amino acid sequence of any one of: SEQ ID Nos.: 102-114;
b. said AAT consists of an amino acid sequence of any one of: SEQ ID Nos.: 130-144; or
c. both (a) and (b).

10. The isolated DNA molecule of claim 1, wherein
a. said AAE consists of an amino acid sequence of any one of SEQ ID Nos.: 12-22;
b. said PKS consists of an amino acid sequence of any one of SEQ ID Nos.: 27-30;
c. said PKC consists of an amino acid sequence of any one of SEQ ID Nos.: 39-46;
d. said PT consists of an amino acid sequence of any one of SEQ ID Nos.: 59-70;
e. said CBCAS consists of an amino acid sequence of any one of SEQ ID Nos.: 80-88;
f. or any combination of (a) to (e).

11. (canceled)

12. The isolated DNA molecule of claim 1, comprising a plurality of isolated DNA molecule types, and optionally wherein each type of said plurality of isolated DNA molecule types encodes a protein or a plurality of proteins belonging to a different enzyme family.

13. (canceled)

14. An artificial nucleic acid molecule, a plasmid, or an agrobacterium comprising the isolated DNA molecule of claim 1.

15. (canceled)

16. A transgenic cell comprising the isolated DNA molecule of claim 1, and optionally wherein said transgenic cell is a transgenic Cannabis sativa cell.

17.-20. (canceled)

21. An extract derived from the transgenic cell of claim 16, or any fraction thereof, and optionally wherein said extract comprises a cannabinoid, a precursor thereof, or a combination thereof.

22. (canceled)

23. A transgenic plant, a transgenic plant tissue or a plant part, comprising the isolated DNA molecule of claim 1, and optionally wherein said plant is a transgenic C. sativa plant.

24. (canceled)

25. A composition comprising the isolated DNA molecule of claim 1, and an acceptable carrier.

26. A method for synthesizing a cannabinoid, a precursor thereof, or any combination thereof, comprising the steps:
a. providing a transgenic cell or a cell transfected with the isolated DNA molecule of claim 1 or an artificial nucleic acid molecule comprising thereof; and
b. culturing said transgenic cell or said transfected cell from step (a) such that at least said first protein and said second protein encoded by said artificial nucleic acid molecule are expressed,
thereby synthesizing the cannabinoid, a precursor thereof, or any combination thereof.

27. The method of claim 26, wherein said precursor is selected from the group consisting of: acyl coenzyme A (CoA), a polyketide, a resorcinoid precursor, and any combination thereof.

28. The method of claim 27, wherein any one of: (i) said acyl is C1-C8 alkyl; (ii) said acyl CoA is hexanoyl CoA: (iii) said polyketide is a tetraketide, and optionally wherein said tetraketide is a linear tetraketide; (iv) said resorcinoid precursor is olivetolic acid: (v) said method further comprises a step of extracting said transgenic cell or said transfected cell, thereby obtaining an extract from the transgenic cell or the transfected cell; and (vi) any combination of (i) to (v).

29.-32. (canceled)

33. The method of claim 26, wherein any one of: (i) said cannabinoid is cannabigerolic acid (CBGA), CBCA, or both; (ii) said artificial nucleic acid molecule is an expression vector; (iii) said transgenic cell or said transfected cell is a prokaryote cell or a eukaryote cell; (iv) said transgenic cell or said transfected cell is a C. sativa cell; (v) said method further comprises a step preceding step (a), comprising introducing or transfecting a cell with said artificial nucleic acid molecule, thereby obtaining the transgenic cell or the transfected cell; and (vi) any combination of (i) to (v).

34.-38. (canceled)

39. An extract of a transgenic cell or a transfected cell obtained according to the method of claim 28, optionally wherein said extract comprises a cannabinoid, a precursor thereof, or any combination thereof, and optionally wherein said extract comprises CBGA, CBCA, or both.

40.-41. (canceled)

42. A composition comprising the extract of claim 39, and an acceptable carrier.


SEQUENCE LISTING (144 sequences)

SEQ ID NO: 1 - DNA, 1986 bp, Helicrysum umbraculigerum
  source 1..1986: mol_type = other DNA
atgacgtcgtcaaagaagtttacagttgaagttgaaccggcgattccggccaaggatggaaaaccgtcggctggaccggtttaccgtagtatctttgctaaagacggttttccagctcatattgacggtttagattcatgttgggatattttccgcctatctgtggagaaataccccaataatcgaatgcttggcacccgtgaatttgtgaatggaaagcatggaccatatgtatggtcgacttacaaacaagtatacgacaaggtgataaaggttggaaatgctatccgtgcgtgtggtgtcgagccaggtggtcggtgtgggatctatggtgccaattgtgcagaatggattatgagcatggaggcatgtaatgctcatgggctttactgtgtacctttatacgataccttaggtgctggtgcaattgaattcattctttgccatgccgaggttacaattgcttttgtagaagagaaaaagatccctgagttgttgaaaacatttccgaaagctggagaatttctgaaaacaattgtgagctttggaaaagttactcctgaacaaagagaacaagctgaaaactttggtttaaaaatacattcatgggatgaattcttgacattgggtgatgataaaaactttgacctgccactgaaggaaaaaactgatatctgtacaataatgtacactagtggaacaactggtgatcctaagggtgttctgatttcaaataacagcatggcaacacttatagctggcgtcaatcgtctactagatagtgcaaaagaatctttgaatcaacatgatgtctatctctcgtttttacctctggcacatatatttgaccgtgtgattgaagaatgttttatcaatcatggagcatctataggattctggcgtggggatgttaaattgctgattgaagacataggggagctgaaacctactattttctgcgctgttcctcgagtgttggataggatttattcaggtttgcaacagaaaatttctgcggggggttttatcaaacgtaacttatttaatctagcctattcatacaaattacgtaatatgaagggagggaaaacacattcagaggcatctccattgagtgacaaaatcgtcttcagtaaggttaagcagggcctaggaggaaatgtacgaattattctatctggagctgctccactagctccacatgtagaagcttacctgaaagtagtggcatgtagtcacgtcctgcaaggatatggcctgacagaaacttgtgctggatcatttgtctcactgccaaacgaaatggagatgctgggtacagtgggcccacctgtaccagttttggatgcccgactggagtctgttccggagatgaactatgatgcttgttcaagcaaaccacaaggagaaatatgtattagaggggatgttctgttttcaggatactacaagcgtgaggaccttacaaaagaagtctttgttgatgggtggttccatacaggtgatatcggtgagtggcaaccagatggaagcatgaaaattattgaccgaaagaaaaacatttttaagctctcacaaggagagtacgtcgcagttgaaaatctggagaatgtttatggaaatgtttctgacattgacacgatatggatatatgggaacagcttcgagttttgtcttgttgctgtggtcaacccaaatgagccagcaatcaaacgttatgctgaagcaaataatatttctggggattttgattcattatgtgaaaatcccaaaattaaagaatacatactcggagagctcgctagaattggaaaagagaaaaagttaaaaggttttgaattcgtcaaagctgttcaccttgaccctgtccctttcgacatggaacgtgaccttctgaccccaacattcaagaagaaaaggccccagatgcttaagtactaccaggatgtaattgataacatgtacaagactattaacaagaagtga

SEQ ID NO: 2 - DNA, 1650 bp, Helicrysum umbraculigerum
  source 1..1650: mol_type = other DNA
atggatgcattgaggaagcctaattctgcgaattcaagccctttaactcctatcggattccttgaaagggcagccgtcgtatttgccaactctccttcgatcgtatacaacaatctcatctacacttggagcgatacttttcatcgttgtctacgattagcttcatccatctctcgtctcgctatacgaaaaggcgacgttgtttcagtactcgcaccaaacatccctgccatttatgagcttcattttggcatcactatgactggggccataatcaacaccatcaatacccgtttggatgcgcgtactatctcaatactcctttgtcacagtgaatccaagctcgtctttgttgattaccagttgactcgtcttatacgagaagcggtttctttgatgccagatgcttgtgttcccccacaactcgtcctcatcgtagatgacggacataatctatctttactttctgatcaatttatcaatacttatgaagctatggttgaaacaggggatcctgggttcaattgggttcgtccagatagcgattgggaccctctaacgttgaattacacttctgggacgacttcttcccccaaaggtgttgttaacagccaccgtggatcgttcatagtagcgtttgattctttactggagtggcacgtaccgaaacagccgatcatgctgtggactctaccaatgttccacgcaaatgggtggagcttcgtttggggtatggcagctgttggtggcaccaatgtttgccttcgtaaattcgatgctactattatttatgacaccattcgtaaccaccatgtgacgcacatgtgtggcgcccctgttgtactcaacatgttatcagaaggtaagccacttgaacacacggttcacataatgacagcaggagcaccacctccagcggccgttttgttgcgaaccgagtcgctagggtttgaggtgactcatgggttcgggatgacagaaacaggcgggttagttgtgtcatgctcatggaagaaagaatggaatcgtctgcccgtgactgagaaagcgagattgaaagcgagacaaggagttagaacacttgggatgacggaagtggatattgtggatcccgagtcaggagtaagtgtgactcgagacgggttaactcagggggaattagtgttgcgaggtgggtctattatgttgggttacttaaaagatccggaaacaacaaataaatccgttaaaaacgggtggttttataccggcgacgtggcggtgatgcatccagatggatatctggaaataaaagatagatcaaaagatgtaataataagtggtggtgagaatataagtagtgtggaggttgagtcaatcttgtatcagcatcctgcgattaacgaggccgcggtggtgggacggcctgatgagttttggggcgagtcgccgtgtgctttcgtgagtttgaaagatgataacgggaaggtggctgtgccaacagcggatgagataatgaagttttgtaaaggaaagttgccgggttacatggtacccaaatcggttgtgtttaagaaggatcttccgaagacatctaccggtaagattcagaaatatgtgcttagaaaacttgctaaagatttgggttttgctgtaaaaagtcgaatttag

SEQ ID NO: 3 - DNA, 1440 bp, Helicrysum umbraculigerum
  source 1..1440: mol_type = other DNA
atgaccgaagaggaaaaaaataaagcagagtccatggggataaaaacgtatgcatggagcgacttccttcatctggggagtaaaaatccttcagaactgcaaacgcctaaagcaactgatatatgtacaatcatgtacactagtggcactagtggagacccaaaaggtgttatattgacacatgaaaatgctacaacaaacatacgaggggttgatcttttcatggaacaattcgaggacaagatgaccgtggatgacgtttatatatctttcttgcctcttgctcacattcttgatcgtatgattgaagaatactttttccgtagtggtgcctctgtcggcttctatcatggggatatcaatgcgttgaaggaggatttggcagagctaaagcctacttttttggctggagtacctcgagttttggaaaagattcacgaaggtgtgcttaaaggactagaagaagttaatccaaggagaaggaaaatatttagcattttatacaatcacaaactaaaatacatgaaagcaggttacaagcataaatatgcatcaccacttgcagatctgcttgcttttagaaaggttaagaacaggcttggtgggcgaattcgtcttatggtatctggaggagctccgttaagcactgagattgaagagttcatgagggttacttcatgtgcttttgtggcgcaaggatatggtttgacggaaacatgtggtttggctactttaggatttccagatgagatgtgcatgattggaacagttggttcgcccttcgtgtatacagaattacgcctcgaagaagtttcagatatgggctatgacccgttggccaatccaccacgtggtgaaatatgtgttaagggaaaaacgcctttcgcaggttactacaagaatccagaactcactaatgaggtcatgaaagatgggtggtttcatacaggtgacataggagagatgcaaccaaacggggtattgaaaatcatcgacagaaagaaacatctgataaaactatctcaaggggagtatatcgcgcttgaatatctagagaaagtttactgcatcactcccattcttgaagacatctgggtatatggggatagcttcaagtcatcattggtcgcggtagctgtaccaaacaaagaaaacgcagaaaagtgggccgatcaaaagggccttaaagtttcttactctgagctctgcacactaacacagttcagagattatatccaatctgaactgaaatctaccgcggagagaaacaagctaagaggttttgagcatataaaggctataattgtggagccacggacgtttgaaggagaccaggaattgttgactgcaacaatgaagaaacgtagaaataaactgcttaaccgttacaaggaggggatcgacaacctttacaagaacttggctgcaaacaaacgctga

SEQ ID NO: 4 - DNA, 2181 bp, Helicrysum umbraculigerum
  source 1..2181: mol_type = other DNA
atggtgtacaagtctttgaattcaatatccatatcagatatagtaaatcttggtatatcacctgaaactgcaactcaacttcatcagaaactaactgaaatcattcagatttatggttttgatgctcctcaaacatggacccagatatccacccggattcttcatccggaccttcccttttgttttcatcagatgatgtattatggatgctatgttgattttggaccggatcctcctgcttggtcacccgacccgaaggatgcaaagttaacaaacataggtagtttattagagagacgcggaaaggagttcttggggcctagttataaagatcccatttcaagctactctgctcttcaggaattttcagccttaaatctagaggtgttttggaaaacaatattggatgaaatgaatataacattttctgtgcctccaaaacgcatattagttgatgacctgtctaaagaaagccagttattgcatccaggtggtcgatggcttcccggagcttatgtaaatccagctagaaattgtttgagtttaagtagcaagagaaggttaagtgatatagcagttatatggcgtgatgaaggaaatgatgatatgccggtcaacaaaatgacgtttcagcagttgcgctcagaggtttggttagttgcatatgcacttgatacattgggagtggaaaaaggatctgcaattgcaatcgatatgcctatggatgtcaaatctgtggtgatttatctagccattgttttagcaggctatgtggttgtatctattgcagatagttttgctgctggtgaaatttcgaccagacttgtattatcaaaagcaaaagcaatttttactcaggatttgatcattcgtggtgacagaagccatcccttgtacagccgagttgttgatgctcaatcacctctagcaattgtcattcctacgagaggctcaagttttagtataaaattacgtgacggtgatatttcttggcatgattttctggaacgagctaacacttacaggaatgttgagtttgttgctgttgaacgacccgttgaagctttctcaaatatccttttctcatcaggaactacaggggaaccgaaggcaattccatggacccttgcaacacctttcaaggctggtgcagacgcttggtgccacatggatgtccacaaaggtgatgttgttgcatggcctactaatcttggatggatgatgggtccttggctaatatatgcttcattgttaaatgggggctcacttgcattatacaacggatctcccctgacttctggatttgccaagtttgttcaggatgcaaaagtaacattgttgggagtgataccaagtattgtgagggcatggagaacaaacaatagtacagccggctttgactggtcaaccatccggtgctttggatcgaccggtgaggcctctaatactgatgaatgtctttggctgatgggaagagctcattacaaaccggtcatcgagtattgcggtggcacagagattggtggtggttttattacaggatctttactgcagcctcagtgtttgtctgctttcagcacaccaagtttgggttgtaaactgttaattcttggcgaagatggaatccctataccacaaaacgctcctggaattggtgaattggctctgaatcccctcatgtttggggcatcgagcacactactaaatgcaaaccactatgatgtctactttaaaggcatgccctcttggaatggtaaggttctaagaaggcatggagatgtatttgagcgcacgtctaaaggatactatcgtgcccatggtcgtgcagatgatactatgaatcttgggggtattaaggtaagttcggttgagattgaacgtgtatgcaactcgattgatgacagaattctcgagacagcggctataggggttacaccttctggtggcgggccagagaggttggtaattgttgttgcttttaaagatggcagtggttcgaaacccgacttaatcaagttgaaggtcacactgaattcagctttacaaaagaatctgaaccctttgtttaaggtttctgatgtggtgccctttccatcacttcctaggacagcaacaaacaaggtaatgagaagggttttgcgacagcagttgactcaaattggtcaaaatagcaagctataa

SEQ ID NO: 5 - DNA, 2175 bp, Helicrysum umbraculigerum
  source 1..2175: mol_type = other DNA
atgggtgattcagagggaagcagcattagtactcctacaactgaacaagttggtttcttgtcaaatatcatggaagacaaatcttatagtgctgcagttgcaattatggttgccattgctgtaccgttggttctttcttcagtgtttgcagcgaagaagaaagtgaaacaacgaggcgttcccgttcaagttggtggtgagccaggttttgccatgcgtaactctagatcaaacaaattagttgatgtcccatgggaaggagctagaacaatggctgctctttttgagcagtcttgtaagaagcattcacagcttcggtttcttggtacaaggaagttgattgaaagaagctttgtgagtggtagtgatgggagaaaattcgagaagttacatcttggggagtatcagtgggagacatatgggcagatatttgaacgtgtttgcaactttgcatctggacttattcagcttggtcatgaccctgatactcgtattgccatcttttctgacacacgagctgaatggttaattgcatttgagggatgcttcaggcagaacatcactgtggttaccatatatgcatcattaggtgatgatgccctcattcactctcttaacgagactaaagtatcgaccttgatttgtgattccaaactattgaaaaaagtggctgcagttagttcaagcctgaaaactgtagaaaacttcatctactttgaaagtgacaacactgaagctttaaatgaaatcggtgattggaaaatatcttctttttctgaagtcgagagcttgggacagaagagtccagtaagtgctagactgcctatcaagaaagacgttgcagtgatcatgtatacaagtggcagcacaggtttaccaaagggggtgatgatgactcatgggaatgtagtagcaactgcagctgcggttatgactgtaatcccaaatattgggaccaatgatgtttatctggcatacttaccattggctcatattttcgagttggctgctgagactgtgatggtaactgcaggtattccaattggttatggttcagcactcactttaacagacacatcaaataaaatcaagaaaggaaccttgggagatgcatccatcttgaagccaacgttaatggcagctgttccagctattttagatcgtgtccgagatggagtattaaagaaggttgaggaaaagggaggtttgacaacaaaaatattcaatatagcctacaaaaggcgtttgctagcagtagatggaagttggctgggtgcatgggggttagagaagctattgtgggatgccattgtttttaagaagattcgttctgtacttggaggagatatccgtttcatgctctgtggtggtgctcctttagctgcagatactcagcgatttataaatgtctgcgttggggctccaattggacaaggatatgggctgaccgaaacatgcgctggagctgctttctctgaggcagatgataattctgttgggcgtgttggtccaccacttccttgtgtctatattaaacttgtttcatgggatgaaggtgggtatttaacatcagacaaaccaatgccgcgaggcgaagttgtagttggtgggtacagtgtaaccgctggttactttaataatgaggaaaagaccaatgaggtttacaaggttgatgaaagtgggatgcgttggttctacactggggacattggaaggtttcatcctgatggatgccttgaaatcattgacaggaagaaggatattgtaaaacttcaacatggagagtacatctccttggggaaggttgaggcagcacttgcgtcaagcaagtatgtagagaatgtaatgttacatgccgaccccttccacacttattgtgtcgccttagttgtccctgcgcgtcaggttatagaacagtgggctcaagatgcgggtattagttaccaagattttgctgagttgtgtgataaaaaggaaactgtctctgaggttcagcaatcccttaccaaggtagcaaaagatgcaaaactagacaagtttgaaacgcctgcaaagataaagctgatgccagatccatggactcctgaatctggattagtaacagcggctcttaagttaaaaagggaacaactgaagtccaaatttaaggatgatctggataagctatatgggtga

SEQ ID NO: 6 - DNA, 1980 bp, Helicrysum umbraculigerum
  source 1..1980: mol_type = other DNA
atgtcggtttacaccgttaaagtcgaggattcacgggcagcttccggagaaaccccgtcagcagggccggtttacaggtgcatttatgccaaggatgctctcatggaactgccccccggttatgaatctccctgggacttctttagtgagtctgttaaaagaaacccaaagaacccagcactaggtcgtcgtcaagtcatcgatggaaaggctggtggttattcatggctttcatatcaagaagcctacaattctgctctacgcattgcttctgccatcagaagccgatctgttaatcctggggatcggtgtggtatatatggacctaactgtcctgaatggataatctcaatggaggcttgtaacagcaatggcataacctatgttcccctatatgatacacttggtgctaatgcggttgaatacatcatcaaccatgcagaaatttctttagtttttgttcaagagaacaagttgtctgctattttatcatgtcttccaaattgctcatcaaatcttaaaacaatcgtcagctttgggaagttctctgaatcacaaaagaacgaagccatggaacatggcgtcgattgcttctcttgggaagagttttcttcgatggggaatttggaagatgaacttcctgcaaaaaataagactgacatttgcaccataatgtatacaagtggaacaacgggagagcctaagggtgtcgtactaagtaacagagctttcatgtccgaagtcttgtctatgcatgaactactcatagaaacagacaaaccgggcacagaagaagatacctacttctcttttcttcctttggcacatatatttgatcaaataatggagacgtatttcatctacagtggtgcttcgatagggttttggcaaggagatatcagatacttgattgaagaccttcttgtgttgcagccaaccatattttgtggtgttccaagagtttatgaccgcatttatacgggcataatggctaagatttcaactggaggtgctattcggaaggcattatttgattttgcatacaactataaattaaggaaccttgaaaagggaatacaacaagacaaatcagctcctcttttggacaagctggtcttcgataagattaaacaagggtttggaggaagggttcgtcttatgttatctggagccgcacctttgccaaaacacgtggaggaatttttaagagtgacgtgctgtaccgttctctcacaaggatacggacttactgaaagttgtggtggatgctttacatccattgcgaatgtgtactctatgatcgggactgttggtgtacccatgacaactattgaagcaagacttgagtcagtgccagagatgggatatgatgcactcagtagtgtgccatgtggcgaaatttgcctcaggggaaacacactattttctgggtaccacaaacgagacgatctaactgatgctgtccttgtagatggctggttccatacaggtgacattggggaatggcaggcagatggagcaatgaaaatcattgacaggaaaaagaatatattcaaattgtctcaaggagaatatgttgcagttgaaagtattgaaagcacctattcacggtgtcctttggttacctcgatttgggtgtacggcaatagttttgaatcttttctagttgcggttgtggttcccgatagagtagcagttgaagagtttgctgcaaagaacaatgaatcaggagattatgcatcgttgtgcaagaacccaaatgtcaggaaatatgttcttgaagagctgaatgctgaagctcaatgcaataaacttcgcgggtttgagatgctaaaagcagttcatttggatccagtcccatttgacttcgagagggatttaataacaccaacctttaaactaaaaagacagcagcttctaaaatactataaggattgcgttgaacaactatatgctgaagcaaagacatccaagaaatga

SEQ ID NO: 7 - DNA, 1872 bp, Helicrysum umbraculigerum
  source 1..1872: mol_type = other DNA
atggaaactcatggaccaaggcttctaggtgcagcttacaaagatcctatcacgagttataaacagttccaaaagttctctgttcaacatctagaggtgtattggtctcttgtgttagaaaagctttcaatccaatttcaggaacgtccaaaatgtatagtagatacttctgacaaatcaaaacacgggggcacatggcttcccggttcagttttgaacattgcggagtgttgtatattgtcaactactgaaacagatgaaaaggttgcgattgtgtggcgggatgaaagatgtgataatctggatgtaaacaagatgacattcaaagaattgcgacaacaagtaatgttggttgcaaatgcattgaagttattgttttcaaaaggagatcctattgcaattgatatgccaatgacagttactgcagtaattctatatttggcgattgtatattctggatttgtggttgtatctatagctgacagttttgcagctaaagagattgcaacacgattacgtgtatctaatgcaaaggctatctttactcaagattacattgttcgaggtggtcgaagatttcctttgtacagtcgagttattgaagccacccaatgtagagccatcgtggttcctgcgataggggaaaacgtagaagttattttaagaaaacaggacatttcatggggcgattttctttctggtgcaaaacagcttcctagcccggattattgctctccagtctatcaatccatagacacgttgacaaacatactcttctcttcgggaacaacaggagacccaaaagctataccatggacgcaaatatctccaatgagatgtgctgctgacggatgggctcatatggatattcaggctggagatgtttattgttggcccacaaatctgggatgggtcatgggacccattgtactttactcgagttttcttaccggtgcaacattggctctttataatggctcccctcttggtcatggttttggaaaatttgttcaggatgcaggagtgacaattttgggcacggttccaagcatagtcaagtcttggaagagtacaagatgtatggaaggactggactggacaaagataaaggcatttgggtcgactggtgaagcttctaatgtcgacgatgacctttggctttcctcaaaggcctactacaaacctgttcttgaatgctgtggaggtaccgagcttgcatcttcttatgttcaagggaatcttctacagccacaagcctttggagcattaagctctgcttcaatgggaaccggatttgtcatatttgacgatcatggagttccttacccggacgatgaaccctgtgttggtgaagtgggtttgtttccagtatatatgggagcatctgatagactactgaatgcagatcatgaaaaaatttacttcaagggaatgccgagttacaaaggaatgcaactaaggagacatggagatatcatcaagagaacaattggaggatatttggttgtacaaggcagggctgatgataccatgaaccttggtggcataaagacgagctcaatagaaattgagcgtgtttgtgaacaagctgatggaagcatcatggaaactgctgcagtcagtgttgcacctgcaaccggtggtccagaactattagccatatttgtggtactaaagaacggttgcaacactcaaccacaggacctaaagatgatattttcaaaggccattcaaaaaaacctcaacccattgttcaaggtgagctttgtaaaggttgttccagagttccctcgaaccgcttctaacaagttattgagaagagttttaaggaatcaagtgaaggaagagcttcaaactcgaagtaaaatataa

SEQ ID NO: 8 - DNA, 2082 bp, Helicrysum umbraculigerum
  source 1..2082: mol_type = other DNA
atggagatcactaaaagcatccaagaattaggattacaagatctactaaacactggattaacacctaatgatgcaaaatcactgcaaatcgagattaaacacatcattaatagtcaaactactaattcaaacccagttgagttatggcgtcaaatcacttctgcaaagctgcttaaaccctcttatcctcattcgttgcaccagctcatctactacgcggtgtactgtaactatgatgcatccatctatggtcctcccctgtattggtttccatctgaaattgattctaaaaggtcaaacttggggaacattatggaaactcatggaccaaggcttctaggtgcagcttacaaagatcctatcacgagttataaacagttccaaaagttctctgttcaacatctagaggtgtattggtctcttgtgttagaaaagctttcaatccaatttcaggaacgtccaaaatgtatagtagatacttctgacaaatcaaaacacgggggcacatggcttcccggttcagttttgaacattgcggagtgttgtatattgtcaactagtgaaacagatgataaggttgcgattgtatggcgggatgaaagatgtgataatctggatgtaaacaagatgacattcaaagaattgcgacaacaagtaatgttggttgcaaatgcattgaagttattgttttcaaaaggagatcctattgcaattgatatgccaatgacagttactgcagtaattctatatttggcgattgtatattctggatttgtggttgtatctatagctgacagttttgcagctaaagagattgcaacacgattacgtgtatctaatgcaaaggctatctttactcaagattacattgttcgaggtggtcgaagatttcctttgtacagtcgagttattgaagccacccaatgtagagccatcgtggttcctgcgataggggaaaacgtagaagttattttaagaaaacaggacatttcatggggcgattttctttctggtgcaaaacagcttcctagcccggattattgctctccagtctatcaatccatagacacgttgacaaacatactcttctcttcgggaacaacaggagacccaaaagctataccatggacgcaaatatctccaatgagatgtgctgctgacggatgggctcatatggatattcaggctggagatgtttattgttggcccacaaatctgggatgggtcatgggacccattgtactttactcgagttttcttaccggtgcaacattggctctttataatggctcccctcttggtcatggttttggaaaatttgttcaggatgcaggagtgacaattttgggcacggttccaagcatagtcaagtcttggaagagtacaagatgtatggaaggactggactggacaaagataaaggcatttgggtcgactggtgaagcttctaatgtcgacgatgacctttggctttcctcaaaggcctactacaaacctgttcttgaatgctgtggaggtaccgagcttgcatcttcttatgttcaagggaatcttctacagccacaagcctttggagcattaagctctgcttcaatgggaaccggatttgtcatatttgacgatcatggagttccttacccggacgatgaaccctgtgttggtgaagtgggtttgtttccagtatatatgggagcatctgatagactactgaatgcagatcatgaaaaaatttacttcaagggaatgccgagttacaaaggaatgcaactaaggagacatggagatatcatcaagagaacaattggaggatatttggttgtacaaggcagggctgatgataccatgaaccttggtggcataaagacgagctcaatagaaattgagcgtgtttgtgaacaagctgatggaagcatcatggaaactgctgcagtcagtgttgcacctgcaaccggtggtccagaactattagccatatttgtggtactaaagaacggttgcaacactcaaccacaggacctaaagatgatattttcaaaggccattcaaaaaaacctcaacccattgttcaaggttttctcctaa

SEQ ID NO: 9 - DNA, 2181 bp, Helicrysum umbraculigerum; identical to SEQ ID NO: 4
  source 1..2181: mol_type = other DNA

SEQ ID NO: 10 - DNA, 1560 bp, Helicrysum umbraculigerum
  source 1..1560: mol_type = other DNA
atgacgtttcagcagttgcgctcagaggtttggttagttgcatatgcacttgatacattgggagtggaaaaaggatctgcaattgcaatcgatatgcctatggatgtcaaatctgtggtgatttatctagccattgttttagcaggctatgtggttgtatctattgcagatagttttgctgctggtgaaatttcgaccagacttgtattatcaaaagcaaaagcaatttttactcaggatttgatcattcgtggtgacagaagccatcccttgtacagccgagttgttgatgctcaatcacctctagcaattgtcattcctacgagaggctcaagttttagtataaaattacgtgacggtgatatttcttggcatgattttctggaacgagctaacacttacaggaatgttgagtttgttgctgttgaacgacccgttgaagctttctcaaatatccttttctcatcaggaactacaggggaaccgaaggcaattccatggacccttgcaacacctttcaaggctggtgcagacgcttggtgccacatggatgtccacaaaggtgatgttgttgcatggcctactaatcttggatggatgatgggtccttggctaatatatgcttcattgttaaatgggggctcacttgcattatacaacggatctcccctgacttctggatttgccaagtttgttcaggatgcaaaagtaacattgttgggagtgataccaagtattgtgagggcatggagaacaaacaatagtacagccggctttgactggtcaaccatccggtgctttggatcgaccggtgaggcctctaatactgatgaatgtctttggctgatgggaagagctcattacaaaccggtcatcgagtattgcggtggcacagagattggtggtggttttattacaggatctttactgcagcctcagtgtttgtctgctttcagcacaccaagtttgggttgtaaactgttaattcttggcgaagatggaatccctataccacaaaacgctcctggaattggtgaattggctctgaatcccctcatgtttggggcatcgagcacactactaaatgcaaaccactatgatgtctactttaaaggcatgccctcttggaatggtaaggttctaagaaggcatggagatgtatttgagcgcacgtctaaaggatactatcgtgcccatggtcgtgcagatgatactatgaatcttgggggtattaaggtaagttcggttgagattgaacgtgtatgcaactcgattgatgacagaattctcgagacagcggctataggggttacaccttctggtggcgggccagagaggttggtaattgttgttgcttttaaagatggcagtggttcgaaacccgacttaatcaagttgaaggtcacactgaattcagctttacaaaagaatctgaaccctttgtttaaggtttctgatgtggtgccctttccatcacttcctaggacagcaacaaacaaggtaatgagaagggttttgcgacagcagttgactcaaattggtcaaaatagcaagctataa

SEQ ID NO: 11 - DNA, 1770 bp, Helicrysum umbraculigerum
  source 1..1770: mol_type = other DNA
atgaatataacattttctgtgcctccaaaacgcatattagttgatgacctgtctaaagaaagccagttattgcatccaggtggtcgatggcttcccggagcttatgtaaatccagctagaaattgtttgagtttaagtagcaagagaaggttaagtgatatagcagttatatggcgtgatgaaggaaatgatgatatgccggtcaacaaaatgacgtttcagcagttgcgctcagaggtttggttagttgcatatgcacttgatacattgggagtggaaaaaggatctgcaattgcaatcgatatgcctatggatgtcaaatctgtggtgatttatctagccattgttttagcaggctatgtggttgtatctattgcagatagttttgctgctggtgaaatttcgaccagacttgtattatcaaaagcaaaagcaatttttactcaggatttgatcattcgtggtgacagaagccatcccttgtacagccgagttgttgatgctcaatcacctctagcaattgtcattcctacgagaggctcaagttttagtataaaattacgtgacggtgatatttcttggcatgattttctggaacgagctaacacttacaggaatgttgagtttgttgctgttgaacgacccgttgaagctttctcaaatatccttttctcatcaggaactacaggggaaccgaaggcaattccatggacccttgcaacacctttcaaggctggtgcagacgcttggtgccacatggatgtccacaaaggtgatgttgttgcatggcctactaatcttggatggatgatgggtccttggctaatatatgcttcattgttaaatgggggctcacttgcattatacaacggatctcccctgacttctggatttgccaagtttgttcaggatgcaaaagtaacattgttgggagtgataccaagtattgtgagggcatggagaacaaacaatagtacagccggctttgactggtcaaccatccggtgctttggatcgaccggtgaggcctctaatactgatgaatgtctttggctgatgggaagagctcattacaaaccggtcatcgagtattgcggtggcacagagattggtggtggttttattacaggatctttactgcagcctcagtgtttgtctgctttcagcacaccaagtttgggttgtaaactgttaattcttggcgaagatggaatccctataccacaaaacgctcctggaattggtgaattggctctgaatcccctcatgtttggggcatcgagcacactactaaatgcaaaccactatgatgtctactttaaaggcatgccctcttggaatggtaaggttctaagaaggcatggagatgtatttgagcgcacgtctaaaggatactatcgtgcccatggtcgtgcagatgatactatgaatcttgggggtattaaggtaagttcggttgagattgaacgtgtatgcaactcgattgatgacagaattctcgagacagcggctataggggttacaccttctggtggcgggccagagaggttggtaattgttgttgcttttaaagatggcagtggttcgaaacccgacttaatcaagttgaaggtcacactgaattcagctttacaaaagaatctgaaccctttgtttaaggtttctgatgtggtgccctttccatcacttcctaggacagcaacaaacaaggtaatgagaagggttttgcgacagcagttgactcaaattggtcaaaatagcaagctataa

SEQ ID NO: 12 - protein, 661 aa, Helicrysum umbraculigerum
MTSSKKFTVEVEPAIPAKDGKPSAGPVYRSIFAKDGFPAHIDGLDSCWDIFRLSVEKYPNNRMLGTREFVNGKHGPYVWSTYKQVYDKVIKVGNAIRACGVEPGGRCGIYGANCAEWIMSMEACNAHGLYCVPLYDTLGAGAIEFILCHAEVTIAFVEEKKIPELLKTFPKAGEFLKTIVSFGKVTPEQREQAENFGLKIHSWDEFLTLGDDKNFDLPLKEKTDICTIMYTSGTTGDPKGVLISNNSMATLIAGVNRLLDSAKESLNQHDVYLSFLPLAHIFDRVIEECFINHGASIGFWRGDVKLLIEDIGELKPTIFCAVPRVLDRIYSGLQQKISAGGFIKRNLFNLAYSYKLRNMKGGKTHSEASPLSDKIVFSKVKQGLGGNVRIILSGAAPLAPHVEAYLKVVACSHVLQGYGLTETCAGSFVSLPNEMEMLGTVGPPVPVLDARLESVPEMNYDACSSKPQGEICIRGDVLFSGYYKREDLTKEVFVDGWFHTGDIGEWQPDGSMKIIDRKKNIFKLSQGEYVAVENLENVYGNVSDIDTIWIYGNSFEFCLVAVVNPNEPAIKRYAEANNISGDFDSLCENPKIKEYILGELARIGKEKKLKGFEFVKAVHLDPVPFDMERDLLTPTFKKKRPQMLKYYQDVIDNMYKTINKK

SEQ ID NO: 13 - protein, 549 aa, Helicrysum umbraculigerum
MDALRKPNSANSSPLTPIGFLERAAVVFANSPSIVYNNLIYTWSDTFHRCLRLASSISRLAIRKGDVVSVLAPNIPAIYELHFGITMTGAIINTINTRLDARTISILLCHSESKLVFVDYQLTRLIREAVSLMPDACVPPQLVLIVDDGHNLSLLSDQFINTYEAMVETGDPGFNWVRPDSDWDPLTLNYTSGTTSSPKGVVNSHRGSFIVAFDSLLEWHVPKQPIMLWTLPMFHANGWSFVWGMAAVGGTNVCLRKFDATIIYDTIRNHHVTHMCGAPVVLNMLSEGKPLEHTVHIMTAGAPPPAAVLLRTESLGFEVTHGFGMTETGGLVVSCSWKKEWNRLPVTEKARLKARQGVRTLGMTEVDIVDPESGVSVTRDGLTQGELVLRGGSIMLGYLKDPETTNKSVKNGWFYTGDVAVMHPDGYLEIKDRSKDVIISGGENISSVEVESILYQHPAINEAAVVGRPDEFWGESPCAFVSLKDDNGKVAVPTADEIMKFCKGKLPGYMVPKSVVFKKDLPKTSTGKIQKYVLRKLAKDLGFAVKSRI

SEQ ID NO: 14 - protein, 479 aa, Helicrysum umbraculigerum
MTEEEKNKAESMGIKTYAWSDFLHLGSKNPSELQTPKATDICTIMYTSGTSGDPKGVILTHENATTNIRGVDLFMEQFEDKMTVDDVYISFLPLAHILDRMIEEYFFRSGASVGFYHGDINALKEDLAELKPTFLAGVPRVLEKIHEGVLKGLEEVNPRRRKIFSILYNHKLKYMKAGYKHKYASPLADLLAFRKVKNRLGGRIRLMVSGGAPLSTEIEEFMRVTSCAFVAQGYGLTETCGLATLGFPDEMCMIGTVGSPFVYTELRLEEVSDMGYDPLANPPRGEICVKGKTPFAGYYKNPELTNEVMKDGWFHTGDIGEMQPNGVLKIIDRKKHLIKLSQGEYIALEYLEKVYCITPILEDIWVYGDSFKSSLVAVAVPNKENAEKWADQKGLKVSYSELCTLTQFRDYIQSELKSTAERNKLRGFEHIKAIIVEPRTFEGDQELLTATMKKRRNKLLNRYKEGIDNLYKNLAANKR

SEQ ID NO: 15 - protein, 726 aa, Helicrysum umbraculigerum
MVYKSLNSISISDIVNLGISPETATQLHQKLTEIIQIYGFDAPQTWTQISTRILHPDLPFCFHQMMYYGCYVDFGPDPPAWSPDPKDAKLTNIGSLLERRGKEFLGPSYKDPISSYSALQEFSALNLEVFWKTILDEMNITFSVPPKRILVDDLSKESQLLHPGGRWLPGAYVNPARNCLSLSSKRRLSDIAVIWRDEGNDDMPVNKMTFQQLRSEVWLVAYALDTLGVEKGSAIAIDMPMDVKSVVIYLAIVLAGYVVVSIADSFAAGEISTRLVLSKAKAIFTQDLIIRGDRSHPLYSRVVDAQSPLAIVIPTRGSSFSIKLRDGDISWHDFLERANTYRNVEFVAVERPVEAFSNILFSSGTTGEPKAIPWTLATPFKAGADAWCHMDVHKGDVVAWPTNLGWMMGPWLIYASLLNGGSLALYNGSPLTSGFAKFVQDAKVTLLGVIPSIVRAWRTNNSTAGFDWSTIRCFGSTGEASNTDECLWLMGRAHYKPVIEYCGGTEIGGGFITGSLLQPQCLSAFSTPSLGCKLLILGEDGIPIPQNAPGIGELALNPLMFGASSTLLNANHYDVYFKGMPSWNGKVLRRHGDVFERTSKGYYRAHGRADDTMNLGGIKVSSVEIERVCNSIDDRILETAAIGVTPSGGGPERLVIVVAFKDGSGSKPDLIKLKVTLNSALQKNLNPLFKVSDVVPFPSLPRTATNKVMRRVLRQQLTQIGQNSKL

SEQ ID NO: 16 - protein, 724 aa, Helicrysum umbraculigerum
MGDSEGSSISTPTTEQVGFLSNIMEDKSYSAAVAIMVAIAVPLVLSSVFAAKKKVKQRGVPVQVGGEPGFAMRNSRSNKLVDVPWEGARTMAALFEQSCKKHSQLRFLGTRKLIERSFVSGSDGRKFEKLHLGEYQWETYGQIFERVCNFASGLIQLGHDPDTRIAIFSDTRAEWLIAFEGCFRQNITVVTIYASLGDDALIHSLNETKVSTLICDSKLLKKVAAVSSSLKTVENFIYFESDNTEALNEIGDWKISSFSEVESLGQKSPVSARLPIKKDVAVIMYTSGSTGLPKGVMMTHGNVVATAAAVMTVIPNIGTNDVYLAYLPLAHIFELAAETVMVTAGIPIGYGSALTLTDTSNKIKKGTLGDASILKPTLMAAVPAILDRVRDGVLKKVEEKGGLTTKIFNIAYKRRLLAVDGSWLGAWGLEKLLWDAIVFKKIRSVLGGDIRFMLCGGAPLAADTQRFINVCVGAPIGQGYGLTETCAGAAFSEADDNSVGRVGPPLPCVYIKLVSWDEGGYLTSDKPMPRGEVVVGGYSVTAGYFNNEEKTNEVYKVDESGMRWFYTGDIGRFHPDGCLEIIDRKKDIVKLQHGEYISLGKVEAALASSKYVENVMLHADPFHTYCVALVVPARQVIEQWAQDAGISYQDFAELCDKKETVSEVQQSLTKVAKDAKLDKFETPAKIKLMPDPWTPESGLVTAALKLKREQLKSKFKDDLDKLYG

SEQ ID NO: 17 - protein, 659 aa, Helicrysum umbraculigerum
MSVYTVKVEDSRAASGETPSAGPVYRCIYAKDALMELPPGYESPWDFFSESVKRNPKNPALGRRQVIDGKAGGYSWLSYQEAYNSALRIASAIRSRSVNPGDRCGIYGPNCPEWIISMEACNSNGITYVPLYDTLGANAVEYIINHAEISLVFVQENKLSAILSCLPNCSSNLKTIVSFGKFSESQKNEAMEHGVDCFSWEEFSSMGNLEDELPAKNKTDICTIMYTSGTTGEPKGVVLSNRAFMSEVLSMHELLIETDKPGTEEDTYFSFLPLAHIFDQIMETYFIYSGASIGFWQGDIRYLIEDLLVLQPTIFCGVPRVYDRIYTGIMAKISTGGAIRKALFDFAYNYKLRNLEKGIQQDKSAPLLDKLVFDKIKQGFGGRVRLMLSGAAPLPKHVEEFLRVTCCTVLSQGYGLTESCGGCFTSIANVYSMIGTVGVPMTTIEARLESVPEMGYDALSSVPCGEICLRGNTLFSGYHKRDDLTDAVLVDGWFHTGDIGEWQADGAMKIIDRKKNIFKLSQGEYVAVESIESTYSRCPLVTSIWVYGNSFESFLVAVVVPDRVAVEEFAAKNNESGDYASLCKNPNVRKYVLEELNAEAQCNKLRGFEMLKAVHLDPVPFDFERDLITPTFKLKRQQLLKYYKDCVEQLYAEAKTSKK

SEQ ID NO: 18 - protein, 623 aa, Helicrysum umbraculigerum
METHGPRLLGAAYKDPITSYKQFQKFSVQHLEVYWSLVLEKLSIQFQERPKCIVDTSDKSKHGGTWLPGSVLNIAECCILSTTETDEKVAIVWRDERCDNLDVNKMTFKELRQQVMLVANALKLLFSKGDPIAIDMPMTVTAVILYLAIVYSGFVVVSIADSFAAKEIATRLRVSNAKAIFTQDYIVRGGRRFPLYSRVIEATQCRAIVVPAIGENVEVILRKQDISWGDFLSGAKQLPSPDYCSPVYQSIDTLTNILFSSGTTGDPKAIPWTQISPMRCAADGWAHMDIQAGDVYCWPTNLGWVMGPIVLYSSFLTGATLALYNGSPLGHGFGKFVQDAGVTILGTVPSIVKSWKSTRCMEGLDWTKIKAFGSTGEASNVDDDLWLSSKAYYKPVLECCGGTELASSYVQGNLLQPQAFGALSSASMGTGFVIFDDHGVPYPDDEPCVGEVGLFPVYMGASDRLLNADHEKIYFKGMPSYKGMQLRRHGDIIKRTIGGYLVVQGRADDTMNLGGIKTSSIEIERVCEQADGSIMETAAVSVAPATGGPELLAIFVVLKNGCNTQPQDLKMIFSKAIQKNLNPLFKVSFVKVVPEFPRTASNKLLRRVLRNQVKEELQTRSKI

SEQ ID NO: 19 - protein, 693 aa, Helicrysum umbraculigerum
MEITKSIQELGLQDLLNTGLTPNDAKSLQIEIKHIINSQTTNSNPVELWRQITSAKLLKPSYPHSLHQLIYYAVYCNYDASIYGPPLYWFPSEIDSKRSNLGNIMETHGPRLLGAAYKDPITSYKQFQKFSVQHLEVYWSLVLEKLSIQFQERPKCIVDTSDKSKHGGTWLPGSVLNIAECCILSTSETDDKVAIVWRDERCDNLDVNKMTFKELRQQVMLVANALKLLFSKGDPIAIDMPMTVTAVILYLAIVYSGFVVVSIADSFAAKEIATRLRVSNAKAIFTQDYIVRGGRRFPLYSRVIEATQCRAIVVPAIGENVEVILRKQDISWGDFLSGAKQLPSPDYCSPVYQSIDTLTNILFSSGTTGDPKAIPWTQISPMRCAADGWAHMDIQAGDVYCWPTNLGWVMGPIVLYSSFLTGATLALYNGSPLGHGFGKFVQDAGVTILGTVPSIVKSWKSTRCMEGLDWTKIKAFGSTGEASNVDDDLWLSSKAYYKPVLECCGGTELASSYVQGNLLQPQAFGALSSASMGTGFVIFDDHGVPYPDDEPCVGEVGLFPVYMGASDRLLNADHEKIYFKGMPSYKGMQLRRHGDIIKRTIGGYLVVQGRADDTMNLGGIKTSSIEIERVCEQADGSIMETAAVSVAPATGGPELLAIFVVLKNGCNTQPQDLKMIFSKAIQKNLNPLFKVFS

SEQ ID NO: 20 - protein, 726 aa, Helicrysum umbraculigerum; identical to SEQ ID NO: 15

SEQ ID NO: 21 - protein, 519 aa, Helicrysum umbraculigerum
MTFQQLRSEVWLVAYALDTLGVEKGSAIAIDMPMDVKSVVIYLAIVLAGYVVVSIADSFAAGEISTRLVLSKAKAIFTQDLIIRGDRSHPLYSRVVDAQSPLAIVIPTRGSSFSIKLRDGDISWHDFLERANTYRNVEFVAVERPVEAFSNILFSSGTTGEPKAIPWTLATPFKAGADAWCHMDVHKGDVVAWPTNLGWMMGPWLIYASLLNGGSLALYNGSPLTSGFAKFVQDAKVTLLGVIPSIVRAWRTNNSTAGFDWSTIRCFGSTGEASNTDECLWLMGRAHYKPVIEYCGGTEIGGGFITGSLLQPQCLSAFSTPSLGCKLLILGEDGIPIPQNAPGIGELALNPLMFGASSTLLNANHYDVYFKGMPSWNGKVLRRHGDVFERTSKGYYRAHGRADDTMNLGGIKVSSVEIERVCNSIDDRILETAAIGVTPSGGGPERLVIVVAFKDGSGSKPDLIKLKVTLNSALQKNLNPLFKVSDVVPFPSLPRTATNKVMRRVLRQQLTQIGQNSKL

SEQ ID NO: 22 - protein, 589 aa, Helicrysum umbraculigerum
MNITFSVPPKRILVDDLSKESQLLHPGGRWLPGAYVNPARNCLSLSSKRRLSDIAVIWRDEGNDDMPVNKMTFQQLRSEVWLVAYALDTLGVEKGSAIAIDMPMDVKSVVIYLAIVLAGYVVVSIADSFAAGEISTRLVLSKAKAIFTQDLIIRGDRSHPLYSRVVDAQSPLAIVIPTRGSSFSIKLRDGDISWHDFLERANTYRNVEFVAVERPVEAFSNILFSSGTTGEPKAIPWTLATPFKAGADAWCHMDVHKGDVVAWPTNLGWMMGPWLIYASLLNGGSLALYNGSPLTSGFAKFVQDAKVTLLGVIPSIVRAWRTNNSTAGFDWSTIRCFGSTGEASNTDECLWLMGRAHYKPVIEYCGGTEIGGGFITGSLLQPQCLSAFSTPSLGCKLLILGEDGIPIPQNAPGIGELALNPLMFGASSTLLNANHYDVYFKGMPSWNGKVLRRHGDVFERTSKGYYRAHGRADDTMNLGGIKVSSVEIERVCNSIDDRILETAAIGVTPSGGGPERLVIVVAFKDGSGSKPDLIKLKVTLNSALQKNLNPLFKVSDVVPFPSLPRTATNKVMRRVLRQQLTQIGQNSKL

SEQ ID NO: 23 - DNA, 1197 bp, Helicrysum umbraculigerum
  source 1..1197: mol_type = other DNA
atggcatcctcaattaatatctccaagatcagagaggctcaacgagcacaaggtccagcctctattcttgctgtcggtaccgcgaatccgtctaattgcgtgtatcaagctgattatcctgattactactttcgaatcactaaaagtgaacacatggttgatctcaaacggaaattcaagcgcatgtgtgaccaatctatgataagaaagcggtacatgcaaattacggaggagtatctgaaagaaaaccccaacatttgtgaatacatggctccatcacttgacgcccgtcaagacgttgtagtcgtcgaagtcccaaaactcggtaaagaagccgcaacaaaagccatcaaagaatggggccaaccaaaatccaaaattacccatctcatcttttgtaccacgtccggtgtcgacatgcccggagcagattaccagctcaccaaactcctcggtctttgtccttcagtcaaacgctttatgatgtaccaacaaggttgttttgctggtggcacggttcttcgtctagctaaggacatcgctgagaacaataaaggtgctcgtgtacttgtcgtttgttccgagattacagctgtcatttttcgtggacccaacgacactcaccttgattcacttatcggtcaagcgttatttggggatggggcatcttcggttatcgtggggtctgacccagacttgacaaccgagcggccattgtttgaaatcatatcggctgcacaaacgattttaccggactctgaaggtgcgatagatggacacttgagggaagctgggttaacttttcatctacttaaagacgtaccgaggttgatttcgaagaatatagagaaagctttaacacaagcattttctcccctgggaattagtgactggaactctatcttttgggtcacgcaccctggtggtccagctatactggaccaagtggaactcaaacttggactcaaagaggagaagatgagaaccactagacatgttctcagtgaatatgggaacatgtctagtgcatgtgttttttttgtacttgatgaaatgagaaagagatcggctaaaggcggtgcgaggaccaccggagaagggttagattggggtgttctgtttgggtttggtccgggtttaacggttgagactgtggtccttcatagtctcccaactactatgtcgattgcgacttaa

SEQ ID NO: 24 - DNA, 1197 bp, Helicrysum umbraculigerum
  source 1..1197: mol_type = other DNA
atggcatcctcaattaatatctccaagatcagagaggctcaacgagcacaaggtccagcctctattcttgctgtcggtactgcgaatccgtctaattgtgtgtatcaagctgattatcctgattactactttcgaatcactaaaagtgaacacatggttgatttgaaagagaaattccagcgcatgtgtgacaaatctatgataagaaagcggcacattcacattacggaggagtttttgaaagaaaacccaaacctttgtgaatacatggctccatcacttgacacccgtcaagacgttgtagtcgtcgaagtcccaaaactcggtaaagaagccgcaacaaaagccatcaaagaatggggccaaccaaaatccaaaattacccatctcatcttttgtaccacgtccggtgtcgacatgcccggagcagattaccagctcaccaaactcctcggtctccatccttcagtcaaacgctttatgatgtaccaacaaggttgttttgctggtggcacggttcttcgtctagctaaggacctcgctgagaacaataaaggtgctcgtgtacttgccgtttgttccgagattacagctgtcacgtttcgtggacccaacgacactcacattgattcacttgtcggtcaagcattatttggggacggggcagctgcggttatcgtggggtctgatcctgacttgacaactgagcggccgttgtttgaaatcatatcggctgcacaaacgattttaccgaactctgaaggtgcgatagatggacatgtgagggaagttggggtaactattcatatacttaaagacgtcccggtgttgatttcgaagaatatagagaaagctttaacacaagcattttctcccttaggaattagtgactggaactcgatcttttgggtcgtacaccctggtggtccagctatactggaccaagtggaactcaaacttggactcaaagaggagaaaatgagaaccactagacatgttctcagtgaatatgggaacatgtctagtgcatgtgttttttttgtacttgatgaaatgagaaagagatcggctaaaggcggtgcgaggaccaccggagaagggttagattggggtgttctgtttgggtttggtccaggtttaacggttgagacggtggtccttcatagtctcccaactactatgtcgattgcaacttaa

SEQ ID NO: 25 - DNA, 1197 bp, Helicrysum umbraculigerum
  source 1..1197: mol_type = other DNA
atggcatcctcaattaatatctccaagatcagagaggctcaacgagcacaaggtccagcctctattcttgctgtcggtaccgcgaatccgtctaattgcgtgtatcaagctgattatcctaattactactttcgaatcactaaaagtgaacacatggttgatctcaaacggaaattcaagcgcatgtgtgaccaatctatgataagaaagcggtacatgcaaattacggaggagtatctgaaagaaaaccccaacatttgtgaatacatggctccatcacttgacgcccgtcaagacgttgtagtcgtcgaagtcccaaaactcggtaaagaagccgcaacaaaagccatcaaagaatggggccaaccaaaatccaaaattacccatctcatcttttgtaccacgtccggtgtcgacatgcccggagcagattaccagctcaccaaactcctcggtctctgtccttcagtcaaacgctttatgatgtaccaacaaggttgttttgctggtggcacggttcttcgtctagctaaggacatcgctgagaacaataaaggtgctcgtgtacttgtcgtttgttccgagattacagctgtcatttttcgtggacccaacgacactcaccttgattcacttatcggtcaagcgttatttggggatggggcatcttcggttatcgtggggtctgacccagacttgacaaccgagcggccattgtttgaaatcatatcggctgcacaaacgattttaccggactctgaaggtgcgatagatggacacttgagggaagctgggttaacttttcatctacttaaagacgtaccggggttgatttcgaagaatatagagaaagctttaacacaagcattttctcccttgggaattagtgactggaactctatcttttgggtcacgcaccctggtggtccagctatactggaccaagtggaactcaaacttggactcaaagaggagaagatgagagcctctagacatgttctcagtgaatacgggaacatgtctagtgcatgtgttttttttatacttgatgaaatgagaaagaaatcggatgaagatggtgcgccgaccactggagaagggttagattggggtgttctgtttgggtttggtccgggtttaacggttgagacggtggtccttcatagtctcccaactactatgtcgattgcgacttaa

SEQ ID NO: 26 - DNA, 1185 bp, Helicrysum umbraculigerum
  source 1..1185: mol_type = other DNA
atggcatcctcaattaatatctctaagatcagagaggctcaacgagcacaaggtccagcctctattcttgctgtcggtactgcgaatccatctaattatgagattcaagctgattttcctgattactactttcgagtcactaaaagtgaacacatggctgatatgaaagggacattccagcgcatgtgtgacaaatctatgataagaaagcggcacatgctcattacggaggagtttttgaaagaaaacccaaacctttgtgaatacatggctccatcacttgacacccgtcaagacgttgtagtcgtcgaagtcccaaaactcggtaaagaagccgcaacaaaagccatcaaagaatggggccaaccaaaatccaaaattacccatctcatcttttgtactacaactggtgtcgacatgcctggagccgattaccagctcaccaagctcctcggcctcgctccttcagtcaaacgctttatgatataccaacaaggttgttttgctggtggcacggttcttcgtcttgctaaagacatagctgagaacaataaaggtgctcgtgtacttgccgtatgttcagagattacagctatgtcgtttcgtgggcccaatgacactcacgttgattcacttgtcggtcaagcattatttggggacggggcagctgcagttatcgtggggtctgatcctgacttgacaaccgagcggccgttgtttgaaatcatatcggctgcacaaacgattttaccaaactctgaaggtgcgatagatggacatgtgagggaagttggtttaactattcatatacttaaagacgtcccggtgttgatatcgaagaatatagagaaagctttgacacaagcattttctcccttaggaattagtgactggaactcgatcttttggatcgtacaccctggtggtccagctatactggaccaagtggaactcaaagttggactcaaaaaggagaaaatggcaaccagtagacatgttctaagtgaatacgggaacatgtctagtgcatgtgttttttttataatggatgaaatgagaaagagatcggctaaaggcggtgcgaggaccaccggagaagggttagattggggtgttttgtttgggtttggtccaggtttaacggttgagacggtggtccttcatagtctcccaactacaatgtag

SEQ ID NO: 27 - protein, 398 aa, Helicrysum umbraculigerum
MASSINISKIREAQRAQGPASILAVGTANPSNCVYQADYPDYYFRITKSEHMVDLKRKFKRMCDQSMIRKRYMQITEEYLKENPNICEYMAPSLDARQDVVVVEVPKLGKEAATKAIKEWGQPKSKITHLIFCTTSGVDMPGADYQLTKLLGLCPSVKRFMMYQQGCFAGGTVLRLAKDIAENNKGARVLVVCSEITAVIFRGPNDTHLDSLIGQALFGDGASSVIVGSDPDLTTERPLFEIISAAQTILPDSEGAIDGHLREAGLTFHLLKDVPRLISKNIEKALTQAFSPLGISDWNSIFWVTHPGGPAILDQVELKLGLKEEKMRTTRHVLSEYGNMSSACVFFVLDEMRKRSAKGGARTTGEGLDWGVLFGFGPGLTVETVVLHSLPTTMSIAT

SEQ ID NO: 28 - protein, 398 aa, Helicrysum umbraculigerum
MASSINISKIREAQRAQGPASILAVGTANPSNCVYQADYPDYYFRITKSEHMVDLKEKFQRMCDKSMIRKRHIHITEEFLKENPNLCEYMAPSLDTRQDVVVVEVPKLGKEAATKAIKEWGQPKSKITHLIFCTTSGVDMPGADYQLTKLLGLHPSVKRFMMYQQGCFAGGTVLRLAKDLAENNKGARVLAVCSEITAVTFRGPNDTHIDSLVGQALFGDGAAAVIVGSDPDLTTERPLFEIISAAQTILPNSEGAIDGHVREVGVTIHILKDVPVLISKNIEKALTQAFSPLGISDWNSIFWVVHPGGPAILDQVELKLGLKEEKMRTTRHVLSEYGNMSSACVFFVLDEMRKRSAKGGARTTGEGLDWGVLFGFGPGLTVETVVLHSLPTTMSIAT

SEQ ID NO: 29 - protein, 398 aa, Helicrysum umbraculigerum
MASSINISKIREAQRAQGPASILAVGTANPSNCVYQADYPNYYFRITKSEHMVDLKRKFKRMCDQSMIRKRYMQITEEYLKENPNICEYMAPSLDARQDVVVVEVPKLGKEAATKAIKEWGQPKSKITHLIFCTTSGVDMPGADYQLTKLLGLCPSVKRFMMYQQGCFAGGTVLRLAKDIAENNKGARVLVVCSEITAVIFRGPNDTHLDSLIGQALFGDGASSVIVGSDPDLTTERPLFEIISAAQTILPDSEGAIDGHLREAGLTFHLLKDVPGLISKNIEKALTQAFSPLGISDWNSIFWVTHPGGPAILDQVELKLGLKEEKMRASRHVLSEYGNMSSACVFFILDEMRKKSDEDGAPTTGEGLDWGVLFGFGPGLTVETVVLHSLPTTMSIAT

SEQ ID NO: 30 - protein, 394 aa, Helicrysum umbraculigerum
MASSINISKIREAQRAQGPASILAVGTANPSNYEIQADFPDYYFRVTKSEHMADMKGTFQRMCDKSMIRKRHMLITEEFLKENPNLCEYMAPSLDTRQDVVVVEVPKLGKEAATKAIKEWGQPKSKITHLIFCTTTGVDMPGADYQLTKLLGLAPSVKRFMIYQQGCFAGGTVLRLAKDIAENNKGARVLAVCSEITAMSFRGPNDTHVDSLVGQALFGDGAAAVIVGSDPDLTTERPLFEIISAAQTILPNSEGAIDGHVREVGLTIHILKDVPVLISKNIEKALTQAFSPLGISDWNSIFWIVHPGGPAILDQVELKVGLKKEKMATSRHVLSEYGNMSSACVFFIMDEMRKRSAKGGARTTGEGLDWGVLFGFGPGLTVETVVLHSLPTTM

SEQ ID NO: 31 - DNA, 321 bp, Helicrysum umbraculigerum
  source 1..321: mol_type = other DNA
atggcggagttcacacatttagtggtggttaagttcaaagaagaggtggttgtggaggatattatgaaagggttggagaaacttgtatctcaacttgatagtgtcaagtcctttgtttggggaaaggatattgaaagcatggagatgttaaggcaaggattcacccatgcaatcatgatgacatttggttctaaagaagattttactgcatttcaatcccacccaaaccatgttgaattctcggctacgttttcagcagcaatcgaaaagatcgttcttcttgatttcccagttgttgctgtcaagactgcaactgcttga

SEQ ID NO: 32 - DNA, 651 bp, Helicrysum umbraculigerum
  source 1..651: mol_type = other DNA
atgtcgtccttacaaaacaaatttatcgaacacattgctcttatcaaaatcaaacccggtgttgagtctaccaccttgatagataaactcaacggcctttcttcgattgaggtgttactgcacttcagcgcgggtgaactcctgggatcatcccacggcttcactcacatcgttcactgccgtgtcagatcaaaggatgatctccaaatctaccttacacatcctatccacttgcatctggctgatgatactttacccttacttgatgacgtcaccgtcgttgactggttttcatccaactctgatattgtggatcctcctaaaccaggatctgcaatgagagttacgctgctgaagttgaaacacgattcgactgaaagtaataagttagtagtgattgaaggaattaaaaatcagtttaaaggaattgaagacgtgatagttacaactacttttggtgagaatttgtttcatgaaatgcatgagaatttctcgattgaaattgacaaaggatactcgattggttcgattgcctttgttcctggatctgcagatttccaggttttaaattcaaaggtagataataataaactcaatgatttaacagaaagtgaagtggtggttgattatgtgtttccatcagccaattaa

SEQ ID NO: 33 - DNA, 639 bp, Helicrysum umbraculigerum
  source 1..639: mol_type = other DNA
atgtcctctgaagagcagatcgtggaacacgtggtcctgttcaaagtgaaacctgatgctgatcctagtaaagtcgcggcttgggtcaatgggctcaacggtttgacctcactccagctcgccctccacctctccgctggacaactcatccggtgtcggtcgtcgtcgctcaccttcactcacatgcttcacagtcgttacagatcaaaggagcatctccggcagtacaccgttcatcccgagcacgtgcgcgtggttacagagggtaaatccatcattgatgacgtcatggcccttgattggatgatatctaacggcgctgctagtagcgtctgtcctaagcctggatcagcggtgagagttgggttttataagttaatggagagtttgggggaaattgagaaagctagggttttggaagtgatgggagggattgaagagttaagtgttggtgagagtttttgtgatgacagggccaagggttatacgattgcttcaaccgccgtgtttcccaatggcaatcctgctgctgatttggatttatatcattccggtgaccagctcctgctgaaagaggaagtgatgaaggattctatacaaagtgtggtggttgttgattacgtaattccatctccctga

SEQ ID NO: 34 - DNA, 318 bp, Helicrysum umbraculigerum
  source 1..318: mol_type = other DNA
atgggagaagtgaagcacatacttttagcgaagtttaaggatggaatctcggaacaacagatccagcatctcatcacaggttatgctaacctcgtcaatctcgttgaacccatgaagtcttttcgatggggaaaagatgtgagcattgagaatctgcaccaaggctttactcatgtgttcgagtcaacctttgaaaccactgaaggcattgcaacttatatatctcatcctgctcatgtcgagttcgccactggtttcctggatcaactggaaaaagtcatagtcatcgactacaaacctacatcagttgacccgtga

SEQ ID NO: 35 - DNA, 789 bp, Helicrysum umbraculigerum
  source 1..789: mol_type = other DNA
atgctatgtgctccagcacgcacacgattacttccatcaatttctctcttaccttcccaacataacatcttccgccgcctgaactgtctcatccaccgtcgcaaccaccaccaaacgccgatcacgatgtctgctcaacaacaaatcgtggaacacgtagtgctcttcaaagtaaaaccggatgttgattctagtaaagttgctgcaatggtcaacggactcaacggattgacctcactcgatcttactctccacctctccgccggacagctcctccggtcacggtcatcatcgctgaccttcactcacatgcttcacagtcgttacagatcaaaggacgatctccgggagtacgctgctcatcctgaccacgtgcgagtcgtgacggagaatataaaaccggttattgatgatatcatggctgttgattggatatctaacgatgccagtgtatcgcctaagccagggtcggcgatgagagtaacatttttgaaattaaaggagaatttgggggaaaatgagaaatctagggttttggaagtgattggaggaatcaaaaatcagtttaaatcaattgaggagttaagtgttggtgagaatttttctcatgatagagccaaggggtatacgattgcttcaattgctgtgttacccgggccttccgagctggaggcattggattcgaatactgagctggtgaagttggaaaaggagaaagtgaaggacttactggagagcgttgtggttgttgattatgtgattccatctctgcaatcggctagtctttaa

SEQ ID NO: 36 - DNA, 417 bp, Helicrysum umbraculigerum
  source 1..417: mol_type = other DNA
atggcagttgctcaactttcttcctccctctgtatctccacacccgctagaatctctactggttctgggttttcgtcatcaggtttgcctcggattgggacaacgtttgtatgcggttcaggttcgcctcttgtgatatctggaacatatcatcagaaggctcgagtacataagcctgcagcattatctgtgagatgtgaacaaagtagtaaggatggaaatggtttaaatgtgtggcttggtcgaacagcaatggttggctttgcagtggcaattagtgttgaagtatcaactgggaaggggcttcttgagaactttgggctcacatcacccttgccaacagtggccttggcactgactgcacttgggggcgttcttacagcacttttcatcttccagtctgcttctgagagttga

SEQ ID NO: 37 - DNA, 612 bp, Helicrysum umbraculigerum
  source 1..612: mol_type = other DNA
atgattgaacacatagtcctcctcaaatttaaatccgacgtcgactctaccaaagtcgagtccatgattaacgaactcaacggattggcttcactcgatgttgcactcgacgtgagtgccggtaaaatcctgcgagtgagtagtacatcatcctcttctctcactttcacccacctctttcgctgttgtttcagatcagccgatgatcagcaagtcttctctactcatcctgaccatctacgagtggccattgaagttcgacccgtaattgaagatatggtagttgttgacttggtatccaaaactacaattgactcaccaaacccaggatctgcaatgaaagttaggatatttaagttgaaagacgatctgatcgaagatagtaagttagtagtgatggaaggaattaaaaatgagttaaaagcagttgaacatattaggtttggtgacaacattaatgttatggcaaagggatactcgattgctatgattgctttttttcctgatttggaatcttcggttgcaggtgcagaaattgttaaggattatatagagagcgagctggtggtggattttgtgtttccaccaccaaacgttacaagtcattcatga

SEQ ID NO: 38 - DNA, 321 bp, Helicrysum umbraculigerum
  source 1..321: mol_type = other DNA
atggcggagttcacacatttagtggtggttaagttcaaagaagaggtggttgtagaggatattatgaaagggttggagaaacttgcatctcaacttgatagtgtcaagtcctttgtttggggaaaggatattgaaagcatggagatgttaaggcaaggattcacccatgcaatcatgatgacatttggttctaaagaagattttactgcatttcaatcccacccaaaccatgttgaattctcggctacgttttcagcagcaatcgaaaagatcgttcttcttgatttcccagttgttgcagtcaagactgcaactgcttga

SEQ ID NO: 39 - protein, 106 aa, Helicrysum umbraculigerum
MAEFTHLVVVKFKEEVVVEDIMKGLEKLVSQLDSVKSFVWGKDIESMEMLRQGFTHAIMMTFGSKEDFTAFQSHPNHVEFSATFSAAIEKIVLLDFPVVAVKTATA

SEQ ID NO: 40 - protein, 216 aa, Helicrysum umbraculigerum
MSSLQNKFIEHIALIKIKPGVESTTLIDKLNGLSSIEVLLHFSAGELLGSSHGFTHIVHCRVRSKDDLQIYLTHPIHLHLADDTLPLLDDVTVVDWFSSNSDIVDPPKPGSAMRVTLLKLKHDSTESNKLVVIEGIKNQFKGIEDVIVTTTFGENLFHEMHENFSIEIDKGYSIGSIAFVPGSADFQVLNSKVDNNKLNDLTESEVVVDYVFPSAN

SEQ ID NO: 41 - protein, 212 aa, Helicrysum umbraculigerum
MSSEEQIVEHVVLFKVKPDADPSKVAAWVNGLNGLTSLQLALHLSAGQLIRCRSSSLTFTHMLHSRYRSKEHLRQYTVHPEHVRVVTEGKSIIDDVMALDWMISNGAASSVCPKPGSAVRVGFYKLMESLGEIEKARVLEVMGGIEELSVGESFCDDRAKGYTIASTAVFPNGNPAADLDLYHSGDQLLLKEEVMKDSIQSVVVVDYVIPSP

SEQ ID NO: 42 - protein, 105 aa, Helicrysum umbraculigerum
MGEVKHILLAKFKDGISEQQIQHLITGYANLVNLVEPMKSFRWGKDVSIENLHQGFTHVFESTFETTEGIATYISHPAHVEFATGFLDQLEKVIVIDYKPTSVDP

SEQ ID NO: 43 - protein, 262 aa, Helicrysum umbraculigerum
MLCAPARTRLLPSISLLPSQHNIFRRLNCLIHRRNHHQTPITMSAQQQIVEHVVLFKVKPDVDSSKVAAMVNGLNGLTSLDLTLHLSAGQLLRSRSSSLTFTHMLHSRYRSKDDLREYAAHPDHVRVVTENIKPVIDDIMAVDWISNDASVSPKPGSAMRVTFLKLKENLGENEKSRVLEVIGGIKNQFKSIEELSVGENFSHDRAKGYTIASIAVLPGPSELEALDSNTELVKLEKEKVKDLLESVVVVDYVIPSLQSASL

SEQ ID NO: 44 - protein, 138 aa, Helicrysum umbraculigerum
MAVAQLSSSLCISTPARISTGSGFSSSGLPRIGTTFVCGSGSPLVISGTYHQKARVHKPAALSVRCEQSSKDGNGLNVWLGRTAMVGFAVAISVEVSTGKGLLENFGLTSPLPTVALALTALGGVLTALFIFQSASES

SEQ ID NO: 45 - protein, 203 aa, Helicrysum umbraculigerum
MIEHIVLLKFKSDVDSTKVESMINELNGLASLDVALDVSAGKILRVSSTSSSSLTFTHLFRCCFRSADDQQVFSTHPDHLRVAIEVRPVIEDMVVVDLVSKTTIDSPNPGSAMKVRIFKLKDDLIEDSKLVVMEGIKNELKAVEHIRFGDNINVMAKGYSIAMIAFFPDLESSVAGAEIVKDYIESELVVDFVFPPPNVTSHS

SEQ ID NO: 46 - protein, 106 aa, Helicrysum umbraculigerum
MAEFTHLVVVKFKEEVVVEDIMKGLEKLASQLDSVKSFVWGKDIESMEMLRQGFTHAIMMTFGSKEDFTAFQSHPNHVEFSATFSAAIEKIVLLDFPVVAVKTATA

SEQ ID NO: 47 - DNA, 1200 bp, Helicrysum umbraculigerum
  source 1..1200: mol_type = other DNA
atggagttatcactctcatcatcttcttcttcatcccttccccaacttcatactcatccttcatcatcatcatcttcttcacattacataaaaaaatcacctttttttattaataaattcaataatcacaccaaatgcaaattccacaattcctctgctctgagaactaatttcttctacactaccataactaaaacctcatcatcaagattcgttctaaacaaaaacccaaaccaattttccgtcaaggcttgcagtcaagttggttctgctggatccgatccagcattgaataaagttgcagactttaaagatgcattttggaggtttctaaggccccatactattcgtgggacagcattaggatcagtgtctttagtaacgagagcactacttgaaaacccaaacttgattcggtggtcacttttgctcaaggcattttcaggtcttgttgctttgatatgtgggaatggttatatagtcgggatcaatcagatctatgatatcggtattgataaggtgaacaaaccatatttacctattgctgcgggagatctttctgtccagtcagcatggtttttggtgttagcatttgcaatggtaggcgttattattgttgggatgaacttcggcccattcatcacctccctttattctctcggtcttttcttgggcaccatctattccgttccaccacttcgaatgaagagatttcctgttgttgcatttcttatcatcgccacggtgagaggttttcttctaaattttggtgtgtattatgcggttagagcagctctgggactaacattccaatggagctcagcagtggcttttatcacaaccttcgttacattatttgctttagtcattgccattactaaagatcttcctgatgtagagggtgaccgaaagtttcaaatttctacttttgcaacaaaacttggagtaagaaacattgcattattagggtcaggacttctgctgatcaattatattgggtctatcgttgcagcactttacatgcctcaggctttcaggagcagcttgatgataccattacataccatattagcttcctgtttgatttaccaggcatggatacttgagcgtgcgaattacacccaggaggcgatagctgggtactaccgatttgtatggaatctgttttattcagagtacatcatatttcctttcatctga

SEQ ID NO: 48 - DNA, 1119 bp, Helicrysum umbraculigerum
  source 1..1119: mol_type = other DNA
atggctactatggcttcttctttgctgaatcctctttcttgttccattaaacccaactcaaacagactaccattaccaacacccatttctctatctcgttcttgtagaaggctaacaatcaaagcaacggagacagatgcaaatgaagtgaagccaaaggcgccagagaaagcaccagctgcaagtggatctggttttaatcaaattcttgggattaaaggggctaaacaagaaactaataaatggaagatccgtgttcaacttacaaagccggttacttggcctccattaatttggggagtcgtatgtggagctgctgcttctggtaacttccaatggactgtggaagatgttgctaaatcaattgtttgcatgttgatgtctggcccatttctaaccggttacacacagacgatcaatgattggtatgatagagacattgatgctattaatgaaccttaccgtccaattccttccggagccatatctgaaaatgaggtcattactcaaatttgggtacttcttttaggaggcatcggattggctggtatattagacgtgtgggcagggcataagtcccctacaatattctatcttgctttgggtggatcattgttatcttatatctactcagctccacctttaaagctcaaacagaatggatggattggcaactttgcattaggagcaagctatattagcttaccatggtgggctggtcaagcattgttcggaactcttacacctgatatagtagttctcacacttttgtacagcatagctgggcttggtattgctatagtaaatgactttaaaagtgttgaaggagacaggaaaatggggcttcagtcccttcccgtggcttttggtgaagagacagctaaatggatatgtgttggtgccattgacataactcaactctctattgcaggttaccttttaggatctggtaaaccatattacgccttagcactcgttgggttgattgttccacaaatcttttttcagttcaagtactttcttaaagatccagttaaatatgatgtcaagtatcaggctagtgctcaaccatttctcattcttggtcttctggtgactgccttagctactagtcactga

SEQ ID NO: 49 - DNA, 1188 bp, Helicrysum umbraculigerum
  source 1..1188: mol_type = other DNA
atgaagtctttgattattgggtctttttctaataaggtttcttgttattccccatcattaccagattcatcttcttcacttataccaacaggttgttatcatgtatcactaagaacatttcagcgtaaccgagccattcaagctcaatcaagtcttgtgagatgcaatattggcaaattcaatgaaacattactactttcgcggaaacgaagtacaaaacatgttgcatgtgcggtttctgaacaacccattgaaccagatgctacaaaccctcaaagttcattaccaaatgctttggatgctttctataggttttcaagacctcatacagttataggaactgcattgagcatagtttcggtttcactcctagcggttcaaaagctttcggatttttctccactattcttcattggcgttttcgaggctattgttgctgccttctttatgaacatatacattgttggcttgaaccagctatccgatattgaaatagacaaggttaacaagccgtaccttccattggcatctggagaatattcagttcaaactggtattatcattgtatcatcatttgcagtcatgagtttctggcttggatggatcgtgggctcatggcctttattttgggcacttttcataagttttcttctagggaccgcatattcaatcaatataccgatgttgagatggaagcgctttgctcttgtggcagcaatgtgtattctagctgtaagagctattatagttcaagttgcattttatttgcacattcagacttttgtgtatggaagactcgccgtgttcccaaaacccgtgatatttgcaaccggatttatgagtttcttctctgttgttatagcattgttcaaggacatacccgacattgttggagacaagatttttggcattcaatcatttactgtccgtatgggtcaaaaacgggtgttttggatttgcatcttattacttgaaatagcttatggtgttgctattctagttggggcatcatctcccttcctttggagccgatacataacggtattgggtcatgcgattcttggtctgattctctggggtcgtgccaagtcaacggatctggagagcaaatcagcaataacctcattttacatgttcatatggcagttgttctatgccgagtatttgctcataccgctcgtgagatga

SEQ ID NO: 50 - DNA, 1167 bp, Helicrysum umbraculigerum
  source 1..1167: mol_type = other DNA
atggagttatcactctcatcatcttcttcttcatcccttccccaacttcatactcatccttcatcatcatcatcttcttcacattacataaaaaaatcacctttttttattaataaattcaataatcacaccaaatgcaaattccacaattcctctgctctgagaactaatttcttctacactaccataactaaaacctcatcatcaagattcgttctaaacaaaaacccaaaccaattttccgtcaaggcttgcagtcaagttggttctgctggatccgatccagcattgaataaagttgcagactttaaagatgcattttggaggtttctaaggccccatactattcgtgggacagcattaggatcagtgtctttagtaacgagagcactacttgaaaacccaaacttgattcggtggtcacttttgctcaaggcattttcaggtcttgttgctttgatatgtgggaatggttatatagtcgggatcaatcagatctatgatatcggtattgataaggtgaacaaaccatatttacctattgctgcgggagatctttctgtccagtcagcatggtttttggtgttagcatttgcaatggtaggcgttattattgttgggatgaacttcggcccattcatcacctccctttattctctcggtcttttcttgggcaccatctattccgttccaccacttcgaatgaagagatttcctgttgttgcatttcttatcatcgccacggtgagaggttttcttctaaattttggtgtgtattatgcggttagagcagctctgggactaacattccaatggagctcagcagtggcttttatcacaaccttcgttacattatttgctttagtcattgccattactaaagatcttcctgatgtagagggtgaccgaaagtttcaaatttctacttttgcaacaaaacttggagtaagaaacattgcattattagggtcaggacttctgctgatcaattatattgggtctatcgttgcagcactttacatgcctcaggctttcaggagcagcttgatgataccattacataccatattagcttcctgtttgatttaccaggcatggatacttgagcgtgcgaattacacccagcgatcacagtactttgacatgtcatcttgcaggaggcgatag

SEQ ID NO: 51 - DNA, 1116 bp, Helicrysum umbraculigerum
  source 1..1116: mol_type = other DNA
atggagttatcactctcatcatcttcttcttcatcccttccccaacttcatactcatccttcatcatcatcatcttcttcacattacataaaaaaatcacctttttttattaataaattcaataatcacaccaaatgcaaattccacaattcctctgctctgagaactaatttcttctacactaccataactaaaacctcatcatcaagattcgttctaaacaaaaacccaaaccaattttccgtcaaggcttgcagtcaagttggttctgctggatccgatccagcattgaataaagttgcagactttaaagatgcattttggaggtttctaaggccccatactattcgtgggacagcattaggatcagtgtctttagtaacgagagcactacttgaaaacccaaacttgattcggtggtcacttttgctcaaggcattttcaggtcttgttgctttgatatgtgggaatggttatatagtcgggatcaatcagatctatgatatcggtattgataaggtgaacaaaccatatttacctattgctgcgggagatctttctgtccagtcagcatggtttttggtgttagcatttgcaatggtaggcgttattattgttgggatgaacttcggcccattcatcacctccctttattctctcggtcttttcttgggcaccatctattccgttccaccacttcgaatgaagagatttcctgttgttgcatttcttatcatcgccacggtgagaggttttcttctaaattttggtgtgtattatgcggttagagcagctctgggactaacattccaatggagctcagcagtggcttttatcacaaccttcgttacattatttgctttagtcattgccattactaaagatcttcctgatgtagagggtgaccgaaagtttcaaatttctacttttgcaacaaaacttggagtaagaaacattgcattattagggtcaggacttctgctgatcaattatattgggtctatcgttgcagcactttacatgcctcaggtgaaaaccacttcgatagaccattacagaccatacagcttcctggttgatttaccaggtcaaaatgggattactttagcagcttga

SEQ ID NO: 52 - DNA, 1125 bp, Helicrysum umbraculigerum
  source 1..1125: mol_type = other DNA
atggctactatggcttcttctttgctgaatcctctttcttgttccattaaacccaactcaaacagactaccattaccattaccaatacccatttctctatctcgttcttgtagaaggctaacaatcaaagcaacggagacagatgcaaatgaagtgaagccaaaggcgccagagaaagcaccagctgcaagtggatctggttttaatcaaattcttgggattaaaggggctaaacaagaaactaataaatggaagatccgtgttcaacttacaaagccggttacttggcctccattaatttggggagtcgtatgtggagctgctgcttctggtaacttccaatggactgtggaagatgttgctaaatcaattgtttgcatgttgatgtctggcccatttctaaccggttacacacagacgatcaatgattggtatgatagagacattgatgctattaatgaaccttaccgtccaattccttccggagccatatctgaaaatgaggtcattactcaaatttgggtacttcttttaggaggcatcggattggctggtatattagacgtgtgggcagggcataagtcccctacaatattctatcttgctttgggtggatcattgttatcttatatctactcagctccacctttaaagctcaaacagaatggatggattggcaactttgcattaggagcaagctatattagcttaccatggtgggctggtcaagcattgttcggaactcttacacctgatatagtagttctcacacttttgtacagcatagctgggcttggtattgctatagtaaatgactttaaaagtgttgaaggagacaggaaaatggggcttcagtcccttcccgtggcttttggtgaagagacagctaaatggatatgtgttggtgccattgacataactcaactctctattgcaggttaccttttaggatctggtaaaccatattacgccttagcactcgttgggttgattgttccacaaatcttttttcagttcaagtactttcttaaagatccagttaaatatgatgtcaagtatcaggctagtgctcaaccatttctcattcttggtcttctggtgactgccttagctactagtcactga

SEQ ID NO: 53 - DNA, 1221 bp, Helicrysum umbraculigerum
  source 1..1221: mol_type = other DNA
atggcatctctagctattggttcacttggtagcccaagctcacgtcagtgttctagccccgttgcatcatcttcttcatttgcgatagggtcacaaatagcttcaaagtttcttcggatatcaaaatttgataagactaagaacagccccttaacattgcaacaaaagcatataaacaaaagcatagatcaaagcttctttgagccgcttccattgcacaaaataaacaaagacaagtttaagttgtatgcaacatctacaaacaatcctcagtttgatgcaactcatgatttgaagactccggaagtatccattatcaactttgtggacgctctttataggttaataaggccgtatacagcagttgtaacgatcgtaagtgtagtcgcgatgtcccttcttacagttaatagcctttcagatttttccccattgttcttcatcaaagtggtacaggctcttattggaggcatattcatgcaaatgtatgttagtggtttcaatcaaatttgtgatatagaactcgacaaggttaacaaacagtctcttccattagcggctggagaactatctatgaaaactgcgatcgtcatcgcatcactatcagctatcatgagcttatcgattggttggtttgttggctccccaccattattgtggtgtcttgtttggtggtttattgttgggactgcatattcggccaacgtgctgccttatttgcgatggaaaaggtttcctttcacagcagcattttgcgccatgacgtctcgggcactagttcttcctattggatattacttgcatatgcagaattccatcccgggagtatctgcattactttcaaggccaatattatttgcagtcgcaatgctcagtgcattttctttatcagcgatgttctttaaggacatccctgatattaagggagataggatgcatggaatcaagtctctagcaattaaactgggtgaaaaacgggtgtattggatttccatttcgattattgaaattgcttatattgctgctgcatttattggagcaacttcacccataagctggagcaagtatgtaacgattatcggtcatcttggaatgggattactactttgggtacgagccagatcagtagatccgacgaacacggtagccgttcaatcgatgtatatgttccttattaagctagtatatgcagaatacggacttatctcgcttgtacgctga

SEQ ID NO: 54 - DNA, 1188 bp, Helicrysum umbraculigerum; identical to SEQ ID NO: 49
  source 1..1188: mol_type = other DNA

SEQ ID NO: 55 - DNA, 1308 bp, Helicrysum umbraculigerum
  source 1..1308: mol_type = other DNA
atgttgattcaccatgaacattttttgacaaccggatttgaaagttcaaacgatcgagctgcttattcaataaacttttcgaaacaacatcacttacacatggcgtctatagctactggttcactttgtaggccaacctcacatcaattttctatccccgttgcatcatcttcttcatttgcgacaggatcacaattcgcttcaaagtttcttcatatatcaatatctgctaaaaaaagctcattgacattgcaacaaaggcatattcataaaaacatagatcaaagcttcttaaagccgcttgcacttcaaaaattgaacaaagacaagtttaagttgaatggaacatctccagacaatcctcagtttgatgcaactcatgatttgaagactcaaatagaatccactatcaactttgtggacgttctttataggttgttaaggccgtatgcattacttcaaatgggtttatgtgtagtcacgatgagtcttcttaccgttgaaagcctttcagatttttccccattgttcttcgtcaaagtggcacaggctcttattggaggcatattcatgcaaatgtatgttaatggttttaatcagatttgtgatatagaactcgacaaggttaacaaaccgtctcttccgttagcatctggggaactatctaagacaactactatagtcgtctcttcactatcagctattacgagcttatcgattggttggtttgttggctccccaccattgttgtggagtcttgttgtgtggtttattgctgggactacatattcggctaatctgccatatttgcgatggaaaaggtttcctttcacaaatatgttttgcaacttgacgatggcactagttgttcctattggaacttacttgcatatggagaattccatccacggagtatccacattactttcaaggccactattatttacagttgcaatgtgcactgtgtttcctgtttcgataatactctttaaggacatccctgatattaagggagaccggatgcatggaatgaagtctctagcaattatactgggtgaaaaacggacgtattggatatgcatttggattcttgaaatcacttatattgctgctgcttttttcggagcaacttcacccatcagctggagcaaatatgtaacgattattagtcatctaggaatggggttcttactttggctacgatccaaatcagtagatgtgaagaacacagtagccgttcaatctatgtatatgttcctttggaagctactctatgcagaatatggccttatcttgcttgtacgctga

SEQ ID NO: 56 - DNA, 1293 bp, Helicrysum umbraculigerum
  source 1..1293: mol_type = other DNA
atgtttattcaccatgaacagtttttgacaaccggatttgaaagttcaaacgatcgagctgcctattcaataaactttttgaaacaacatcacttacacatggtgtctatagctactggttcactttgtaggccaacctcacatcgattctctatccccgttgcatcatcttcttcatttgcgacaggatcacaattcgcttcaatatctgctaaaaaaagctcattgacattgaaacaaaggcatactcataaaaacatagatcaaagcttcttcaagccgcttgcacttcaaaaaatgaacaaaggcaagtttaagttgaatgcaacatctccagacaattctcagttggatgcaactcatgatttgaagactcaaatagaatccattatcaactttgtggacgttctttataggttgataaggccgtatgtagtacttggaatgggtgtaactatagtcacgatgtgtcttcttaccgttgatagcctttcagatttttccccattgttcttcgtcaaagtggcacaggctcttattggaagcatattcatggcaatgtatgttaatagttttaatgagatttgtgatatagaactcgacaaggttaacaaaccgtctcttccgttagcgtctggggaactatctatgacaactgctattgtcgtctcttcactatcagctatcatgagcttatcgattggttggtttgttggctccccaccattgttgtggagtcttgttgtgtggtttattcttgggactgcatattcggctaatctgccatatttgcgatggaaaaggtttcctttaacaacactgtcttccgccctgacgatgggggcactagttattcctattggaaattacatgcatatggagaattccatccgcggagtaaccacattactttcaaggccactattatttgcagttgcaatgtgcgctgcgtttcatgtttcgacgatactctttaaggacatccctgatattaagggagaccggatgcatggaatgaagtctctagcaattaaactgggtgaaaaacggatgtattggatatgcatttggattcttgaaatcgcttatattgctgctgcttttttcggagcaacttcacccatcagctggagcaaatatgtaacgattattagtcatctaggaatggggttcttactttggctacgatccaaatcagtagatgtgaagaacacagtagccgttcaatctatgtatatgttcctttggaagctattctatgtagaacatggtcttatcttgcttgtacgttga

SEQ ID NO: 57 - DNA, 1209 bp, Helicrysum umbraculigerum
  source 1..1209: mol_type = other DNA
atggcgtctatagctactggttcactttgtaggccaacctcacatcgattttctatccacgttgcatcatcttcttcatttgcgacaggatcacagtttgcttcaaagattcttcagatatcaatatctgctaaaaaaagctcattgacattgcaacaaaggcatattcataaaaacatagatcaaagcttcttcaagccgcttgcacttcaaaaaatgaacaaagacaagtttaagttgaatgcaacatctccagacaatccacagtttgatgcaactcgtgatttgaagactcaaatagaatccattatcaagtttgtggacgttctttataggttgttaaggccgtacgcaatacttgaaatgggtttaagtgtagtcacgatgagtcttcttaccgttgaaagcctttcagatttttccccgttgttcttcgtcaaagtggcacaagctcttattggaggcatattcatgcaaatgtatgttaatggttttaatcagatttgtgatatagaactcgacaaggttaacaaaccgtctcttccgttagcgtctggggaactatctacgacaactactatagtcgtctcttcactatcagctattatgagcttatcgattggttggtttgttggctccccaccattgttgtggagtcttgttgtgtggtttattgttgggacaacatattcgactaatctgccatatttgcgatggaaaaggtttcctttcacagcaatgttttgcaacctgacgagggcactagttgttcctattggaacttacttgcatatgaagaattccatccacgaagtatccacattactttcaaggccactgttatttgcagttgcaatgtgcactgtgtttcctatttcgataatactctttaaggacatccctgatattaagggagaccggatgcatggaatgaagtctctagcaattatactgggtgaagaacggacgtattggatatgcatttggattcttgaaatcgcttatattgctgctgcttttttcggagcaacttcacccatcagctggagcaaatatgtaatgattattagtcatctaggaatggggttcttactttggctacgatccaaatcagtagatgtgaagaacacagtagccgttcaatctatgtatatgttcctttggaagctactctatgcagaatatggccttattttgcttgtacgctga

SEQ ID NO: 58 - DNA, 1221 bp, Helicrysum umbraculigerum
  source 1..1221: mol_type = other DNA
atggcatctctagctattggttcacttggtagcccaagctcacgtcagtgttctagccccgttgcatcatcttcttcatttgcgatagggtcacaaatagcttcaaagtttcttcggatatcaaaatttgataagactaagaacagccccttagcattgcaacaaaagcatataaacaaaagcatagatcaaagcttctttgagccgcttccattgcacaaaataaacaaagacaagtttaagttgtatgcaacatctacaaacaatcctcagtttgatgcaactcatgatttgaagactccggaagtatccattatcaactttgtggacgctctttataggttaataaggccgtatacagcagttgtaacgatcgtaagtgtagtcgcgatgtcccttcttacagttaatagcctttcagatttttccccattgttcttcatcaaagtggtacaggctcttattggaggcatattcatgcaaatgtatgttagtggtttcaatcaaatttgtgatatagaactcgacaaggttaacaaacagtctcttccattagcggctggagaactatctatgaaaactgcgatcgtcatcgcatcactatcagctatcatgagcttatcgattggttggtttgttggctccccaccattattgtggtgtcttgtttggtggtttattgttgggactgcatattcggccaacgtgctgccttatttgcgatggaaaaggtttcctttcacagcagcattttgcgccatgacgtctcgggcactagttcttcctattggatattacttgcatatgcagaattccatcccgggagtatctgcattactttcaaggccaatattatttgcagtcgcaatgctcagtgcattttctttatcagcgatgttctttaaggacatccctgatattaagggagataggatgcatggaatcaagtctctagcaattaaactgggtgaaaaacgggtgtattggatttccatttcgattattgaaattgcttatattgctgctgcatttattggagcaacttcacccataagctggagcaagtatgtaacgattatcggtcatcttggaatgggattactactttgggtacgagccagatcagtagatccgacgaacacggtagccgttcaatcgatgtatatgttccttattaagctagtatatgcagaatacggacttatctcgcttgtacgctga

SEQ ID NO: 59 - protein, 399 aa, Helicrysum umbraculigerum
MELSLSSSSSSSLPQLHTHPSSSSSSSHYIKKSPFFINKFNNHTKCKFHNSSALRTNFFYTTITKTSSSRFVLNKNPNQFSVKACSQVGSAGSDPALNKVADFKDAFWRFLRPHTIRGTALGSVSLVTRALLENPNLIRWSLLLKAFSGLVALICGNGYIVGINQIYDIGIDKVNKPYLPIAAGDLSVQSAWFLVLAFAMVGVIIVGMNFGPFITSLYSLGLFLGTIYSVPPLRMKRFPVVAFLIIATVRGFLLNFGVYYAVRAALGLTFQWSSAVAFITTFVTLFALVIAITKDLPDVEGDRKFQISTFATKLGVRNIALLGSGLLLINYIGSIVAALYMPQAFRSSLMIPLHTILASCLIYQAWILERANYTQEAIAGYYRFVWNLFYSEYIIFPFI

SEQ ID NO: 60 - protein, 372 aa, Helicrysum umbraculigerum
MATMASSLLNPLSCSIKPNSNRLPLPTPISLSRSCRRLTIKATETDANEVKPKAPEKAPAASGSGFNQILGIKGAKQETNKWKIRVQLTKPVTWPPLIWGVVCGAAASGNFQWTVEDVAKSIVCMLMSGPFLTGYTQTINDWYDRDIDAINEPYRPIPSGAISENEVITQIWVLLLGGIGLAGILDVWAGHKSPTIFYLALGGSLLSYIYSAPPLKLKQNGWIGNFALGASYISLPWWAGQALFGTLTPDIVVLTLLYSIAGLGIAIVNDFKSVEGDRKMGLQSLPVAFGEETAKWICVGAIDITQLSIAGYLLGSGKPYYALALVGLIVPQIFFQFKYFLKDPVKYDVKYQASAQPFLILGLLVTALATSH

SEQ ID NO: 61 - protein, 395 aa, Helicrysum umbraculigerum
MKSLIIGSFSNKVSCYSPSLPDSSSSLIPTGCYHVSLRTFQRNRAIQAQSSLVRCNIGKFNETLLLSRKRSTKHVACAVSEQPIEPDATNPQSSLPNALDAFYRFSRPHTVIGTALSIVSVSLLAVQKLSDFSPLFFIGVFEAIVAAFFMNIYIVGLNQLSDIEIDKVNKPYLPLASGEYSVQTGIIIVSSFAVMSFWLGWIVGSWPLFWALFISFLLGTAYSINIPMLRWKRFALVAAMCILAVRAIIVQVAFYLHIQTFVYGRLAVFPKPVIFATGFMSFFSVVIALFKDIPDIVGDKIFGIQSFTVRMGQKRVFWICILLLEIAYGVAILVGASSPFLWSRYITVLGHAILGLILWGRAKSTDLESKSAITSFYMFIWQLFYAEYLLIPLVR

SEQ ID NO: 62 - protein, 388 aa, Helicrysum umbraculigerum
MELSLSSSSSSSLPQLHTHPSSSSSSSHYIKKSPFFINKFNNHTKCKFHNSSALRTNFFYTTITKTSSSRFVLNKNPNQFSVKACSQVGSAGSDPALNKVADFKDAFWRFLRPHTIRGTALGSVSLVTRALLENPNLIRWSLLLKAFSGLVALICGNGYIVGINQIYDIGIDKVNKPYLPIAAGDLSVQSAWFLVLAFAMVGVIIVGMNFGPFITSLYSLGLFLGTIYSVPPLRMKRFPVVAFLIIATVRGFLLNFGVYYAVRAALGLTFQWSSAVAFITTFVTLFALVIAITKDLPDVEGDRKFQISTFATKLGVRNIALLGSGLLLINYIGSIVAALYMPQAFRSSLMIPLHTILASCLIYQAWILERANYTQRSQYFDMSSCRRR

SEQ ID NO: 63 - protein, 371 aa, Helicrysum umbraculigerum
MELSLSSSSSSSLPQLHTHPSSSSSSSHYIKKSPFFINKFNNHTKCKFHNSSALRTNFFYTTITKTSSSRFVLNKNPNQFSVKACSQVGSAGSDPALNKVADFKDAFWRFLRPHTIRGTALGSVSLVTRALLENPNLIRWSLLLKAFSGLVALICGNGYIVGINQIYDIGIDKVNKPYLPIAAGDLSVQSAWFLVLAFAMVGVIIVGMNFGPFITSLYSLGLFLGTIYSVPPLRMKRFPVVAFLIIATVRGFLLNFGVYYAVRAALGLTFQWSSAVAFITTFVTLFALVIAITKDLPDVEGDRKFQISTFATKLGVRNIALLGSGLLLINYIGSIVAALYMPQVKTTSIDHYRPYSFLVDLPGQNGITLAA

SEQ ID NO: 64 - protein, 374 aa, Helicrysum umbraculigerum
MATMASSLLNPLSCSIKPNSNRLPLPLPIPISLSRSCRRLTIKATETDANEVKPKAPEKAPAASGSGFNQILGIKGAKQETNKWKIRVQLTKPVTWPPLIWGVVCGAAASGNFQWTVEDVAKSIVCMLMSGPFLTGYTQTINDWYDRDIDAINEPYRPIPSGAISENEVITQIWVLLLGGIGLAGILDVWAGHKSPTIFYLALGGSLLSYIYSAPPLKLKQNGWIGNFALGASYISLPWWAGQALFGTLTPDIVVLTLLYSIAGLGIAIVNDFKSVEGDRKMGLQSLPVAFGEETAKWICVGAIDITQLSIAGYLLGSGKPYYALALVGLIVPQIFFQFKYFLKDPVKYDVKYQASAQPFLILGLLVTALATSH

SEQ ID NO: 65 - protein, 406 aa, Helicrysum umbraculigerum
MASLAIGSLGSPSSRQCSSPVASSSSFAIGSQIASKFLRISKFDKTKNSPLTLQQKHINKSIDQSFFEPLPLHKINKDKFKLYATSTNNPQFDATHDLKTPEVSIINFVDALYRLIRPYTAVVTIVSVVAMSLLTVNSLSDFSPLFFIKVVQALIGGIFMQMYVSGFNQICDIELDKVNKQSLPLAAGELSMKTAIVIASLSAIMSLSIGWFVGSPPLLWCLVWWFIVGTAYSANVLPYLRWKRFPFTAAFCAMTSRALVLPIGYYLHMQNSIPGVSALLSRPILFAVAMLSAFSLSAMFFKDIPDIKGDRMHGIKSLAIKLGEKRVYWISISIIEIAYIAAAFIGATSPISWSKYVTIIGHLGMGLLLWVRARSVDPTNTVAVQSMYMFLIKLVYAEYGLISLVR

SEQ ID NO: 66 - protein, 395 aa, Helicrysum umbraculigerum; identical to SEQ ID NO: 61

SEQ ID NO: 67 - protein, 435 aa, Helicrysum umbraculigerum
MLIHHEHFLTTGFESSNDRAAYSINFSKQHHLHMASIATGSLCRPTSHQFSIPVASSSSFATGSQFASKFLHISISAKKSSLTLQQRHIHKNIDQSFLKPLALQKLNKDKFKLNGTSPDNPQFDATHDLKTQIESTINFVDVLYRLLRPYALLQMGLCVVTMSLLTVESLSDFSPLFFVKVAQALIGGIFMQMYVNGFNQICDIELDKVNKPSLPLASGELSKTTTIVVSSLSAITSLSIGWFVGSPPLLWSLVVWFIAGTTYSANLPYLRWKRFPFTNMFCNLTMALVVPIGTYLHMENSIHGVSTLLSRPLLFTVAMCTVFPVSIILFKDIPDIKGDRMHGMKSLAIILGEKRTYWICIWILEITYIAAAFFGATSPISWSKYVTIISHLGMGFLLWLRSKSVDVKNTVAVQSMYMFLWKLLYAEYGLILLVR

SEQ ID NO: 68 - protein, 430 aa, Helicrysum umbraculigerum
MFIHHEQFLTTGFESSNDRAAYSINFLKQHHLHMVSIATGSLCRPTSHRFSIPVASSSSFATGSQFASISAKKSSLTLKQRHTHKNIDQSFFKPLALQKMNKGKFKLNATSPDNSQLDATHDLKTQIESIINFVDVLYRLIRPYVVLGMGVTIVTMCLLTVDSLSDFSPLFFVKVAQALIGSIFMAMYVNSFNEICDIELDKVNKPSLPLASGELSMTTAIVVSSLSAIMSLSIGWFVGSPPLLWSLVVWFILGTAYSANLPYLRWKRFPLTTLSSALTMGALVIPIGNYMHMENSIRGVTTLLSRPLLFAVAMCAAFHVSTILFKDIPDIKGDRMHGMKSLAIKLGEKRMYWICIWILEIAYIAAAFFGATSPISWSKYVTIISHLGMGFLLWLRSKSVDVKNTVAVQSMYMFLWKLFYVEHGLILLVR

SEQ ID NO: 69 - protein, 402 aa, Helicrysum umbraculigerum
MASIATGSLCRPTSHRFSIHVASSSSFATGSQFASKILQISISAKKSSLTLQQRHIHKNIDQSFFKPLALQKMNKDKFKLNATSPDNPQFDATRDLKTQIESIIKFVDVLYRLLRPYAILEMGLSVVTMSLLTVESLSDFSPLFFVKVAQALIGGIFMQMYVNGFNQICDIELDKVNKPSLPLASGELSTTTTIVVSSLSAIMSLSIGWFVGSPPLLWSLVVWFIVGTTYSTNLPYLRWKRFPFTAMFCNLTRALVVPIGTYLHMKNSIHEVSTLLSRPLLFAVAMCTVFPISIILFKDIPDIKGDRMHGMKSLAIILGEERTYWICIWILEIAYIAAAFFGATSPISWSKYVMIISHLGMGFLLWLRSKSVDVKNTVAVQSMYMFLWKLLYAEYGLILLVR

SEQ ID NO: 70 - protein, 406 aa, Helicrysum umbraculigerum
MASLAIGSLGSPSSRQCSSPVASSSSFAIGSQIASKFLRISKFDKTKNSPLALQQKHINKSIDQSFFEPLPLHKINKDKFKLYATSTNNPQFDATHDLKTPEVSIINFVDALYRLIRPYTAVVTIVSVVAMSLLTVNSLSDFSPLFFIKVVQALIGGIFMQMYVSGFNQICDIELDKVNKQSLPLAAGELSMKTAIVIASLSAIMSLSIGWFVGSPPLLWCLVWWFIVGTAYSANVLPYLRWKRFPFTAAFCAMTSRALVLPIGYYLHMQNSIPGVSALLSRPILFAVAMLSAFSLSAMFFKDIPDIKGDRMHGIKSLAIKLGEKRVYWISISIIEIAYIAAAFIGATSPISWSKYVTIIGHLGMGLLLWVRARSVDPTNTVAVQSMYMFLIKLVYAEYGLISLVR

SEQ ID NO: 71 - DNA, 1593 bp, Helicrysum umbraculigerum
  source 1..1593: mol_type = other DNA
atggggctaaacatttgcactagatttataccttgtttggtagtggttctcatgtttttgttcacttcaacatattcagctacaccagaagacaaattccttcaatgcatatctcaaaaattaaatatcacaaactcagatgaagtgttcactcaatcaaacacacgatattcatctgttcttgagtcaacaatagttaaccttagatttgccacttctacaacgccaaaaccatttgctataatcacacctttgtcatattcacatgtacaatctgctgtagtttgtgctaaaaaagccggaatccgaattagaatcagaagtggtggccatgactatgtgggcctttcatatacttcatctgataatgtcccttttgttgttcttgaccttaaacagctgcagaatgttacggtcgagtatagtaagaaaacggcttgggttgaatctggtgcaaccatcggtcaactgtattattgggtgtctcagaaaagtaaaaatctaggattcccgggtgggacctgcgcaactataggggtcggagggcacctaagtggtgggggttttggtactttggtaagaaagtatggtctatcggctgataacgttattgatgctaagatagttgatgtcaatggtagacttcttgatagaaagtctatgggggaagatttgttttgggcaattagaggaggcggtggaggaagtttcggtgttgtagtagcttggatggtcaatcttgttcatgttcctgaaaaagttacagcttttactattgtcaggactttggaacaaggtggttcggatcttttcaacaagtggcagcacgttgggcccaaattaaccaaagatttgttcattagtgttataatacagcccatttctgtttggaatggaaacggaacagttcaagttatattcaactcgatgtatcttgggacggttgataagctcatgaagaccgtcaacagtagctttccggagttggggttacaagcaaaagactgcactgagatgagttggattcagtcagtactttattttgcgggttaccctatagaaggaagtatggatgttcttaaagataggaaaccccagaccagaagatactttaataataaatcagatcacgtgaaagaaccgatacccaaagaaagattagaagatttatggaaatggtgtatggaaggtgattttccgattcttctaatggacccactcggtggaaagatgaacgagattgacacaacaagaattccgtacccttatagaaatggttattcgtatatgatacaatacgttgagacctgggaaaacattggggactcagaaaagcgtataagttggatgagacagatgtatgagaatatgacaccgtatgtgtcgaagaatccaaggtcagcttatgtgaattatagggatttggatttaggtaaaaacgataacgctaaaaacacgagttacttggaagccatgaaatggggaagcaagtactttggtgacaatttcaagaggttggctatggtgaaaggtgtagttgatccagacaatttcttctttcatgaacaaagcatcccacctctgaaagtgtga

SEQ ID NO: 72 - DNA, 1719 bp, Helicrysum umbraculigerum
  source 1..1719: mol_type = other DNA
atggggtgtaatctcttgcaaaaacttactatttttgttttctttatcatgtctatttccataccttctttcgcttacgaacacgagcacgagcatgagcacgaacacgaaaatgatcaagatcgagtacaggatgaaaaggaacctacggatgtcttcacttcgtgtttaactcggttcggtgttcataattttacaactcattccaagtcgaataatgataattcggtttactatgagcttcttaatttttcaattcaaaatcttagatttacgggtttatcgatgcctaaaccggttgttatcgtgttcccggagacgaaagaacagttagcaaaaaccgtggtttgtgctcgagaatcgtcgctagaaattcgggttcggtgtggtggtcatagctatgaagggacatcatccgtctccacggacggacgtccatttgtggtgattgatatgacgagattagacaatgtttcggtggacgtgaactcgggaaccgcatgggttgaagctggcgcgacacttggtcaaatgtactgcgcgatagcagagtcgagcacggtccatggtttctcggcagggtcatgccccactgtcggaacaggtggtcatatttcgggtggtgggtttgggttattgtcgcgaaaatacgggctggctgcggataatgtagtcgatgcggttttagtaaccgcagatggtgaattactgaaccgcgacacgatgggtgaggatgttttttgggcgattagaggtggtggtggcggggtttggggaattgtgtacgcttttaatgttaaattatcaagcgtaccaaaaacagtcactaatttcgtcgtgtctaggccaggcacgaagggacaagtgactgatttggtatataaatggcagcatgttgcgcctaaattgcccgacgacttctacttatcctctttcgttggtgcgggtttgcctgaacgaaaaaataaaccgggtttatcggctacgttcaaaggtttttatttgggatcgaaaagcaaagctttatcgatcatgaaccaaactttccccgagctaaaagtcatggaaaacgactgtaaagaaacaagttggattgagtctattcttttcttctcgggttatggagatgaaagctcggtttctgacttgaaaaatcgcttcttacaagataaattgtattacaaggccaaatcggactatgttcggaaacctattccaagattcggtctaactacggcactagaaatactcgagaaacaaccaaaagggtatgtgatcttggacccatatggtggcgcaatgcaaacgataagtagtgactcgatcccgttccctcataggaaaggtaatattttcactattcaatatctagtggaatggaaagaaccggataacgataaaacgaatgattacttagcgtggatacgagactttcatggctcgatgacgccctatgtggcacaagacccacgagccgcatacattaactacatggatgttgatattggagtcatgaattggatcaaaactagagtggactcagatgatgcagttgagatgggtcgagaatggggggagaagtacttttataagaattacgatcggctagtgagagcgaagacacaaatcgatccgtacaatgtttttaggcatcaacaaagcatccctccaatgtctttggagaacaagaatcgcaggggaagtatatctagtgagtag

SEQ ID NO: 73 - DNA, 1641 bp, Helicrysum umbraculigerum
  source 1..1641: mol_type = other DNA
atgaaaacatcatcaaatatgctttccgtattactcattctattctttatcacatgctcaaaagcagctctggatcctgattccgtctatcaatcatttctccaatgtttaccgttatactcaccggagtccgcggaggaactctccaaggtcgtatacagctccaccttgaacaccacaacatacgaaaccgtactccaagagtacataaaaaacgaacgatttaacaccaccgcgacacccaaaccgtcggttatcataaccccgacaaccgaatctcaagtccaagcggccgtcctatgcgcgaaaaaaaccggtgtccaaattaaaattcgtagcggcggacacgactacgaaggaatatcgtatatttcatccgaacctgattttatcgtacttgacatgtttaactttcggtcgataaatgttaatgtagcggacgaaaccgcggttgtgggcgccggcgcgcagttgggcgagctttattataggatttacgaaaaaagtaaaactctcgggttccccgcgggagtttgtcagacggttggcgtgggaggtcatctgagcggcggtggttacggaactatgctgcgaaaatacgggttgtcagttgatcatgtgattgatgcgaaaattgttgatgtgaatggtcaggttttggatcggaaatcgatgggtgaggatctattttgggcgatacgaggtggcggtggcggtagttttggtgtgattttgtcgtatactgtgaagttggtttcggttcccgaggttaacacggtctttcgcgtgctgaaaacgacgtcggaaaatgcttctgaactgatttataagtggcagtcgattatgccggatattgataacgatttgtttatcagagttttgttacaaccggttacggtgaataaacagaaagttggtcgggctacgtttatagcgcattttttaggtgattctgatagattggtggcgttgatgagtaaaaacttcccggaattgggtttaaagaaagaggattgtatcgaggtgagttggatagaatcggtactttattgggctaactttgatttgaatacgacgaagccagagattcttctagatcgacattccgacagtgtgagctatggtaaacgaaagtcggactatgtgcaaaccccgattcctgaatccgggttggaatcgatttttgaaaagttagtcgaattgggtaaaatcgggttggtttttaactcgtatggcgggagaatgtcggaggttgcggctgacgcaacaccattccctcaccgagctgggaacattttcaagattcagtattcggttaattggaatgatgcggaccctgaactagaagcgaattacttaaatcaaagtagggttatgtacgacttcatgacaccatttgtatcgaagaatccgagagctgcattcttgaattatcgggatctcgatattggagtaatgactcctggcaagaacagttatagtgaaggtgaagtttatggtgagaaatacttcatgggaaatttcgaaagattggtgaagataaaaaccgcggttgatcccgataatttctttagaaatgaacaaagtattccgactcgggccgcgaaaaattcaggcaagtcaagaaagatgatgaagtaa

SEQ ID NO: 74 - DNA, 1593 bp, Helicrysum umbraculigerum; identical to SEQ ID NO: 71
  source 1..1593: mol_type = other DNA

SEQ ID NO: 75 - DNA, 771 bp, Helicrysum umbraculigerum
  source 1..771: mol_type = other DNA
atggaccaatatgtcataactaaatttatatcatatcttctggcggtttttatggctttattctgttcagatccaacggctgataaatttcttcaatgcttcactaaagattcaaatgcaacagattcaaactttgtgttcacccaagaaaacacacaatattcatctgttcttgagtcaactatcataaaccttagatttgcaacctccataactccaaaaccaatagctgtaatcacaccattatcatattcccatgtacaatcagcaatactttgttccaaaaaaatcggatatcgaattagaatcagaagtggtgggcatgactatgcaggagtttcatacacttcatatgatcatgatcataccccttttgttgttcttgatcttaaagagctgaggacgataacaatcgattcgggtgagaacacttcatgggttgaatctggtgcaactgttggtgaactgtattattgggtgtcccaaaaaagtcgaaatcttgggttcccagctgggatttgtccaactgttggggtaggtggtcatttaagtggaggtggggttggtactatggtaagaaagtatggtctagcggctgataatgtaatcgatgctaggattattgatgtaaatgggcgaattcttgataggaaatcgatgggggaagatttgttttgggcgattagaggtggtgggggagctagttttggtgttatagtagcttggaaggtaaatcttgtttatgttcctgaaaaaagtttcggtttttag

SEQ ID NO: 76 - DNA, 1593 bp, Helicrysum umbraculigerum
  source 1..1593: mol_type = other DNA
atggagttgtatattagcactagatttatactatgttttctagtggttcttatgcttatgttctcttcaacatattcagatccactagaagataaatttcttcgatgtctatctcaaaattcaaatgccacaaattcagacaatgtgttcactcaagaaaacacacagtattcatctgttcttgagtcaactatcataaaccttagatttgcaacctctacaactccgaaaccgttagctataatcacaccgttgtcatgttcccatgtacaatctgctgtactttgtgccaaaaaagtcggaatccgaattagaatcagaagtggtggccatgactatgcaggcctttcatacacttcatctgagaatgccccttttgttgttcttgatcttaaacagctgcagaatgttacggtcgagtctagtaagaaaacggcttgggttgaatctggtgcaaccatcggtcaattgtattattgggtgtctcaaaaaagtaaaaatctaggattcccagctgggacctgcgcgactataggggtcggagggcacctaagtggtgggggtttcggtactttggtaagaaagtatggtctatcggctgataacgtcatcgatgctaagatagttgatgtcaatggtagacttcttgatagaaagtctatgggggaagatttgttttgggcaattagaggaggcggtggaggaagtttcggtgttgtagtagcttggaaggtcaatcttgttcatgttcccgaaaaagttacggcttttactattgtcaggactttggaacaaggtggttcggatattttcaacaaatggcagcacattgggcacaaattaactaaagatttgttcattagagttataatacagcctatttctgtttcgaatggaaacagaacagttcaagttatattcaactcgatgtatctggggacggttgataagctcatgaagaccgtcaacagtagcttcccggagttgggcttacaagaaaaagactgcactgagatgagttggattcagtcagtactttattttgcgggttacccaatagaaggaagtatggatgttcttaaagataggaaacccgacacccgaaattactttgataataaatcagatcacgtgaaagaaccgatacccaaagaaagattagaagatctatggaaatggtgtatggaagttgattttccgattcttataatggagccactcggtggaaagatgaacgagattgacacaacaagaattccatacccttatagaaaaggttattcgtatatgatacaatatgttgaggcttgggataacattggggactcggaaaaacatataagttggttgagacagatgtatgagaatatgacaccatatgtgtcgaagaatccaaggtcagcttatgtgaattaccgggatttggatttaggtaaaaacgataacgctaaaaacacgagttacttggaagccatgaaatggggaagcaagtactttggtgacaatttcaagaggttggctatggtgaaaggtgtagttgatccagacaatttcttctttcatgaacaaagcatcccacctctgaaagtgtga

SEQ ID NO: 77 - DNA, 1593 bp, Helicrysum umbraculigerum; identical to SEQ ID NO: 71
  source 1..1593: mol_type = other DNA

SEQ ID NO: 78 - DNA, 933 bp, Helicrysum umbraculigerum
  source 1..933: mol_type = other DNA
atgggggaagatttgttttgggcaattagaggaggcggtggaggaagtttcggtgttgtagtagcttggatggtcaatcttgttcatgttcctgaaaaagttacagcttttactattgtcaggactttggaacaaggtggttcggatcttttcaacaagtggcagcacgttgggcccaaattaaccaaagatttgttcattagtgttataatacagcccatttctgtttggaatggaaacggaacagttcaagttatattcaactcgatgtatcttgggacggttgataagctcatgaagaccgtcaacagtagctttccggagttggggttacaagcaaaagactgcactgagatgagttggattcagtcagtactttattttgcgggttaccctatagaaggaagtatggatgttcttaaagataggaaaccccagaccagaagatactttaataataaatcagatcacgtgaaagaaccgatacccaaagaaagattagaagatttatggaaatggtgtatggaaggtgattttccgattcttctaatggacccactcggtggaaagatgaacgagattgacacaacaagaattccgtacccttatagaaatggttattcgtatatgatacaatacgttgagacctgggaaaacattggggactcagaaaagcgtataagttggatgagacagatgtatgagaatatgacaccgtatgtgtcgaagaatccaaggtcagcttatgtgaattatagggatttggatttaggtaaaaacgataacgctaaaaacacgagttacttggaagccatgaaatggggaagcaagtactttggtgacaatttcaagaggttggctatggtgaaaggtgtagttgatccagacaatttcttctttcatgaacaaagcatcccacctctgaaagtgtga

SEQ ID NO: 79 - DNA, 1581 bp, Helicrysum umbraculigerum
  source 1..1581: mol_type = other DNA
atggagttgaagttgtttacatgtaaactcgtaacaattattctagctctgtccctcagttttttcacatcaacaagctctagtgactttcttgattgcatctctcaaaaaaacttatcaaatattattttcactcctaatgacacttcatactcaactattctccaatttaccatcccaaatcttagatttaacacgcctaaaaccacaaaaccattagcaataatcacacctacaacgtattctcacgtacaatctactataatatgcagcgtgcaattcaagcaccatgttcgcatccgaagtggtggtcatgactacgaaggtctttcgtatacttctttcaataacaccccttttatacttcttgatctcaaccaacttcggtcagtaacggttgatttagatagtaataccacatgggtcgaatctggtgccactctaggtgaacttttgtattgggtgtctcgaaaaagtaatattcttgggatcccaaccggcgagtgtacatcggtgggcgttgggggacaattaagtggaggagggtttggaaatatggctagaaaatatggattattttcggataatgcggttgacgcacttatcattgatgtaaatggacgaatactggatagagattccatgggtgaagatttgttttgggcaattagaggaggtgggggtggaaattttggagttgtattatcttggaagattaatctagtttatgttccacctaaagttacggtttttactgtttctaagatgttagatgaaaatggtaccaagattgttcacaagtggcaatatattgcgcataatataacgcaagatttgttcattaatcttatagtaagtccggttaccgtgtcaaatacaacgattctagcagtaacaattaactcgttgtttttggggatgaaaaacgagcttgtagcaacaatggatgtaatatttccggaattagggttacaagaaaaggattgcatcgaaatgagttggatagaatcggtggtttaccattcggtttatttaagaggacaaagtgttgatgctctaatagaaagaagaccatggcctaaaagttacaacaagtataaatcagattatgtgaagaaacctatgtcagagaaagcgcttgaaaaactgtggaaatggtgtttggaagagaatttgattctggcgatcgagccacatggtggaaagatgagcgagatcgatgagagttcgactccgtatccgcatagaaaagggaatttgtacatcatacaatatgtcatgcaatgggatgaagggtataacacaactcaaaagcatgttgcttccataagaagggtatataagaaaatggcaccttttgtgtccaagaaccctagggaagcttatgtgaactttagagatttggatttgggtactaatggtaatgcatgtggtacaagtggtgcaagctatgtgcaagcattgagatggggaaaaaagtattttaagggaaattttaagaggttggcaatagtgaaaggtagagttgacccaactaatttcttctgtaatgaacaaagcatcccaccttattcgtattag

SEQ ID NO: 80 - protein, 530 aa, Helicrysum umbraculigerum
MGLNICTRFIPCLVVVLMFLFTSTYSATPEDKFLQCISQKLNITNSDEVFTQSNTRYSSVLESTIVNLRFATSTTPKPFAIITPLSYSHVQSAVVCAKKAGIRIRIRSGGHDYVGLSYTSSDNVPFVVLDLKQLQNVTVEYSKKTAWVESGATIGQLYYWVSQKSKNLGFPGGTCATIGVGGHLSGGGFGTLVRKYGLSADNVIDAKIVDVNGRLLDRKSMGEDLFWAIRGGGGGSFGVVVAWMVNLVHVPEKVTAFTIVRTLEQGGSDLFNKWQHVGPKLTKDLFISVIIQPISVWNGNGTVQVIFNSMYLGTVDKLMKTVNSSFPELGLQAKDCTEMSWIQSVLYFAGYPIEGSIDVLKDRKPDTRNYFDNKSDHVKEPIPKERLEDLWKWCMEGDFPILLMDPLGGKMNEIDTTRIPYPYRNGYSYMIQYVETWENIGDSEKRISWMRQMYENMTPYVSKNPRSAYVNYRDLDLGKNDNAKNTSYLEAMKWGSKYFGDNFKRLAMVKGVVDPDNFFFHEQSIPPLKV

SEQ ID NO: 81 - protein, 572 aa, Helicrysum umbraculigerum
MGCNLLQKLTIFVFFIMSISIPSFAYEHEHEHEHEHENDQDRVQDEKEPTDVFTSCLTRFGVHNFTTHSKSNNDNSVYYELLNFSIQNLRFTGLSMPKPVVIVFPETKEQLAKTVVCARESSLEIRVRCGGHSYEGTSSVSTDGRPFVVIDMTRLDNVSVDVNSGTAWVEAGATLGQMYCAIAESSTVHGFSAGSCPTVGTGGHISGGGFGLLSRKYGLAADNVVDAVLVTADGELLNRDTMGEDVFWAIRGGGGGVWGIVYAFNVKLSSVPKTVTNFVVSRPGTKGQVTDLVYKWQHVAPKLPDDFYLSSFVGAGLPERKNKPGLSATFKGFYLGSKSKALSIMNQTFPELKVMENDCKETSWIESILFFSGYGDESSVSDLKNRFLQDKLYYKAKSDYVRKPIPRFGLTTALEILEKQPKGYVILDPYGGAMQTISSDSIPFPHRKGNIFTIQYLVEWKEPDNDKTNDYLAWIRDFHGSMTPYVAQDPRAAYINYMDVDIGVMNWIKTRVDSDDAVEMGREWGEKYFYKNYDRLVRAKTQIDPYNVFRHQQSIPPMSLENKNRRGSISSE

SEQ ID NO: 82 - protein, 546 aa, Helicrysum umbraculigerum
MKTSSNMLSVLLILFFITCSKAALDPDSVYQSFLQCLPLYSPESAEELSKVVYSSTLNTTTYETVLQEYIKNERFNTTATPKPSVIITPTTESQVQAAVLCAKKTGVQIKIRSGGHDYEGISYISSEPDFIVLDMFNFRSINVNVADETAVVGAGAQLGELYYRIYEKSKTLGFPAGVCQTVGVGGHLSGGGYGTMLRKYGLSVDHVIDAKIVDVNGQVLDRKSMGEDLFWAIRGGGGGSFGVILSYTVKLVSVPEVNTVFRVLKTTSENASELIYKWQSIMPDIDNDLFIRVLLQPVTVNKQKVGRATFIAHFLGDSDRLVALMSKNFPELGLKKEDCIEVSWIESVLYWANFDLNTTKPEILLDRHSDSVSYGKRKSDYVQTPIPESGLESIFEKLVELGKIGLVFNSYGGRMSEVAADATPFPHRAGNIFKIQYSVNWNDADPELEANYLNQSRVMYDFMTPFVSKNPRAAFLNYRDLDIGVMTPGKNSYSEGEVYGEKYFMGNFERLVKIKTAVDPDNFFRNEQSIPTRAAKNSGKSRKMMK

SEQ ID NO: 83 - protein, 530 aa, Helicrysum umbraculigerum
MGLNICTRFIPCLVVVLMFLFTSTYSATPEDKFLQCISQKLNITNSDEVFTQSNTRYSSVLESTIVNLRFATSTTPKPFAIITPLSYSHVQSAVVCAKKAGIRIRIRSGGHDYVGLSYTSSDNVPFVVLDLKQLQNVTVEYSKKTAWVESGATIGQLYYWVSQKSKNLGFPGGTCATIGVGGHLSGGGFGTLVRKYGLSADNVIDAKIVDVNGRLLDRKSMGEDLFWAIRGGGGGSFGVVVAWMVNLVHVPEKVTAFTIVRTLEQGGSDLFNKWQHVGPKLTKDLFISVIIQPISVWNGNGTVQVIFNSMYLGTVDKLMKTVNSSFPELGLQAKDCTEMSWIQSVLYFAGYPIEGSMDVLKDRKPQTRRYFNNKSDHVKEPIPKERLEDLWKWCMEGDFPILLMDPLGGKMNEIDTTRIPYPYRNGYSYMIQYVETWENIGDSEKRISWMRQMYENMTPYVSKNPRSAYVNYRDLDLGKNDNAKNTSYLEAMKWGSKYFGDNFKRLAMVKGVVDPDNFFFHEQSIPPLKV

SEQ ID NO: 84 - protein, 256 aa, Helicrysum umbraculigerum
MDQYVITKFISYLLAVFMALFCSDPTADKFLQCFTKDSNATDSNFVFTQENTQYSSVLESTIINLRFATSITPKPIAVITPLSYSHVQSAILCSKKIGYRIRIRSGGHDYAGVSYTSYDHDHTPFVVLDLKELRTITIDSGENTSWVESGATVGELYYWVSQKSRNLGFPAGICPTVGVGGHLSGGGVGTMVRKYGLAADNVIDARIIDVNGRILDRKSMGEDLFWAIRGGGGASFGVIVAWKVNLVYVPEKSFGF

SEQ ID NO: 85 - protein, 530 aa, Helicrysum umbraculigerum
MELYISTRFILCFLVVLMLMFSSTYSDPLEDKFLRCLSQNSNATNSDNVFTQENTQYSSVLESTIINLRFATSTTPKPLAIITPLSCSHVQSAVLCAKKVGIRIRIRSGGHDYAGLSYTSSENAPFVVLDLKQLQNVTVESSKKTAWVESGATIGQLYYWVSQKSKNLGFPAGTCATIGVGGHLSGGGFGTLVRKYGLSADNVIDAKIVDVNGRLLDRKSMGEDLFWAIRGGGGGSFGVVVAWKVNLVHVPEKVTAFTIVRTLEQGGSDIFNKWQHIGHKLTKDLFIRVIIQPISVSNGNRTVQVIFNSMYLGTVDKLMKTVNSSFPELGLQEKDCTEMSWIQSVLYFAGYPIEGSMDVLKDRKPDTRNYFDNKSDHVKEPIPKERLEDLWKWCMEVDFPILIMEPLGGKMNEIDTTRIPYPYRKGYSYMIQYVEAWDNIGDSEKHISWLRQMYENMTPYVSKNPRSAYVNYRDLDLGKNDNAKNTSYLEAMKWGSKYFGDNFKRLAMVKGVVDPDNFFFHEQSIPPLKV

SEQ ID NO: 86 - protein, 530 aa, Helicrysum umbraculigerum; identical to SEQ ID NO: 83

SEQ ID NO: 87 - protein, 310 aa, Helicrysum umbraculigerum
MGEDLFWAIRGGGGGSFGVVVAWMVNLVHVPEKVTAFTIVRTLEQGGSDLFNKWQHVGPKLTKDLFISVIIQPISVWNGNGTVQVIFNSMYLGTVDKLMKTVNSSFPELGLQAKDCTEMSWIQSVLYFAGYPIEGSMDVLKDRKPQTRRYFNNKSDHVKEPIPKERLEDLWKWCMEGDFPILLMDPLGGKMNEIDTTRIPYPYRNGYSYMIQYVETWENIGDSEKRISWMRQMYENMTPYVSKNPRSAYVNYRDLDLGKNDNAKNTSYLEAMKWGSKYFGDNFKRLAMVKGVVDPDNFFFHEQSIPPLKV

SEQ ID NO: 88 - protein, 526 aa, Helicrysum umbraculigerum
MELKLFTCKLVTIILALSLSFFTSTSSSDFLDCISQKNLSNIIFTPNDTSYSTILQFTIPNLRFNTPKTTKPLAIITPTTYSHVQSTIICSVQFKHHVRIRSGGHDYEGLSYTSFNNTPFILLDLNQLRSVTVDLDSNTTWVESGATLGELLYWVSRKSNILGIPTGECTSVGVGGQLSGGGFGNMARKYGLFSDNAVDALIIDVNGRILDRDSMGEDLFWAIRGGGGGNFGVVLSWKINLVYVPPKVTVFTVSKMLDENGTKIVHKWQYIAHNITQDLFINLIVSPVTVSNTTILAVTINSLFLGMKNELVATMDVIFPELGLQEKDCIEMSWIESVVYHSVYLRGQSVDALIERRPWPKSYNKYKSDYVKKPMSEKALEKLWKWCLEENLILAIEPHGGKMSEIDESSTPYPHRKGNLYIIQYVMQWDEGYNTTQKHVASIRRVYKKMAPFVSKNPREAYVNFRDLDLGTNGNACGTSGASYVQALRWGKKYFKGNFKRLAIVKGRVDPTNFFCNEQSIPPYSY

SEQ ID NO: 89 - DNA, 1401 bp, Helicrysum umbraculigerum
  source 1..1401: mol_type = other DNA
atgaccaactcggaacttgttttcatcccatctccgggagccggccacctaccacctacggtggagctagcaaagctcctcctccaccgcgaaccacagctttcggttaccatcatcatcatgaacctccctcatgaaacaaaacccactactgaaactcgaatgtccactcctcgtctacgctttattgacatacctaaagacgagtcaacaaaagatcttatctcacgccacacattcatatccgccttccttgaacaccaaaagccacatgttcgaaacattgtccgttcaatcaccgagtctgactcggttcggttagttgggttcgtcgtagacatgttttgtattgccatgatggacgtcgcaaacgagctgggtgctccaacttatctttatttcacctcctctgccgcttcacttggcctcatgttttgcctacaggccaaacgagacgacgaggagtttgatgtgaccgagttgaaggacaaagattcggaactctccattccgtgttacaccaacccactcccagctaagttgttaccttcggtactatttgataagagaggtgggtcaaaaacatttattgacctcgctagaaagtatcgcgagtcgaggggtatagttgtaaatacttttcaagaactcgaaagctatgctattgagtatcttgcaagtagtaatgctaacgtcccaccggtgtttccggtgggggcgatactaaaccaagaaaaaaaggtaaatgatgataagacggaggagattatgacatggttaaacgagcaaccggagagttcggtggtgtttctatgcttcgggagcatgggaagcttcggtgaggatcaaattaaggaaatagcgcttgctatcgaagaaagcggacaaaggtttttgtggtcactacgtcgtcccccttcgaacgaaaataagtacccgaaagaatacgaaaattttggagaggttcttccggaaggtttccttgaacgaacatcgagtgtagggaaagtgataggatgggccccacaaatggcagtgttgtcccattcttcagttggtgggtttgtgtcacattgcggatggaactcgacactcgagagcatatggtgtggtgtaccggtagctgcgtggccattatatgcagaacaacaacttaatgcttttaaactagtggtggagttgggcttagcggtcgagattaagattgattataggagtgagaacgagattattttgacatcgaaagaaatcgagagtgggattaggaggttgatgaatgatgaagagttgaggatgaaagtgaaagagatgaaggggaatagtaggtttgcagtttcagagggtggatcttcttacgtatccattaggcgttttatcgaccttgtgatgactaaggagtaa

SEQ ID NO: 90 - DNA, 1428 bp, Helicrysum umbraculigerum
  source 1..1428: mol_type = other DNA
atgccgacctcagaacttgttttcatcccatcccccggtgtcggccacctgtcgcctaccatcgaactcgtcaatcaactcctccaccgcgaccagcgcctgtctgtcacaatcatcgtcatgaagttctctcttgaatcaaaacacgatacagaaactcctacatccactcctcgattacgcttcattgatatcccttatgacgagtccgctatggctctcattaacccgaacacgttcctctccgctttcgtcgagcacaacaaacctcatgttcgaaacattgttcgtgacatttccgagtctaactcggttcggctcgcggggtttgttgtggacatgttttgtgtagctatgacggatgtagtgaacgagtttgaaattccaacctatatttattttacctcgaccgcgaacttactcggactcatgttttaccttcaggccaagcgtgacgacgagggttttgatgtcaccgtgttgaaagactcagaatcagagtttttgtctgttccgagttatgtcaacccggttccagctaaggttttacctgatgcagttttggataagaatggtgggtctcaaatgtgtctggatcttgcaaaagggtttcgtgagtcgaagggcataatagtaaatacatttcaagaactcgaaaggcgtggaatcgagcaccttttaagtagtaacatgaacctcccacctgtgtttcctgtggggcctatattgaacttgagaaatgcgccaaacgatggtaaaacggccgatatcatgacatggttaaatgaccatccagagaactcggttgtgttcttgtgtttcggaagtatgggaagcttcgagaaagaacaagtgaaggagatagcgattgccatcgaacagagtgggcaacggtttctatggtcactccgtcgtccaacatcgctagaaaagtttgagtttccaaaggattacgagaacccggaggaggttttgccaaagggatttcttgaaaggacaaaaggtgtgggaaaggttatcgggtgggccccacaaatggcggtgttgtctcacccgtcagtgggagggttcgtgtcccactgtgggtggaactccacattggagagcatatggtgtggggtcccaatagcggcttggccactatatgcggaacaaaaaattaatgcttttcaattggtggtagagatgggaatggcagctgagattaggatcgactatcggactaatacgagaccgggtggtggtaaagagatgatggtaatggctgaagagattgagagtggtattaggaagttgatgagcgatgatgagatgagaaagaaagtgaaaggtatgaaggataaaagtagggctgctgttcttgaaggtggatcatctcacacatcaattgggattttaattgagaatttggtgagtataacgatctag

SEQ ID NO: 91 - DNA, 858 bp, Helicrysum umbraculigerum
  source 1..858: mol_type = other DNA
atggtgggtctcaaatgtttttggatcttgcaaaaaggttttcgtgagtcgaagggcataatagtaaatacatttcaagaactcgaaaggcgtggaatcgagcaccttttaagtagtaacatggacctcccacctgtgtttcctgtggggccgatattgaacttgagaaatgcgcgaaacgatggtaaaatggccgatatcatgacatggttaaatgaccagccagagaactcggttgtgttcttgtgtttcggaagtaggggaagcttcaaggaggaacaagtgaaggagatagcaattgccatcgaacaaagtgggcaacggtttctatggtcactccgtcgtccaacatcgatagaaacgtttgagtttccaaagtattacgagaacccggaggaggttttgccaaagggatttcttgaaaggacaaaaagtgtgggaaaggttatcgggtgggccccacaaatggcggtattgtctcacccgtcagtgggagggttcgtgtcccactgtgggtggaactccacattggagagcatatggtgtggggtcccaatagcggcttggccactatatgcggaacaacaaactaatgcttttcaattggtggtcgagatgggaatggcagcagagattaggatcgactatcggactaatacaccactggttggtggtaaagacatgatggtaacggctgaagagattgagagaggtattaggaagttgatgagcgatgatgagatgcgaaagaaagtgaaagacatgaaggataagagtagaggtgcagttttagagggtgggtcatctcatacatcaattgggaatttaattgatgttttggtgagtataacgatctag

SEQ ID NO: 92 - DNA, 1467 bp, Helicrysum umbraculigerum
  source 1..1467: mol_type = other DNA
atggcgaccaacaacctccatttccttctaattccccatataggtccaggccacactattcccatgatagatatggctaaacttcttgcaaaacaaccaaatgtaatggttacaatagctacaacacctcttaatatcacccgttacgggcacactctcgcagacgccatcaactcgtttcgcttctttgaggttccatttccggcagttgaggctggattacctgaaggatgtgaaagcacggataaaatcccaagtatggatctagtaccgaactttttaaccgcgattggtatgctagaacaaaagctagaagagcattttcacttgctagagcctcgtccgaattgtattatttctgataagtacatgtcgtggacgggtgattttgctgataagtatcggatccctagaattatgtttgatggaatgagctgttttaacgagttatgttacaacaatttgtatgaaaacaaggtgtttgaagggatgcatgaaacagaaccatttgttgtccctggtttacccgataaaattgagctaacacgaaaacagctcccacctgagtttaacccgagctcgattgatacaagtgagtttcgtcagcgggctagggacgctgaggtgagggcttatggagttgtgatcaatagttttgaggagttggaacaagaatatgttaatgagtataagaagttaagaaagggtaaggtttggtgtatcggcccgctgtcactgtgcaatagtgacaattcggataaagcccaaagaggaaatatagcgtcagtcgatgaagaaaaatgtttaaaatggcttgattctcatgaagccgactcagtagtttacgcttgttttggtagccttgttcgggtcaacaccccacaactaattgagcttggtttaggcctagaagcatcaaatcgcccgttcatttgggtggttagatcggttcatagagaaaaagaggtcgaggaatggctagtggaaagtggttttgaggagagaattaaagatagaggtttaataatccgaggttgggccccacaagtacttatcttgtctcacccttctattggagggtttttaacgcattgcggttggaactcgaccctagaatcagtctgtgcaggtgttccaatgatcacatggcctcaatttgcagagcaatttatcaacgagaagctaatagtgcaagtgttggggattggtgtgggtgttggagttgattctgttgtccatgtgggcgaagaagatagatctggggtgaaagtgaagagggagagtgttacgaaggctattgagaaagtcatggatgacgagattgatggaaatgagagacggaggagatcgaaagagtttggaaagatagctaataacgcgattaaagagggagggtcttcataccttaacttgactctgctaattcaggacataatgcgttatgcaaatgcagatgcttcaagctaa

SEQ ID NO: 93 - DNA, 1407 bp, Helicrysum umbraculigerum
  source 1..1407: mol_type = other DNA
atggaaaaaacacctcatatagccattgtaccaagtccaggaatgggccacttgatccctttagttgagtttgctaaaaaactaaaaaatcaccacaacatacatgcaactttcatcatcccaaatgatggacctttatctatttctcaaaaggtttttcttgattcacttcctaatggtttaaactatctcattctacctccggtaaattttgatgatttaccacaagatacccaaatcgaaactcgaattagtctaatggtaacacggtctcttgattcgctacgtgaagtgtttaagtcattagttgtggaaaaaaatatggttgctttgtttattgatctttttgggacagatgcatttgatgttgctattgaatttggtgtttcaccttatgtgttctttccatcaactgctatggctttatctttgtttctatatttgcctaaacttgatcagatggtttcatgtgagtatagggagcttcctgaaccggttcaaattccaggttgtataccggttcgtggacaagacttggttgacccggttcaagatagaaagaatgatgcatacaaatgggtgcttcataatgcaaagaagtattcaatggctaagggtatagcggtaaatagcttcaaggagttagaaggtggagctttgaatgctttgctagaagatgaaccgggtaagccaaaagtttatccggtcggaccgttagtacaaaccggttttagttgtgatgttgattcgatagagtgcttgaagtggttagatggtcagccatgtggttctgttttgtatatatcttttggaagcggtgggaccctttcatccagtcaacttaatgagttagctatgggtttggagttgagtgaacaacggttcatatgggtggttagaagcccgaacgatcaaccaaacgccacgtactttgattctcatggtcacaaagaccctcttggttttttgcccaaagggttcttggaaagaaccaaaggaattgggtttgtgatcccttcttgggctccacaagcccagatcctgagtcacagtgccacaggtggatttttaacccactgtggttggaactcaattctcgagactgtagtccatggtgtgccggtgattgcttggccactttatgccgagcaaaagatgaatgcagtgtctttaaccgagggtataaaaatggcgttaagacccacggttggtgaaaatgggattgtgggtcgcttagaggttgcgagagttgtgaagagtttactggaaggagaagaagggaaggcgattaggagtcgagttcgtgatctcaaggatgctgctgctaatgttcttagtaaagatgggtcttctacaaaaactttagatcaattggctgtacagttgaaaaaacaagaattaagctag

SEQ ID NO: 94 - DNA, 1434 bp, Helicrysum umbraculigerum
  source 1..1434: mol_type = other DNA
atgactcaaaagcaaatgcaaatgcaacctcactttctcttagtaacatatcccgcacaaggtcatattaacccgtctctccagttcgctgaacgtctcattcggttgggtgtcaaagtcaccttcacaacaactgtctctgcttaccgccgaatgagtaaagcgggcaacatctcagagtttttaaattttgctgctttttcagacggctttgatgacggtttcaacttcgaaacagacgatcatggtctcttcttaactcaattgagaagcaggggaaaagatagcttgaaagaaacaattctttcaaatgctaaaaatggaactccaattagttgtttggtttacacactcctactcccttgggctcctgaggtggcacgtggcctaaacgtgccctcagcctttctttggattcaaccagcttctgttttacgactttactattactacttcaatgggtacaatgaactcatcggtgacgattgtaatgaaccttcatggtccattcaattaccagggttaccattgctcaaaagtcatgaccttccctccttttgtctcccttcaaatccttacagtaatgtactggctctagtcaaagagcatttagatatgctggatctggaagagaagcctaaaatacttgtgaatagttttgatgagttggagagggaggcgttgaatgaaattaatggaaaactaaaaatggtcgccgtagggcctttgattccatcagcttttttggatggacaagatgcatctgacaaatcttttaggggagatttgtttgaaacatccaaagattatttggaatggatgaatacaaagcctgaagggtccattgtttacatatcttttggtagtcttttagtgttctcaaagatacaaaaggaggcaatggcacatgctttgttagagtgcgggaggccgttcttgtgggtgataagagatggagaacaaggagaacaactaagttgtattgagaaattggaacaattaggtttgatagtcccatggtgtagtcaactagaggtattatcacacccttctttaggttgttttgtgacacattgtggttggaactcgactttagagagtatagtttgtggagttcctgtggtggcatttcctcaatggacagatcagacgacaaatgcaaagcttctagaagacgtatggggaacaggggtgagagtgacaactaatgaagacggggttgttgaaagcgaggagataagaaggtgcatcgaaatggtaatgggaggccgtgatagtgaatcaacaatgagaaagaatgctaagaagtggaaggatgtgggaagagaggctatgaaagaaacaggatcttcttatatgaatctcaaggcttttattaaagaagtgaatgatggtgaatcaaccatcaaaactgaaattgtttcaactatatga

SEQ ID NO: 95 - DNA, 762 bp, Helicrysum umbraculigerum
  source 1..762: mol_type = other DNA
atgactaaaatacaacagcaacctcactttctcttagtaacatatcccgcacaaggtcatattaacccgtctctccggttcgccgaacgactcattcggttgggtgtcaaagtcaccttcacaataactgtctctgcttaccgccgaatgagtaaagcgggccacatctcagagtttttaaattttgctgttttttcagacggctttgatgacggtttcaactccaaaacagacgattatggtctcttcttaactcaattcagaagcaggggaaaagatagcttgaaagaaacaattctttcaaatgctaaaaacggaactccagttagttgtttggtttacacactcctactcccttgggctcctgaggtggcacgtggcctaaacgtgccctcagcctttctttggattcaaccagcttctgttttacgactttactattactacttcaatgggtacaatgaactcatcggcgacgattgtaacgaaccttcatggtccattcaattaccagggttaccattgctcaaaagtcgtgaccttccctccttttgtctcccttcaaatccttacgctgatgtactgactttagtcaaagagcatttagatgtgttggatttggaagagaagcctaaaatacttgtgaatagttttgatgagttggagagggaggcgttgaatgaaattgatgggaaactaaaaatggttgccgtagggcctttgattccatcagctttttttggatggacaggatgcatctga

SEQ ID NO: 96 - DNA, 1014 bp, Helicrysum umbraculigerum
  source 1..1014: mol_type = other DNA
atgggttcatggcggaattcaagaacaacgtctacaaagtttttatggttgattttaccgttgatggtggtgacggtgattataggggtaaaaaagtcaaattatgggtcgaagtataattatccttgggtttggagttcagtgattaattcttattcttcttctgcggttaaagaagatgtaacggtggtggctgaaggtcctgttgaatcatttgggttgcggtcaacggtggtcaacggtggtggtgtggtggcggaagggccgtcggaagattttggttttaattcttcttatccaccgttggctatggaagatgaaatggatgttgagctacctgctattgccaaggaagatgacttgaacgcgacgttgagtggacccgacctttttgtgtctgcaaatcaaactggcggacttcatgttgatattggaatcaacagtaagtataccagtttggataagcttgaagcccgcttaggtcaggttcgagctgcaataaaagaagccgaatcaggaaatagaacttacgatccggattatgtaccagagggtcctatgtactggcatgcagcctcatttcacaggagttatttggagatggaaaagcaatttaaggtgtttgtatatgaagaaggagaaccaccaatatttcataacggtccttgcaaaaacatatatgcaatggaaggtaactttatctaccatatggaaacaaccaagtttaggacaaaaaaccccgaaaaagctcacacgttttttctcccaatgagtgctgcaatgatggtgaggtttatctttgagcgtgatccaaatgttgaccattggcgtcctatgaagcaaacaattaaagattatgttgatcttgtgggtggtaagtacccattttggaatcgaagcttaggagccgatcactttactgttgcgtgccacgattgggtgagtaaagtcttttatcccatcattttcatgcttttactagtatttatcttcagaatgtcgactggatgctga

SEQ ID NO: 97 - DNA, 1410 bp, Helicrysum umbraculigerum
  source 1..1410: mol_type = other DNA
atgtcaaccgttgaggttgcaaagttacttgtgaatcgagatcatcgtctcttcataacattccttatcattcagcctcctagctcgggttctggctcagctatcaccacctacatcgaatcattagctgagaaagctatggaccgcatatccttcattgagctacctcaagataaaatcccaccaccacgttacccgaaatccctgccaactgcagaatcgaaagctcatccccttattttcatgattgagttcattaagtgtcactgcaaatatgttagaaacattgtatctgacatgataagtcaaccgagttcgggtcgggtagctgggttggtaatcgacatgctttgtttcagcatgatggatgtcgctaatgagttcaacattccaacctatgtatttgtcacttctaatgctgcttttcttggattttatttatatgtccagatactctctaatgatcagaaccaagacgttgttgagctgagcaaatctgataccgagatatcggttccaggttttgtaaagccggtgccaacgaaagtcttctggactgttgtccgcactaaagaaggactggactttgttttgtcatctgcccagaaacttagacaagccaaagcaatcatggttaataccttcttggagttggaaacacacgcaatcaagtcgctgtctgatgacaccagcatcccgcctgtgtatccagtgggaccgatactcaatttagaaggtggtgctggcaaaacgttcgacaatgacattagcaggtggttggacagtcaaccgccttcctcggtggtgttcttgtgctttggaagccacggatgttttgatgagatccaagtgaaggagatagcacatgctttagagcagagtggccaccgtttcttgtggtccctacgtcgacctccatcagatcaaacattaaaagttcccggtgattacgaggatccaggagtggtattaccggaaggatttcttgagcgaactgctggacgtgggaaagtaattgggtgggccccgcaggtgatggtgctggctcaccgtgcagttggaggcttcgtgtcccactgtgggtggaactcgttgttggagagtttgtggttcggcgtaccaacggcaacatggccgatctatgctgagcagcagatgaatgcgtttgaaatggtggtggagctgggactggctgtggagataacattggattataggaatgatatggatatgttcattgtcaccgcacaggagatagaaagtggtataagaaaggtgatggaggataatgaggtaagaacaaaagtgaaagagagaagtgagaagagtagagcagcagtggcggagggggggtcatcgtatgcatctgttggtcatcttattaaagaatttacaggaaacatctcctaa

SEQ ID NO: 98 - DNA, 1173 bp, Helicrysum umbraculigerum
  source 1..1173: mol_type = other DNA
atgtcatcattcatcaactttgttgaatccacaacacaacttcaaccacaattcgaacaactcatccaaacacttcttcccataactgcgataatatcggatggttttttgatgtggacacaagattccgccgaaaaattcaatatcccacgtctggttttttatgggacaaacatatttttcatgactatgtgtaacattatggcacaatttaagccacatgcggctgttaattctgatgatgaggcgtttgatgtacccggtttcaccaggtttaagttgacggctaatgattttgagccgccttttaatgaggttgaaccgaaaggttcaatgttggattttttattggagcaacaaaaggctatggttaggagccatgggttggtggttaatagtttttatgagattgaacatgagtttaatgtttattggaatcagaactatggacctaaagcttggttaatgggaccattttgtgtagctaagccatatgcatcaaacgtcatggattccgagatatcgactaaggtggtgaaaaaatcagcatggatccagtggcttgacaggaagcttgcagcgaacgagccagtgttatacatctcatttggaacacaggcagaggcgtctatggagcacttacacgaggtcgctattggtttggaacgatcaaatgtaagcttcatttgggtggtaaaagcgaagcagatgcaattaattggagcagggtttgaagagagggtgaaggggagaggaaaagtggtgacagaatgggtggatcagatggaaatcttgaaacatgaaattgtaagcgggtttttaagtcattgtgggtggaactcactgctagagagtatgtgtgtgggtgtgccggtgcttgcaatgccgttgatggcggatcaactcttaaatgcaaggttggttgtggaggagattgggatggggctacggttgtggccgaggggtatggtggcacgtgggatagttggggcggaggaagtcgagaaaatggtggtggagttgatggaaggggaaggtgggagaagggtgcggaaaagggtcatcgaggttagagaaatggcatatggtgcgatgaaggaaggagggtcatcatcgaggacattagactcgttgattgatcatgtttgtgaagcctttcataagacggtttaa

SEQ ID NO: 99 - DNA, 1380 bp, Helicrysum umbraculigerum
  source 1..1380: mol_type = other DNA
atggggagcttgaagaaaggtgcacatatactaatattcccattcccagcacaaggtcatatgctcccactcctagacctaactcaccacctagccaccaatgggttaaccataaccatattagtcacacccaaaaacctaccaatcttgaacccacttttatcttcatctccaaacatccaaccactagtcttccctttcccacctcacccaagacttccaccacatgttgaaaatgttaaagacataggtaaccatgcaaatgtcccaatcacaaactcactagccaaattacaagaccaaataatccagtggtttaactcccaccataaccctcctgttgccatcatctcagatttctttcttggatggacccaacaccttgcaaacaaacttggtatccctcgtgtcgggtttttttcttctggtgcttacttgactgctgttcttgattatgtttgtcataatattaaaactgttaggtctcaagaggagactgtttttcatgacttgccaaattctccttgttttaaattcgagcatcttccgggtttggcccagatttataaagagtccgacccggaatgggaattggttcttgatggtcatattgcgaatgggttaagttggggttggattgtgaatacttttgatgggttggagtctcggtatatggagtatctgacaaagaaaatgggtgtcggacgggtttttggtgtcgggccagttaatttgttaaacgggtcggatcccatgacccgtgggaaatcggaatccgggtctgattccggtgtgttgaactggctcgatggaaaacccgatgggtcggttttgtatgtgtgttttggaagtcaaaagtttcttactaatgaccaaatggagggattgtcaattgggcttgaacaaagtggggtccattatgtttgggttgtgaaagacgaacaaggtgatgcaattaggtccgggtcgggtagaggactagtggtaacgggttgggccccgcaagtttcaatattgggtcatggagcggtgggtgggtttttgagtcattgcgggtggaactctgttttggaagcaattgtaaatggagttatgatattggcttggccaatggaggctgatcaatttgttaatgctaagttgttagtggatgaccatggtataggggtgtgggtttgtgaggggccgaatacggttcctgattcaaccgagttggctcgtaaaattggtgagtcaatgagtacggataagagtgagaaggtaaaggcgaaagaaatgaaaaacaaagcaaatgaagcagttaaagaaggtgggagctcatcaatggaattaagcaggcttgttaaggagctgtctaactttgagacaaatgggccatga

SEQ ID NO: 100 - DNA, 1611 bp, Helicrysum umbraculigerum
  source 1..1611: mol_type = other DNA
atggatacccaaacacaagtcaagaaacaaaaacttgaaaccatggaacataaaacatcatccgccgaaatcttcgtgctaccattttttggtacgggtcatataaacccagcaatggagctttgccggaacatttcatcacataattacaaaactaccctcatcatcccttcacatctttcttcatctattccttctcccttttcttcaactttacttcatgttgctgagatccctttcactgcttctgacccggaacccggatccggaagagggaacccacttgatgcccagaacaagcaaatgggtgaagggattaaggcgtttatgtctgcaagatctgacggatcaaaactacccacgtgtgttgttattgatgtcatgatgaactggagtaaagagatatttgttgattaccagattcctattgtctctttttttacttctggagctactaatactgctatgggttatggtaggtggaaagctaaaattggtgatctgaagcccggggagacccgtgtgatccccggacttcctactgaaatggccgttacttttgcggatttaaatcaaggtcctagaggccgtgggcctcggccggatgggtcaaggcctgacgggccaaggtctggaccacctggtgggatgaggtccggaccacctcacgggatgaggggtgggggacgaggtgggcggggcggtggacgacccggcccggatgcgaaaccacgttgggtagatgaagtggacgggtcggtagctttgcttatcaacacgtgtgacaatctcgagcgtgtgtttattgattacattgctgaagaaaccaagattcccgtttatggtgttggcccgttgctgcccgaaaagtattggaagtcagcgggttcgttgcttcgtgatcatgaaatgaggtctaaccataaagcgaattactcggaagatgaggtgtttcaatggctagaatcgaaaccagttgggtcggttatttacatatcgtttgggagtgaagttggcccgactatagacgagtataaagagttagctggatcattggaaggatcgaatcagaatttcatttgggtgatccagcccggttcggggataacgggcatgccaagatcgtttttgggcccggttaatacggatagtgaggaagaagaggaagggtattatcctgagggattagatgttaaagttgggaacaggggtttgatcatcactggatgggctccacagttgttgattttgagccacccatctacaggcgggttcttatcacattgtgggtggaattcaactgttgaggcgattgggcgaggtgttccgatattgggttggcccttgaggggtgatcagtttgataatgcgaaacttgtggcgaatcatttgaaaattgggtttgcgatgtcaagtgtggcgagtgaaggcggacgacctgggaagttcaacaaggagactataacagcagggattgagaaactaatgaatgatgaagatgtgcataaacaggcaaagaaacttagtaaagaatttgagagtgggtttccagtgagttcagttaaagcattgggtgctttcgtggagtctattagccagaaagcaacctaa

SEQ ID NO: 101 - DNA, 1378 bp, Helicrysum umbraculigerum
  source 1..1378: mol_type = other DNA
atgtcactcgtgactaataacccacatttactagtctacccattacctacctccggccatatcattccgttactcgacctgaccgaccttcttctccgccgtggcctcaccatcaccgtcgtgatatccaccacagaccttacgcttctcgacactctcctatcctcacaccccacgtctctacacaaactttacttccccgaccccgaaatcggcccatcttctcatcccgttattgccagaataattgccacccaaaaactatttgatccaattgttaaatggtttgaatcgcacccttcgcctccagtcgccatcatttccgacttctttcttgggtggactaatgaacttgcatcacgtttaggtattcgacgtgtggtgttttcaccttcgggagctcttggtcattccattttacaaagtttgtggcgtgacgtggcggagatcaatgcaaaaaatgttgatggaaatggaaactactcgatttcttttaccgatataccaaactcgcccgaatttcattggtggcagttgtcacaacttttgcgtgttcatagggagggagatccggacttcgaattttttaggaatggaatgttggctaatacgaaaagttggggtattgtttacaacacatttgaaaggattgaaaaggtttacattgaccatgtgaagaaacaaataggtcatgatcgggtatgggcaataggcccattacttcccgaagaacatggcccagttggtagcaccgcacgtggtgggtccagtgtagtgccacctcatgaccttctcacgtggttggacaaaaagccccatgactcggtcgtatatatatgttttgggagtcgattgacgttaagtgagaagcaaatgagtgcattagcaagtgcactcgagctcagtaacgttgattttattttgtgtgtgaaggcaagtggttcgagcttcattcctagtgggttcgaagatcgagtggtgggtcgggggttcgtaatcaaaggttgggccccacagttggcgatattgagacatcgggctgtggggtcgtttgtgactcattgtgggtggaactcaacattggaaggtgtttcatcaggagtgatgatgttgacgtggccaatgggtgcagaccaatatgcaaatgctaagctattggtcgaccagttaggtgttgggaaacgagtttgtgaaggtggacccgagagtgttcctgattcaactgagttggctcggttgttggaagagtcactgagtggtgatacatccgagcgagttaaagtcaaggagctaagtcgggaagctaacacagctgtgaaagaaggaacttcaataagagatttrgaacatgttcgttaaccttttatccgagctctaa

SEQ ID NO: 102 - protein, 466 aa, Helicrysum umbraculigerum
MTNSELVFIPSPGAGHLPPTVELAKLLLHREPQLSVTIIIMNLPHETKPTTETRMSTPRLRFIDIPKDESTKDLISRHTFISAFLEHQKPHVRNIVRSITESDSVRLVGFVVDMFCIAMMDVANELGAPTYLYFTSSAASLGLMFCLQAKRDDEEFDVTELKDKDSELSIPCYTNPLPAKLLPSVLFDKRGGSKTFIDLARKYRESRGIVVNTFQELESYAIEYLASSNANVPPVFPVGAILNQEKKVNDDKTEEIMTWLNEQPESSVVFLCFGSMGSFGEDQIKEIALAIEESGQRFLWSLRRPPSNENKYPKEYENFGEVLPEGFLERTSSVGKVIGWAPQMAVLSHSSVGGFVSHCGWNSTLESIWCGVPVAAWPLYAEQQLNAFKLVVELGLAVEIKIDYRSENEIILTSKEIESGIRRLMNDEELRMKVKEMKGNSRFAVSEGGSSYVSIRRFIDLVMTKE

SEQ ID NO: 103 - protein, 475 aa, Helicrysum umbraculigerum
MPTSELVFIPSPGVGHLSPTIELVNQLLHRDQRLSVTIIVMKFSLESKHDTETPTSTPRLRFIDIPYDESAMALINPNTFLSAFVEHNKPHVRNIVRDISESNSVRLAGFVVDMFCVAMTDVVNEFEIPTYIYFTSTANLLGLMFYLQAKRDDEGFDVTVLKDSESEFLSVPSYVNPVPAKVLPDAVLDKNGGSQMCLDLAKGFRESKGIIVNTFQELERRGIEHLLSSNMNLPPVFPVGPILNLRNAPNDGKTADIMTWLNDHPENSVVFLCFGSMGSFEKEQVKEIAIAIEQSGQRFLWSLRRPTSLEKFEFPKDYENPEEVLPKGFLERTKGVGKVIGWAPQMAVLSHPSVGGFVSHCGWNSTLESIWCGVPIAAWPLYAEQKINAFQLVVEMGMAAEIRIDYRTNTRPGGGKEMMVMAEEIESGIRKLMSDDEMRKKVKGMKDKSRAAVLEGGSSHTSIGILIENLVSITI

SEQ ID NO: 104 - protein, 285 aa, Helicrysum umbraculigerum
MVGLKCFWILQKGFRESKGIIVNTFQELERRGIEHLLSSNMDLPPVFPVGPILNLRNARNDGKMADIMTWLNDQPENSVVFLCFGSRGSFKEEQVKEIAIAIEQSGQRFLWSLRRPTSIETFEFPKYYENPEEVLPKGFLERTKSVGKVIGWAPQMAVLSHPSVGGFVSHCGWNSTLESIWCGVPIAAWPLYAEQQTNAFQLVVEMGMAAEIRIDYRTNTPLVGGKDMMVTAEEIERGIRKLMSDDEMRKKVKDMKDKSRGAVLEGGSSHTSIGNLIDVLVSITI

SEQ ID NO: 105 - protein, 488 aa, Helicrysum umbraculigerum
MATNNLHFLLIPHIGPGHTIPMIDMAKLLAKQPNVMVTIATTPLNITRYGHTLADAINSFRFFEVPFPAVEAGLPEGCESTDKIPSMDLVPNFLTAIGMLEQKLEEHFHLLEPRPNCIISDKYMSWTGDFADKYRIPRIMFDGMSCFNELCYNNLYENKVFEGMHETEPFVVPGLPDKIELTRKQLPPEFNPSSIDTSEFRQRARDAEVRAYGVVINSFEELEQEYVNEYKKLRKGKVWCIGPLSLCNSDNSDKAQRGNIASVDEEKCLKWLDSHEADSVVYACFGSLVRVNTPQLIELGLGLEASNRPFIWVVRSVHREKEVEEWLVESGFEERIKDRGLIIRGWAPQVLILSHPSIGGFLTHCGWNSTLESVCAGVPMITWPQFAEQFINEKLIVQVLGIGVGVGVDSVVHVGEEDRSGVKVKRESVTKAIEKVMDDEIDGNERRRRSKEFGKIANNAIKEGGSSYLNLTLLIQDIMRYANADASS

SEQ ID NO: 106 - protein, 468 aa, Helicrysum umbraculigerum
MEKTPHIAIVPSPGMGHLIPLVEFAKKLKNHHNIHATFIIPNDGPLSISQKVFLDSLPNGLNYLILPPVNFDDLPQDTQIETRISLMVTRSLDSLREVFKSLVVEKNMVALFIDLFGTDAFDVAIEFGVSPYVFFPSTAMALSLFLYLPKLDQMVSCEYRELPEPVQIPGCIPVRGQDLVDPVQDRKNDAYKWVLHNAKKYSMAKGIAVNSFKELEGGALNALLEDEPGKPKVYPVGPLVQTGFSCDVDSIECLKWLDGQPCGSVLYISFGSGGTLSSSQLNELAMGLELSEQRFIWVVRSPNDQPNATYFDSHGHKDPLGFLPKGFLERTKGIGFVIPSWAPQAQILSHSATGGFLTHCGWNSILETVVHGVPVIAWPLYAEQKMNAVSLTEGIKMALRPTVGENGIVGRLEVARVVKSLLEGEEGKAIRSRVRDLKDAAANVLSKDGSSTKTLDQLAVQLKKQELS

SEQ ID NO: 107 - protein, 180 aa, Helicrysum umbraculigerum
MTQKQMQMQPHFLLVTYPAQGHINPSLQFAERLIRLGVKVTFTTTVSAYRRMSKAGNISEFLNFAAFSDGFDDGFNFETDDHGLFLTQLRSRGKDSLKETILSNAKNGTPISCLVYTLLLPWAPEVARGLNVPSAFLWIQPASVLRLYYYYFNGYNELIGDDCNEPSWSIQLPGLPLLKS

SEQ ID NO: 108 - protein, 253 aa, Helicrysum umbraculigerum
MTKIQQQPHFLLVTYPAQGHINPSLRFAERLIRLGVKVTFTITVSAYRRMSKAGHISEFLNFAVFSDGFDDGFNSKTDDYGLFLTQFRSRGKDSLKETILSNAKNGTPVSCLVYTLLLPWAPEVARGLNVPSAFLWIQPASVLRLYYYYFNGYNELIGDDCNEPSWSIQLPGLPLLKSRDLPSFCLPSNPYADVLTLVKEHLDVLDLEEKPKILVNSFDELEREALNEIDGKLKMVAVGPLIPSAFFGWTGCI

SEQ ID NO: 109 - protein, 337 aa, Helicrysum umbraculigerum
MGSWRNSRTTSTKFLWLILPLMVVTVIIGVKKSNYGSKYNYPWVWSSVINSYSSSAVKEDVTVVAEGPVESFGLRSTVVNGGGVVAEGPSEDFGFNSSYPPLAMEDEMDVELPAIAKEDDLNATLSGPDLFVSANQTGGLHVDIGINSKYTSLDKLEARLGQVRAAIKEAESGNRTYDPDYVPEGPMYWHAASFHRSYLEMEKQFKVFVYEEGEPPIFHNGPCKNIYAMEGNFIYHMETTKFRTKNPEKAHTFFLPMSAAMMVRFIFERDPNVDHWRPMKQTIKDYVDLVGGKYPFWNRSLGADHFTVACHDWVSKVFYPIIFMLLLVFIFRMSTGC

SEQ ID NO: 110 - protein, 469 aa, Helicrysum umbraculigerum
MSTVEVAKLLVNRDHRLFITFLIIQPPSSGSGSAITTYIESLAEKAMDRISFIELPQDKIPPPRYPKSLPTAESKAHPLIFMIEFIKCHCKYVRNIVSDMISQPSSGRVAGLVIDMLCFSMMDVANEFNIPTYVFVTSNAAFLGFYLYVQILSNDQNQDVVELSKSDTEISVPGFVKPVPTKVFWTVVRTKEGLDFVLSSAQKLRQAKAIMVNTFLELETHAIKSLSDDTSIPPVYPVGPILNLEGGAGKTFDNDISRWLDSQPPSSVVFLCFGSHGCFDEIQVKEIAHALEQSGHRFLWSLRRPPSDQTLKVPGDYEDPGVVLPEGFLERTAGRGKVIGWAPQVMVLAHRAVGGFVSHCGWNSLLESLWFGVPTATWPIYAEQQMNAFEMVVELGLAVEITLDYRNDMDMFIVTAQEIESGIRKVMEDNEVRTKVKERSEKSRAAVAEGGSSYASVGHLIKEFTGNIS

SEQ ID NO: 111 - protein, 390 aa, Helicrysum umbraculigerum
MSSFINFVESTTQLQPQFEQLIQTLLPITAIISDGFLMWTQDSAEKFNIPRLVFYGTNIFFMTMCNIMAQFKPHAAVNSDDEAFDVPGFTRFKLTANDFEPPFNEVEPKGSMLDFLLEQQKAMVRSHGLVVNSFYEIEHEFNVYWNQNYGPKAWLMGPFCVAKPYASNVMDSEISTKVVKKSAWIQWLDRKLAANEPVLYISFGTQAEASMEHLHEVAIGLERSNVSFIWVVKAKQMQLIGAGFEERVKGRGKVVTEWVDQMEILKHEIVSGFLSHCGWNSLLESMCVGVPVLAMPLMADQLLNARLVVEEIGMGLRLWPRGMVARGIVGAEEVEKMVVELMEGEGGRRVRKRVIEVREMAYGAMKEGGSSSRTLDSLIDHVCEAFHKTV

SEQ ID NO: 112 - protein, 459 aa, Helicrysum umbraculigerum
MGSLKKGAHILIFPFPAQGHMLPLLDLTHHLATNGLTITILVTPKNLPILNPLLSSSPNIQPLVFPFPPHPRLPPHVENVKDIGNHANVPITNSLAKLQDQIIQWFNSHHNPPVAIISDFFLGWTQHLANKLGIPRVGFFSSGAYLTAVLDYVCHNIKTVRSQEETVFHDLPNSPCFKFEHLPGLAQIYKESDPEWELVLDGHIANGLSWGWIVNTFDGLESRYMEYLTKKMGVGRVFGVGPVNLLNGSDPMTRGKSESGSDSGVLNWLDGKPDGSVLYVCFGSQKFLTNDQMEGLSIGLEQSGVHYVWVVKDEQGDAIRSGSGRGLVVTGWAPQVSILGHGAVGGFLSHCGWNSVLEAIVNGVMILAWPMEADQFVNAKLLVDDHGIGVWVCEGPNTVPDSTELARKIGESMSTDKSEKVKAKEMKNKANEAVKEGGSSSMELSRLVKELSNFETNGP

SEQ ID NO: 113 - protein, 536 aa, Helicrysum umbraculigerum
MDTQTQVKKQKLETMEHKTSSAEIFVLPFFGTGHINPAMELCRNISSHNYKTTLIIPSHLSSSIPSPFSSTLLHVAEIPFTASDPEPGSGRGNPLDAQNKQMGEGIKAFMSARSDGSKLPTCVVIDVMMNWSKEIFVDYQIPIVSFFTSGATNTAMGYGRWKAKIGDLKPGETRVIPGLPTEMAVTFADLNQGPRGRGPRPDGSRPDGPRSGPPGGMRSGPPHGMRGGGRGGRGGGRPGPDAKPRWVDEVDGSVALLINTCDNLERVFIDYIAEETKIPVYGVGPLLPEKYWKSAGSLLRDHEMRSNHKANYSEDEVFQWLESKPVGSVIYISFGSEVGPTIDEYKELAGSLEGSNQNFIWVIQPGSGITGMPRSFLGPVNTDSEEEEEGYYPEGLDVKVGNRGLIITGWAPQLLILSHPSTGGFLSHCGWNSTVEAIGRGVPILGWPLRGDQFDNAKLVANHLKIGFAMSSVASEGGRPGKFNKETITAGIEKLMNDEDVHKQAKKLSKEFESGFPVSSVKALGAFVESISQKAT

SEQ ID NO: 114 - protein, 458 aa, Helicrysum umbraculigerum
MSLVTNNPHLLVYPLPTSGHIIPLLDLTDLLLRRGLTITVVISTTDLTLLDTLLSSHPTSLHKLYFPDPEIGPSSHPVIARIIATQKLFDPIVKWFESHPSPPVAIISDFFLGWTNELASRLGIRRVVFSPSGALGHSILQSLWRDVAEINAKNVDGNGNYSISFTDIPNSPEFHWWQLSQLLRVHREGDPDFEFFRNGMLANTKSWGIVYNTFERIEKVYIDHVKKQIGHDRVWAIGPLLPEEHGPVGSTARGGSSVVPPHDLLTWLDKKPHDSVVYICFGSRLTLSEKQMSALASALELSNVDFILCVKASGSSFIPSGFEDRVVGRGFVIKGWAPQLAILRHRAVGSFVTHCGWNSTLEGVSSGVMMLTWPMGADQYANAKLLVDQLGVGKRVCEGGPESVPDSTELARLLEESLSGDTSERVKVKELSREANTAVKEGTSIRDLNMFVNLLSEL

SEQ ID NO: 115 - DNA, 1368 bp, Helicrysum umbraculigerum
  source 1..1368: mol_type = other DNA
atggcaacccaagtcaaaaccgaggagaagcatttgaaggtagaaatcataaacaaaacctatgtgaaacctgaaacaccactaggaagaaaagagtgtcaattggtcacatttgatcttccttatatagccttctactacaaccaaaagttgatcatctataaaggtggtgtcgaggagttcgaggataccgtcgagaaactgaaagacgggttaaaggtagttttgggagagtttcatcaattggctggaaaattagacaaagatgatgacggggtgtttaaggtagtgtacgatgatgacatggatggggtggaggtgctttctgcggtcgcggaagacactgcgaccgcagatttgatggacgaagaagggaccatcaagcttaaggagttggtcccttataatagtgttttgaacatagaggggcttcatcgtccgcttttatcgattcagataacaaaactaaaagatgggcttgtactgggctgtgcgttcaaccacgcgattttagacggtacatccacctggcacttcatgagctcctgggcccaaatttgctccggatccaaatccatttcagcggcgcctttccttgaccgtacccaagcgcgtaacacgcgcgtgaaactcgatctcacccctcccgcccaaactaacggcaattcaaacggcgacactaacggtgatgcgagcgccacgaagccaccagcaccggcaccgttaagagaaaaaatcttcaaattctcagagtcagcaatcgacaaaatcaaagcaaaaatcaatgcgaatccaccggaaggatcaaccaagccattctccacatttcaatcgctctccacacacatatggcacgcagttacacgcgctcgcaatctaaaaccggaagactacaccgttttcactgttttcgccgattgccggaaacgtgtcgatcctccgatgccggatagctatttcggaaacctaattcaagcgatcttcaccgtcaccgctgccggattattgcaggcgaatccaccggaattcgcggcgtcaatgatacaaaaagcgattgatatgcacgatgcgaaagcaattgaagcgcgtaacaaagaatgggaaagtaatccgattatatttcaatacaaagacgccggagttaattgtgttgcggttgggagttcacctaggtttaaggtttatgatgtggatttcgggtttggtaaacccgaaagtgttcggagcggggcgaataaccggtttgatggtatggtttatttgtatcagggaaaaagtggtggaaggagtattgatgtggagattagtttggatgcaagtgcaatgggaaatcttgaaaaggataaggaatttcttatccaagaataa

SEQ ID NO: 116 - DNA, 1353 bp, Helicrysum umbraculigerum
  source 1..1353: mol_type = other DNA
atggcttctcttcctctcttaactgttcttgaacaatcccatgtatcaccaccgccagccaccgtagtcgataaatcgttgtcgctaacctttttcgatttcctgtggctaactcaacctccaattcacaatcttttcttttacgagttttcaatcgacgaaactcagttcgtggaaactatcgttcctagtcttaaaaactcgttatcaatcactcttcaacatttttacccgttcgccggtaaccttatcttatttcctgataacaaaaggcctgaaattcgttacgttgaaggtgattatgtcatggttacatttgcaaaatctagccttgacttcaatgaactagtaggaaaccatcctagagattgtgaccagttttatgatcttattcctccattaggtgaaagtgtgaaaacttctgaatttcgaaaaatcccactcttttcggtccaggtgacgttttttccacaaaaaggcgtatcgattggtatgacgaatcatcatagtcttggcgatgctagcactcggttttgtttcttgaacgcgtggacatcgatttctagatctagttcagatgagtcatttctagcaaacggaactaaaccgttttacgatagagtgataagtaacccgaaactagatcaaagttatctaaaattttccaagatcgatactctttacgagaagtatcaacctttaagcctctctagaccatctaataaacttcgtggcacgtttatcttgacgcgaaaaatcctaaacgagttgaaaaaaagtgtgtcaattaaactaccaactttatcatatgtatcatcttttacggttgcatgtggttatatttggagttgcatagcgaaatcacgaaacgatgatctacaactattcgggttcactattgattgtagggcacgtttggatccaccggttccatcaacttattttgggaattgtgtcgggggttgtatggcgatggcaaaaacaacgttgttaaccgaagacgatggatttataacggctgctaaattgcttggagaaagtttacacaagacgttgaccgaatcgggtggaatcgtgaaagatatagaagtgtttgaagatttgtttaaggatggattaccaacaactatgataggagttgcgggaacaccaaagcttaagttttatgagacggatttcgggtgggggaacccgaaaaaggtggaaacgatttcgattgattataacatgtcgatttctatgaacgcttgtagagaatcgaaggatgatttggagattggtgtttgccttatgaatactgaaatggaagcttttgttcgtttatttgatgaaggattagaatcatacgtttag

SEQ ID NO: 117 - DNA, 1329 bp, Helicrysum umbraculigerum
  source 1..1329: mol_type = other DNA
atgggaagtgaaaatgttcacaaaataatgaaaatcaacatcactaaatcatcatttgtacaaccctcaaagcctacagtactacccactaaccacatatggacttctaacttagatttagttgtgggtagaattcatattttaaccgtttacttttaccgtccaaatggtgcttcgaatttttttgatccaattgttatgaaaaaagctttagctgatgtgcttgtttctttttatccgatggccggaagaataagtaaagatgataatggtagagttgtaattaattgtaatgatgaaggtgttttgtttgttgaagctgagtcagattccacgttggatgacttcggtgagtttacaccgtctccggagctccgacaacttaccccgacgattgattactccggtgacatttcaacgtacccgctattttttgcacaggtaacgcatttcaagtgtggaggagttggttttggttgtggtgtgtttcatacacttgcagatggtctatcctctatacatttcatcaacacatggtcggacatggctcgtggtctctcgatagccatcccgccattcactgaccggacccttcttcgtgcacgtgaaccacccactcccacttttgaccacgtagagtaccacctccctccgtccatgaaaactacctcacaaaccaacaaatccagaaagccttccacggccatgttaaagcttacgcttgatcaactaaatgctctcaaagctgctgctaagaatgaaggcggcaacaccaattatagcacgtacgagatcctggcggctcatttatggcggtgtgcctgcaaggctcgaggactccctgatgaccaactaaccaaattgtacgtggcaacagatggacggtccagattgagccctcaactcccaccaggctatctaggcaatgttgtgttcaccgccaccccagttgccaaatcagctgacctcacgactcaaccattgtctaatgcagcatctttgatccgaaccacattgacaaaaatggataacgactatttgagatctgccattgattaccttgaggtgcagccagatctatctgctttaattcgtggtcctagttactttgctagcccgaatttgaacataaacacgtggacccggttgccagtacatgatgcggatttcgggtggggtcggcctgttttcatgggaccagcagtgatattgtatgagggcaccatctatgttctaccaagcccaaacaatgataggagtatgtcattggcagtctgtttagatgcagatgaacaaccatcgtttgagaagttcctgtatgacttttaa

SEQ ID NO: 118 - DNA, 1425 bp, Helicrysum umbraculigerum
  source 1..1425: mol_type = other DNA
atgccttcatcatcatcatcgccttcttcaacagctgattcagttaccataatctcaaaatgcacagtctacccacatatgaaaaactcaacaccagaatccttgcagctctctgtttctgatctcccaatgctttcatgtcaatacatacaaaaaggtgtcttactttctcaaccgccacccaatcacaccaacaatatcatttcccacttaaaactctctctctctaaaaccctctctcacttcccacctctcgccggccgtctttcgaccgactctcacggccacgtctctatcatctgcaacgattccggcgtcgaattcgttcactccaccgctaaccacctccacacccaccaaatcttacccctcaattccgacgttcacccatgttttaaaaccttttttgcttttgataaaactctgagttacgccggccaccaccaaccaatcgccgccgtgcaagtcacggagcttgctgatggactctttattgggtgtacggtaaatcatgctgtcgttgacgggacttctttttggaacttttttaatacttttgctgagatcacaaaagggtgtcagaaagtaacgaacttgccggattttagccgggaaaatgttttcatttctccggttgttttgcctcttccctccggcggcccgtcggcgacgttctcaggtgatgagccgttgagggaaaggatcattcatttcagtagagacgcgattctgaagatgaaattcagagctaataatcctttatggcggcaaccacaaaattcggatctggatgatacagagatttacgggaaagtgtgtaacgacattaacggcaaagttaacggggcgtttaaacccaaaagtgaaatttcgtccttccagtctttatgtggtcagttatggcgtgcggttacacgcgcgcgtaaattcaacgaccctataaaaacgacgacgtttcgaatggcggtgaattgtaggcataggctagacccaaaggtcgacaaactttatttcgggaacttgatccaaagcatcccgaccgttgcttcagttggggagttgttatcacatgatttgtcgtgggcagccaatgagcttcaccaaaatgtggtggcgcatgataatgctaccgtgcgcaggggtgttaaggattgggagaataatccaaagttgtttcctttggggaattttgatggtgctatgatcacaatgggaagttctcctaggtttccaatgtataataacgatttcgggtggggccgcccaatggcggttcgtagtggtaaagctaataagtttgatggaaagatttcggcttttccgggacgtgatggtgatggtagtgtcgatcttgaggttgttttagctcccgaaaccatggcatgtcttgaacgtgaccatgaatttatgcaatatgtatcttaa

SEQ ID NO: 119 - DNA, 1608 bp, Helicrysum umbraculigerum
  source 1..1608: mol_type = other DNA
atgaagtggtttttcataacccataaagcaacccagcgttgccttaattctaaacaatttcatcttcacggaggttcgaatttcgtttccggtaatagatgttttcttgcatcacactcaatggagcggccaaaattcatgttgataccatattatccctaccaaattcggtccttaaattcgagtcaccgatatagtagtacgtcacccagcggatcccctcacagttttctgaatggtactaagaatgaaaactatacgaagaaggtagatcttgaaataatttcaagagaaatcatcaaaccagcttctccaactccacatcatttaagaaacttcaacttatcacttttggaccaaatagtatttgattgctacacccctgtaatcctctttattccaaatagtaataaggctactgttacggatgtcatgatcaaaagattgaaacatctcaaggagactttatctcgaattctaagtcaattttatccctttgcgggagaagttaaggacagattgcatatcgaatgcaatgacaagggagtcaattacatcgaggctcaaatcaatgagacattggaagaatttctatgtcatccagataacgaaaaggcgagggagcttatgcccgaaagccctcatgttcaagaatctgcaataggaaactatgctatgggtattcagataaacattttcagttgcggagggattggactttccatgagcatggcacacaagatcatggacttctacacatatacgatcttcatgaaagcatgggctgcagctgttcgaggttcaccagatacaattatttcaccaagttttgtggcttctgaggtctttcctaatgatcccagccaagaagattcaattcctatcgagttaaagtctagtaatttgcttagcacaaaaagatttgagtttgatcctactgcgttggctctcctaaagggacaagttgtcgccagcggatcacctccccaacgaggaccaagtcgtatggaggcgacaacagccgttatttggaaggccgctgcaaaagctgcatcgactgtcagaagattcgatccaaagtcacctcatgcgctggcgttaccagtaaatatacgtaaaagggcatcacctgctctcccagacaattccataggaaacatagttatgcgaggtatagcaatttgttttcctgagagccaaccggacttgccaactcttatgggtaaagtgagagaatcaatagcgaaacttaactcagattacattgagtccctgaaaggtgaaaaggggcatgagacagttaataagatgttgaaggagttgaagcttcggacgaatatgacaaaggtaggagggaaattcgttgctagttgcatatttaatagtggaatatatgagttggatttcgggtggggaaaaccgatatggttctatgttgtgaatccaggaagcgatagttgtgtggttttgactgatacgctgaagggtggtggtgttgaagccacaattacactaccaccagatgaaatggagatattcgaacgtgatcatgagcttctatcctatactaccatcaaccctagtccactgcgatttcttgaccattga

SEQ ID NO: 120 - DNA, 1395 bp, Helicrysum umbraculigerum
  source 1..1395: mol_type = other DNA
atggaggtgcctgaccaattccacctaaacattcttgaacaatgccacgtttcaccatcaccaaattccatcataccttcattttcactacccttaacattcttagacatcccatggcttttttacccttcaaatcaaaccctttttttcttcccagaaccaccacccaaaaccaccatcatcaccacccttaaacaatcactctctcttaccctccaccacttccaccctctcgccggaaacctctcacttccatcacctccggcggaaccccacattgtttacaccaaaaatgactcaattgcactcacaattgctcaaacaaacaccaacatccaccatctttcttgcaatcacccaagaagtgtaaaaaatctttactctcttttacccaaactcccatctccatccatgtcacgtgaaactcacgtgggccttgttatcccccttcttaccatccaaattacggtttttgctgatttggggtattcgatcggagtcactatgcaacatgcagcagttgatgaacggacatttgatcagtttatgaaatgttgggcgtctgtttgtacatctttgttgaaaaatgactcactttttacattcaagtctacaccttggtacgataggagcgtaattatcgaccccaaatcgctgaaaacaacgtttttaaagcaatggtggaaccgatctaattctctcaatgagtcacatgatcaagaaaatgatgatcatgatcttgttctagcaacttttgttttgagttcattagatattaacatgatcaagaatcatattcttgcaaaatgcaagatgataaatgaggatccaccactacatttatctccttatgttagtgcatgtgcttatttatggaaatgtttaatcaaaattcaagaaacccatgattctattaagggtggtcctctctatttagggtttaatgccggtgggattactcgattagggtacgacataccttcaacttattttgggaattgtatagcttttgggagatgcaaggcatttgagagtgaattattgggtgataatggtattgttttcgcggcaaaatcgattggaaaagagatcaagaggcttgataaggatgttttaggaggtgctaataagtggattagtgattgggatgaattaaccattaggcttcttggttcaccaaaagttgattcatatggtatggattttggatggggtaaagttgagaaggttgaaaaaatatcaagtatttcaaatcacggtagggttaatgtaatttctttgagtggatgtaaggattttaaaggtggaatagagataggggttgttctttctgtggctaaaatgaatgttttcacttccctctttcatggaggtttaatggagtttgcatattga

SEQ ID NO: 121 - DNA, 1218 bp, Helicrysum umbraculigerum
  source 1..1218: mol_type = other DNA
atgaaaaataagaacccgactagtgtgatcagagaggctttagctaaggtattggtgttttattatccgtttgctggccggctcaaggaagggccggccaggaaactgatggtggattgttctggtgaaggtgtgttgtttattgaggcagaagctgatgtcacgttgaaacaatttggtgacgcacttcaaccgccatttccttgtttagaagagcttctttacgatgttcctggatctactggtattctagatacaccattattgctgattcaggtgacacgattgttatgtggaggttttatctttgctctacgactcaaccacaccatgagcgacgcagcaggtctcgttcaattcatgacagggcttggtgaaatggcacaaggtgcatcaaggccatcaacgttgcctgtatggcaaagggagttgctttttgcaagggacccaccacgcgtgacttgtactcatcacgagtatactgaagtggaagacaccaatggtacaatcattccgctagatgacatggcacataaatcatttttctttggaccttctgagatatcagcgttgcgaaggttcgttccatcatacctaaaaaagtgttctacttttgaggtcttaaccgcttgcctatggcgttgtcgtacaattgcactccagccagatcccgaagaagagatgcgcatgatatgcattgttaatgcgcgtggaaagtttaatcctcccctattacccaaaggatattatggaaatggtttcgctataccagtggccatttcaacagctggagacctatctagcaaaccattaggtcacgcattggaacttgtaatgaaagccaaatccaatgtcactgaggagtatatgagatcagtagccgacttaatggtaatcaagggacgaccccactatacggttgtccgaagctaccttgtatcggatgtgactcacgctggatttgatgttgttgatttcgggtgggggaaagcgtcctatggaggacctgcaaaagggggagtaggtgctattcccggagttgttactttctttataccttttacaaaccataaaggcgagtctggaattgtgctacctatatgtttgccgagtgcagccatggataagtttgttgaagagttaaataagatgttggtcccagacaacaacgaacaagtactccgagaacacaagttactagttctcgctagattgtaa

SEQ ID NO: 122 - DNA, 1386 bp, Helicrysum umbraculigerum
  source 1..1386: mol_type = other DNA
atggcacaaatcgacactccattgacattcaaagtccggagacatgcaccggagctgatcgctccagcgaaacctacgccacgagaactaaaacctctatccgacattgatgatcaagaaggccttaggtttcatatcccagtgattcaattctatcgtagcgatccaaagatgaaaaataagaacccggctagtgtgatcagagaggctttagctaaggtgttggtgttttactatccgtttgctggccggctcaaggaagggcctgccaggaaactgatggtagattgctctggtgaaggtgtgttgtttattgaggcggaagctgatgtcacgttgaaacaatttggtgacgcccttcaaccgccgtttccttgtttggaagagcttctttacgatgttcctggatctactggcgttctagatacaccgttattgctgattcaggtgacacgattgttatgtggaggttttatctttgctctacgactcaatcacaccatgagcgacgcaccaggtctcgttcaattcatgacagggctcggtgaaatggcacaaggtgcatcaaggccatctacgttgcctgtatggcaaagggagttgcttttagcaagggacccaccacgcgtgacatgtactcatcacgagtatactgaagtggaagacaccaagggtacaatcattccgctagatgacatggcacataaatcatttttctttggaccttctgagatatcagcattgcgaaggttcgttccatcatacctaaaaaagtgttctacttttgaggtcttaaccgcttgcctatggcgttgtcgtacaattgcactccagccagatcccgaagaagagatgcgcataatatgcattgttaatgcgcgcggaaagtttaatccaccccttcctaaaggttattatggaaatggttttgctttcccagtggccatttcaacagctggagatctatccagcaaaccattaggtcatgcattggaacttgtaatgaaagccaaatccgatgtcactgaggagtatatgagatcaatagccgacttaatggtaatcaagggacgtccccactttacggttgtcagaagctaccttgtctcggatgtgactcacgctggatttgatgttgttgatttcgggtgggggaaagcggcctatggaggacccgctaaagggggagtaggtgctatcccaggtgttgctagtttctatataccttttacaaaccataaaggcgagtctggaattgtgctacctatatgtttgccgagtgcggccatggataagtttgttgaagagttaaataagatgttggtcccagacaacaacgaacaagtactccgagaacacaagttactagttcttgctagattgtaa

SEQ ID NO: 123 - DNA, 1332 bp, Helicrysum umbraculigerum
  source 1..1332: mol_type = other DNA
atggaaatacaagtaataaactactcatcaaagctagtaaaacccttgacaccaacacccaccgcaaatcgttactataacatttctttcaccgatgagctcgtcccaaccatttacgtcccactcattctctactacgcaacaccgaaaaacccaaatggtgatcactttgaaaacatttgtgaccgtctggaggagtcgttatcgaaaacgttaagtgatttttacccactggccgcgagattcattcgtaaactctccttaattgattgtaacgatcaaggggttttgtttgtcctaggcaatgtaaatatccgactttcggatgttacaggcctaggactgacgtttaaaaccagtgttttaaatgattttctcccgtgtgagattggaggagcggatgaagtcgatgatcctatgctttgtgtcaaagtcaccacttttgagtgtggtggttttgcaattggtatgtgtttttcgcataggctttcggatatgggtaccatgtgtaactttattaacaattgggctgctagaactattggtgaatatgataatgaaaaacatactcctatttttaattcgccgttgtacttcccgcaacgaggattacctgaacttgacctaaaagtacctaggtcaagtattggtgtgaaaaatgcagcacgcatgtttcactttaatgggaaggcaatatcatccatgagagaagtttttggagttgatgaaaatgggtctcgtagactctcaaaggttcaacttgttgtagccttgttgtggaaggcctttgttcgcatagatgatgtgaacgatggccaatctaaggcgtcttttctgatccaaccagttgggttgagggacaaagttgtccctccattaccatcaaactcatttgggaatttttggggtctagcgacttcccaacttggtcctggtgagggtcacaaaatcggtttccaagaatatttttacattttgcgtgaatctattaagaaaagagctagggattgcgctaaaatattgacacacggtgaagaaggatatggggttgtaatcgatccatatcttgagtcgaatcaaaagatagctgataatggtacaaacttttacttgttcacttgttggtgcaagttttcgttctacgaagctgattttggttgtggtaagccgatttgggctagcaccggaaagtttccggttcaaaatttggtgatcatgatggatgataatgagggtgatggtgtagaagcgtgggttcatttagacgataaacgcatgaatgagttagaacaagatcctgatgttaaactctacgcatgcaatttagcttaa

SEQ ID NO: 124 - DNA, 1305 bp, Helicrysum umbraculigerum
  source 1..1305: mol_type = other DNA
atgaaattagcagtgaaggaatcagtgatagtaaaaccatccaaaacgacaccgtgtcagcaaatatggacatcaaatcttgatttagtggtgggtcggatccatatattaaccgtttacctttacagaccaaatgggtcttcaaatttctttgattccatggttttaaagaaggctctagccgacgttttagtttctttttttccggtggccggacggttggataaagacggtgacggcagagttgtaatagattgtaacggtgagggtgttttgtttgtggaagctgaagctgattgttgcattgatgattttggtgagattactccgtcgccggagttacgacggttggtgccgacggtggattattccggtgatatgtcttcttatccgttatttattacgcaggttacacggttcaagtgtgggggagtttcgttaggctgtggactacaccatacgttatcggatggactctcagcacttcacttcatcaacacatggtctgatgtagctagaggcctatcggtggcaatcccaccgttcattgaccgctcccttcttcgagctcgtgatccaccatcccctgtgtttgaccacatcgaataccacccaccaccgtcactgatcactccgttgcaaaaccaaaagaacgcgtcacattcgaggtctgcttcaactttaatcctacggctcacactccatcaaataaacaatcttaaatcaaaggctaaaggcgatgggagcatgtaccatagcacgtacgagatcctagctgctcatctatggcgatgtgcgtgcaaagcacgtggactagcaaacgatcaaccaaccaaattgtatgtggccaccgatggacggtcaagattgattcctccactccctccgggctaccttgggaatgtcgttttcacggctactcctgtcgctaaatcgggagatttcgaatctgaatccttggcagagacagcaaggaggattcgcagtgagttgggtaaaatgaacgatgagtatcttagatcagctattgactacttagagtcggtatctgatatttcgacccttgttagagggccgacttactttgcgagtccaaatctgaatgtaaacagttggactcggttaccaatatacgaatctgacttcggttggggtcgacctattttcatgggacccgcaagtatactttacgagggtacgatttacatcataccgagccctagtggtgatcggagtgtgtctctggccgtgtgcttggaccctgatcacatggctttgtttaaagaatgcttgtacgttttttag

SEQ ID NO: 125 - DNA, 1305 bp, Helicrysum umbraculigerum
  source 1..1305: mol_type = other DNA
atgaagctagcagtgaaggaatcagtgatagtaaaaccatccaaaacgacaccgtgtcagcaaatacggacatcaaatcttgatttagtggcgggtcggatccatatattagtcgttttcttttacagaccaaatgggtcttcgaatttctttgattccttggttttaaagaaggctctcgccgacgttttagttcctttttttccggtggccggacggttcagtgaagacggtgacggcagagttgtaattgattgtaacggtgagggtgttttgtttgtggaatctgaagctgattgttgcattgatgattttggtgagattactctgtcgccggagttacaacagttggtgccgacggtggattattccggtgatatgtcttcttatccgttatttattgcgcaggtcacacggttcaagtgtgggggagtttcgttaggttggggactacaccatacattattggatggactctcagcacttcacttcgtcaacacatggggtgatgtagctagaggcctatcggtggcaatccaaccgttcattgaccgctcccttcttcgagctcgtgatccaccgacccctgtgtttgaccacatcgaataccacccaccaccgtcactgatcactccattgcaaaaccaaaagaacgcatcacattcgaggtctgcttcaactttaatcctacagctcacacccgatcaaataaagaatcttaaatcaaaggctaaaggcgatgggagcatgtaccatagcacatacgagatcctagctgctcatctatggcgatgtgcgtgcaaagcgcgtggactagcaaacgatcaaccaaccaaattgtatgtggccgccaatggacggtcaagattgattcctccactccctccgggctaccttgggaatgtcgttttcaacgctactcatgtcgctaaatcgggggattttgaatctgaatccttggcagagactgcaaggaggattcactgtgagttgggtaaaatgaacgatgagtattttagatcagctatcgactacttagagtcggtagatgatatttcaacccttgtcaaagggccgacttactttgcgagtccaaatctgaatgtatacagttggattgggataccaatatatgcatgtgacttcggatggggtcaacctattttcatgagacccgcaagtttcctttacgatggttccatttacatcataccgagccctagtggtgatcggagtgtgttgttggccgtgtgcttggaccctgatcacatggatttgtttaaagaatgcttgtacgctttttag

SEQ ID NO: 126 - DNA, 1383 bp, Helicrysum umbraculigerum
  source 1..1383: mol_type = other DNA
atggtgatgattagcaagcttttacgattaggtagaagaaaacttcacacaattgtatcaagagataccattagaccttcttctccaactccctctcattccaaaacatataatctctccttgctcgatcaaatagctgtaaattcatacgtgccgattgttgctttttacccaagctcaaatgtttgtcgaagttccgatgataagacgctggagttgaagaactcattatcgaaaatattaactcattactatccgtttgccggtagaatgaagaagaatcgccctaccgtcgttgattgcaatgatgaaggggttgagttcgttgaagcacgtaataccaactcgttatcagatttcctccaacaatcggagcacgaagatctagatcaactctttccagatgattgtgtatggttcaaacaaaaccttaaaggttctattaatgacgcaaataatagtagcgtatgtccattgagcattcaagtcaaccatttcgcgtgtggaggtgtagcagttgcaacttcgttacgccacaagattggagacggaagcagtgcgttaaatttcattaaacactgggctgcagttacgtcacactctcgagcagggaatcatcaaattgatgcgacatcacccatcattaatccccatttcatttcttacccaactagaacttttaaattgccagataggtcaccatacataccacctagtgatgttgtgtcaaaaagttttgttttccccaacacaaatataaaggacctccaagccaaggtggtaaccatgaccatgggctctagacaacctatcgtgaaccctacccgagctgatgtcgtatcatggcttctacataagtgtgtagtagcagcagctaccaaaaggatatcgggaaattttaaagaaagttgcgtgatctcgccattaaatctgagaaacaagttagaagagccattgcctgaaacaagcataggaaatattttctatctgataacctttccaataagcaataatcatggcgatctcatgcccgatgacttcattagccaactcaggctaggaatacgtaagtttcaaaatatacgaaatttggaaactgcattacgaaccgttgaagagatgatatctgaaacttttatcttgggtacggcagaaagcatggatactagttatgtatattcgagcatccgtgggtttccgatgtatgatattgattttgggtgggggaagcccgtaaaagtaaccgttgggggagcccttaagaacttaagtattctgatggacactcctgatgtcaatggcatcgaagcactagtgtctttggataaacaagacatgaagatacttctaaacgaccctgagttgttggccttttgcttgtaa

SEQ ID NO: 127 - DNA, 1323 bp, Helicrysum umbraculigerum
  source 1..1323: mol_type = other DNA
atgagtactagtgacaaaatgaagataacaataagagaatcatcaatgataaaaccatccaaaccgacgccggatcaacggatatggaactcaaatcttgatttggtagtgggtcggatccatatcttgaccctttacttttttaggccaaatgggtcttcggatttctttgattctgaggttttaaagcaatcacttgccgacgttcttgtttctttttttccgatggccggacgattgggattagacggcgatggcagagttgaaattaattgcaacggtgaaggtgttttgtttgttgaagctgaagcggattgtagtattgatgattttggtgagattactccgtcgccggagctacggcggttggcgccaacagtggattattccggcgatatctcatcttatccactcgttattacccaggtaacacatttcaaatgtggtggagtttctcttgggtgtggactacaccatacattatccgatggactttcatctcttcacttcatcaacacatggtccgatgttacccgaggcttacccgttgcgatcccgccattcgtagatcgtacggttcttcgtgctagggacccgccaaccgtggtctttgatcacgtggaataccacactcctccttccatgacctcaagtttggacaaagacaaacctcaatccgaagatgttcatgtttccacttccatgctacggctcacactcgatcaaataaatgcactaaaagcaaaaggcaaaggtgacggaattgtgtaccatagcacatatgaaatcctagctgctcatttatggcgatgtgcgtgtaaagcacgtgggctcctgaatgatcaaatgactaaattgtatgtagctaccgatggacggtccagattgattcccccactcccaccggggtacttaggcaatgtggtcttcaccgccacaccaattgccaaatccggcgagctccaacaggaaccactagctaccactgcaagaaaaattcatacagagttggccaaaatggatgacaagtacctcaggtcggccctcgactacttagagtcacaacaggacttgtcagcactaattcgagggccagcctattttgcgtgccctaacctcaacatcaatagttggactcgccttccaatatatgatgcggactttgggtggggtcggcccatatttatgggacccgccagcatactttacgagggcacgatttacattattccgagccctagtggtgaccgaagtgtgtcgttggctgtgtgcttagacccctctcatatgcctctcttccaaaagtacttgtatgaactttaa

SEQ ID NO: 128 - DNA, 1326 bp, Helicrysum umbraculigerum
  source 1..1326: mol_type = other DNA
atggtgaatgttgagatcatttctaatgaatacataaaaccatcctccccaacaccaccacatcttaaaatatacaatctttccatcttagatcaactcattcctgccccctatgcacctatcatactatattatccgaatcaagatcacattaacgattttgaggttcacgaacggttgaaactactaaaagattcgttatcgaaaacgctaactcgtttttacccattagccggaaccatcaaaggcgatctttccattgattgtaacgatattggtgcttactttgcagtagctcatgtaaatactcgccttgatgtgttcctgaaccatcctgatcttgacctaataaactgttttcttccacgtgggccttacttgaatggttctagtgaaggaagttgtgtgagtaatgttcaagtgaacatttttgagtgttgtgggattgcaattagtttatgcatttctcacaagattcttgatggtgctgcgttgagtacttttcttaaagcatgggcagggacaagttacgggtcgaaagaagtagtgtatccaaacatgagtgcaccatctttatttcctgctaaagatttgtggcttaaagattcatcaatggtcatgtttgggtctttgtttaagatgggtaagtgtagtactaaaagatttgtttttgattcatcaaaattatccttcctcaaagctaaggcatcgctaaatgggctaaaagacccaacccgcgtagaggtggtgtctgctttactatggaagtgtatcatggctgcatctgaagaaaacactggttcttggaagccatctctgttaagccatgtagttaaccttcgcaaaaggttggtttcaactttatcagaagactcaattgggaacttaatttggttagcaagcgcagaatgtagaaccaacgctcaatcccgattgagtgatcttgttgaaaaggtacgtgatagtgtgtcgaaaatcaatagtgagtttgtgaagaaaatacaaggcgataaagggacaaaagtgatggaagagtctctcaagagtatgaaagattgtgcggattatatcgggtttacgagttggtgtaagatggggttttacgatgtggattttggttggggaaagcctgtatgggtttgtggtagcgtttgtgaaggtagcccggtgttcatgaattttgtcatattaatggacacaaaatatggtgatggaatagaagcatgggtgagcttggatgaacacgaaatgcatatcttaaagcataatcccgagctcttggaatatgcatcaatcgatccaagtcctctgcaaatgaataagtga

SEQ ID NO: 129 - DNA, 1365 bp, Helicrysum umbraculigerum
  source 1..1365: mol_type = other DNA
atgggaactatttatcaatctcccatgatcaaatcttctactcccaaaataattgaagacctcaaagttatcatccatgacacattcacaatcttcccacctcacgaaaccgaaaagcggtccatgttcttatcgaacattgaccaagttcttactttcaacgttgaaacggtccatttttttgcagccaaccctgactttccgccacaagtagtggcggaaaagctcaagttggctctaagtaaggcgctggtgccatatgattttttggcagggaggttgaagttgaaccatgagtcgcaacggtttgagtttgattgtaatggtgctggggctcggttcgtggtgggttcgagtgagtttgagttgggtgagattggtgacttggtgtatccaaaccctgggtttagacaattggttcaaaagagttatgataacttggagttacatgaaaagccactatgcattttacagctgacatccttcaagtgtggaggatttgcacttggtgtagcaacaaatcatgccacttttgatggcttaagtttcaaaacatttcttcaaaatcttggttctttggctgctgatcaaccacttgccgtcgatccctgcaacgatcgccacctattggcagcacgatcaccaccaaaagtccaatttgaccaccctgaactcctcaaaatcccaacaggaacagacatcccaaacccaacagtctttgactgcccagaaagtcaacttgacttcaagattttcaacttgacctcagatgacatagcccacttaaaaacgaaagccaaagatgggcctgggtcaaccaatgcaaaaatcactggattcaatgtggttgcagcccatgtatggcggtgcaaagcgttgtcctcagggtcagaatatgaccccgagagagtgtcaaccgtgttatatgctgttgacattcggtcaagattgaacttaccattatcattagctggcaatgcagttcttagtgcatacgcctcggccaaatgcaaagagattgaagaaggcccgttgtcaagactagtggaaatggtgaccgaaggtactaacagaatgactggtgagtatgcaagatcggtgatcgattggggagaggtgaataaagggtttccaaatggggagtttctgatatcgtcatggtggcgattggggtttgctgacgtggaatatccgtggggtaaacctaggtatagttgtcccgtggtttatcataggaaagatataatattactctttccggatattgttggtgccgataacaacaatgaagtgaatgtgttggtggctttgcctggcaaagaaatggagaaatttgagactttatttcataagtttttggcatga

SEQ ID NO: 130 - protein, 455 aa, Helicrysum umbraculigerum
MATQVKTEEKHLKVEIINKTYVKPETPLGRKECQLVTFDLPYIAFYYNQKLIIYKGGVEEFEDTVEKLKDGLKVVLGEFHQLAGKLDKDDDGVFKVVYDDDMDGVEVLSAVAEDTATADLMDEEGTIKLKELVPYNSVLNIEGLHRPLLSIQITKLKDGLVLGCAFNHAILDGTSTWHFMSSWAQICSGSKSISAAPFLDRTQARNTRVKLDLTPPAQTNGNSNGDTNGDASATKPPAPAPLREKIFKFSESAIDKIKAKINANPPEGSTKPFSTFQSLSTHIWHAVTRARNLKPEDYTVFTVFADCRKRVDPPMPDSYFGNLIQAIFTVTAAGLLQANPPEFAASMIQKAIDMHDAKAIEARNKEWESNPIIFQYKDAGVNCVAVGSSPRFKVYDVDFGFGKPESVRSGANNRFDGMVYLYQGKSGGRSIDVEISLDASAMGNLEKDKEFLIQE

SEQ ID NO: 131 - protein, 450 aa, Helicrysum umbraculigerum
MASLPLLTVLEQSHVSPPPATVVDKSLSLTFFDFLWLTQPPIHNLFFYEFSIDETQFVETIVPSLKNSLSITLQHFYPFAGNLILFPDNKRPEIRYVEGDYVMVTFAKSSLDFNELVGNHPRDCDQFYDLIPPLGESVKTSEFRKIPLFSVQVTFFPQKGVSIGMTNHHSLGDASTRFCFLNAWTSISRSSSDESFLANGTKPFYDRVISNPKLDQSYLKFSKIDTLYEKYQPLSLSRPSNKLRGTFILTRKILNELKKSVSIKLPTLSYVSSFTVACGYIWSCIAKSRNDDLQLFGFTIDCRARLDPPVPSTYFGNCVGGCMAMAKTTLLTEDDGFITAAKLLGESLHKTLTESGGIVKDIEVFEDLFKDGLPTTMIGVAGTPKLKFYETDFGWGNPKKVETISIDYNMSISMNACRESKDDLEIGVCLMNTEMEAFVRLFDEGLESYV

SEQ ID NO: 132 - protein, 442 aa, Helicrysum umbraculigerum
MGSENVHKIMKINITKSSFVQPSKPTVLPTNHIWTSNLDLVVGRIHILTVYFYRPNGASNFFDPIVMKKALADVLVSFYPMAGRISKDDNGRVVINCNDEGVLFVEAESDSTLDDFGEFTPSPELRQLTPTIDYSGDISTYPLFFAQVTHFKCGGVGFGCGVFHTLADGLSSIHFINTWSDMARGLSIAIPPFTDRTLLRAREPPTPTFDHVEYHLPPSMKTTSQTNKSRKPSTAMLKLTLDQLNALKAAAKNEGGNTNYSTYEILAAHLWRCACKARGLPDDQLTKLYVATDGRSRLSPQLPPGYLGNVVFTATPVAKSADLTTQPLSNAASLIRTTLTKMDNDYLRSAIDYLEVQPDLSALIRGPSYFASPNLNINTWTRLPVHDADFGWGRPVFMGPAVILYEGTIYVLPSPNNDRSMSLAVCLDADEQPSFEKFLYDF

SEQ ID NO: 133 - protein, 474 aa, Helicrysum umbraculigerum
MPSSSSSPSSTADSVTIISKCTVYPHMKNSTPESLQLSVSDLPMLSCQYIQKGVLLSQPPPNHTNNIISHLKLSLSKTLSHFPPLAGRLSTDSHGHVSIICNDSGVEFVHSTANHLHTHQILPLNSDVHPCFKTFFAFDKTLSYAGHHQPIAAVQVTELADGLFIGCTVNHAVVDGTSFWNFFNTFAEITKGCQKVTNLPDFSRENVFISPVVLPLPSGGPSATFSGDEPLRERIIHFSRDAILKMKFRANNPLWRQPQNSDLDDTEIYGKVCNDINGKVNGAFKPKSEISSFQSLCGQLWRAVTRARKFNDPIKTTTFRMAVNCRHRLDPKVDKLYFGNLIQSIPTVASVGELLSHDLSWAANELHQNVVAHDNATVRRGVKDWENNPKLFPLGNFDGAMITMGSSPRFPMYNNDFGWGRPMAVRSGKANKFDGKISAFPGRDGDGSVDLEVVLAPETMACLERDHEFMQYVS

SEQ ID NO: 134 - protein, 535 aa, Helicrysum umbraculigerum
MKWFFITHKATQRCLNSKQFHLHGGSNFVSGNRCFLASHSMERPKFMLIPYYPYQIRSLNSSHRYSSTSPSGSPHSFLNGTKNENYTKKVDLEIISREIIKPASPTPHHLRNFNLSLLDQIVFDCYTPVILFIPNSNKATVTDVMIKRLKHLKETLSRILSQFYPFAGEVKDRLHIECNDKGVNYIEAQINETLEEFLCHPDNEKARELMPESPHVQESAIGNYAMGIQINIFSCGGIGLSMSMAHKIMDFYTYTIFMKAWAAAVRGSPDTIISPSFVASEVFPNDPSQEDSIPIELKSSNLLSTKRFEFDPTALALLKGQVVASGSPPQRGPSRMEATTAVIWKAAAKAASTVRRFDPKSPHALALPVNIRKRASPALPDNSIGNIVMRGIAICFPESQPDLPTLMGKVRESIAKLNSDYIESLKGEKGHETVNKMLKELKLRTNMTKVGGKFVASCIFNSGIYELDFGWGKPIWFYVVNPGSDSCVVLTDTLKGGGVEATITLPPDEMEIFERDHELLSYTTINPSPLRFLDH

SEQ ID NO: 135 - protein, 464 aa, Helicrysum umbraculigerum
MEVPDQFHLNILEQCHVSPSPNSIIPSFSLPLTFLDIPWLFYPSNQTLFFFPEPPPKTTIITTLKQSLSLTLHHFHPLAGNLSLPSPPAEPHIVYTKNDSIALTIAQTNTNIHHLSCNHPRSVKNLYSLLPKLPSPSMSRETHVGLVIPLLTIQITVFADLGYSIGVTMQHAAVDERTFDQFMKCWASVCTSLLKNDSLFTFKSTPWYDRSVIIDPKSLKTTFLKQWWNRSNSLNESHDQENDDHDLVLATFVLSSLDINMIKNHILAKCKMINEDPPLHLSPYVSACAYLWKCLIKIQETHDSIKGGPLYLGFNAGGITRLGYDIPSTYFGNCIAFGRCKAFESELLGDNGIVFAAKSIGKEIKRLDKDVLGGANKWISDWDELTIRLLGSPKVDSYGMDFGWGKVEKVEKISSISNHGRVNVISLSGCKDFKGGIEIGVVLSVAKMNVFTSLFHGGLMEFAY

SEQ ID NO: 136 - protein, 405 aa, Helicrysum umbraculigerum
MKNKNPTSVIREALAKVLVFYYPFAGRLKEGPARKLMVDCSGEGVLFIEAEADVTLKQFGDALQPPFPCLEELLYDVPGSTGILDTPLLLIQVTRLLCGGFIFALRLNHTMSDAAGLVQFMTGLGEMAQGASRPSTLPVWQRELLFARDPPRVTCTHHEYTEVEDTNGTIIPLDDMAHKSFFFGPSEISALRRFVPSYLKKCSTFEVLTACLWRCRTIALQPDPEEEMRMICIVNARGKFNPPLLPKGYYGNGFAIPVAISTAGDLSSKPLGHALELVMKAKSNVTEEYMRSVADLMVIKGRPHYTVVRSYLVSDVTHAGFDVVDFGWGKASYGGPAKGGVGAIPGVVTFFIPFTNHKGESGIVLPICLPSAAMDKFVEELNKMLVPDNNEQVLREHKLLVLARL

SEQ ID NO: 137 - protein, 461 aa, Helicrysum umbraculigerum
MAQIDTPLTFKVRRHAPELIAPAKPTPRELKPLSDIDDQEGLRFHIPVIQFYRSDPKMKNKNPASVIREALAKVLVFYYPFAGRLKEGPARKLMVDCSGEGVLFIEAEADVTLKQFGDALQPPFPCLEELLYDVPGSTGVLDTPLLLIQVTRLLCGGFIFALRLNHTMSDAPGLVQFMTGLGEMAQGASRPSTLPVWQRELLLARDPPRVTCTHHEYTEVEDTKGTIIPLDDMAHKSFFFGPSEISALRRFVPSYLKKCSTFEVLTACLWRCRTIALQPDPEEEMRIICIVNARGKFNPPLPKGYYGNGFAFPVAISTAGDLSSKPLGHALELVMKAKSDVTEEYMRSIADLMVIKGRPHFTVVRSYLVSDVTHAGFDVVDFGWGKAAYGGPAKGGVGAIPGVASFYIPFTNHKGESGIVLPICLPSAAMDKFVEELNKMLVPDNNEQVLREHKLLVLARL

SEQ ID NO: 138 - protein, 443 aa, Helicrysum umbraculigerum
MEIQVINYSSKLVKPLTPTPTANRYYNISFTDELVPTIYVPLILYYATPKNPNGDHFENICDRLEESLSKTLSDFYPLAARFIRKLSLIDCNDQGVLFVLGNVNIRLSDVTGLGLTFKTSVLNDFLPCEIGGADEVDDPMLCVKVTTFECGGFAIGMCFSHRLSDMGTMCNFINNWAARTIGEYDNEKHTPIFNSPLYFPQRGLPELDLKVPRSSIGVKNAARMFHFNGKAISSMREVFGVDENGSRRLSKVQLVVALLWKAFVRIDDVNDGQSKASFLIQPVGLRDKVVPPLPSNSFGNFWGLATSQLGPGEGHKIGFQEYFYILRESIKKRARDCAKILTHGEEGYGVVIDPYLESNQKIADNGTNFYLFTCWCKFSFYEADFGCGKPIWASTGKFPVQNLVIMMDDNEGDGVEAWVHLDDKRMNELEQDPDVKLYACNLA

SEQ ID NO: 139 - protein, 434 aa, Helicrysum umbraculigerum
MKLAVKESVIVKPSKTTPCQQIWTSNLDLVVGRIHILTVYLYRPNGSSNFFDSMVLKKALADVLVSFFPVAGRLDKDGDGRVVIDCNGEGVLFVEAEADCCIDDFGEITPSPELRRLVPTVDYSGDMSSYPLFITQVTRFKCGGVSLGCGLHHTLSDGLSALHFINTWSDVARGLSVAIPPFIDRSLLRARDPPSPVFDHIEYHPPPSLITPLQNQKNASHSRSASTLILRLTLHQINNLKSKAKGDGSMYHSTYEILAAHLWRCACKARGLANDQPTKLYVATDGRSRLIPPLPPGYLGNVVFTATPVAKSGDFESESLAETARRIRSELGKMNDEYLRSAIDYLESVSDISTLVRGPTYFASPNLNVNSWTRLPIYESDFGWGRPIFMGPASILYEGTIYIIPSPSGDRSVSLAVCLDPDHMALFKECLYVF

SEQ ID NO: 140 - protein, 434 aa, Helicrysum umbraculigerum
MKLAVKESVIVKPSKTTPCQQIRTSNLDLVAGRIHILVVFFYRPNGSSNFFDSLVLKKALADVLVPFFPVAGRFSEDGDGRVVIDCNGEGVLFVESEADCCIDDFGEITLSPELQQLVPTVDYSGDMSSYPLFIAQVTRFKCGGVSLGWGLHHTLLDGLSALHFVNTWGDVARGLSVAIQPFIDRSLLRARDPPTPVFDHIEYHPPPSLITPLQNQKNASHSRSASTLILQLTPDQIKNLKSKAKGDGSMYHSTYEILAAHLWRCACKARGLANDQPTKLYVAANGRSRLIPPLPPGYLGNVVFNATHVAKSGDFESESLAETARRIHCELGKMNDEYFRSAIDYLESVDDISTLVKGPTYFASPNLNVYSWIGIPIYACDFGWGQPIFMRPASFLYDGSIYIIPSPSGDRSVLLAVCLDPDHMDLFKECLYAF

SEQ ID NO: 141 - protein, 460 aa, Helicrysum umbraculigerum
MVMISKLLRLGRRKLHTIVSRDTIRPSSPTPSHSKTYNLSLLDQIAVNSYVPIVAFYPSSNVCRSSDDKTLELKNSLSKILTHYYPFAGRMKKNRPTVVDCNDEGVEFVEARNTNSLSDFLQQSEHEDLDQLFPDDCVWFKQNLKGSINDANNSSVCPLSIQVNHFACGGVAVATSLRHKIGDGSSALNFIKHWAAVTSHSRAGNHQIDATSPIINPHFISYPTRTFKLPDRSPYIPPSDVVSKSFVFPNTNIKDLQAKVVTMTMGSRQPIVNPTRADVVSWLLHKCVVAAATKRISGNFKESCVISPLNLRNKLEEPLPETSIGNIFYLITFPISNNHGDLMPDDFISQLRLGIRKFQNIRNLETALRTVEEMISETFILGTAESMDTSYVYSSIRGFPMYDIDFGWGKPVKVTVGGALKNLSILMDTPDVNGIEALVSLDKQDMKILLNDPELLAFCL

SEQ ID NO: 142 - protein, 440 aa, Helicrysum umbraculigerum
MSTSDKMKITIRESSMIKPSKPTPDQRIWNSNLDLVVGRIHILTLYFFRPNGSSDFFDSEVLKQSLADVLVSFFPMAGRLGLDGDGRVEINCNGEGVLFVEAEADCSIDDFGEITPSPELRRLAPTVDYSGDISSYPLVITQVTHFKCGGVSLGCGLHHTLSDGLSSLHFINTWSDVTRGLPVAIPPFVDRTVLRARDPPTVVFDHVEYHTPPSMTSSLDKDKPQSEDVHVSTSMLRLTLDQINALKAKGKGDGIVYHSTYEILAAHLWRCACKARGLLNDQMTKLYVATDGRSRLIPPLPPGYLGNVVFTATPIAKSGELQQEPLATTARKIHTELAKMDDKYLRSALDYLESQQDLSALIRGPAYFACPNLNINSWTRLPIYDADFGWGRPIFMGPASILYEGTIYIIPSPSGDRSVSLAVCLDPSHMPLFQKYLYEL

SEQ ID NO: 143 - protein, 441 aa, Helicrysum umbraculigerum
MVNVEIISNEYIKPSSPTPPHLKIYNLSILDQLIPAPYAPIILYYPNQDHINDFEVHERLKLLKDSLSKTLTRFYPLAGTIKGDLSIDCNDIGAYFAVAHVNTRLDVFLNHPDLDLINCFLPRGPYLNGSSEGSCVSNVQVNIFECCGIAISLCISHKILDGAALSTFLKAWAGTSYGSKEVVYPNMSAPSLFPAKDLWLKDSSMVMFGSLFKMGKCSTKRFVFDSSKLSFLKAKASLNGLKDPTRVEVVSALLWKCIMAASEENTGSWKPSLLSHVVNLRKRLVSTLSEDSIGNLIWLASAECRTNAQSRLSDLVEKVRDSVSKINSEFVKKIQGDKGTKVMEESLKSMKDCADYIGFTSWCKMGFYDVDFGWGKPVWVCGSVCEGSPVFMNFVILMDTKYGDGIEAWVSLDEHEMHILKHNPELLEYASIDPSPLQMNK

SEQ ID NO: 144 - protein, 454 aa, Helicrysum umbraculigerum
MGTIYQSPMIKSSTPKIIEDLKVIIHDTFTIFPPHETEKRSMFLSNIDQVLTFNVETVHFFAANPDFPPQVVAEKLKLALSKALVPYDFLAGRLKLNHESQRFEFDCNGAGARFVVGSSEFELGEIGDLVYPNPGFRQLVQKSYDNLELHEKPLCILQLTSFKCGGFALGVATNHATFDGLSFKTFLQNLGSLAADQPLAVDPCNDRHLLAARSPPKVQFDHPELLKIPTGTDIPNPTVFDCPESQLDFKIFNLTSDDIAHLKTKAKDGPGSTNAKITGFNVVAAHVWRCKALSSGSEYDPERVSTVLYAVDIRSRLNLPLSLAGNAVLSAYASAKCKEIEEGPLSRLVEMVTEGTNRMTGEYARSVIDWGEVNKGFPNGEFLISSWWRLGFADVEYPWGKPRYSCPVVYHRKDIILLFPDIVGADNNNEVNVLVALPGKEMEKFETLFHKFLA